SEQUENCE LISTING (4 sequences)

SEQ ID NO: 1 - protein, 21 aa, Synthetic construct
MGSSHHHHHHSSGENLYFQGS

SEQ ID NO: 2 - protein, 43 aa, Aspergillus fumigatus
MSGSHHHHHHHHGGENLYFQGSGLNDIFEAQKIEWHEGSSGSS

SEQ ID NO: 3 - protein, 22 aa, Aspergillus fumigatus
MSGSHHHHHHHHGGENLYFQGS

SEQ ID NO: 4 - protein, 17 aa, Synthetic construct
GGGSSGGSTSGGSSGGG

(19) United States
(12) Patent Application Publication
(10) Pub. No.: US 2026/0315446 A1
(43) Pub. Date: Oct. 15, 2026

(54) ANTIFUNGAL COMPOUNDS AND USES THEREOF

(71) Applicants: Praveen R JUVVADI, Durham, NC (US); William STEINBACH, Durham, NC (US); Maria CARDENAS CORONA, Durham, NC (US); Benjamin BOBAY, Durham, NC (US); Ronald VENTERS, Durham, NC (US); Patrick DOME, Durham, NC (US); Angela RIVERA, Durham, NC (US); Pyeonghwa JEONG, Durham, NC (US); Hongjun JANG, Durham, NC (US); DUKE UNIVERSITY, Durham, NC (US)

(72) Inventors: Praveen R. JUVVADI, Durham, NC (US); William STEINBACH, Durham, NC (US); Maria CARDENAS, Durham, NC (US); Benjamin BOBAY, Durham, NC (US); Ronald VENTERS, Durham, NC (US); Patrick DOME, Durham, NC (US); Angela RIVERA, Durham, NC (US); Pyeonghwa JEONG, Durham, NC (US); Hongjun JANG, Durham, NC (US); Joseph HEITMAN, Durham, NC (US); Michael J. HOY, Durham, NC (US); Jiyong HONG, Durham, NC (US); Won Young LIM, Durham, NC (US)

(21) Appl. No.: 19/482,364

(22) PCT Filed: May 8, 2024
(86) PCT No.: PCT/US2024/028390

§ 371 Date: Nov. 7, 2025

Related U.S. Application Data
(60) Provisional application No. 63/464,699, filed on May 8, 2023.

(51) Int. Cl. A61K 31/439; A61K 31/137; A61K 31/192; A61K 31/343; A61K 31/4174; A61K 31/4196; A61K 31/4418; A61K 31/496; A61K 31/506; A61K 31/7048; A61K 38/12; A61P 31/10; C07D 498/18

(52) U.S. Cl. CPC A61K 31/439; A61K 31/137; A61K 31/192; A61K 31/343; A61K 31/4174; A61K 31/4196; A61K 31/4418; A61K 31/496; A61K 31/506; A61K 31/7048; A61K 38/12; A61P 31/10; C07D 498/18

(57) ABSTRACT

This invention is in the field of medicinal chemistry. In particular, the present disclosure provides methods for treating or preventing a fungal infection with one or more macrolide compounds, or pharmaceutically acceptable salts thereof, or compositions comprising the same and an additional anti-fungal agent, and pharmaceutical compositions comprising one or more macrolide compounds and at least one additional anti-fungal agent.

### CROSS-REFERENCE TO RELATED APPLICATIONS

The present application claims priority to U.S. Provisional Application No. 63/464,699, filed May 8, 2023, which is incorporated herein by reference in its entirety.

### SEQUENCE LISTING

The text of the computer readable sequence listing filed herewith, titled “DUKE_43040_601_SequenceListing.xml” created May 8, 2024, having a file size of 4,557 bytes, is hereby incorporated by reference in its entirety.

### FIELD OF THE INVENTION

This invention is in the field of medicinal chemistry. In particular, the present disclosure provides methods for treating or preventing a fungal infection with one or more macrolide compounds, or pharmaceutically acceptable salts thereof, or compositions comprising the same and an additional anti-fungal agent, and pharmaceutical compositions comprising one or more macrolide compounds and at least one additional anti-fungal agent.

### INTRODUCTION

Fungal infections, which result in more than 1.5 million deaths per year, are recognized as a serious global health threat (see, Bongomin F, Gago S, Oladele R O, Denning D W. 2017. J Fungi (Basel) 3:57). The World Health Organization recently released a prioritized list of fungal pathogens for global research (see, World Health Organization. 2022. WHO fungal priority pathogens list to guide research, development and public health action. World Health Organization, Geneva). The critical priority group consists of four life-threatening fungal pathogens: Cryptococcus neoformans, Candida albicans, Candida auris, and Aspergillus fumigatus (see, World Health Organization. 2022. WHO fungal priority pathogens list to guide research, development and public health action. World Health Organization, Geneva). Underscoring the global severity of C. neoformans infection, cryptococcal meningoencephalitis, is responsible for ~20% of all AIDS-related deaths and is among the deadliest fungal infections of global impact (see, Rajasinghan R, et al., 2022. Lancet Infect Dis 22:1748-1755). These infections have a further devastating impact on sub-Saharan African populations (see, Bongomin F, Gago S, Oladele R O, Denning D W. 2017. J Fungi (Basel) 3:57; Rajasingham R, et al, 2022. Lancet Infect Dis 22:1748-1755: Park B J, et al., 2009. AIDS 23:525-530). In high-income countries, the current treatment for cryptococcal infection consists of combination therapy with amphotericin B and flucytosine; however, those most impacted by this health burden have fewer effective treatment options (see, Perfect J R., et al., 2010. Clin Infect Dis 50:291-322). Flucytosine is typically not available in these regions, and often fluconazole alone is prescribed due to its oral availability. However, fluconazole's antifungal activity against C. neoformans is only fungistatic, and resistance frequently develops in the treated patient as a result of aneuploidy (see, Sionov E, Lee H, Chang Y C, Kwon-Chung K J. 2010. PLoS Pathog 6:e1000848). Resistance to fluconazole is rapidly emerging in multiple other fungal species, including Candida auris and Aspergillus fumigatus, in part due to the environmental use of azoles in agriculture, further limiting this therapeutic option and driving the need for newer antifungal drugs (see, Rocha M F G, et at, 2016. Mycoses 59:281-290; Berkow E L, Lockhart S R. 2017. Infec Drug Resist 10:237-245; Arendrup M C. 2014. Clin Microbiol hnfect 20 Suppi 6:42-48).

As such, there is an urgent need to develop new antifungal strategies and identify novel targets to address the increasing global threat of deadly invasive fungal diseases.

The present invention addresses this need.

### SUMMARY OF THE INVENTION

One promising antifungal drug target under investigation is the protein phosphatase calcineurin. Calcineurin is highly conserved across pathogenic organisms, including fungi and parasites (see, Naderer T, Dandash O, McConville M J. 2011. Mol Microbiol 80:471-480; Matsumoto Y, et al., 2022. Sci Rep 12:16126; Kurakado S, et al., 2023. Microbiol Immunol 67:49-57). Calcineurin mediates stress responses in C. neoformans, C. albicans, and A. fumigatus (see, Chow E W L, et al., 2017. PLoS Genet 13:e1006667; Lev S, et al., 2012. PLoS One 7:e51403; Cruz M C, et al., 2002. EMBO J 21:546-559; Shwab E K, et al., 2019. Mol Microbiol 112:62-80). Calcineurin inhibition by FK506 prevents C. neoformans growth at high temperatures (see, Arndt C, Cruz M C, Cardenas M E, Heitman J. 1999. Microbiology 145:1989-2000; Odom A, Del Poeta M, Perfect J, Heitman J. 1997. Antimicrob Agents Chemother 41:156-161). In fact, calcineurin is essential for virulence in C. neoformans and also in several other pathogenic fungal species, including Candida and Aspergillus, and therefore is an attractive antifungal drug target (see, Cruz M C, et al., 2002. EMBO J 21:546-559: Breuder T, Hemenway C S, Movva N R, Cardenas M E, Heitman. 1994. Proc Natl Acad Sci USA 91:5372-5376; Odom A, et al., 1997. EMRO J 16:2576-2589). The high degree of conservation of calcineurin across human fungal pathogens supports the approach of targeting calcineurin to develop a pan-antifungal therapeutic.

However, calcineurin is also conserved in humans (see, Klee C B, Crouch T H, Krinks M H. 1979. Proc Nat Acad Sci USA 76:6270-6273). Inhibiting calcineurin in humans results in immunosuppression. Specifically, the natural calcineurin inhibitor FK506 (also known as tacrolimus) induces immune suppression by inhibiting T cell activation and interleukin-2 (IL-2)-dependent responses mediated by the nuclear factor of activated T cells (NFAT) transcription factor (see, Kino T, et al., 1987. J. Antibiot 40:1249-1255; Tanaka H, et al. 1987. J Am Chem Soc 109:5031-5033; Dumont F J, et al., 1990. J Immunol 144:251-258; O'Keefe S J, et al, 1992. Nature 357:692-694; Clipstone N A, Crabtree G R. 1992. Nature 357:695-697; Bierer B E, et al., 1990. Proc Nat Acad Sci USA 87:9231-9235; Gwack Y, et al, 2007. Cell Calcium 42:145-156). Therefore, experiments described herein monitored IL-2 expression as a measure of mammalian calcineurin inhibition. The FK506 ligand first binds to the FK506-binding protein (FKBP12), and the FKBP12-FK506 complex then binds to and inhibits calcineurin (see, Liu J, et al., 1992. Biochemistry 31:3896-3901; Foor F, et al., 1992. Nature 360:682-684; Liu J, et al., 1991, Cell 66:807-815). FK506 is a Food and Drug Administration (FDA)-approved therapeutic initially deployed to prevent graft rejection following organ transplants (see, Starzl T E, et al., 1989 Lancet 2: 1000-1004; Ochiai T, et al., 1987. Transplantation 44:734-738). FK506 has also been researched for use in additional applications, including the treatment of hepatic fibrosis and glioblastoma, as well as aging and bone degeneration in Alzheimer's disease (see, Manojlovic Z, Blackmon J, Stefanovic B. 2013. PLoS One 8:e65897; Ge J, et al., 2020. Front Cell Der Biol 8:608308; Torres Á, et al., 2018. Int J Mol Sci 19:2697; Gant J C, et al., 2014 Eur J Pharmacol 739:74-82). Currently, FK506 has received additional FDA approval for use in atopic dermatology applications (see, Alaiti S, et al., 1998. J Am Acad Dermatol 38:69-76).

Humans and fungi share a high degree of conservation of cellular machinery, making it difficult to identify fungal-specific drug targets. As such, immunosuppression by FK506 precludes its use as an antifungal therapeutic, as immune suppression is a factor that increases the risk of fungal infection (see, Stein D K, Sugar A M. 1989. Diagn Microbiol Infect Dis 12:221S-228S). A goal of the experiments described herein was to modify the structure of the calcineurin inhibitor FK520 (or ascomycin, a natural product analog of FK506) to preserve antifungal activity and reduce or eliminate immunosuppressive activity. Both FK506 and FK520 inhibit C. neoformans growth under stress conditions, and the synthesis of less or non-immunosuppressive analogs that retain antifungal activity is a validated antifungal drug development approach (see, Odom A, Del Poeta M, Perfect J, Heitman J. 1997. Antimicrob Agents Chemother 41:156-161; Beonm J Y, et al., 2019. J Nat Prod 82:2078-2086; Lee Y, et al., 2018. Antimicrob Agents Chemother 62:e01627-18; Rotonda J, et al., 1993. J Biol Chem 268:7607-7609). High-resolution X-ray crystal structures of the calcineurin-FK506-FKBP12 complexes were previously solved from fungal pathogens, including A. fumigatus, C. albicans, and C. neoformans. Using structure-guided drug design, the carbon at position 22 (C-22) on FK506 was implicated as a critical point for chemical modification to achieve differential ligand targeting of human and fungal calcineurin (see, Juvvadi P R, et al., 2019. Not Commun 10:4275). Thus far, it has been demonstrated how modifications at this site on both FK506 and its natural analog FK520, differing by a single allyl to ethyl group substitution at the C-21 position (see, Aria T, Kouam a Y, Suenaga T, Honda H. 1962. J Antibiot 15:231-232), resulted in decreased immune suppression while maintaining sufficient antifungal activity to be of benefit in animal infection models (see, Hoy M J, et al., 2022. nBio 13:e0104922). Developing FK506 analogs as an antifungal therapeutic approach is further bolstered by the research describing the synergistic activity of calcineurin inhibitors with additional antifungal agents currently being investigated in clinical trials (see, Liston S D, et al., 2022. J Fungi 8:1102).

Indeed, experiments conducted during the course of developing embodiments for the present invention developed an expanded library of FK506/FK520 analogs modified at one or more of the C-18 position, the C-21 position, and the C-22 position. These efforts have resulted in the generation of compound JH-FK-08 as a novel antifungal therapeutic effective at significantly reducing lung fungal burden in a murine cryptococcosis model compared to untreated infected animals. Furthermore, treatment with JH-FK-08 significantly prolonged murine survival and surpassed the antifungal efficacy achieved in previous efforts. These advances identify an FK520 analog with the potential to be utilized to treat invasive fungal infections.

Accordingly, the present disclosure provides methods for treating or preventing a fungal infection with one or more macrolide compounds, or pharmaceutically acceptable salts thereof, or compositions comprising the same and an additional anti-fungal agent, and pharmaceutical compositions comprising one or more macrolide compounds and at least one additional anti-fungal agent.

Certain macrolide (and structurally related) small-molecule compounds as described herein may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art. Indeed, the term “compound” or “small-molecule compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described.

In certain aspects, the present disclosure provides compounds encompassed within one of the following formulas:

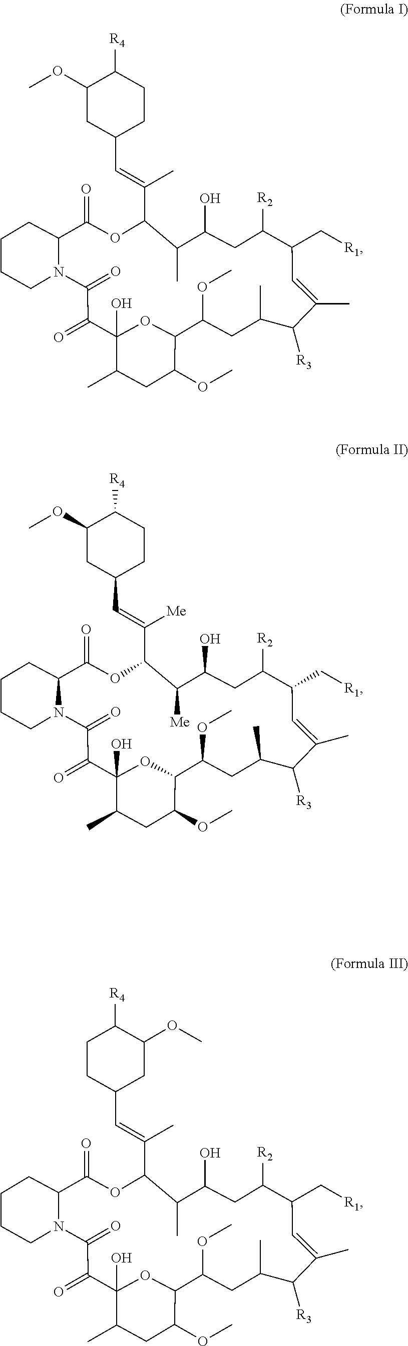

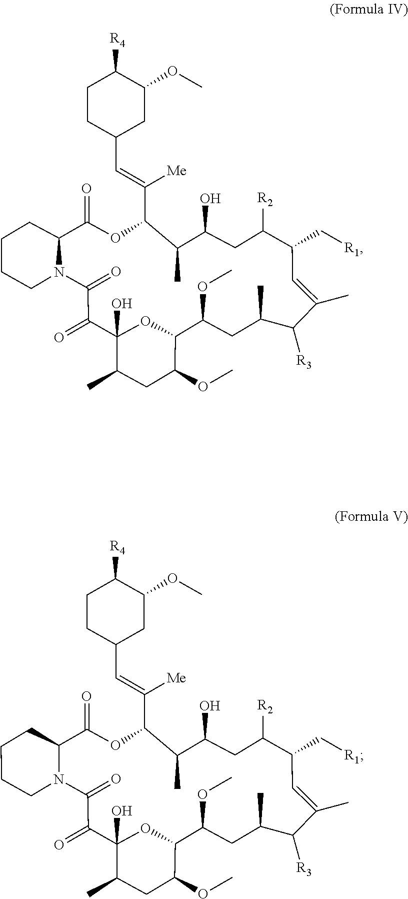

including pharmaceutically acceptable salts, hydrates, solvates, polymorphs, prodrugs, triphenylphosphonium (TPP) tagged derivatives, proteolysis targeting chimera (PROTAC) derivatives, mitochondria tagged derivatives, biotin tagged derivatives, boron-dipyrromethene (BODIPY) tagged derivatives, stereoisomers, and/or deuterated analogues thereof; wherein each of R₁, R₂, R₃, and R₄ independently include any chemical moiety that renders the resulting compound capable of inhibiting fungal activity.

In some aspects, each of R₁, R₂, R₃, and R₄ independently include any chemical moiety that renders the resulting compound capable of one or more of the following: inhibiting calcineurin;
inhibiting fungal activity associated with calcineurin;
preventing fungal growth;
inhibiting calcineurin activity;
associating with (e.g., binding with) FKBP12;
associating with (e.g., binding with) FKBP12 to form a complex that is capable of inhibiting calcineurin activity;
associating with (e.g., binding with) FKBP12 to form a complex that is capable of inhibiting fungal activity;
associating with (e.g., binding with) FKBP12 to form a complex that is capable of inhibiting Cryptococcus neoformans activity.

In some aspects, R1 is selected from hydrogen,

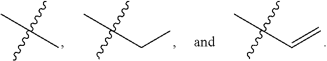

In some aspects, R2 is selected from hydrogen,

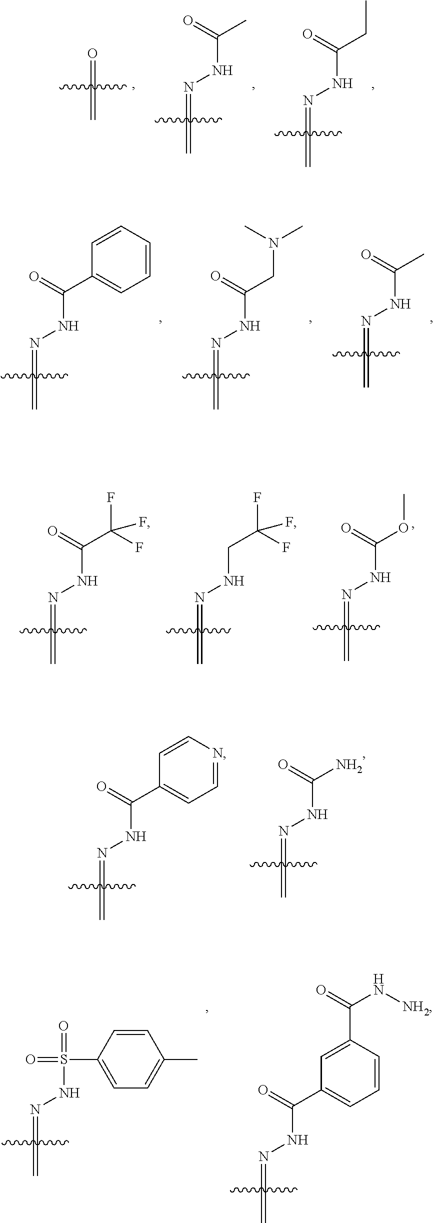

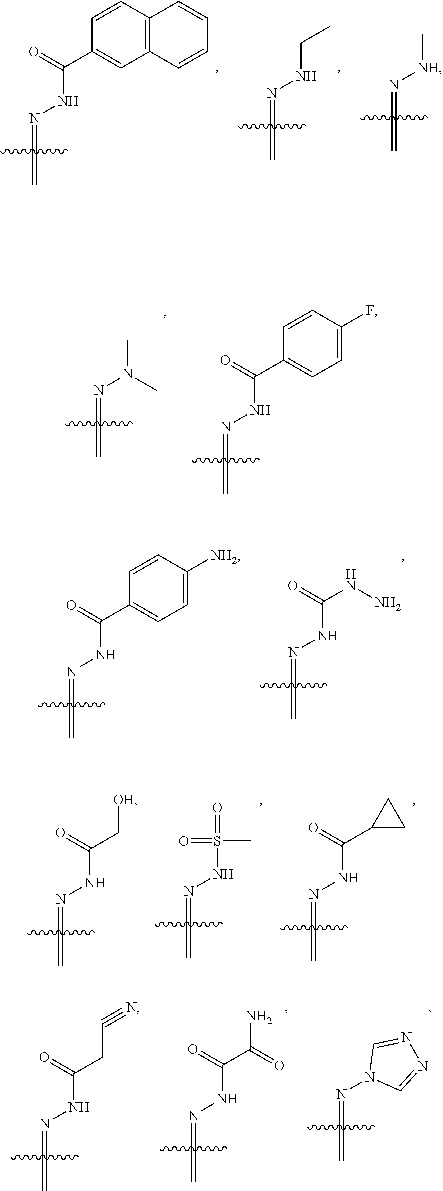

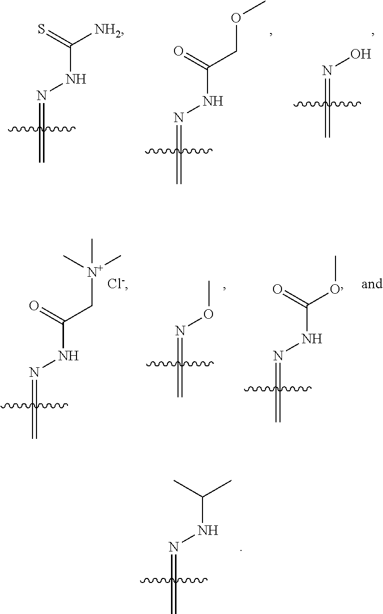

In some aspects, R3 is selected from hydrogen, and hydroxyl (e.g., OH).

In some aspects, R4 is selected from hydrogen, hydroxyl (e.g., OH),

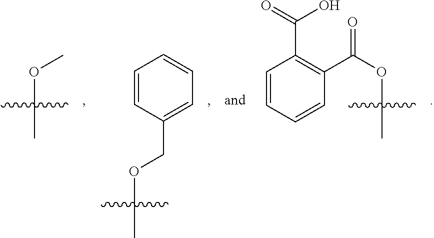

In some aspects, the compound is selected from:

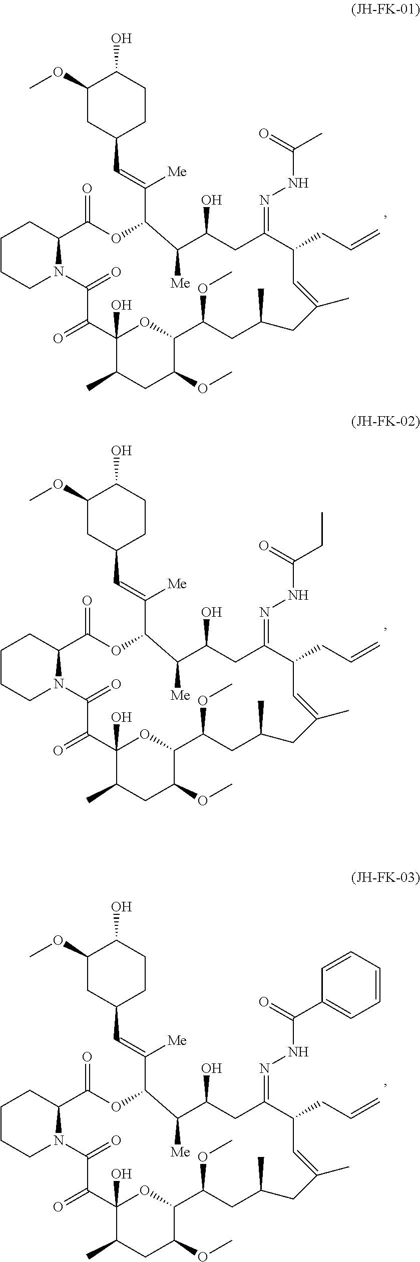

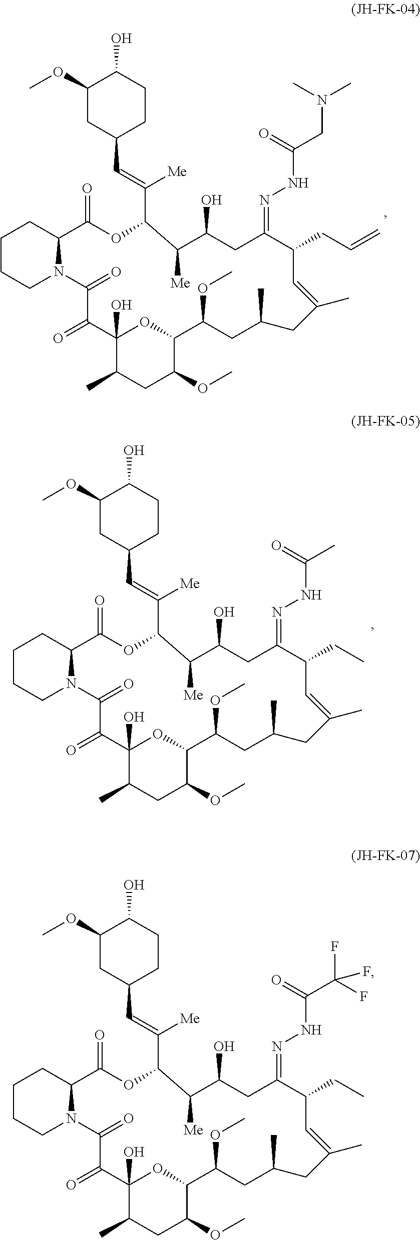

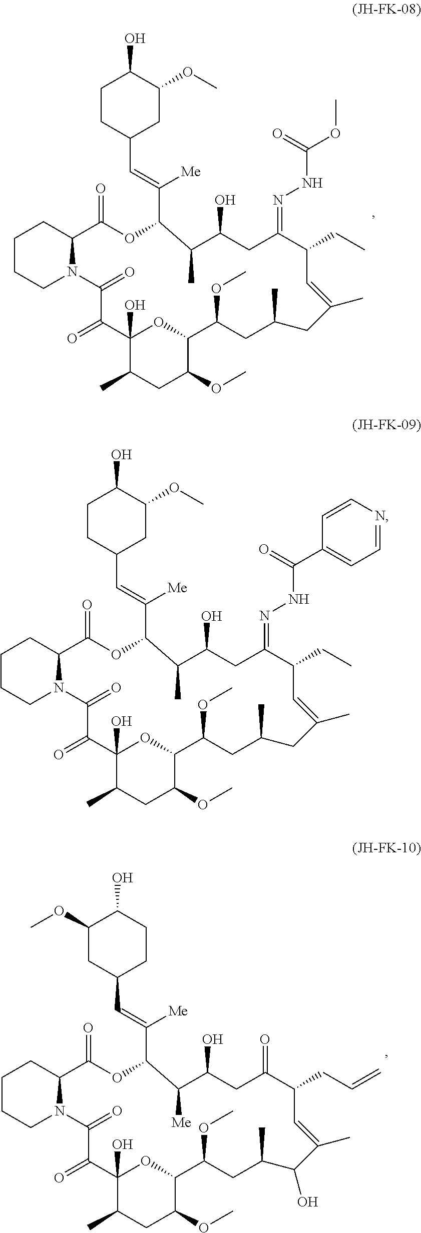

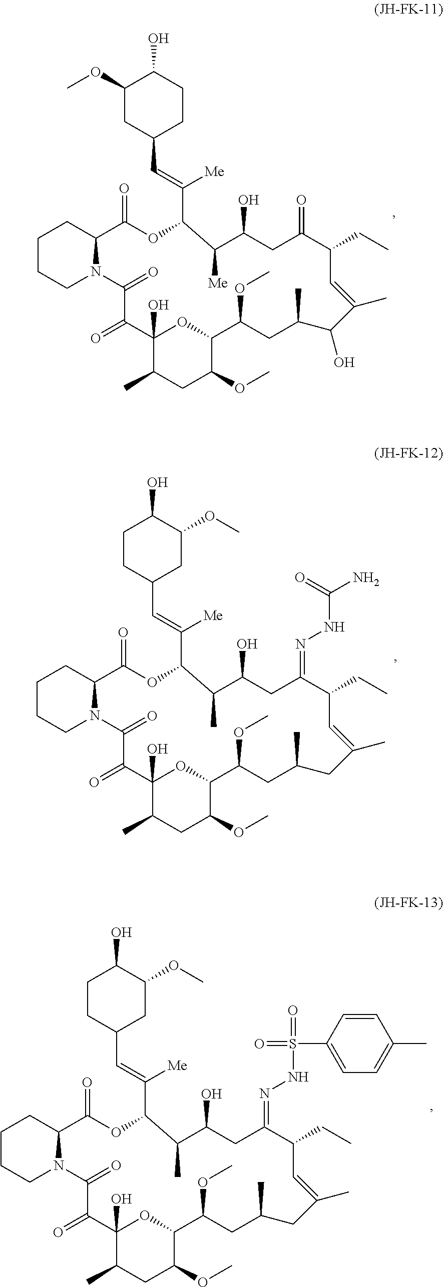

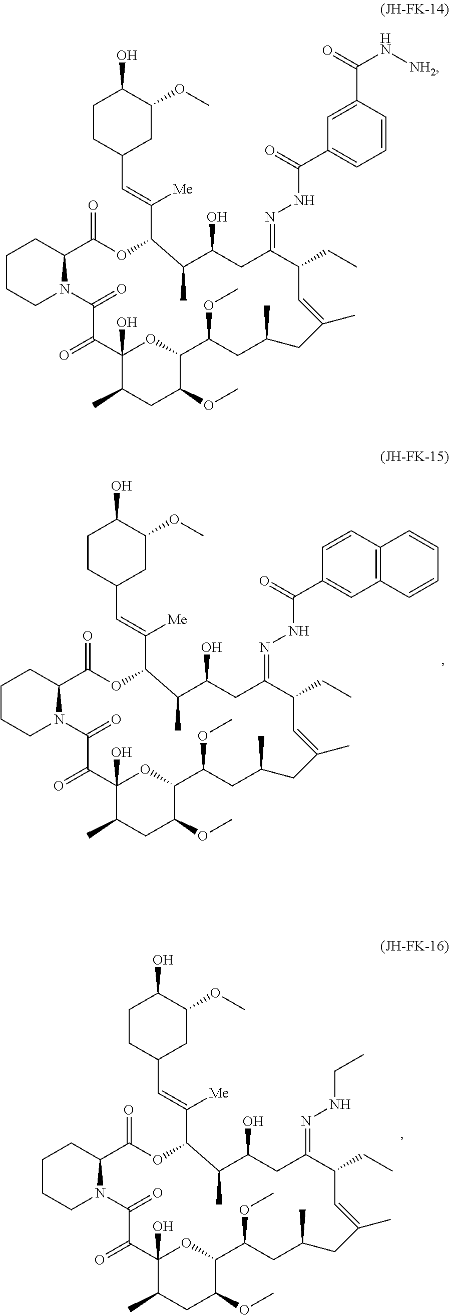

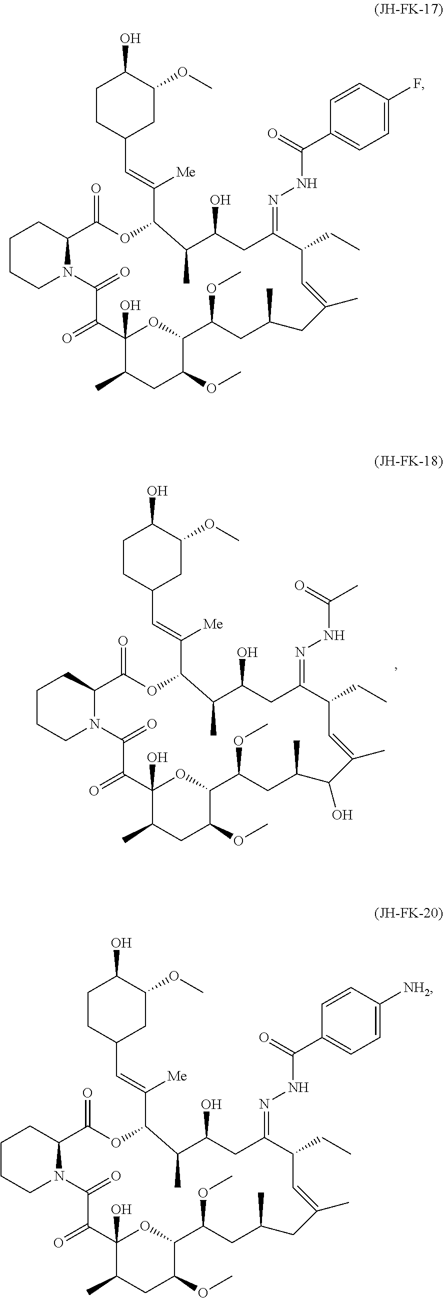

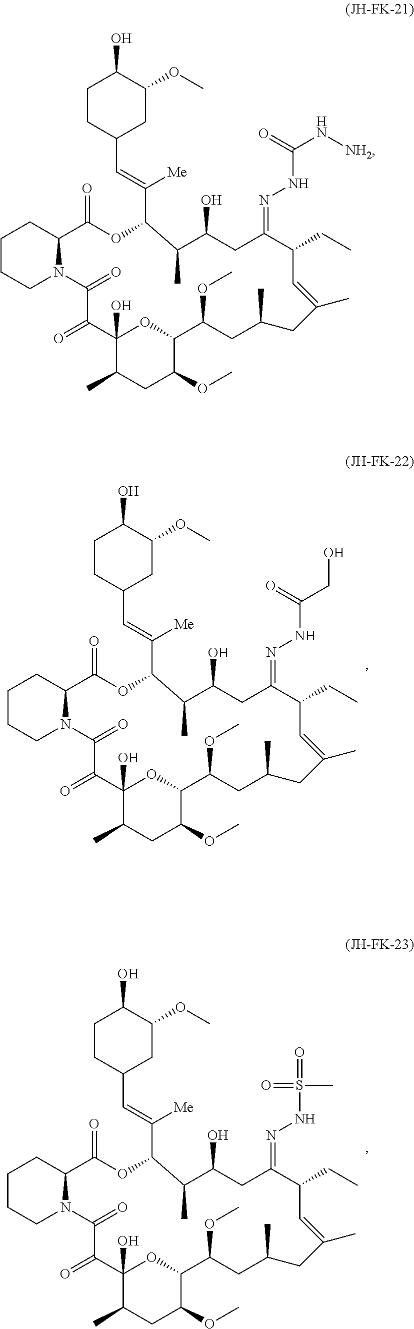

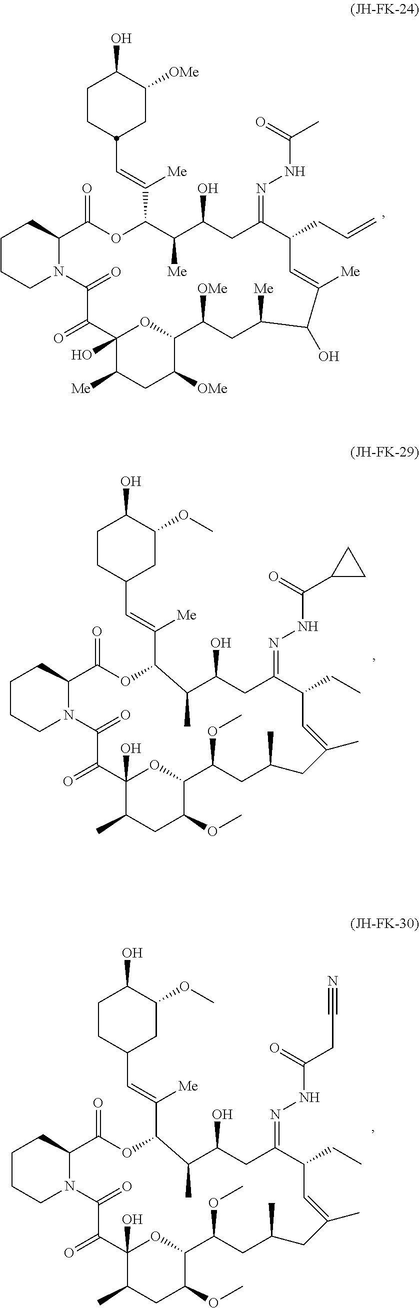

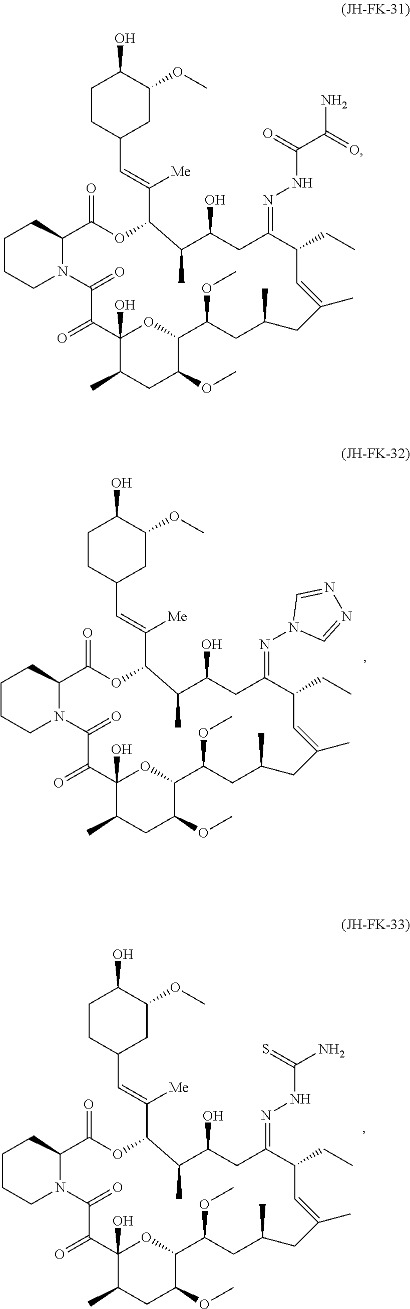

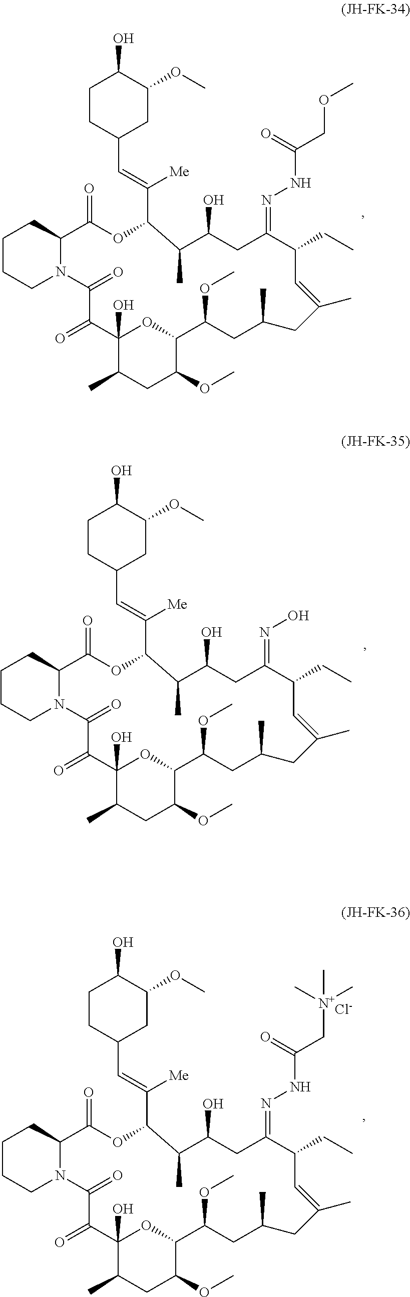

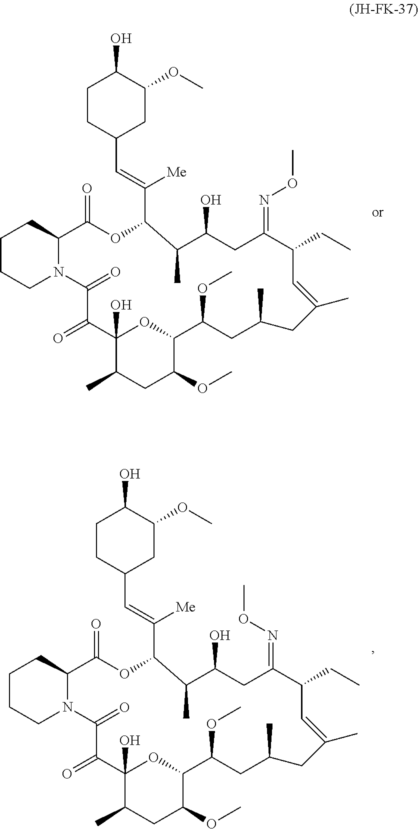 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/OC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N\OC)[C@H](C)C[C@@H]2OC]

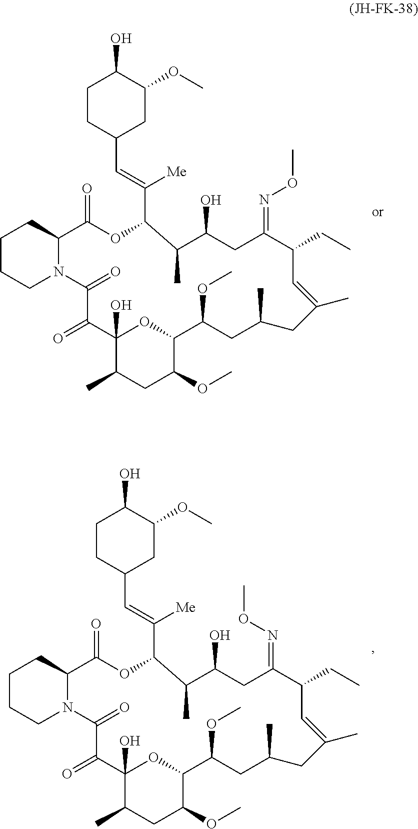 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/OC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N\OC)[C@H](C)C[C@@H]2OC]

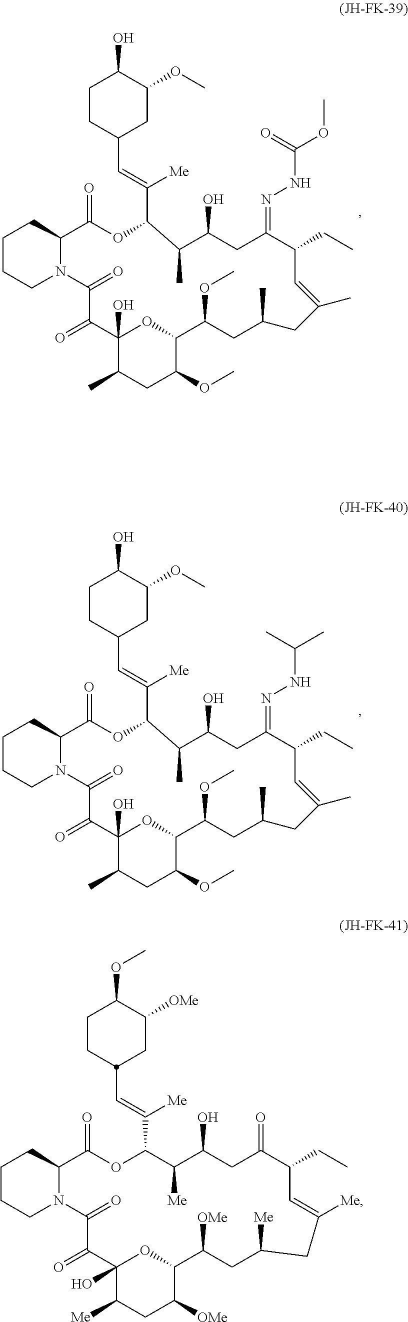

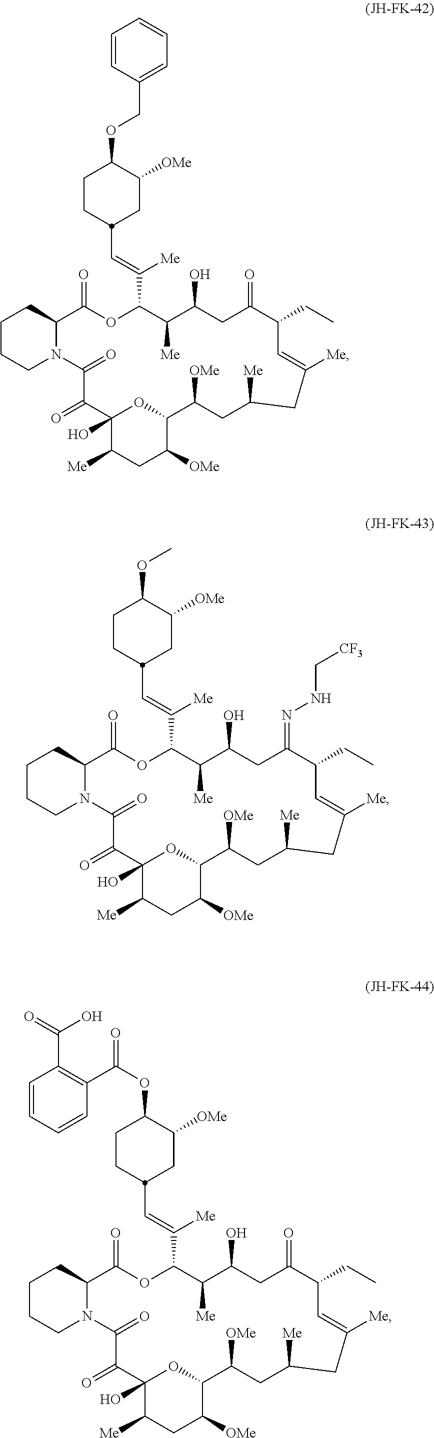

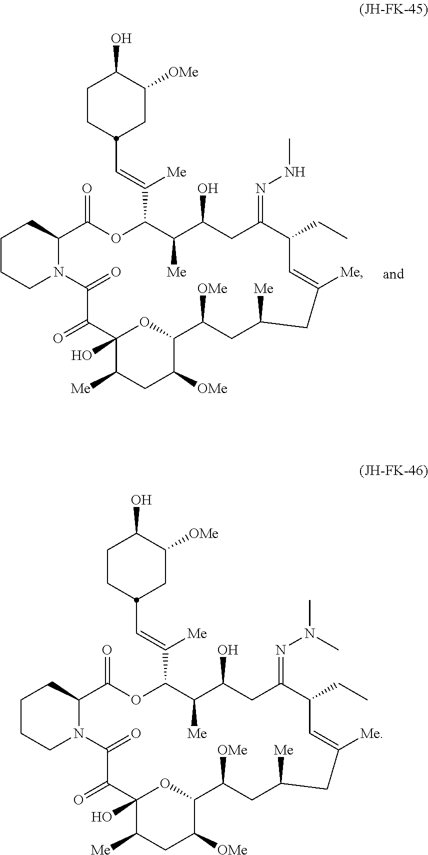 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/NC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/N(C)C)[C@H](C)C[C@@H]2OC]

In certain aspects, the present disclosure provides pharmaceutical compositions comprising an effective amount of one or more of the compounds encompassed within Formulas I, II, III, IV, and/or V, or a pharmaceutically acceptable salt thereof, and a pharmaceutically acceptable carrier.

In some aspects, the composition further comprises at least one additional therapeutic agent. In some aspects, the at least one additional therapeutic agent comprises any type or kind of therapeutic agent capable of inhibiting fungal activity. In some aspects, the at least one additional therapeutic agent is selected from the following: a polyene, imidazole, triazole, thiazole, allylamine, echinocandin, among others. Examples include Amphotericin B, Candicidin, Filipin, Hamycin, Natamycin, Nystatin, Rimocidin, Bifonazole, Butoconazole, Clotrimazole, econazole, fenticonazole, isoconazole, kentoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, polygodial, tolnaftate, undecylenic acid, and crystal violet.

In certain aspects, the present disclosure provides methods of treating or preventing a fungal infection in a mammal comprising administering to the mammal in need thereof a therapeutically effective amount of one or more of the compounds encompassed within Formulas I, II, III, IV, and/or V. In some embodiments, the one or more compounds are encompassed within a pharmaceutical composition. In some aspects, the mammal is a human being. In some aspects, the mammal is a human being suffering from or at risk of suffering from a fungal infection.

In some aspects, the fungal infection is an Aspergillus spp., Fusarium spp., Malassezia spp., Candida spp., or Cryptococcus spp. infection.

In some aspects, the fungal infection is an Aspergillus spp. infection. In some aspects, the Aspergillus spp. infection is selected from an Aspergillus fumigatus infection, an Aspergillus flavus infection, an Aspergillus niger infection, an Aspergillus terreus infection.

In some aspects, the fungal infection is a Fusarium spp. infection. In some aspects, the Fusarium spp. infection is selected from a Fusarium solani infection, a Fusarium moniliforme infection, and a Fusarium proliferatum infection.

In some aspects, the fungal infection is an Malassezia spp. infection. In some aspects, the Malassezia spp. infection is a Malassezia pachydermatis infection.

In some aspects, the fungal infection is a Candida spp. infection. In some aspects, the Candida spp. infection is selected from a Candida albicans infection, a Candida glabrata infection, a Candida tropicalis infection, a Candida krusei infection, and a Candida auris infection.

In some aspects, the fungal infection is a Cryptococcus spp. infection. In some aspects, the Cryptococcus spp. infection is a Cryptococcus neofornans infection.

In some aspects, the fungal infection is a Chrysosporium parvum, Metarhizium anisopliae, Phaeoisaria clematidis, or Sarcopodium oculorum infection.

In some aspects, the fungal infection is a Mucorales infection. In some aspects, the Mucorales infection is a Mucor spp., Rhizopus spp., Lichtheimia spp., or Rhizomucor spp. infection. In some aspects, the Mucor spp. infection is a M. circinelloides infection; wherein the Rhizopus spp. infection is a Rhizopus delemar infection or a Rhizopus oryzae infection; wherein the Lichtheimia spp. infection is a Lichtheimia corymbifera infection.

In some aspects, the methods further comprise administering to the mammal in need thereof at least one additional therapeutic agent capable of inhibiting fungal activity. In some aspects, the at least one additional therapeutic agent is selected from the following: a polyene, imidazole, triazole, thiazole, allylamine, echinocandin, among others. Examples include Amphotericin B, Candicidin, Filipin, Hamycin, Natamycin, Nystatin, Rimocidin, Bifonazole, Butoconazole, Clotrimazole, econazole, fenticonazole, isoconazole, kentoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, polygodial, tolnaftate, undecylenic acid, and crystal violet.

In certain aspects, the present disclosure provides methods of killing or inhibiting the growth of a fungus comprising contacting the fungus with a therapeutically effective amount of one or more of the compounds encompassed within Formulas I, II, III, IV, and/or V. In some embodiments, the one or more compounds are encompassed within a pharmaceutical composition.

In some aspects, the fungus is selected from Aspergillus spp., Fusarium spp., Malassezia spp., Candida spp., or Cryptococcus spp. In some aspects, the fungus is selected from Aspergillus fumigatus, Aspergillus flavus, Aspergillus niger, Aspergillus terreus, Fusarium solani, Fusarium moniliforme, Fusarium proliferatum, Malassezia pachydermatis, Candida albicans, Candida glabrata infection, Candida tropicalis, Candida krusei, Candida auris, Cryptococcus neoformans, Chrysosporium parvum, Metarhizium anisopliae, Phaeoisaria clematidis, Sarcopodium oculorum, M. circinelloides, Rhizopus delemar, Rhizopus oryzae, and Lichtheimia corymbifera.

In certain aspects, the present disclosure provides uses of a compound encompassed within Formulas I, II, III, IV, and/or V in the manufacture of a medicament for the treatment or prevention of a fungal infection.

In some aspects, the fungal infection is related to one or more of: Aspergillus spp., Fusarium spp., Malassezia spp., Candida spp., and Cryptococcus spp.

In some aspects, the fungal infection is related to one or more of: Aspergillus fumigatus, Aspergillus flavus, Aspergillus niger, Aspergillus terreus, Fusarium solani, Fusarium moniliforme, Fusarium proliferatum, Malassezia pachydermatis, Candida albicans, Candida glabrata infection, Candida tropicalis, Candida krusei, Candida auris, Cryptococcus neoformans, Chrysosporium parvum, Metarhizium anisopliae, Phaeoisaria clematidis, Sarcopodium oculorum, M. circinelloides, Rhizopus delemar, Rhizopus oryzae, and Lichtheimia corymbifera.

In certain aspects, the present disclosure provides kits comprising one or more compounds encompassed within Formulas I, II, III, IV, and/or V, and one or more additional kit components. In some embodiments, the one or more additional kit components. In some aspects, the one or more additional kit components include agents capable of inhibiting fungal activity. In some aspects, the one or more additional kit components are selected from the following: a polyene, imidazole, triazole, thiazole, allylamine, echinocandin, among others. Examples include Amphotericin B, Candicidin, Filipin, Hamycin, Natamycin, Nystatin, Rimocidin, Bifonazole, Butoconazole, Clotrimazole, econazole, fenticonazole, isoconazole, kentoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, polygodial, tolnaftate, undecylenic acid, and crystal violet. In some aspects, the one or more additional kit components include instructions for using the components of the kit.

### BRIEF DESCRIPTION OF THE DRAWINGS

FIG. 1: Crystal structure of Aspergillus fumigatus FKBP12 bound to FK520. (A) Chemical structures of FK506 (tacrolimus) and FK520 (ascomycin). Structural differences at the C-21 residue are highlighted in red. (B) Protein X-ray crystal structure of A. fumigatus FKBP12 (purple) bound to FK520 (gray) characterized at a resolution of 1.7 Å. (C) Higher-resolution focus of FK520 bound to the FKBP12 ligand-binding pocket. Both C-22 and C-21 of FK520 approach key fungus-specific residue F88 in the 80s loop of FKBP12.

FIG. 2: Mini-calcineurin crystal structures of human and Aspergillus fumigatus protein bound to FK520 and FK506. Protein crystal structures for each organism presented with overlay of bovine full-length calcineurin ternary complex structure CN-FKBP12-FK506 in gray. (A) Structure of human mini-calcineurin complex consisting of CnB (red) and CnA B binding helix (BBH; pink), FKBP12 (blue), and FK520 (green). (B) Structure of A. fumigatus mini-calcineurin complex consisting of CnB (red) and CnA B binding helix (BBH; pink), FKBP12 (blue), and FK506 (green). (C) Protein expression construct of full-length calcineurin subunits A and B and mini-calcineurin containing CnB and CnA BBH linked by 17 amino acids.

FIG. 3: Chemical synthesis of novel FK506 and FK520 analogs. (A) One-step synthesis for C-22-modified analogs from FK506 or FK520 as the starting material. (B) Panel of six FK506/FK520 analogs with acylhydrazone groups at C-22. Acylhydrazones differ in the R² group. Compounds JH-FK-01, JH-FK-02, JH-FK-03, and JH-FK-04 are derivatives of FK506. Compounds JH-FK-05 and JH-FK-07 are derivatives of FK520.

FIG. 4: ¹H nuclear magnetic resonance (NMR) spectra of FK506/FK520 analogs. All six synthesized FK506/FK520 analogs were subjected to ¹H NMR analysis. All integrations are indicated. Chemical structure for each compound analyzed is presented at the left.

FIG. 5: BLI sensorgrams for compound-mediated binding interactions between A. fumigatus and Candida albicans CnA-CnB-calmodulin ternary complex and biotinylated (Avi-tagged)-FKBP12. Experiments were performed using a constant compound concentration of 10 μM and various concentrations of analyte protein (CnA-CnB-calmodulin, 2,500 to 39.0625 nM by serial 1:1 dilution, plus a 0 nM sample blank). All data are double referenced using sample blanks and parallel reference sensors. Three sample replicates were acquired in each experiment (light gray). Analysis was performed using 1:1 models and global analysis (calculated model traces in red). Biolayer inferometry (BLI) kinetics and steady-state analysis results are summarized in the table (bottom). Kinetics data were analyzed using 1:1 models and global analysis. Steady-state data were analyzed using binding responses in the 290 to 295 s range of sample association.

FIG. 6: JH-FK-05 shows reduced immunosuppressive activity in vitro and in vivo and demonstrates fungal specificity. (A) In vitro immunosuppressive activity of calcineurin inhibitors was measured in a primary murine T-cell model. Dose response curves were generated from IL-2 expression in cells exposed to increasing concentrations of inhibitors. All analogs were reduced for immunosuppression compared to controls FK506 (black) and FK520 (pink). Error bars indicate standard error of the mean (SEM). (B) Therapeutic index scores for FK506/FK520 analogs were calculated by comparing the ratio of antifungal (MIC for C. neoformans) to immunosuppressive activity (IL-2 IC₅₀) to the same ratio for FK506. FK506 therapeutic score was set to 1.0, and increasing scores indicate increasing fungal specificity. (C) In vivo immunosuppression of FK506 and JH-FK-05 was measured in a murine model of a T cell-dependent response. Animals were treated daily via i.p. injection with vehicle, FK506 (5 mg/kg), or JH-FK-05 (60 mg/kg) beginning on day ~1. NP-OVA was administered subcutaneously on day 0 to promote T cell-dependent proliferation of T follicular helper cells (Tfh) and germinal center B (GC B) cells. The proportions of proliferated Tfh cells and GC B cells were measured in animal lymph nodes on day 7. Tfh and GC B cells were measured from TCRβ⁺ CD4⁺ CD44^{hi} cells and TCRβ⁻ CD19⁺ cells, respectively. (D) Representative flow cytometry plots are presented from individual animals in each treatment group. Gating indicates a region containing Tfh cells (left, oval) or GC B cells (right, square). Error bars indicate standard deviation. For all plots, **, P<0.01; *, P<0.05.

FIG. 7: JH-FK-05 treatment reduces organ fungal burden in multiple models of C. neoformans infection. (A) Experimental timelines for intranasal and intravenous models of C. neoformans infection. For both models, treatment groups included vehicle (gray), JH-FK-05 60 mg/kg (blue), fluconazole 12 mg/kg (red), and combination (purple). Treatment began on day 0 via i.p. injection and continued daily until the indicated endpoint. Each treatment group contained 5 female A/J mice. (B) Lung tissue fungal burdens from animals in the intranasal model. (C) Brain tissue fungal burdens from animals in the intravenous model. For all plots, ****, P<0.0001; ***, P<0.001; **, P<0.01; *, P<0.05. Error bars indicate standard deviation.

FIG. 8: JH-FK-05 treatment extends animal survival in murine model of cryptococcosis. (A) Survival plot of animals infected with C. neoformans H99 through intranasal instillation. Treatment groups included vehicle (black), JH-FK-05 60 mg/kg (blue), fluconazole 12 mg/kg (red), and combination (purple). Animals received treatment via i.p. injection daily for 14 days, and animals were monitored for health following treatment termination. Each treatment group consisted of 10 A/J female mice. (B) Survival curve pairwise comparisons by log-rank (Mantel-Cox) test.

FIG. 9: Ligand atoms and protein residue contacts observed in MD simulations. Analysis of the significance of the observed contacts of human FKBP12 (hFKBP12) and C. neoformans FKBP12 (cnFKBP12) with JH-FK-05. (A) Stick representation of JH-FK-05 labeled for atoms making more significant contacts to hFKBP12 (magenta circles) and C. neoformans (cyan circles). (B) Residues of hFKBP12 (magenta) and C. neoformans (cyan) making more significant interactions with the ligand are shown in stick format on the ribbon protein structures. (C) Z-score graphs. Black circles represent Z-scores of C. neoformans FKBP12 contacts minus hFKBP12 contacts. More significant contacts for C. neoformans (ΔZ-score >1) are labeled in cyan, while contacts more significant for the human protein (ΔZ-score <1) are labeled in magenta.

FIG. 10: Analysis of 500-ns molecular dynamic (MD) simulation stability. MD simulations were run for JH-FK-05 binding either human FKBP12 (hFKBP12) or C. neoformans FKBP12. (Top) Figure plotting the alpha carbon (C-α) root mean square deviation (RMSD) for each MD simulation over the length of the simulation. (Middle) Figure plotting the radius of gyration (Rg) for each MD simulation over the length of the simulation. (Bottom) Figure plotting the center of mass (COM; the three-dimensional [3D] point of mass balance for each monomer) between the protein and ligand for each MD simulation over the length of the simulation. MD simulation 3 of hFKBP12-JH-FK-05 was removed from the analysis due to unstable COM values observed during the length of the simulation.

FIG. 11: Chemical structures of novel C-22-modified FK520 analogs synthesized in this study. (A) One-step synthesis for C-22-modified FK520 analogs. Condition A: RNHNH2, EtOH, 90° C., 48 h; Condition B: RNHNH₂, MeOH, TFA (cat), 25° C., 48 h. (B) Structures of 12 analogs modified with a hydrazide or a substituted hydrazine at the C-22 position of FK520 are depicted.

FIG. 12: Low-solubility compounds in MIC assay. Representative image of MIC assay read by plate reader with growth indicated on a range from 0.04 to 0.1 OD, black to green coloration. Starting concentration 50 μg/mL for all drugs. (A) H99 cultured in wells, and (B) wells contain RPMI media and no fungal cells with green coloration indicative of false positive growth caused by drug insolubility.

FIG. 13: In vitro immunosuppressive activity of FK506, FK520, and analogs and pharmacokinetic profile of JH-FK-08 compared to FK506. (A) The percentage of IL-2-producing CD4⁺ T cells is plotted versus drug concentration as a measure of the immunosuppressive activity of calcineurin inhibitors. Newly synthesized analogs demonstrate reduced immunosuppression compared to controls FK506 and FK520. [n=3, error bars represent the standard error of the mean (SEM), IC₅₀ values determined by non-linear regression with variable slope analysis.](B) Whole blood drug concentrations are plotted over time following a single dose of 40 mg/kg of FK506 or JH-FK-08 (n=3, no statistically significant difference between groups, repeated measures ANOVA with Greisser-Greenhouse correction test).

FIG. 14: In vivo tolerability of JH-FK-08. Maximum tolerated dose study of female A/J mice (n=3). Animals received once daily (A) and twice daily (B) dosing, and weight over time was plotted as percent change from initial body weight.

FIG. 15: In vivo immunosuppression activity of JH-FK-08 is reduced compared to FK506. (A) Shown here is the production of Tfh cells during immune response in C57BL/6 female mice. (B) Shown here are representative flow cytometry plots showing the frequencies of Tfh cells among CD44^{hi} CD4⁺ T cells. Mice received twice daily treatment with vehicle, FK506 (2.5 mg/kg), JH-FK-08 (20 mg/kg), or JH-FK-08 (40 mg/kg) (n=5; **, P<0.001; ns, not significant; ordinary one-way ANOVA with Tukey's test).

FIG. 16: In vivo immunosuppression of JH-FK-08 compared to FK506. (A) Shown here is the production of germinal center B (BC) cells during immune response in C57BL/6 female mice. (B) Shown here is a representative flow cytometry plots showing gating to indicate region containing BC cells among CD19+B cells. Mice received twice daily treatment of vehicle, FK506 (2.5 mg/kg), JH-FK-08 (20 mg/kg), or JH-FK-08 (40 mg/kg) (n=5; **, p<0.001, ordinary One-way ANOVA with Tukey's test).

FIG. 17: JH-FK-08 and fluconazole treatment reduced organ fungal burden in infected animals. Female A/J mice (n=5) were infected with C. neoformans strain H99 through intranasal inoculation and treated daily with vehicle, 12 mg/kg fluconazole, 40 mg/kg JH-FK-08, or combination through i.p. dosing. Fungal burden levels in lung (A), brain (B), and spleen (C) were assessed after 14 days of treatment. (*, p=0.02; **, p=0.008; ***, p=0.0005, ordinary One-way ANOVA with Tukey's test, error bars represent SEM).

FIG. 18: JH-FK-08 treatment significantly reduced the organ fungal burden in infected animals. Female A/J mice (n=5) were infected with C. neoformans strain H99 through intranasal inoculation and treated twice daily with vehicle, 6 mg/kg fluconazole, 40 mg/kg JH-FK-08, or a combination through i.p. dosing. Fungal burden levels in the lung (A), brain (B), and spleen (C) were assessed after 14 days of treatment. (***, P<0.0001; **, P=0.0018, ordinary one-way ANOVA with Dunnet's test; error bars represent SEM)

FIG. 19: Histopathological analysis of lung tissue from animals treated with JH-FK-08 alone or in combination with fluconazole. Representative images of lung tissue from infected airways in various treatment groups stained with (A) periodic acid Schiff (PAS) staining or (B) hematoxylin and eosin (H&E). The inflammatory response to Cryptococcus is similar in all treated groups and composed of a prominent granulomatous reaction, better seen at lower magnification (100×) on the left column. Detail of the immune cell types present within this inflammatory response is better seen at higher magnification (630×) in the right column. Black arrows indicate lymphocytes, red arrows macrophages, and green arrows granulocytes (n=2).

FIG. 20: Murine survival is significantly extended by treatment with JH-FK-08 alone or in combination with fluconazole. Female A/J mice (n=10) were intranasally infected with C. neoformans strain H99 and received twice daily dosing of 6 mg/kg fluconazole, 40 mg/kg JH-FK-08, or a combination of the two drugs for the first 2 weeks post infection. Survival was monitored following the treatments (*, P=0.0065 found between JH-FK-08 and combination treatment groups; ***, P<0.0001 found between all other groups, Mantel-Cox test).

FIG. 21: In vitro immunosuppression of C18-modified FK506/FK520 analogs. Dose response curves generated from % IL-2 expression in cells exposed to increasing concentrations of FK506 and FK520 analogs. All analogs were reduced for immunosuppression compared to controls FK506 (black) and FK520 (gray).

FIGS. 22, 23 and 24 present antifungal activity for JH-FK-29, JH-FK-30, JH-FK-31, JH-FK-33, JH-FK-35, JH-FK-37, JH-FK-38, and JH-FK-40.

FIG. 25 provides NMR spectra data for JH-FK-8-13, 14-18, 20-24, 29-46, 41A, 41B, 41C, 42A, and 44A.

FIG. 26 provides the calculated mass and observed mass for JH-FK-8-18, 20-24, and 29-46.

FIG. 27 provides measured therapeutic index values for JH-FK-41, 42, 43, 44, 45, 46, FK506, and FK520 against Cryptococcus and Candida.

### DETAILED DESCRIPTION OF THE INVENTION

Fungal infections are of mounting global concern, and the current limited treatment arsenal poses challenges when treating such infections. In particular, infections by Cryptococcus neoformans are associated with high mortality, emphasizing the need for novel therapeutic options. Calcineurin is a protein phosphatase that mediates fungal stress responses, and calcineurin inhibition by the natural product FK506 blocks C. neoformans growth at 37° C. Calcineurin is also required for pathogenesis. However, because calcineurin is conserved in humans and inhibition with FK506 results in immunosuppression, the use of FK506 as an anti-infective agent is precluded. The structures of multiple fungal calcineurin-FK506-FKBP12 complexes were previously elucidated and implicated the C-22 position on FK506 as a key point for differential modification of ligand inhibition of mammalian versus fungal target proteins. Through in vitro antifungal and immunosuppressive testing of FK520 (a natural analog of FK506) derivatives, experiments described herein resulted in the identification of JH-FK-08 as a lead candidate for further antifungal development. JH-FK-08 exhibited significantly reduced immunosuppressive activity and both reduced fungal burden and prolonged survival of infected animals. JH-FK-08 exhibited additive activity in combination with fluconazole in vivo. These findings further advance calcineurin inhibition as an antifungal therapeutic approach.

Indeed, fungal infections cause significant morbidity and mortality globally. The therapeutic armamentarium against these infections is limited, and the development of antifungal drugs has been hindered by the evolutionary conservation between fungi and the human host. With rising resistance to the current antifungal arsenal and an increasing at-risk population, there is an urgent need for the development of new antifungal compounds. The FK520 analogs described herein display potent antifungal activity as a novel class of antifungals centered on modifying an existing orally active FDA-approved therapy. This research advances the development of much-needed newer antifungal treatment options with novel mechanisms of action.

Experiments conducted during the course of developing embodiments for the present invention developed an expanded library of FK506/FK520 analogs modified at one or more of the C-18 position, the C-21 position, and the C-22 position. These efforts have resulted in the generation of compound JH-FK-08 as a novel antifungal therapeutic effective at significantly reducing lung fungal burden in a murine cryptococcosis model compared to untreated infected animals. Furthermore, treatment with JH-FK-08 was shown to significantly prolonged murine survival and surpassed the antifungal efficacy achieved in previous efforts. These advances identify an FK520 analog with the potential to be utilized to treat invasive fungal infections.

Accordingly, the present disclosure provides methods for treating or preventing a fungal infection with one or more macrolide compounds, or pharmaceutically acceptable salts thereof, or compositions comprising the same and an additional anti-fungal agent, and pharmaceutical compositions comprising one or more macrolide compounds and at least one additional anti-fungal agent.

Section headings as used in this section and the entire disclosure herein are merely for organizational purposes and are not intended to be limiting.

### 1. Definitions

Certain terms employed in the specification, examples, and appended claims are further described here in the present invention. These definitions should be read in light of the entire invention and as would be understood by a person skilled in art.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular. All publications, patent applications, patents and other references mentioned herein are incorporated by reference in their entirety.

As used herein, the terms “a” or “an” means “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, “treat,” “treating,” and the like means a slowing, stopping, or reversing of progression of a disease or disorder when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” and “introducing,” are used interchangeably herein and refer to the placement of the compounds or compositions of the disclosure into a subject by a method or route which results in at least partial localization of the compounds or composition to a desired site. The compounds or compositions can be administered by any appropriate route which results in delivery to a desired location in the subject.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

“Cycloalkyl” refers to a saturated hydrocarbon ring that is not aromatic. Cycloalkyl rings are monocyclic, or are fused, spiro, or bridged bicyclic or polycyclic ring systems. Monocyclic cycloalkyl rings contain from about 3 to about 12 carbon atoms, preferably from 3 to 7 carbon atoms, in the ring. Bicyclic cycloalkyl rings contain from 7 to 17 carbon atoms, preferably from 7 to 12 carbon atoms, in the ring. Preferred bicyclic cycloalkyl rings comprise 4-, 5-, 6- or 7-membered rings fused to 5-, 6- or 7-membered rings. Cycloalkyl rings may be unsubstituted or substituted with from 1 to 4 substituents on the ring. Cycloalkyl may be substituted with halogen, cyano, nitro, alkyl, heteroalkyl, haloalkyl, cycloalkyl, heterocycloalkyl, aryl, heteroaryl, keto, hydroxy, carboxy, amino, acylamino, aryloxy, heteroaryloxy, alkoxy, arylalkyl, heteroarylalkyl or any combination thereof. Examples of cycloalkyl rings include cyclopropyl, cyclopentyl, cyclohexyl, cycloheptyl, cyclooctyl and cyclononyl rings.

“Heterocycloalkyl” is a saturated or unsaturated ring containing carbon atoms and from 1 to 4 (preferably 1 to 3) heteroatoms in the ring. Heterocycloalkyl rings are monocyclic, or are fused, spiro, or bridged bicyclic or polycyclic ring systems. Monocyclic heterocycloalkyl rings contain from about 3 to about 9 member atoms (including both carbons and heteroatoms), preferably from 5 to 7 member atoms, in the ring. Bicyclic heterocycloalkyl rings may be fused, spiro, or bridged ring systems. Preferred bicyclic heterocycloalkyl rings comprise 5-, 6- or 7-membered rings fused to 5-, 6- or 7-membered rings. Heterocycloalkyl rings may be unsubstituted (i.e., contain hydrogen) or substituted (on either carbons or heteroatoms or both) with from 1 to 4 substituents selected from halogen, cyano, nitro, alkyl, heteroalkyl, haloalkyl, cycloalkyl, heterocycloalkyl, aryl, heteroaryl, keto, hydroxy, carboxy, amino, acylamino, aryloxy, heteroaryloxy, alkoxy, arylalkyl, heteroarylalkyl or any combination thereof.

“Aryl” refers to aromatic monocyclic or multicyclic groups containing from 3 to 16 carbon atoms. Aryl may be unsubstituted or substituted with from 1 to 4 substituents. Preferred substituted aryls are mono-, di, or tri-substituted. Aryls may be substituted with halogen, cyano, nitro, alkyl, heteroalkyl, haloalkyl, cycloalkyl, heterocycloalkyl, aryl, heteroaryl, keto, hydroxy, carboxy, amino, acylamino, aryloxy, heteroaryloxy, alkoxy, arylalkyl, heteroarylalkyl or any combination thereof.

“Heteroaryl” or Het refers to a monocyclic or multicyclic aromatic ring system, of about 5 to about 15 members where one or more of the atoms in the ring system is a heteroatom, that is, an element other than carbon, including but not limited to nitrogen, oxygen or sulfur. The heteroaryl group may be optionally fused to a benzene ring. Heteroaryl may be unsubstituted or substituted with from 1 to 4 substituents. Preferred substituted heteroaryls are mono-, di, or tri-substituted. Heteroaryls may be substituted with halogen, cyano, nitro, alkyl, heteroalkyl, haloalkyl, cycloalkyl, heterocycloalkyl, aryl, heteroaryl, keto, hydroxy, carboxy, amino, acylamino, aryloxy, heteroaryloxy, alkoxy, arylalkyl, heteroarylalkyl or any combination thereof.

“Halo” or “halogen” is fluoro, chloro, bromo or iodo.

“Alkyl” means a saturated hydrocarbon radical having 1 to 15 carbon atoms, preferably 1 to 10 carbon atoms, more preferably 1 to 5 carbon atoms, most preferably 1 to 3 carbon atoms, that may be branched or unbranched. Non-limiting example of alkyl radicals include methyl, ethyl, n-propyl, isopropyl, n-butyl, isobutyl, sec-butyl, tert-butyl, pentyl, hexyl, heptyl, octyl and the like, wherein methyl, ethyl, n-propyl and isopropyl represent specifically preferred examples.

“Heteroalkyl” is a saturated or unsaturated chain carbon and at least one heteroatom, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more carbon-carbon double bounds and/or one or more carbon-carbon triple bounds. Preferred unsaturated heteroalkyl have one or two carbon-carbon double bounds or one carbon-carbon triple bound, more preferably one double bound. Heteroalkyl chains may be unsubstituted or substituted with from 1 to 4 substituents. Preferred substituted heteroalkyl are mono-, di, or tri-substituted. Heteroalkyl may be substituted with halogen, cyano, nitro, alkyl, heteroalkyl, haloalkyl, cycloalkyl, heterocycloalkyl, aryl, heteroaryl, keto, hydroxy, carboxy, amino, acylamino, aryloxy, heteroaryloxy, alkoxy, arylalkyl, heteroarylalkyl or any combination thereof.

“Alkoxy” means an oxygen radical having a hydrocarbon chain substituent, where the hydrocarbon chain is an alkyl or alkenyl (i.e., —O-alkyl or —O-alkenyl). Examples of alkoxy include methoxy, ethoxy, n-propoxy, isopropoxy, n-butoxy, isobutoxy, sec-butoxy, tert-butoxy, allyloxy and the like.

“Arylalkyl” alone or in combination, refers to an alkyl radical in which one hydrogen atom is replaced by an aryl radical, for example, benzyl and the like.

“Heteroarylalkyl” refers to an alkyl radical in which one hydrogen atom is replaced by a heteroaryl radical.

“Independently” groups are groups present in the same structure that need not all represent the same substitution.

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogen atoms on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, sulfonamido, thiol, thione, thioxo, or combinations thereof.

In some instances, the number of carbon atoms in a hydrocarbyl substituent (e.g., alkyl alkenyl) is indicated by the prefix “Cₓ-C_{y}”, wherein x is the minimum and y is the maximum number of carbon atoms in the substituent. Thus, for example, “C₁-C₃ alkyl” refers to an alkyl substituent containing from 1 to 3 carbon atoms.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— is intended to encompass —OCH₂—, and —C(O)NH— is intended to encompass —NHC(O)—.

“Pharmacological composition” refers to a mixture of one or more of the compounds described herein or pharmaceutically acceptable salts thereof, with other chemical components, such as pharmaceutically acceptable carriers and/or excipients. The purpose of a pharmacological composition is to facilitate administration of a compound to an organism.

“Pharmaceutically acceptable salts” is a cationic salt formed at any acidic (e.g., carboxylic acid) group, or an anionic salt formed at any basic (e.g., amino) group.

“Solvate” is a physical association of a compound of the invention with one or more solvent molecules, whether organic or inorganic. This physical association often includes hydrogen bonding. In certain instances, the solvate is capable of isolation, for example, when one or more solvate molecules are incorporated in the crystal lattice of the crystalline solid. “Solvate” encompasses both solution-phase and isolable solvates. Exemplary solvates include hydrates, ehanolates, and methanolates.

“Prodrug” refers to a pharmacologically inactive derivative of a parent “drug” molecule which requires biotransformation within the target physiological system to release, or to convert the prodrug into the active drug. Prodrugs can address the problems associated with solubility, stability, cell permeability or bioavailability. Prodrugs usually comprise an active drug molecule and a chemical masking group. Prodrugs can be readily prepared from the parent compounds with well-known methods.

### 2. Compounds

In a particular embodiment, compounds encompassed within the following formulas are provided:

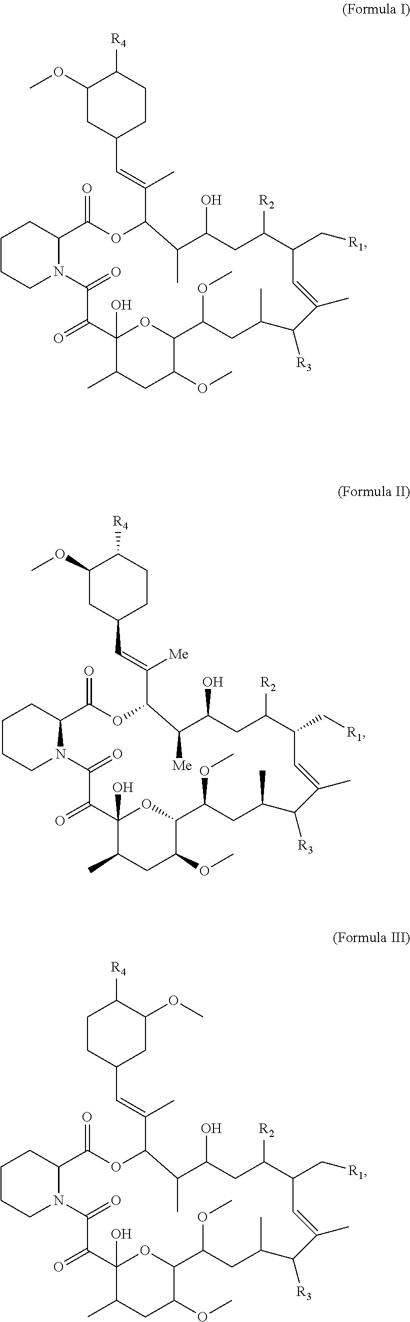

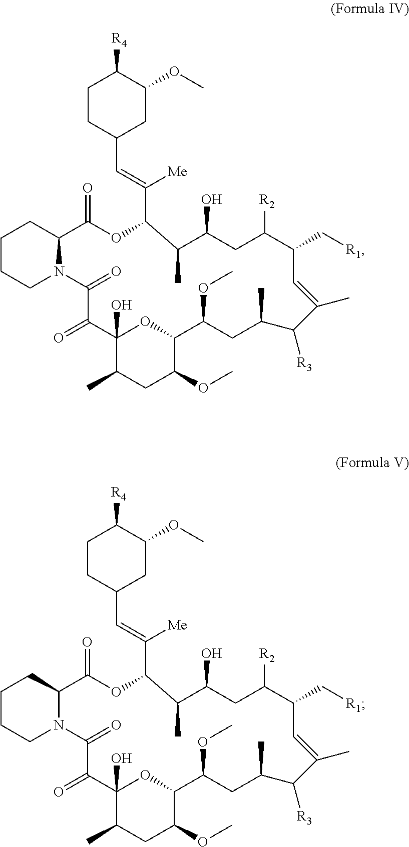

including pharmaceutically acceptable salts, hydrates, solvates, polymorphs, prodrugs, triphenylphosphonium (TPP) tagged derivatives, proteolysis targeting chimera (PROTAC) derivatives, mitochondria tagged derivatives, biotin tagged derivatives, boron-dipyrromethene (BODIPY) tagged derivatives, stereoisomers, and/or deuterated analogues thereof.

Formula I is not limited to a particular chemical moiety for R₁, R₂, R₃, and R₄. In some aspects, the particular chemical moiety for R₁, R₂, R₃, and R₄ independently include any chemical moiety that renders the resulting compound capable of inhibiting fungal activity. In some aspects, the particular chemical moiety for R₁, R₂, R₃, and R₄ independently include any chemical moiety that renders the resulting compound capable of inhibiting calcineurin. In some aspects, the particular chemical moiety for R₁, R₂, R₃, and R₄ independently include any chemical moiety that renders the resulting compound capable of inhibiting fungal activity associated with calcineurin. In some aspects, the particular chemical moiety for R₁, R₂, R₃, and R₄ independently include any chemical moiety that renders the resulting compound capable of preventing fungal growth. In some aspects, the particular chemical moiety for R₁, R₂, R₃, and R₄ independently include any chemical moiety that renders the resulting compound capable of inhibiting fungal activity. In some aspects, the particular chemical moiety for R₁, R₂, R₃, and R₄ independently include any chemical moiety that renders the resulting compound capable of inhibiting calcineurin activity. In some aspects, the particular chemical moiety for R₁, R₂, R₃, and R₄ independently include any chemical moiety that renders the resulting compound capable of associating with (e.g., binding with) FKBP12. In some aspects, the particular chemical moiety for R₁, R₂, R₃, and R₄ independently include any chemical moiety that renders the resulting compound capable of associating with (e.g., binding with) FKBP12 to form a complex that is capable of inhibiting calcineurin activity. In some aspects, the particular chemical moiety for R₁, R₂, R₃, and R₄ independently include any chemical moiety that renders the resulting compound capable of associating with (e.g., binding with) FKBP12 to form a complex that is capable of inhibiting fungal activity. In some aspects, the particular chemical moiety for R₁, R₂, R₃, and R₄ independently include any chemical moiety that renders the resulting compound capable of associating with (e.g., binding with) FKBP12 to form a complex that is capable of inhibiting Cryptococcus neoformans activity. In some aspects, the particular chemical moiety for R₁, R₂, R₃, and R₄ independently include any chemical moiety that renders the resulting compound effective as a therapeutic for the treatment and/or prevention of fungal infection (e.g., fungal infections including, but not limited to, Candida, Aspergillus, Microsporum, Trichophyton, Cryptococcus, and Epidermophyton). In some aspects, the particular chemical moiety for R₁, R₂, R₃, and R₄ independently include any chemical moiety that renders the resulting compound effective as a therapeutic for the treatment and/or prevention of fungal infection associated with calcineurin activity. In some aspects, the particular chemical moiety for R₁, R₂, R₃, and R₄ independently include any chemical moiety that renders the resulting compound effective as a therapeutic for the treatment and/or prevention of Cryptococcus neoformans infection.

In some aspects, R1 is selected from hydrogen,

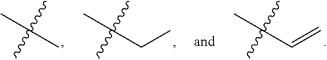

In some aspects, R₂ is selected from hydrogen,

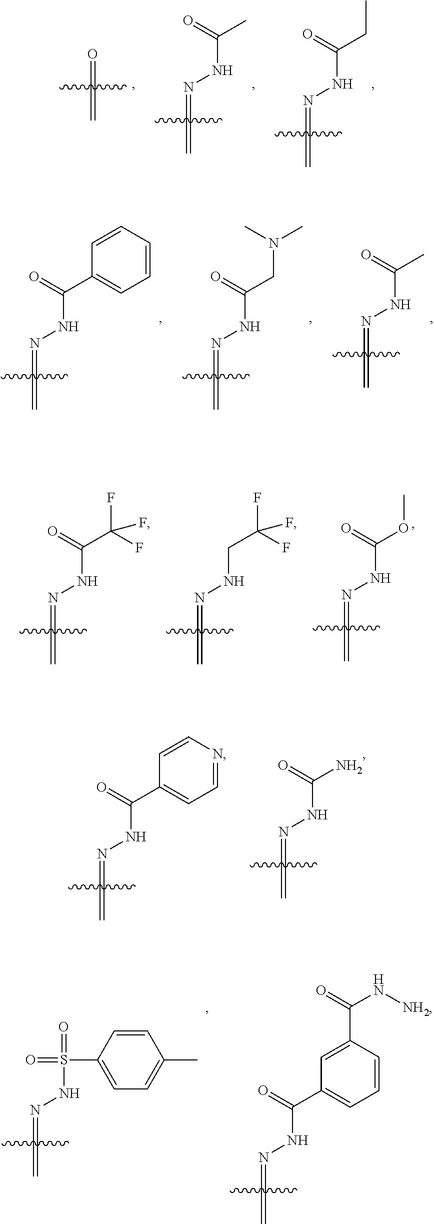

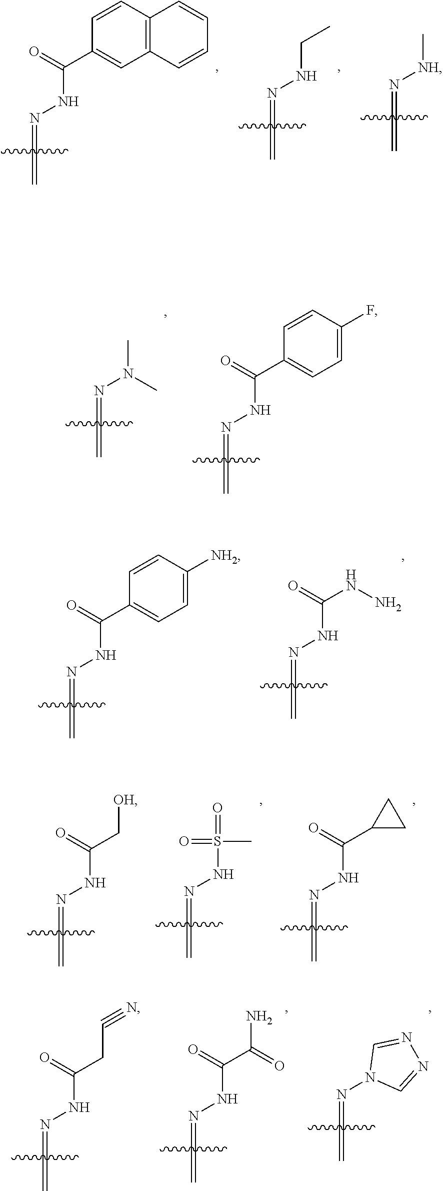

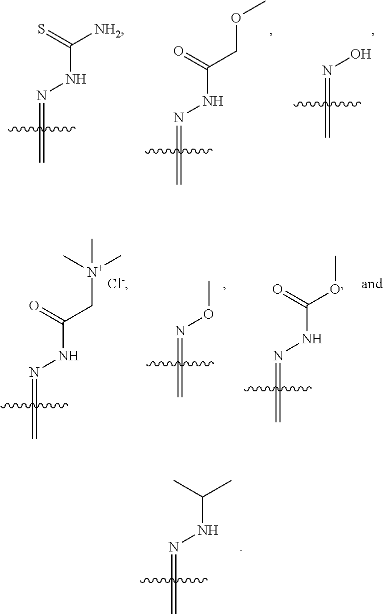

In some aspects, R₃ is selected from hydrogen, and hydroxyl (e.g., OH).

In some aspects, R₄ is selected from hydrogen, hydroxyl (e.g., OH),

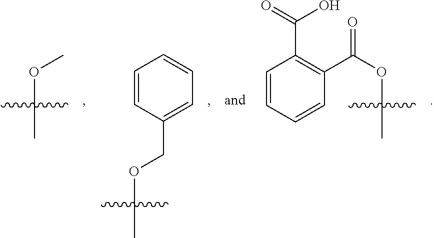

In some embodiments, the compound encompassed within Formulas I, II, III, and/or IV is selected from:

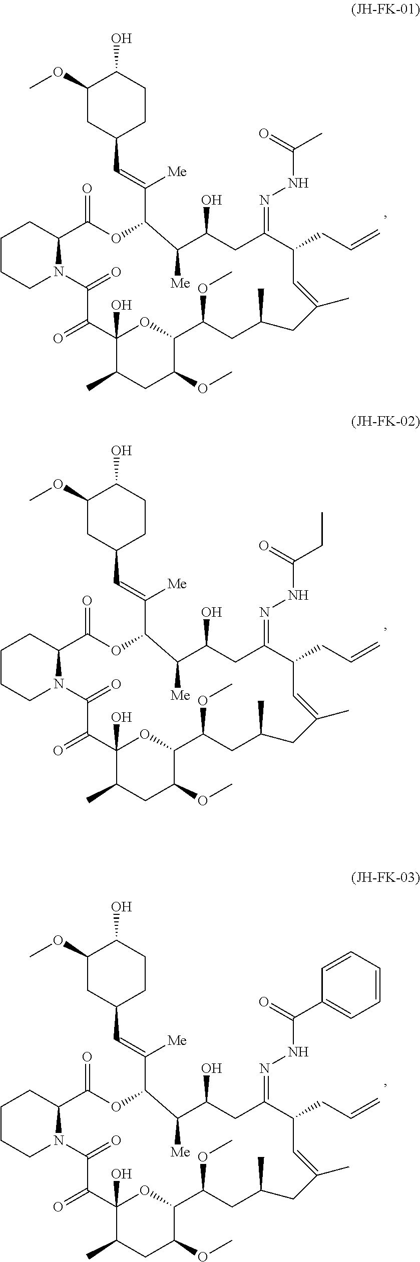

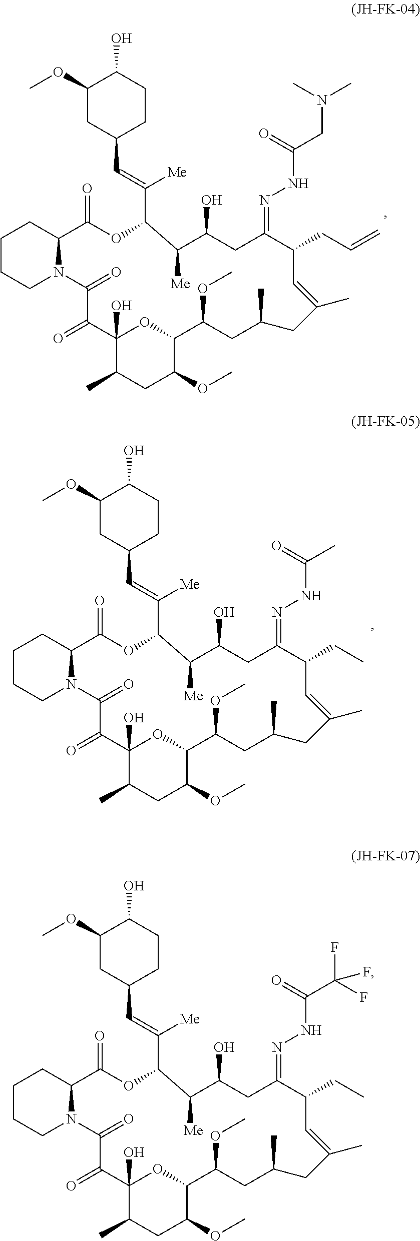

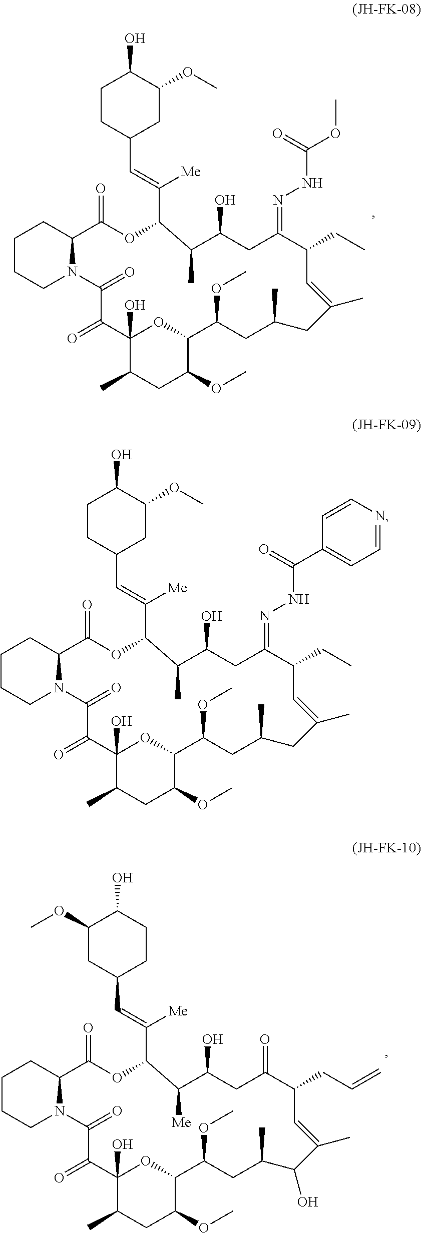

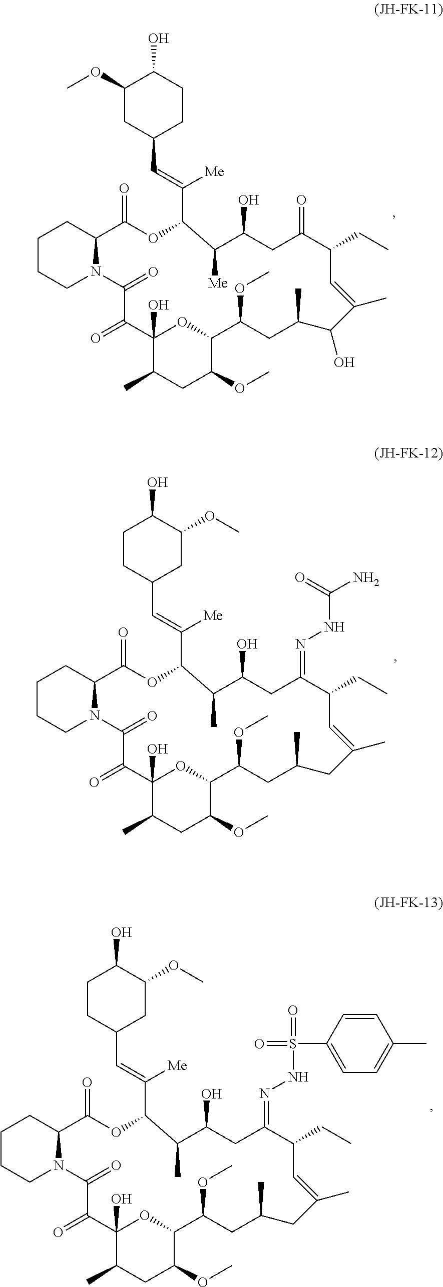

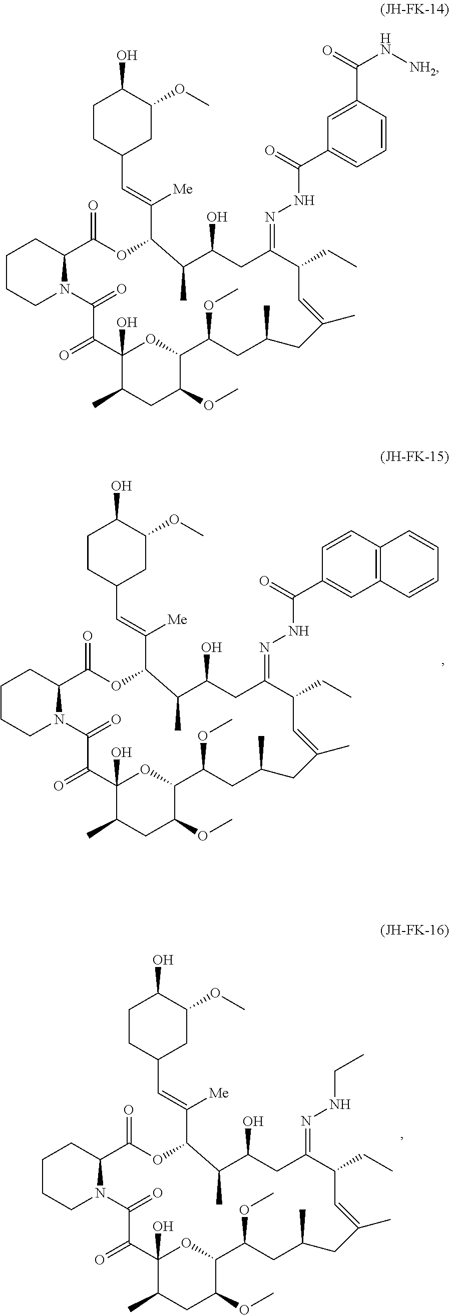

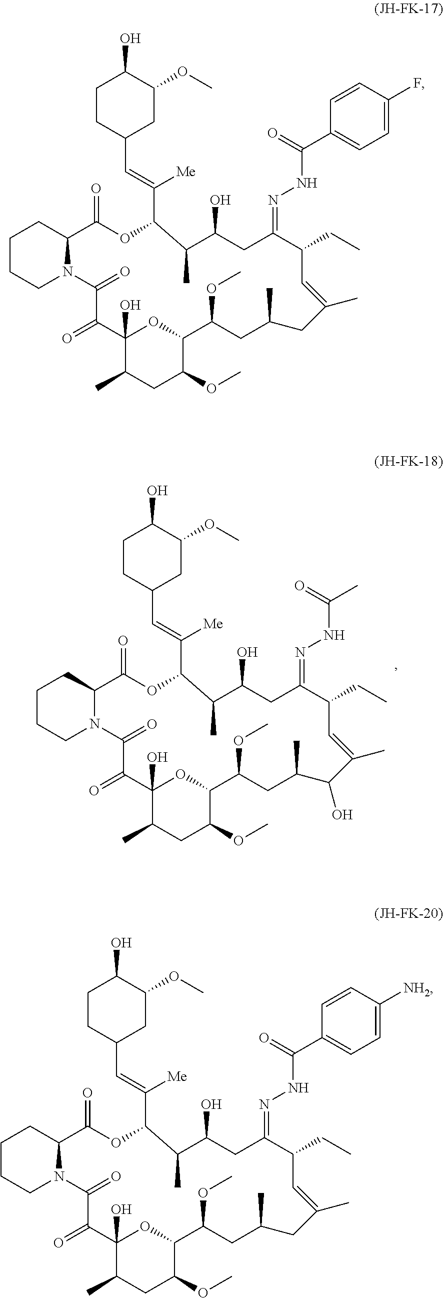

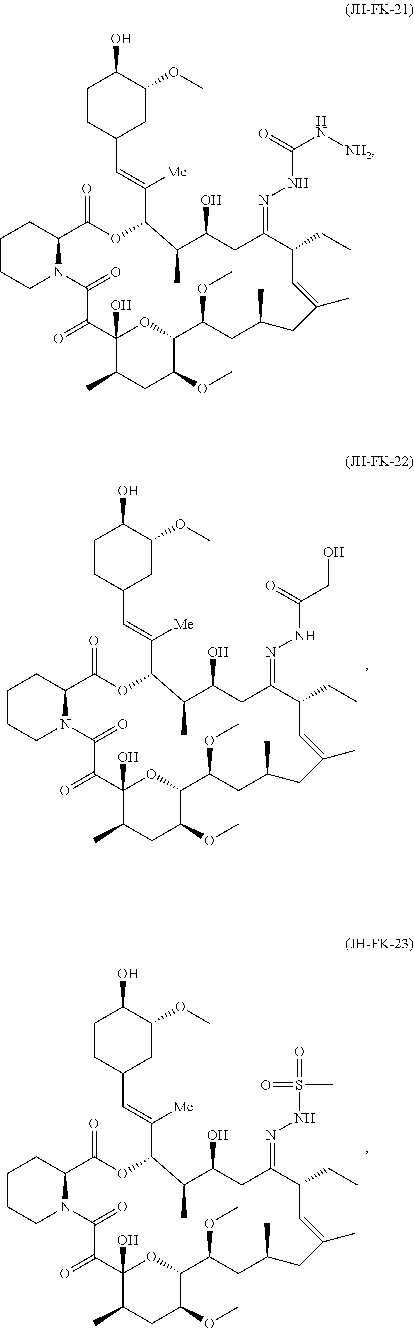

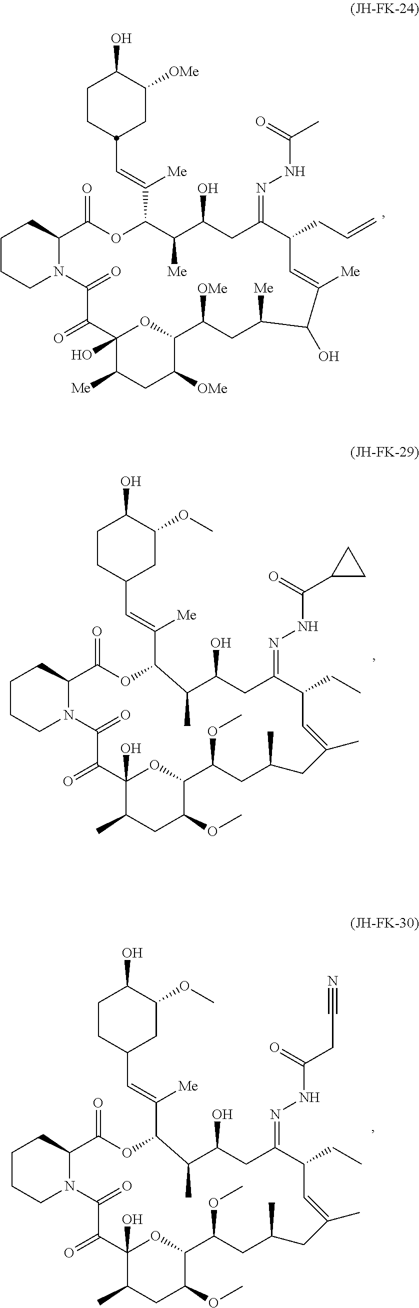

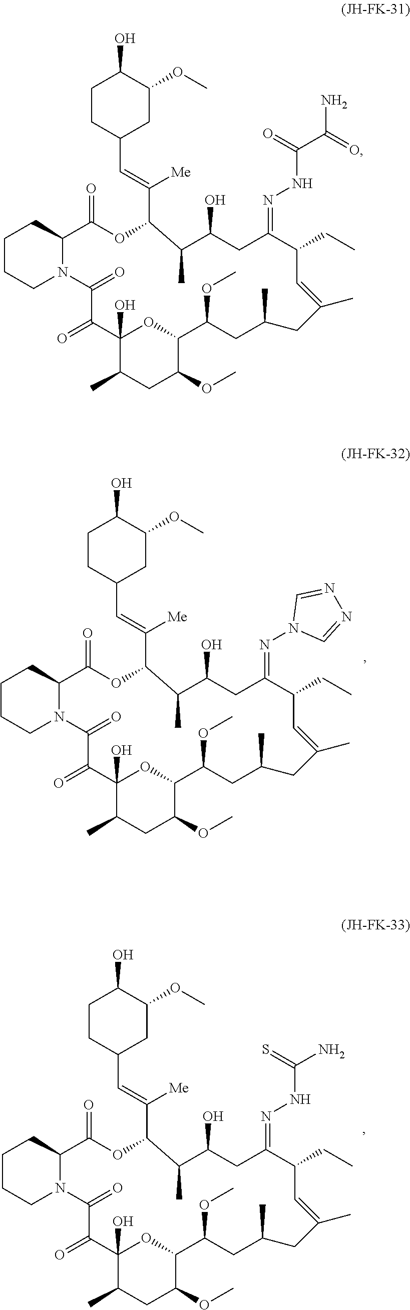

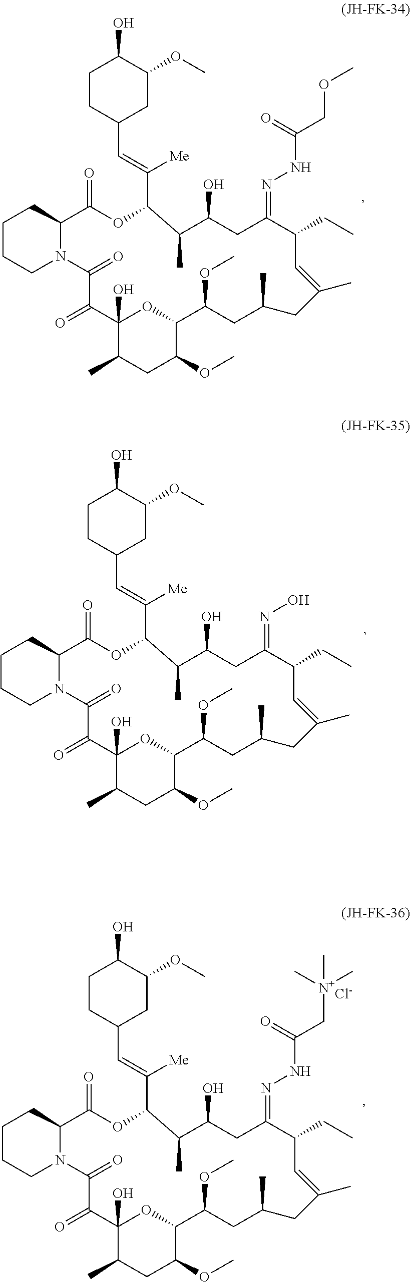

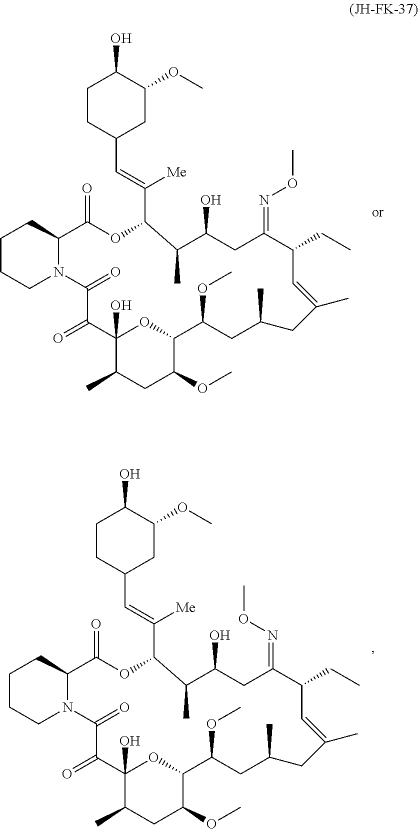 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/OC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N\OC)[C@H](C)C[C@@H]2OC]

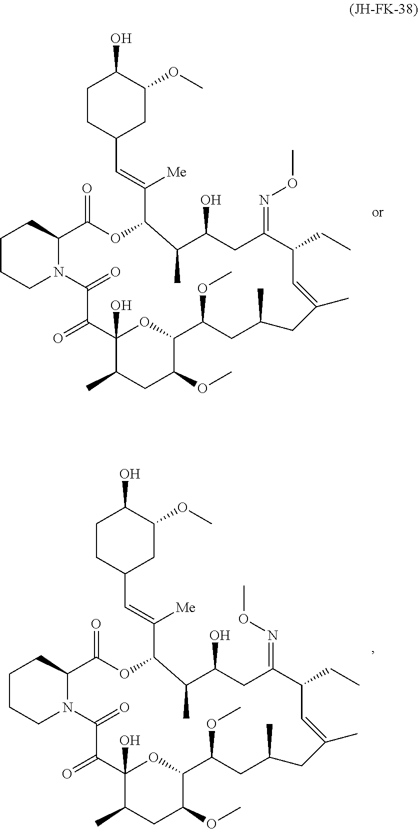 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/OC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N\OC)[C@H](C)C[C@@H]2OC]

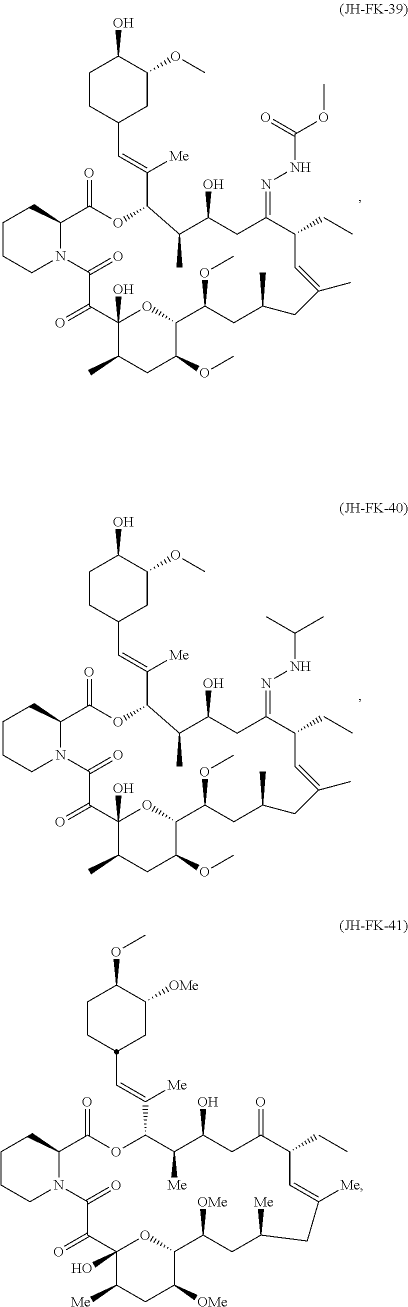

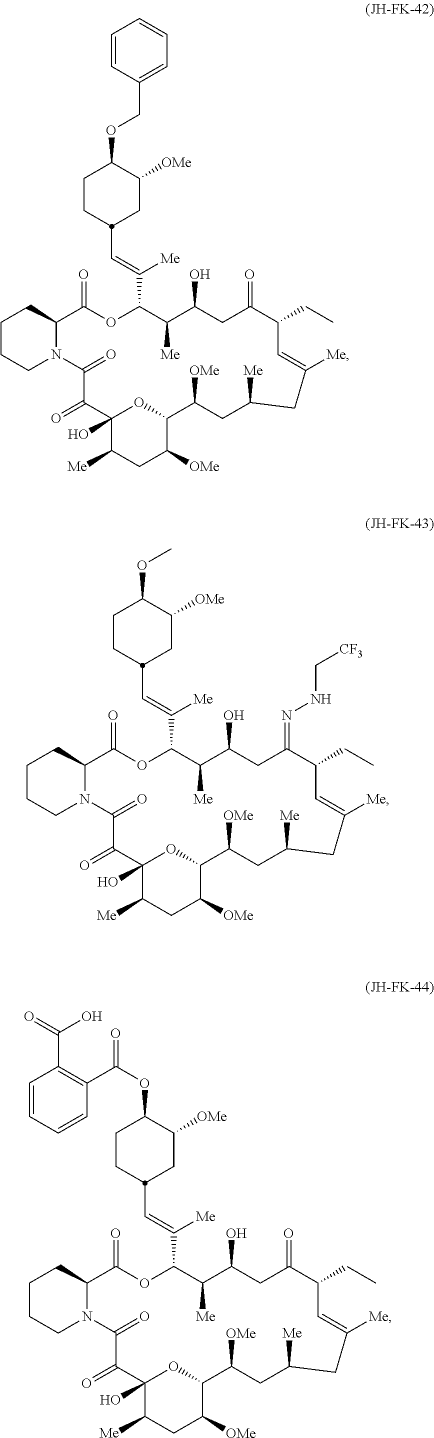

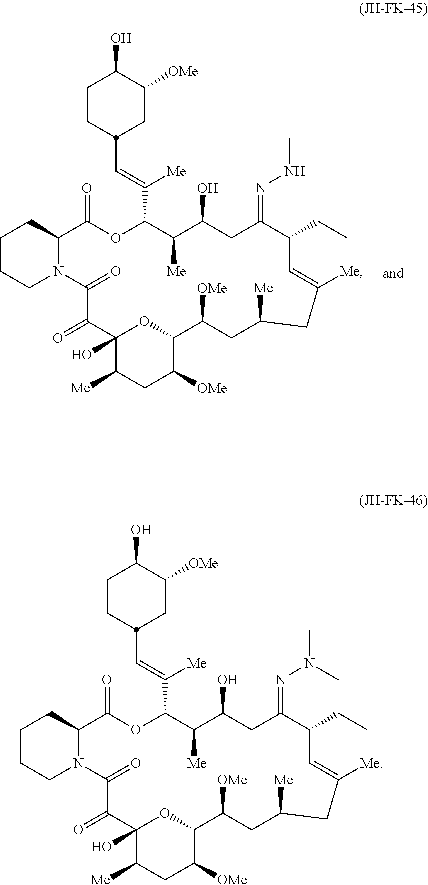 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/NC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/N(C)C)[C@H](C)C[C@@H]2OC]

The compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England (or more recent versions thereof), or (2) direct separation of the mixture of optical enantiomers on chiral chromatographic columns, or (3) fractional recrystallization methods.

It should be understood that the compounds may possess tautomeric forms, as well as geometric isomers, and that these also constitute embodiments of the disclosure.

The present disclosure also includes isotopically-labeled compounds, which is identical to those recited in Formulas I, II, III, and IV but for the fact that one or more atoms are replaced by an atom having an atomic mass or mass number different from the atomic mass or mass number usually found in nature. Examples of isotopes include those for hydrogen, carbon, nitrogen, oxygen, phosphorus, sulfur, fluorine, and chlorine, such as, but not limited to ²H, ³H, ¹³C, ⁴C, ¹⁵N, ¹⁸O, ¹⁷O, ³P, ³²P, ³⁵5, ¹⁸F, and ³⁶Cl, respectively. Substitution with heavier isotopes such as deuterium, for example, ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Compounds may be synthesized according to a variety of methods. Reaction conditions and reaction times for each individual step can vary depending on the particular reactants employed and substituents present in the reactants used. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described scheme.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the disclosure can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

In some embodiments, the compounds described herein are derivatives referred to as prodrugs. The expression “prodrug” denotes a derivative of a known direct acting drug. which derivative has enhanced delivery characteristics and therapeutic value as compared to the drug, and is transformed into the active drug by an enzymatic or chemical process.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

It can be appreciated that the synthetic schemes and specific examples as described are illustrative and are not to be read as limiting the scope of the disclosure as it is defined in the appended claims. All alternatives, modifications, and equivalents of the synthetic methods and specific examples are included within the scope of the claims.

### 3. Compositions

The disclosed compounds may be incorporated into compositions that may be suitable for administration to a subject (such as a patient, which may be a human or non-human).

3a. Pharmaceutical Compositions

The disclosed compounds may be incorporated into pharmaceutically acceptable compositions. The pharmaceutical compositions may include a “therapeutically effective amount” or a “prophylactically effective amount” of the compound(s). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis). In some embodiments, the composition is for oral administration. In some embodiments, the composition is for subcutaneous administration. In some embodiments, the composition is for intravenous administration.

Carriers for systemic administration typically include at least one of diluents, lubricants, binders, disintegrants, colorants, flavors, sweeteners, antioxidants, preservatives, glidants, solvents, suspending agents, wetting agents, surfactants, cyclodextrins combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, dimethyl sulfoxide, N-methyl-2-pyrrolidone, dimethylacetamide and phosphate (or other suitable buffer). The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Suitable cyclodextrins include alpha-CD, beta-CD, gamma-CD, hydroxypropyl betadex (HP-beta-CD), sulfobutyl-ether β-cyclodextrin (SBE-beta-CD). The amount of cyclodextrins in the systemic or topical composition is typically about 0% to about 40%.

Although the amounts of components in the systemic compositions may vary depending on the type of systemic composition prepared, in general, systemic compositions include 0.01% to 50% of an active compound (e.g., a compound as described herein) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

3a. Additional Therapeutic Agents

Any of the above compositions or formulations disclosed herein may further comprise at least one additional therapeutic agent. In some embodiments the at least one additional therapeutic agent is one or more other anti-fungal agents. In some embodiments, the composition comprises any one or more of the compounds of Formula I, or a pharmaceutically acceptable salt thereof, and one or more other anti-fungal agent(s). In some embodiments, the composition comprises any one or more of the compounds of Formula II, or a pharmaceutically acceptable salt thereof, and one or more other anti-fungal agent(s). In some embodiments, the composition comprises any one or more of the compounds of Formula III, or a pharmaceutically acceptable salt thereof, and one or more other anti-fungal agent(s). In some embodiments, the composition comprises any one or more of the compounds of Formula IV, or a pharmaceutically acceptable salt thereof, and one or more other anti-fungal agent(s). In some embodiments, the composition comprises any one or more of the compounds of Formula V, or a pharmaceutically acceptable salt thereof, and one or more other anti-fungal agent(s).

In some embodiments, the other anti-fungal agent is an azole or an echinocandin. In some embodiments, the other anti-fungal agent is an azole. In some embodiments, the azole is itraconazole, posaconazole, voriconazole (VOR), or isavuconazole. In some embodiments, the azole is itraconazole. In some embodiments, the azole is posaconazole. In some embodiments, the azole is voriconazole. In some embodiments, the azole is isavuconazole. In some embodiments, the other anti-fungal agent is an echinocandin. In some embodiments, the echinocandin is caspofungin (CAS). In some embodiments, the other anti-fungal agent is nystatin, miconazole, Gentian violet, or amphotericin B. In some embodiments, the other anti-fungal agent is nystatin. In some embodiments, the other anti-fungal agent is miconazole. In some embodiments, the other anti-fungal agent is Gentian violet. In some embodiments, the other anti-fungal agent is amphotericin B. Additional anti-fungal agents include, but are not limited to, fosmanogepix, ibrexafungerp, olorofim, opelconazole, and rezafungin. In some embodiments, the other anti-fungal agent is fosmanogepix. In some embodiments, the other anti-fungal agent is ibrexafungerp. In some embodiments, the other anti-fungal agent is olorofim. In some embodiments, the other anti-fungal agent is opelconazole. In some embodiments, the other anti-fungal agent is rezafungin. In some embodiments, the other anti-fungal agent is Nikkomycin Z. Other anti-fungal agents include VT-1129, VT-1161, VT-1598, PC1244, SUBA-ITC, CAMB, MGCD290, T-2307, and VL-2397. Additional anti-fungal agents are disclosed in, for example, PCT Publication No. WO 2021/247781.

In embodiments, the antifungal agent is a known antifungal and not a compound according to the formulae herein, such as a polyene, imidazole, triazole, thiazole, allylamine, echinocandin, among others. Examples include Amphotericin B, Candicidin, Filipin, Hamycin, Natamycin, Nystatin, Rimocidin, Bifonazole, Butoconazole, Clotrimazole, econazole, fenticonazole, isoconazole, kentoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, polygodial, tolnaftate, undecylenic acid, and crystal violet, among others.

In some embodiments, any one or more of the compounds of Formula I, Formula II, Formula III, Formula IV, or Formula V, or a pharmaceutically acceptable salt thereof, can be combined with a Protein Kinase C inhibitor, such as chelerythrine or calphostin C.

In some embodiments, the at least one additional therapeutic agent comprises a polynucleotide or nucleic acid (e.g., ribonucleic acid or deoxyribonucleic acid). The term “polynucleotide,” in its broadest sense, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc.

In some embodiments, the at least one additional therapeutic agent is an RNA. RNAs useful in the compositions and methods described herein can be selected from the group consisting of, but are not limited to, shortmers, antagomirs, antisense RNAs, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and mixtures thereof.

In certain embodiments, the at least one additional therapeutic agent is an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

In other embodiments, the at least one additional therapeutic agent is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, the at least one additional therapeutic agent is an shRNA or a vector or plasmid encoding the same. An shRNA may be produced inside a target cell upon delivery of an appropriate construct to the nucleus. Constructs and mechanisms relating to shRNA are well known in the relevant arts.

### 4. Methods of Use

The disclosure further provides methods for treating a disease or disorder comprising administration of a compound or composition as disclosed herein, to a subject in need thereof. In some embodiments, the subject is a human.

The present disclosure provides methods of treating or preventing a Cryptococcus fungal infection in a mammal comprising administering to the mammal in need thereof a compound of Formula I, Formula II, Formula III, Formula IV, or Formula V. In some embodiments, the methods of treating or preventing a Cryptococcus fungal infection in a mammal comprise administering to the mammal in need thereof a compound of Formula I. In some embodiments, the methods of treating or preventing a Cryptococcus fungal infection in a mammal comprise administering to the mammal in need thereof a compound of Formula II. In some embodiments, the methods of treating or preventing a Cryptococcus fungal infection in a mammal comprise administering to the mammal in need thereof a compound of Formula III. In some embodiments, the methods of treating or preventing a Cryptococcus fungal infection in a mammal comprise administering to the mammal in need thereof a compound of Formula IV. In some embodiments, the methods of treating or preventing a Cryptococcus fungal infection in a mammal comprise administering to the mammal in need thereof a compound of Formula V.

The present disclosure also provides methods of killing or inhibiting the growth of a Cryptococcus species comprising contacting the Cryptococcus species with a compound of Formula I, Formula II, Formula III, Formula IV, or Formula V.

The present disclosure provides methods of treating or preventing a fungal infection in a mammal comprising administering to the mammal in need thereof a compound of Formula I, Formula II, Formula III, Formula IV, or Formula V in combination with one or more other anti-fungal agent(s) (i.e., in the same pharmaceutical composition or in separate pharmaceutical compositions).

The present disclosure also provides methods of killing or inhibiting the growth of a fungus comprising contacting the fungus with a compound of Formula I, Formula II, Formula III, Formula IV, or Formula V in combination with one or more other anti-fungal agent(s) (i.e., in the same pharmaceutical composition or in separate pharmaceutical compositions).

In some embodiments, the fungus is, or the fungal infection is caused by, Aspergillus spp. (e.g., Aspergillus fumigatus, Aspergillus flavus, Aspergillus niger, and Aspergillus terreus), Fusarium spp. (e.g., Fusarium solani, Fusarium moniliforme, and Fusarium proliferatum), Malassezia spp. (e.g., Malassezia pachydermatis), Candida spp. (e.g., Candida albicans, Candida glabrata, Candida tropicalis, Candida krusei, and Candida auris), or Cryptococcus spp. (e.g., Cryptococcus neoformans), Mucorales such as Mucor spp. (e.g., M. circinelloides), Rhizopus spp. (e.g., Rhizopus delemar and Rhizopus oryzae), Lichtheimia spp. (e.g., Lichtheimia corymbifera), and Rhizomucor spp., or Chrysosporium parvum, Metarhizium anisopliae, Phaeoisaria clematidis, or Sarcopodiurm oculorum. In some embodiments, the fungus is, or the fungal infection is caused by, Aspergillus spp., Fusarium spp., Malassezia spp., Candida spp., or Cryptococcus spp. In some embodiments, the fungus is, or the fungal infection is caused by, Aspergillus spp. In some embodiments, the fungus is, or the fungal infection is caused by, Aspergillus fumigatus. In some embodiments, the fungus is, or the fungal infection is caused by, Aspergillus flavus. In some embodiments, the fungus is, or the fungal infection is caused by, Aspergillus niger. In some embodiments, the fungus is, or the fungal infection is caused by, Aspergillus terreus. In some embodiments, the fungus is, or the fungal infection is caused by, Fusarium spp. In some embodiments, the fungus is, or the fungal infection is caused by, Fusarium solani. In some embodiments, the fungus is, or the fungal infection is caused by, Fusarium monihforme. In some embodiments, the fungus is, or the fungal infection is caused by, Fusarium proliferatum. In some embodiments, the fungus is, or the fungal infection is caused by, Malassezia spp. In some embodiments, the fungus is, or the fungal infection is caused by, Malassezia pachydermatis. In some embodiments, the fungus is, or the fungal infection is caused by, a Mucorales. In some embodiments, the fungus is, or the fungal infection is caused by, Mucor spp. In some embodiments, the fungus is, or the fungal infection is caused by, M. circinelloides. In some embodiments, the fungus is, or the fungal infection is caused by, Rhizopus spp. In some embodiments, the fungus is, or the fungal infection is caused by, Rhizopus delemar. In some embodiments, the fungus is, or the fungal infection is caused by, Rhizopus orvzae. In some embodiments, the fungus is, or the fungal infection is caused by, Lichtheimia spp. In some embodiments, the fungus is, or the fungal infection is caused by, Lichtheimia corymbifera. In some embodiments, the fungus is, or the fungal infection is caused by, Rhizomrucor spp. In some embodiments, the fungus is, or the fungal infection is caused by, Candida spp. In some embodiments, the fungus is, or the fungal infection is caused by, Candida albicans. In some embodiments, the fungus is, or the fungal infection is caused by, Candida glabrata. In some embodiments, the fungus is, or the fungal infection is caused by, Candida tropicalis. In some embodiments, the fungus is, or the fungal infection is caused by, Candida krusei. In some embodiments, the fungus is, or the fungal infection is caused by, Candida auris. In some embodiments, the fungus is, or the fungal infection is caused by, Cryptococcus spp. In some embodiments, the fungus is, or the fungal infection is caused by, Cryptococcus neoformans. In some embodiments, the fungus is, or the fungal infection is caused by, Chrysosporium parvum. In some embodiments, the fungus is, or the fungal infection is caused by, Metarhizium anisopliae. In some embodiments, the fungus is, or the fungal infection is caused by, Phaeoisaria clematidis. In some embodiments, the fungus is, or the fungal infection is caused by Sarcopodium oculorum.

Additional pathogenic fungi include the genus Candida (examples include C. albicans, C. glabrata, C. krusei, C. tropicalis, C. guilliermondii, C. parapsilosis, C. dubliniensis and C. auris), the genus Cryptococcus (examples include C. neoformans and C. gatti), the genus Trichosporon (examples include T. asahii, T. asteroides, T. cutaneum, T. dermatis, T. dohaense, T. inkin, T. loubieri, T. mucoides, and T. ovoides), the genus Malassezia (examples include M. globose and M. restricta), the genus Aspergillus (examples include A. fumigatus, A. flavus, A. terreus and A. niger), the genus Fusarium (examples include F. solani, F. falciforme, F. oxysporum, F. verticillioides, and F. proliferatum), the genus Mucor (examples include M. circinelloides, M. ramosissimus, M. indicus, M. rasemosus, and M. piriformis), the genus Blastomyces (examples include B. dermatitidis and B. brasiliensis), the genus Coccidioides (examples include C. immitis, and C. posadasii), the genus Pneumocystis (examples include P. carinii and P. jiroveci), the genus Histoplasma (examples include H. capsulatum), the genus Trichophyton (examples include T. schoenleinii, T. mentagrophytes, T. verrucosum, and T. rubrum), the genus Rhizopus (examples include R. oryzae and R. stolonifera), the genus Apophysomyces (examples include A. variabilis), the genus Rhizomucor (examples include R. pusillus, R. regularior, and R. chlamydosporus), the genus Lichtheimia (examples include L. ramose and L. corymbifera), the genus Scedosporium (examples include S. apiospermum), and the genus Lomentospora (examples include L. prolificans).

In some embodiments, the fungi is Mucorales (for which conventional therapy results are poor), and other lethal pathogens for which current therapy is poor or lacking (Fusarium, Scedosporium, Lomentospora, Acremonium, and Exserohilum).

In some embodiments, the fungal species is resistant to a therapeutic agent. In some embodiments, the fungal species is resistant to an azole. In some embodiments, the fungal species is resistant to an echinocandin. In some embodiments, the fungal species is CAS-resistant. In some embodiments, the fungal species is VOR-resistant.

In some embodiments, suitable dosage ranges for intravenous (i.v.) administration are 0.01 mg to 500 mg per kg body weight, 0.1 mg to 100 mg per kg body weight, 1 mg to 50 mg per kg body weight, or 10 mg to 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of 0.001 mg to 200 mg per kg of body weight, 0.01 mg to 100 mg per kg of body weight, 0.1 mg to 50 mg per kg of body weight, or 1 mg to 20 mg per kg of body weight. Effective doses may be extrapolated from dose-response curves derived from in vitro or animal model test systems. Such animal models and systems are well known in the art.

The compounds described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds described herein (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. The selection of the specific route of administration and the dose regimen is to be adjusted or titrated by the clinician according to methods known to the clinician to obtain the desired clinical response. The amount of compounds described herein to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The amount of a compound described herein that will be effective in the treatment and/or prevention of a fungal infection will depend on the nature of the fungal infection, and can be determined by standard clinical techniques. In addition, in vitro or in vivo assays may optionally be employed to help identify optimal dosage ranges. The precise dose to be employed in the compositions will also depend on the route of administration, and the seriousness of the fungal infection, and should be decided according to the judgment of the practitioner and each patient's circumstances. However, a suitable dosage range for oral administration is, generally, from about 0.001 milligram to about 200 milligrams per kilogram body weight. In some embodiments, the oral dose is from about 0.01 milligram to 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

The compounds and compositions disclosed herein can be evaluated for efficacy and toxicity using known methods. For example, the toxicology of a particular compound or a subset of the compounds sharing certain chemical moieties, or a composition thereof, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs, or monkeys, may be determined using known methods. Efficacy may be established using several recognized methods, such as in vitro methods, animal models, or human clinical trials. When selecting a model to determine efficacy, the skilled artisan can be guided by the state of the art to choose an appropriate model, dose, route of administration and/or regime.

A wide range of second therapies may be used in conjunction with the compounds of the present disclosure. The second therapy may be administration of an additional therapeutic agent or may be a second therapy not connected to administration of another agent. Such second therapies include, but are not limited to, surgery, immunotherapy, radiotherapy.

The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months.

In some embodiments, a therapeutically effective amount of a compound disclosed herein, or compositions thereof, is administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time or separated by a time interval, wherein one composition includes a compound of this invention, and the other includes the at least one additional therapeutic agent.

### 5. Kits

In another aspect, the disclosure provides kits comprising at least one disclosed compound or a pharmaceutically acceptable salt thereof, or a composition comprising the compound or a pharmaceutically acceptable salt thereof, and instructions for using the compound or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed compound and/or product and another agent for delivery to a patient.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like. Individual member components of the kits may be physically packaged together or separately.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example 1

### A. Fumigatus FKBP12-Bound FK520 X-Ray Crystal Structure Reveals Conserved Binding Pocket Structure

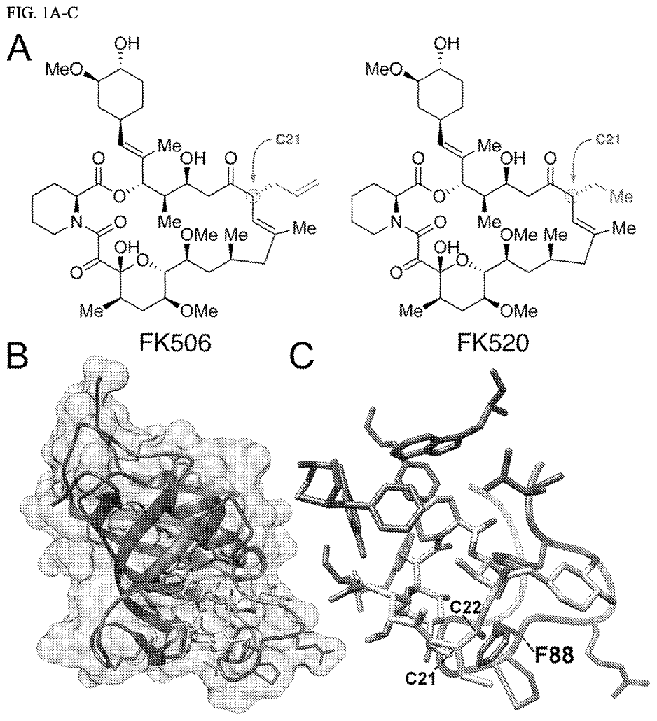

X-ray crystal structures of FKBP12-FK506-calcineurin ternary complexes from divergent human-pathogenic fungi were previously solved (see, Juvvadi P R, et al., 2019, Nat Commun 10:4275). FK506 and FK520 are structurally similar but differ at the C-21 position, where FK520 lacks the terminal alkene of FK506 (FIG. 1A). To gain better insight into the interactions between FK520 and fungal FKBP12, the X-ray crystal structure at 1.7 Å of A. fumigatus FKBP12 bound to FK520 was first determined. A. fumigatus was selected for FKBP12 structure analysis due to the readiness with which protein crystals form and the presence of key conserved fungal residues in the 80s loop. The FKBP12-binding motif of FK506 and FK520 is conserved and thus FK520 binds identically to A. fumigatus FKBP12 (FIGS. 1B and C). It has been previously shown that the 80s loop of FKBP12 in fungi is a critical region for facilitating the formation of FKBP12-inhibitor-calcineurin complex (see, Juvvadi P R, et al., 2019, Nat Commun 10:4275). Phe88 is a conserved residue in many pathogenic fungi that differs from the mammalian His88 residue. Both C-21 and C-22 of FK520/FK506 converge on this differential residue within 5 to 8 Å and represent excellent targets for synthetic modification.

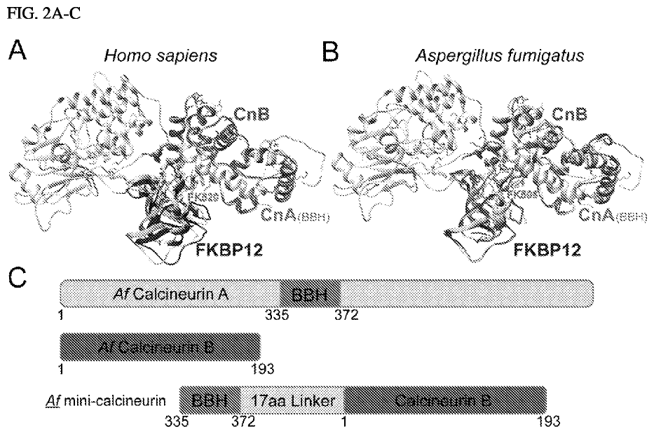

Due to inherent challenges in forming the full-length ternary complex for crystallography, a novel “mini-calcineurin” was generated as an alternative approach to capture FK520 bound to human FKBP12 and calcineurin (see FIG. 2 in the supplemental material). The mini-calcineurin construct consists of the full-length CnB subunit fused C-terminally to the calcineurin B binding helix (BBH; A. fumigatus residues 335 to 370, Homo sapiens residues 337 to 372) of CnA, lacking the CnA catalytic domain present in previous structures. Constructs for both human and A. fumigatus were generated and high-resolution ternary structures of each protein bound to FK520 and FK506, respectively, were characterized (Table 1). Remarkably, overlays with bovine FKBP12-FK506-calcineurin reveal consistent conformation of the mini-calcineurin structure, demonstrating that the key molecular interactions between FKBP12-FK520 and calcineurin are largely attributable to the hydrophobic interface presented by CnB bound to the extended CnA alpha-helical arm. The root mean square deviation (RMSD) value for the overlay of the A. fumigatus mini-calcineurin was 0.90 Å, consistent with previously reported values of structural similarity (see, Juvvadi P R, et al., 2019, Nat Commun 10:4275). The human mini-calcineurin RMSD value was 0.57 Å, indicative of a high degree of structural similarity and that the truncation of CnA did not significantly alter the overall binding structure of the complex. Each of these novel FK520-bound structures represents a key addition to the tools necessary for structure-guided design of both FK506/FK520 analogs that show increased fungal selectivity.

TABLE 1
Crystallographic data and refinement statistics

| | | | Homo | | Aspergillus | |
|---|---|---|---|---|---|---|
| | Aspergillus | | sapiens | | fumigatus | |
| | fumigatus | | miniCN-FKBP12- | | miniCN-FKBP12- | |
| | FKBP12-FK520 | | FK520 | | FK520 | |
| Data name | PDB 7U0S | | PDB 7U0T | | PDB 7U0U | |
| Data collection | | | | | | |
| Wavelength (Å) | 0.97872 | | 0.9786 | | 0.9786 | |
| Space group | P 2₁ 2₁ 2 | | C 1 2 1 | | I 2 2 2 | |
| Cell dimensions | | | | | | |
| a, b, c (Å) | 89.81 91.47 35.53 | | 156.1 33.3 64.35 | | 75.01 83.7 137.58 | |
| α, β, γ (°) | 90.0 90.0 90.0 | | 90.0 103.8 90.0 | | 90.0 90.0 90.0 | |
| Resolution (Å) | 50-1.70 | (1.74-1.70)^{a} | 50-2.45 | (2.51-2.45) | 50-1.90 | (1.95-1.90) |
| No. of unique reflections | 32,881 | (2,347) | 11,961 | (757) | 34,390 | (2,485) |
| R_{merge} (%) | 6.6 | (53.0) | 7.1 | (37.0) | 4.8 | (57.0) |
| I/σ(I) | 13.78 | (2.90) | 13.22 | (2.64) | 19.94 | (2.34) |
| Completeness (%) | 99.5 | (99.2) | 97.8 | (85.0) | 99.6 | (98.5) |
| Redundancy | 4.8 | (4.9) | 3.5 | (2.7) | 5.9 | (4.6) |
| CC1/2 | 99.8 | (88.2) | 99.7 | (83.6) | 99.9 | (76.5) |
| Refinement | | | | | | |
| Resolution (Å) | 50-1.70 | | 50-2.45 | | 50-1.90 | |
| No. of unique reflections | 32,871 | | 11,959 | | 34,387 | |
| R_{work}/R_{free}^{b} | 0.168 | (0.233) | 0.203 | (0.274) | 0.169 | (0.246) |
| | 0.192 | (0.269) | 0.259 | (0.338) | 0.199 | (0.261) |
| No. of atoms | | | | | | |
| Protein | 1,845 | | 2,274 | | 2,415 | |
| Ligand | 194 | | 64 | | 79 | |
| Solvent/Water | 227 | | 62 | | 174 | |
| B factors (Å²) (overall) | | | | | | |
| Protein | 23.56 | | 51.35 | | 43.17 | |
| Ligand | 29.24 | | 31.23 | | 32.12 | |
| Solvent/Water | 39.25 | | 38.81 | | 41.99 | |
| RMSD | | | | | | |

| | | | |
|---|---|---|---|
| Bond lengths (Å) | 0.006 | 0.002 | 0.006 |
| Bond angles (°) | 0.94 | 0.59 | 0.85 |
| Ramachandran favored (%) | 97.84 | 97.94 | 98.34 |
| Ramachandran allowed (%) | 2.16 | 2.06 | 1.66 |
| Ramachandran outliers (%) | 0 | 0 | 0 |
| Rotamer outliers (%) | 1.02 | 1.26 | 1.52 |

RMSD, root-mean-square deviation.
^{a}Highest-resolution shell shown in parentheses.
^{b}R_{free} was calculated with 10% of the data

### Chemical Synthesis of Novel FK506 and FK520 Analogs Modified at the C-22 Position.

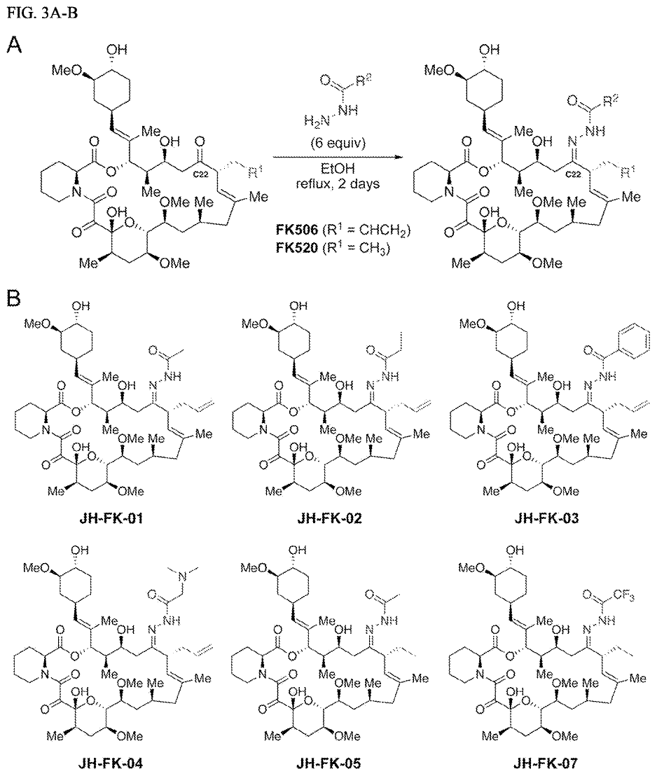

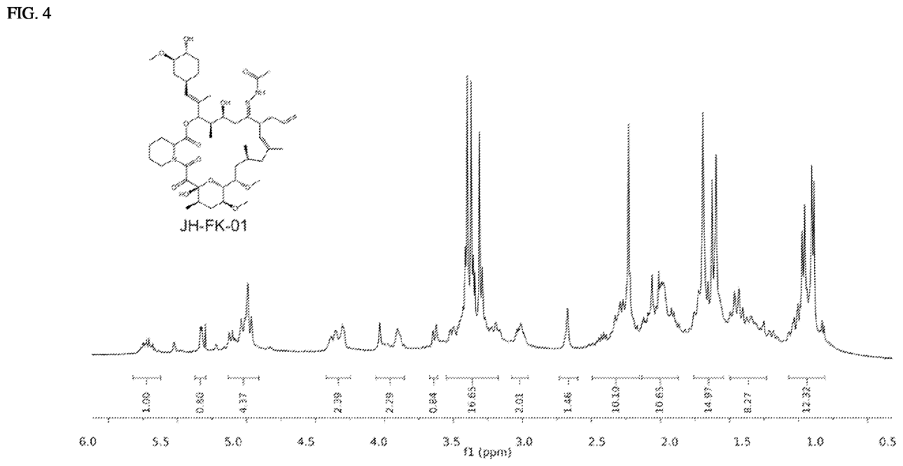

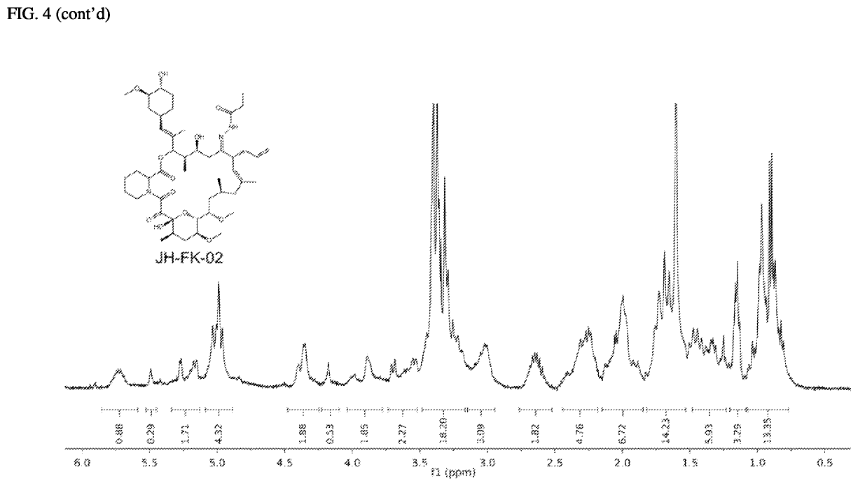

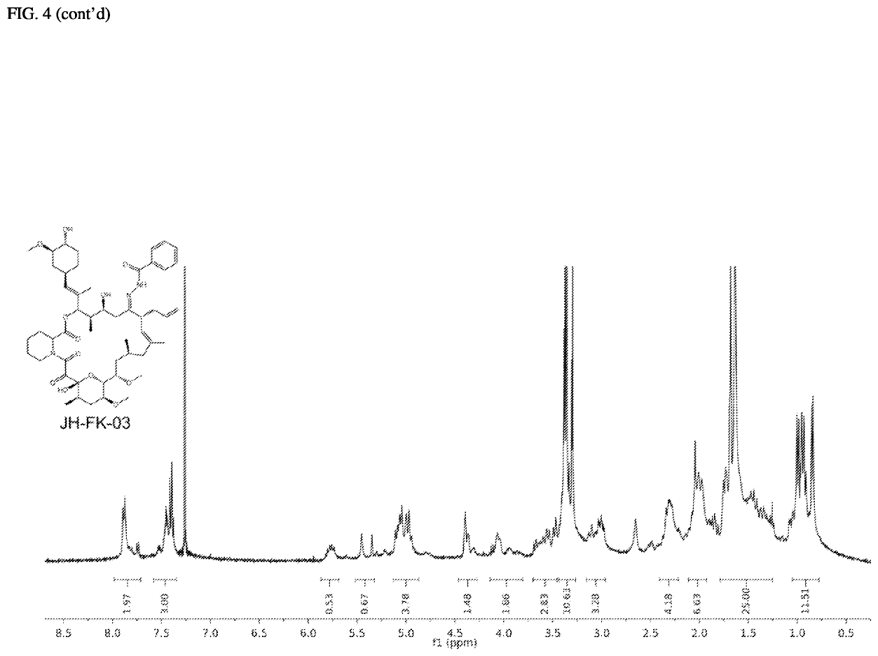

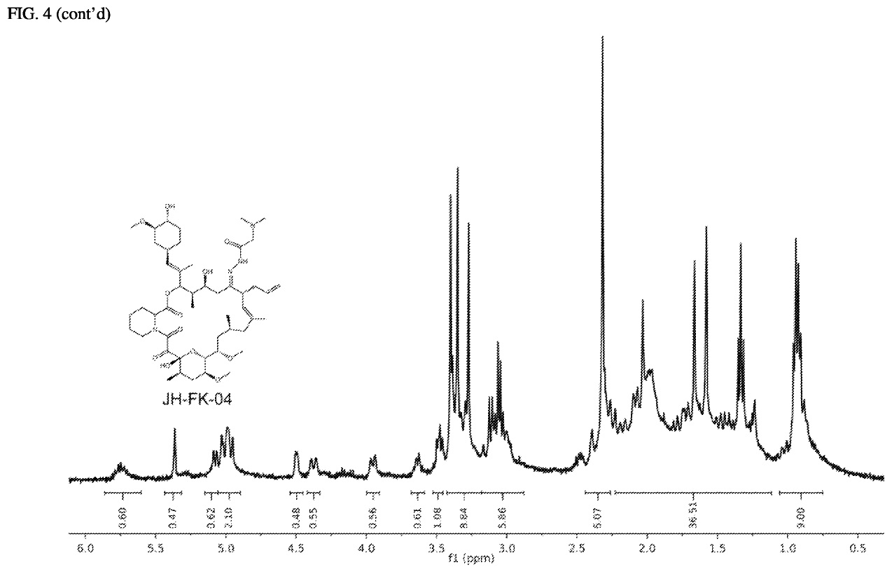

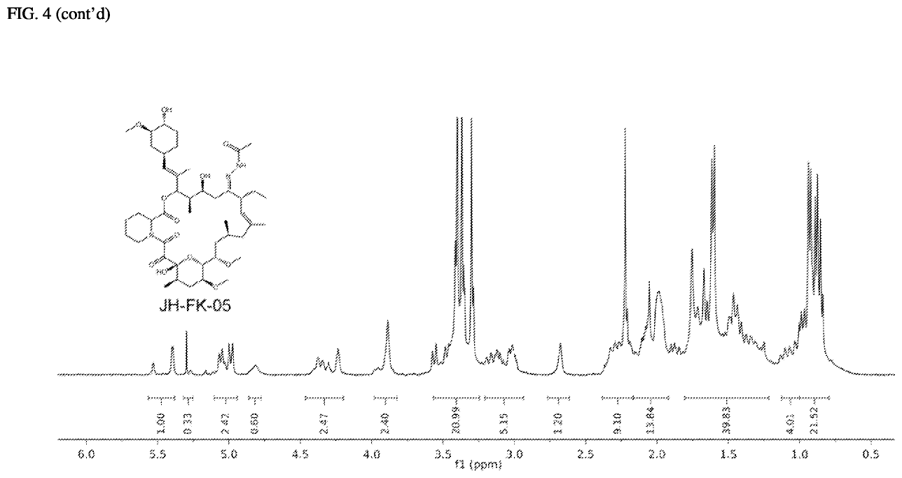

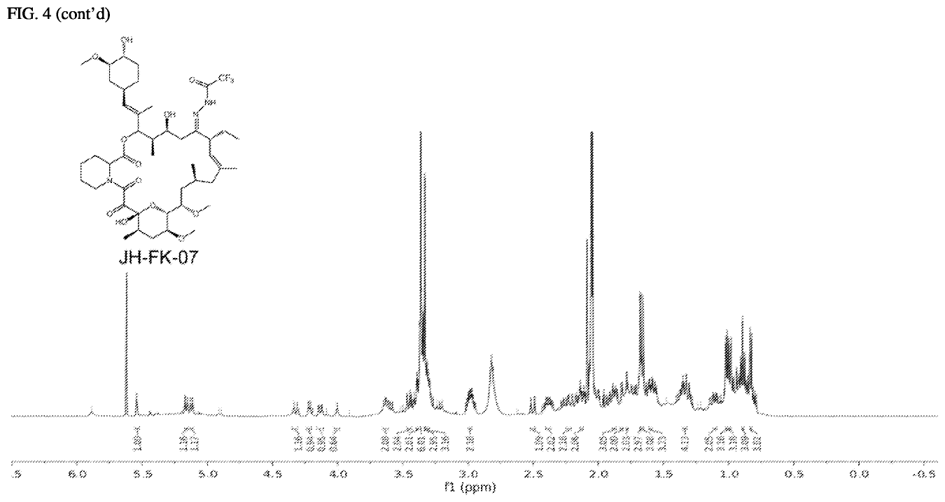

Our previous study demonstrated that C-22 modifications of FK506 can introduce fungal selectivity (see, Juvvadi P R, et al., 2019, Nat Commun 10:4275). To leverage the protein crystal structures of FKBP12 bound to both FK506 and FK520, a one-step synthetic protocol for C-22 modification compatible with either starting material was developed (FIG. 3A). The C-22 position of FK506 and FK520 can be selectively targeted through an easily scalable and high-yield condensation reaction with an acylhydrazone under reflux conditions. Inspired by the success of the first-generation FK506 analog APX879, this compound (JH-FK-0) was resynthesized and generated five additional C-22-modified compounds (FIG. 3B). Of these five second-generation analogs, three were derived from FK506 (JH-FK-02, JH-FK-03, and JH-FK-04) and two were derived from FK520 (JH-MK-05 and JH-MK-07). Each analog was validated with high-resolution mass spectrometry (HRMS) and ¹H1 nuclear magnetic resonance (NMR) (Table 2 and FIG. 4).

TABLE 2
High-resolution mass spectrometry (HRMS) data for FK506/FK520 analogs

| | Elemental | Calculated | Observed | Error | |
|---|---|---|---|---|---|
| | Composition | Mass | Mass | (ppm) | Ion |
| JH-FK-01 | C₄₆H₇₃N₃O₁₂ | 860.5267 | 860.5276 | −1 | [M + H]+ |
| | | 882.5087 | 882.5091 | −0.5 | [M + Na]+ |
| JH-FK-02 | C₄₇H₇₅N₃O₁₂ | 874.5424 | 874.5429 | −0.6 | [M + H]+ |
| | | 896.5243 | 896.5247 | −0.4 | [M + Na]+ |
| JH-FK-03 | C₅₁H₇₅N₃O₁₂ | 922.5424 | 922.5416 | 0.8 | [M + H]+ |
| | | 944.5243 | 944.5236 | 0.8 | [M + Na]+ |
| JH-FK-04 | C₄₈H₇₈N₄O₁₂ | 903.5689 | 903.5697 | −0.9 | [M + H]+ |
| | | 925.5509 | 925.5507 | 0.1 | [M + Na]+ |
| JH-FK-05 | C₄₅H₇₃N₃O₁₂ | 848.5267 | 848.5273 | −0.7 | [M + H]+ |
| | | 870.5087 | 870.5089 | −0.3 | [M + Na]+ |
| JH-FK-07 | C₄₅H₇₀F₃N₃O₁₂ | 902.0592 | 902.498 | 0.5 | [M + H]+ |
| | | 924.4804 | 924.4792 | 1.3 | [M + Na]+ |

### Second-Generation FK506 and FK520 Analogs Display Broad-Spectrum Antifungal Activity.

MICs/minimum effective concentrations (MECs) were determined for each of the novel analogs against the three major human fungal pathogens, A. fumigatus, C. neoformans, and C. albicans. Although all six analogs showed reduced antifungal activity compared to that of FK506, they were each effective at inhibiting growth in broth microdilution assays (Table 3). Along with their robust immunosuppressive activity, FK506 and FK520 exhibited remarkably potent antifungal activity in the range of 0.016 to 0.05 μg/mL, which is far below the single-digit micrograms per milliliter MIC threshold typically necessary to translate to in vivo antifungal efficacy. Thus, analogs of FK506 and FK520 can tolerate up to 100-fold reduction in antifungal activity and still be considered for in vivo testing. For C. neoformans, MICs were the most uniform and ranged from 0.2 to 2 μg/mL, which is between 4- and 40-fold reduced for antifungal activity. C. albicans MICs ranged from 0.2 to 6.25 g/mL, which is between 10- and 312.5-fold reduced for activity. Despite being one of the most difficult to treat fungal infections, all six analogs were active against A. fumigatus with MECs ranging from 1 to 2 μg/mL or a 62.5- to 125-fold reduction in activity.

TABLE 3
MIC/MECs for FK506, FK520, and their analogs

| | MIC (μg/mL) against: | | MEC (μg/mL) against: |
|---|---|---|---|
| | Cryptococcus | Candida | Aspergillus |
| Drug | neoformans | albicans | fumigatus |
| candidate | (H99)^{a} | (SC5314)^{b} | (Af293) |
| FK506 | 0.05 | 0.02 | 0.016 |
| FK520 | 0.05 | 0.02 | —^{c} |
| JH-FK-01 | 1 | 0.4 | 1 |
| JH-FK-02 | 2 | 1.6 | 2 |
| JH-FK-03 | 1 | 0.8 | 1 |
| JH-FK-04 | 2 | 6.25 | 2 |
| JH-FK-05 | 1 | 0.8 | 2 |
| JH-FK-07 | 0.2 | 0.2 | 1.25 |

^{a}C. neoformans MICs determined at 37° C.
^{b}C. albicans MICs determined in the presence of 1 μg/mL fluconazole.
^{c}—, Not tested.

Two FK520 analogs, JH-FK-05 and JH-FK-07, maintained high levels of broad-spectrum antifungal activity across the three species, and JH-FK-07 had the lowest MICs for both C. neoformans and C. albicans. To assess the antifungal activity against other human fungal pathogens, JH-FK-05 was selected to test against a panel of pathogenic molds and drug-resistant A. fumigatus strains (Table 4). JH-FK-05 registered MECs as low as 1 μg/mL for some of these highly recalcitrant species, such as Mucor circinelloides, Rhizopus oryzae, and pan azole-resistant A. fumigatus (strain F14946).

TABLE 4
Antifungal activity of JH-FK-05 against molds.

| | | MIC/MEC |
|---|---|---|
| Species | Strain | (μg/mL) |
| Aspergillus fumigatus (Wild-type) | (akuBKu80) | 1 |
| Aspergillus fumigatus | (akuBKu80 | >8 |
| | hFKBP12) | |
| Azole-Resistant Strains | | |
| Aspergillus fumigatus | F12776 | 2 |
| Aspergillus fumigatus | F14946 | 1 |
| Aspergillus fumigatus | F16314 | 1 |
| Aspergillus fumigatus | F16216 | 1 |
| Aspergillus fumigatus | F7075 | 2 |
| Echinocandin-Resistant Strain | | |
| Aspergillus fumigatus | EMFR-S678P | 2 |
| Other Aspergillus Species | | |
| Aspergillus calidoustus | WT | >8 |
| Aspergillus flavus | WT | 1 |
| Aspergillus niger | WT | <1 |
| Aspergillus terreus | WT | 1 |
| Other molds | | |
| Fusarium solani | WT | >8 |
| Mucor circinelloides | WT | 1 |
| Paecilomyces variotti | WT | >8 |
| Rhizomucor pusilus | WT | >8 |
| Rhizopus oryzae | WT | 1 |
| Scedosporium apiospermum | WT | >8 |
| Scedosporium prolificans | WT | >8 |

To confirm that JH-FK-05 was fungal FKBP12-dependent, a humanized A. fumigatus strain (the akuB^{Ku80} hFKBP12 strain) expressing a codon-optimized human FKBP12 gene from the Affkbp12 native locus was utilized. It has been previously shown that this hFKBP12-expressing strain is completely resistant to FK506 and that the activity of FK506 in Aspergillus is dependent on the presence of an FKBP12 that can form the FKBP12-inhibitor-calcineurin complex (see, Juvvadi P R, et al., 2019, Nat Commun 10:4275). Similar to FK506, JH-FK-05 has no detectable antifungal activity against the hFKBP12-expressing A. fumigatus strain, suggesting that JH-FK-05 works via the same mechanism of action (Table 4).

FK506 and FK520 Analogs Bind FKBP12 and Form a Ternary Inhibitory Complex with Calcineurin.

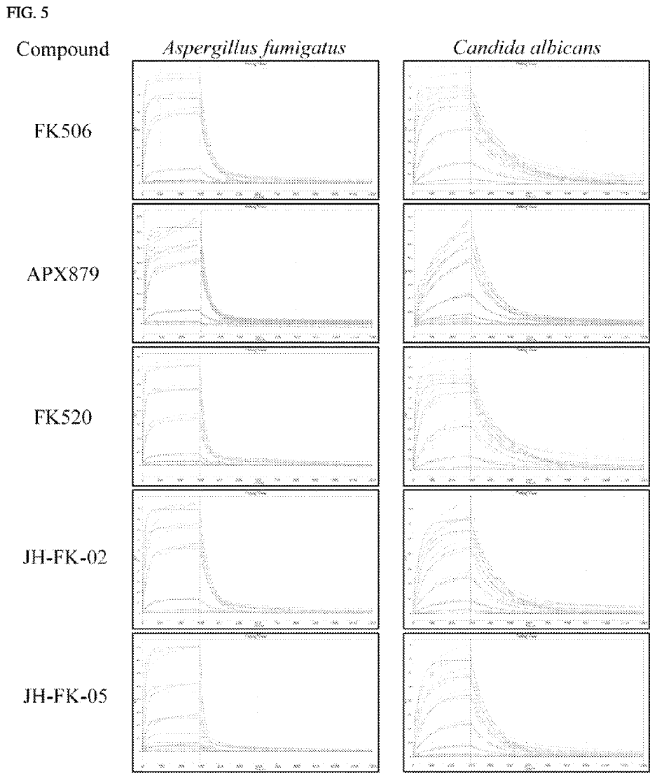

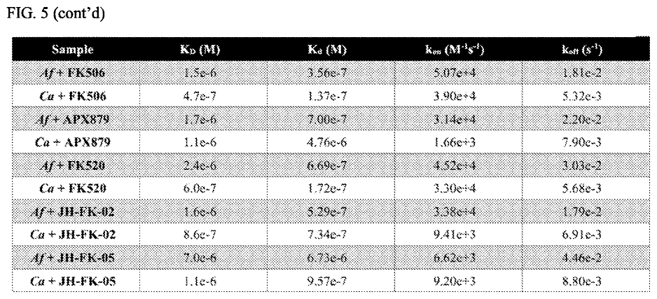

To better understand the relationship between structure and activity of these analogs, FK506 analog, JH-FK-02 was tested, and one FK520 analog, JH-FK-05 was tested, for their binding dynamics to CnA and CnB by biolayer interferometry (BLI). Purified and biotinylated (Avi-tagged) FKBP12 from A. fumigatus and C. albicans were loaded onto streptavidin sensors in the presence of an inhibitor, followed by titration of the respective CnA-CnB-calmodulin complex, also in the presence of an inhibitor. Data were double referenced using parallel reference sensors and sample blanks. Steady-state and kinetic analyses confirm that the target complexes form in the presence of the analogs with affinities similar to those of the parent compounds, suggesting that the in vivo observations are due to inhibitory effects of the compound-mediated calcineurin-FKBP12 complexes (FIG. 5).

### Some Second-Generation FK506 and FK520 Analogs Show Increased Fungal Specificity.

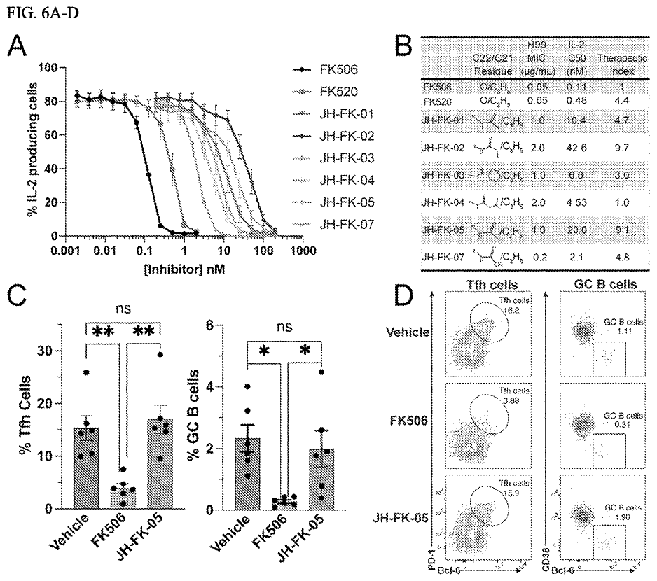

The potent immunosuppressive activity of FK506 and FK520 is what precludes these highly antifungal compounds from being utilized clinically to treat invasive fungal infections. Therefore, it is essential to understand the level of immunosuppressive activity for each of these analogs to determine if these C-22 modifications improve the fungal selectivity. Utilizing a primary murine T-cell model, IL-2 expression was assessed following growth in the presence of calcineurin inhibitors (FIG. 6A). Naive, primary CD4⁺ T cells were collected from mice and grown for 72 h in the presence of FK506, FK520, or an analog. Following stimulation with phorbol 12-myristate 13-acetate (PMA) and ionomycin, the proportion of cells producing IL-2, a key calcineurin-dependent cytokine, was measured with flow cytometry. All compounds generated a dose-dependent reduction in IL-2 production. FK506 and FK520 demonstrated the highest level of IL-2 inhibition, and the effect of FK520 on IL-2 production was slightly reduced compared to that of FK506. JH-FK-02 and JH-FK-05 were the least immunosuppressive second-generation analogs, with 50% inhibitory concentration (IC₅₀) values of 42.6 nM (>380-fold increase compared to FK506) and 20.0 nM (>180-fold increase compared to FK506), respectively (FIG. 6A).

To assess the therapeutic potential for these compounds, it was important to develop an approach to score and rank them based on antifungal and immunosuppressive activities. The therapeutic index score (TI_{score}) is a ratio of the immunosuppressive fold change to the antifungal fold change, with all values relative to the baseline FK506 activity (FIG. 6B). A TI_{score} of >1.0 indicates an increase in fungal specificity because the fold change of the immunosuppressive reduction is greater than the antifungal activity reduction. As an example, JH-FK-05 is 20-fold reduced for antifungal activity but about 180-fold reduced for immunosuppression. Therefore, the TI_{score} of JH-FK-05 is 9.1 (TI_{score}=182/20). The top two analogs were JH-FK-02 (TI_{score}=9.7) and JH-FK-05 (TI_{score}=9.1). Due to the similar TI_{score}, JH-FK-05 was prioritized to advance to animal trials on the merit of more pronounced antifungal activity compared to JH-FK-02.

### JH-FK-05 is Nonimmunosuppressive in a Murine Model of T Cell-Dependent Response.

Based on the in vitro evidence of reduced immunosuppressive activity of JH-FK-05, it was hypothesized that this reduction would translate into reduced immunosuppression in an in vivo model. The murine model for immunosuppression measures the T cell-dependent proliferation of two immune cell subtypes in response to immunization with an antigen. Animals were treated daily via intraperitoneal (i.p.) injection with vehicle, 5 mg/kg FK506, or 60 mg/kg JH-FK-05, beginning on day −1 and ending on day 7. JH-FK-05 treatment was tolerated at a dose as high as 60 mg/kg, and the final dose of JH-FK-05 was selected based on the maximum compound solubility (data not shown). On day 0, animals were immunized with NP-OVA to stimulate T cell-dependent signaling that would result in proliferation of T follicular helper (Tfh) cells and germinal center (GC) B cells. Animals were sacrificed on day 7, and the proportions of Tfh and GC B cells in the lymph nodes were measured. Treatment with 5 mg/kg of FK506 significantly reduced the proliferation of both Tfh and GC B cells (FIG. 6B). However, despite the much larger daily dose of 60 mg/kg, JH-FK-05 treatment did not reduce the presence of either cell type and was statistically indistinguishable from the vehicle treatment. Representative flow cytometry plots capture the lack of both immune cell subtypes in the FK506-treated animals only (FIG. 6D). In addition to its lack of immunosuppressive activity in vivo, JH-FK-05 was well tolerated by animals throughout the course of treatment.

### JH-FK-05 Reduces Pulmonary and Brain Fungal Burden in Models of Cryptococcosis.

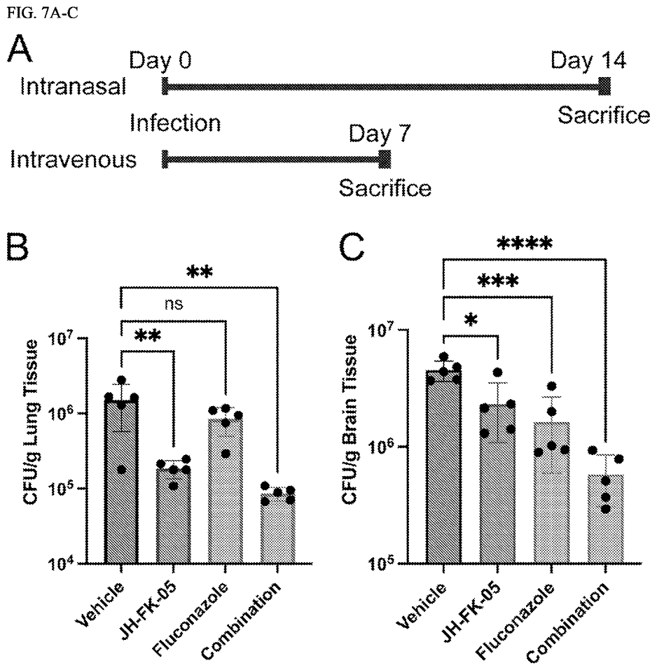

The high in vitro antifungal activity of JH-FK-05 presented promising evidence that it might effectively reduce fungal burden in the context of murine infection. Two models of C. neoformans infection were employed to test this hypothesis (FIG. 7A). First, in the intranasal model, animals were anesthetized and infected with 10⁵ cells of wild-type H99 C. neoformans via instillation in the animal's nares. These animals were monitored for 14 days and then sacrificed. This model tests the efficacy of JH-FK-05 to treat a pulmonary infection, which is the most common manifestation of early-stage cryptococcosis in patients. Second, the intravenous (i.v.) model approximates the disseminated stage of disease. Animals were infected with 10⁴ cells via injection into the lateral tail vein. Due to the advanced nature of the model and the rapidly disseminated disease, animals were sacrificed at 7 days and found to have a substantial central nervous system (CNS) fungal burden. In both models, animals were treated once daily via i.p. injection beginning 3 h after infection with either vehicle, 60 mg/kg JH-FK-05, 12 mg/kg fluconazole, or a combination of the two monotherapies. Fluconazole is a widely used antifungal drug targeting ergosterol biosynthesis for cell membrane integrity. Previous work has shown that calcineurin inhibitors are synergistic with fluconazole (see, Del Poeta M, et al., 2000, Antimicrob Agents Chemother 44:739-746; Lee Y, et al., 2018, Antimicrob Agents Chemother 62:e01627-18). Additionally, the first-generation FK506 analog, APX879, benefits from this synergistic interaction when analyzed in vivo in combination therapy with fluconazole in the pulmonary cryptococcosis model (see, Juvvadi P R, et al., 2019, Nat Commun 10:4275).

Like the immunosuppressive murine model, animals tolerated JH-FK-05 treatment for the full treatment period. Analysis of lung tissue collected from animals at day 14 showed that JH-FK-05 treatment alone reduced fungal burden by nearly 10-fold compared to that in vehicle-treated animals (FIG. 7B). The subtherapeutic dose of fluconazole alone did not reduce the fungal burden but combination therapy with JH-FK-05 significantly reduced the lung fungal burden by over 10-fold. To assess if JH-FK-05 treatment could display therapeutic levels of activity after the infection had fully disseminated, the intravenous model was employed. A modest but significant 2-fold reduction (P<0.05) in brain fungal burden was detected in animals treated with JH-FK-05 alone (FIG. 7C). However, in combination, JH-FK-05 and fluconazole treatment resulted in a nearly 10-fold reduction in fungal burden after only 7 days of treatment. This demonstrates that not only can JH-FK-05 treat an aggressive pulmonary-stage infection but it can cross the blood-brain barrier to directly treat a disseminated CNS infection. This is in accord with previous studies and the known pharmacodynamic properties of FK506, which is known to cross the blood brain barrier (see, Odom A, Del Poeta M, Perfect J, Heitman J. 1997, Antimicrob Agents Chemother 41:156-161). Together, these results demonstrate that JH-FK-05 is effective at reducing fungal burden in multiple stages of C. neoformans infection, both as a monotherapy and in combination with the FDA-approved antifungal drug fluconazole.

Treatment with JH-FK-05 in Murine C. neoformans Infection Extends Survival.

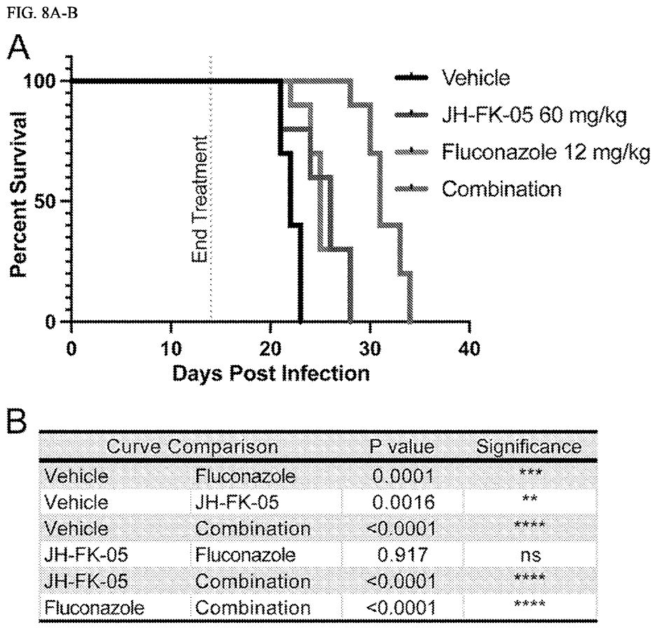

To verify if this reduction seen in the fungal burdens of JH-FK-05 and combination-treated animals could translate into a meaningful therapeutic effect, the survival of animals infected via the intranasal route was assessed (FIG. 8A). As in the fungal burden model, these animals received daily treatment for only 14 days. Following the termination of treatment, animals were monitored for signs of decline, lethargy, and ultimately, moribundity. Untreated animals in this model typically survive for about 3 weeks, and similar results were observed here (vehicle treatment median survival, 22 days). Corroborating the results of the fungal burden study, a significant extension of survival for both JH-FK-05 (median survival, 26 days) and fluconazole (median survival, 25 days) was observed. As with the fungal burden analysis, combination of JH-FK-05 and fluconazole had a potent therapeutic effect and extended survival of these animals to a maximum of 34 days (median survival, 31 days). Both monotherapy survival curves were significantly different from the combination therapy, suggesting an additive or synergistic effect in vivo (FIG. 8B).

### Molecular Dynamic Simulations Identify Novel JH-FK-05 Residues for Future Medicinal Chemistry.

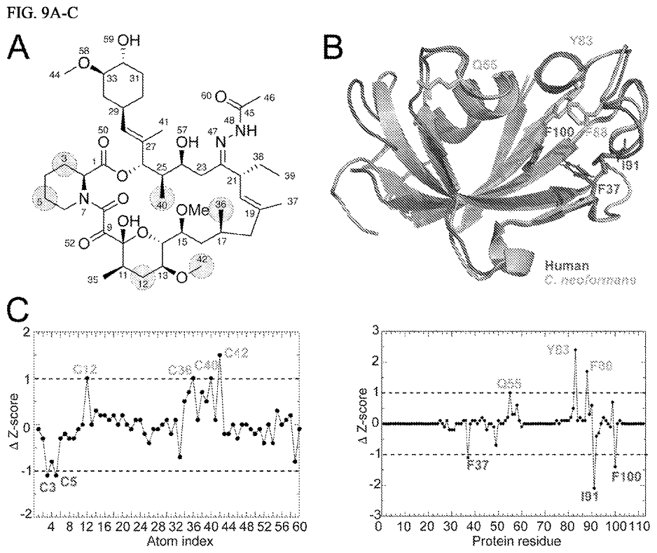

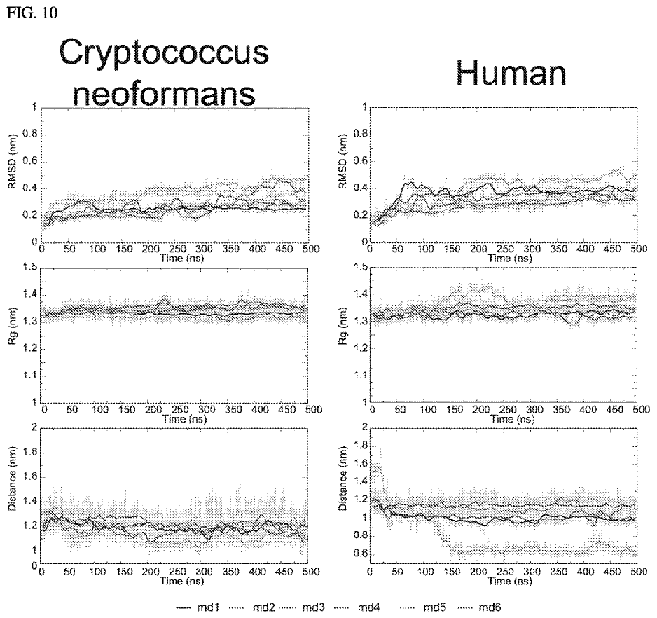

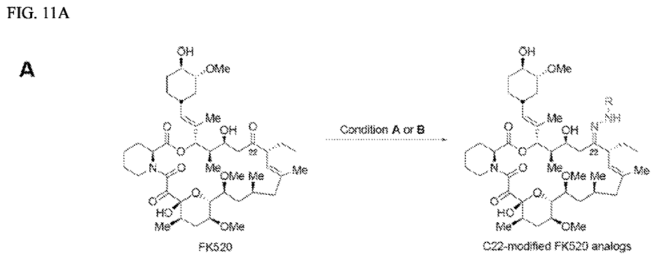

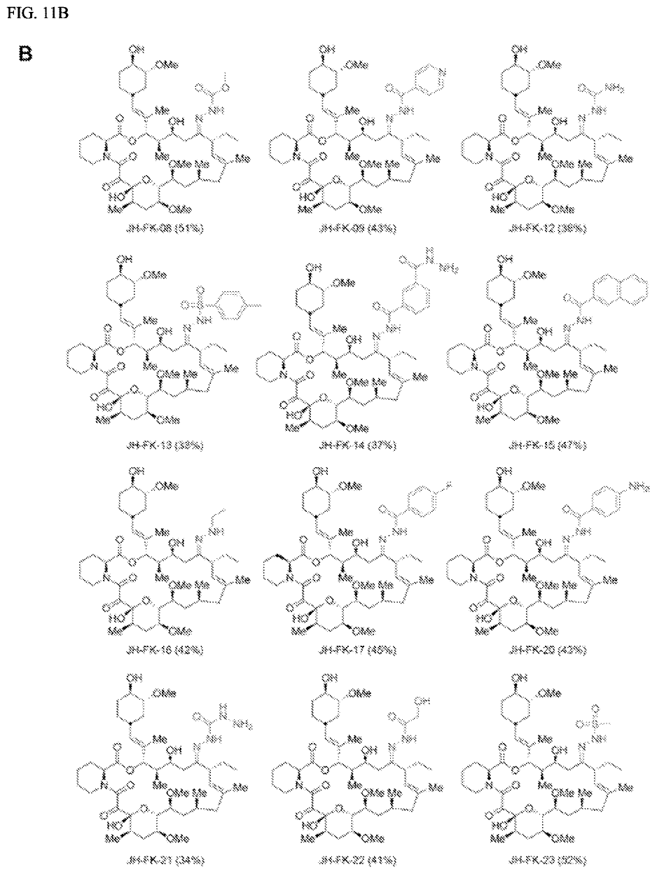

Although JH-FK-05 has demonstrated robust in vitro and in vivo antifungal activity, additional improvements can be made to elevate the TI_{score} of this compound. To address this, molecular dynamic (MD) simulations of JH-FK-05 physical interactions with human versus fungal FKBP12 were conducted to identify contact residues that are more important for either species (FIG. 9) (see, Gobeil S M, et al., 2021, mBio 12:e0300021). A series of 500-ns MD simulations between hFKBP12 and JH-FK-05 or C. neoformans FKBP12 and JH-FK-05 were simulated, and differential contacts between JH-FK-05 and the two FKBP12 proteins were observed. Simulation stability was monitored with alpha carbon (C-α) RMSD, radius of gyration, and center of mass distance (FIG. 10). Both the significant protein residues and the JH-FK-05 molecular residues that were identified are labeled on the respective structures (FIG. 9 and Table 5). There were 4 residues of JH-FK-05 that were more significant for C. neoformans contact (FIG. 9, cyan label). Interestingly, there were only 2 residues that were more significant for the JH-FK-05 and human FKBP12 (hFKBP12) interaction (FIG. 9, magenta labels). These residues are both in the C-2-N-7 ring of the molecule and represent potential future target areas for medicinal chemistry to introduce further fungal specificity to JH-FK-05 and design a new generation of analogs.

TABLE 5
Atom index for JH-FK-05 MD analysis.

| Index | Type | Atoms |
|---|---|---|
| 1 | C | C01 |
| 2 | CH | C02/H02 |
| 3 | CH₂ | C03/H03/H44 |
| 4 | CH₂ | C04/H04/H45 |
| 5 | CH₂ | C05/H05/H46 |
| 6 | CH₂ | C06/H06/H47 |
| 7 | N | N45 |
| 8 | C | C07 |
| 9 | C | C08 |
| 10 | C | C09 |
| 11 | CH | C10/H07 |
| 12 | CH₂ | C11/H08/H48 |
| 13 | CH | C12/H09 |
| 14 | CH | C13/H10 |
| 15 | CH | C14/H11 |
| 16 | CH₂ | C15/H12/H49 |
| 17 | CH | C16/H13 |
| 18 | CH₂ | C17/H14/H50 |
| 19 | C | C18 |
| 20 | CH | C19/H15 |
| 21 | CH | C20/H16 |
| 22 | C | C21 |
| 23 | CH₂ | C22/H17/H51 |
| 24 | CH | C23/H18 |
| 25 | CH | C24/H19 |
| 26 | CH | C25/H20 |
| 27 | C | C26 |
| 28 | CH | C27/H21 |
| 29 | CH | C28/H22 |
| 30 | CH₂ | C29/H23/H52 |
| 31 | CH | C30/H24 |
| 32 | CH | C31/H25 |
| 33 | CH₂ | C32/H26/H53 |
| 34 | CH₂ | C33/H27/H54 |
| 35 | CH₃ | C34/H28/H55/H66 |
| 36 | CH₃ | C35/H29/H56/H67 |
| 37 | CH₃ | C36/H30/H57/H68 |
| 38 | CH₂ | C37/H31/H58 |
| 39 | CH₃ | C38/H32/H59/H33 |
| 40 | CH₃ | C40/H34/H60/H69 |
| 41 | CH₃ | C41/H35/H61/H70 |
| 42 | CH₃ | C42/H36/H62/H71 |
| 43 | CH₃ | C43/H37/H63/H72 |
| 44 | CH₃ | C44/H38/H64/H73 |
| 45 | C | C59 |
| 46 | CH₃ | C60/H01/H43/H65 |
| 47 | N | N54 |
| 48 | NH | N55/H40 |
| 49 | O | O46 |
| 50 | O | O47 |
| 51 | O | O48 |
| 52 | O | O49 |
| 53 | O | O50 |
| 54 | OH | O51/H39 |
| 55 | O | O52 |
| 56 | O | O53 |
| 57 | OH | O56/H41 |
| 58 | O | O57 |
| 59 | OH | O58/H42 |
| 60 | O | O61 |

### Example 2

This example provides the materials and methods related to Example 1.

Aspergillus fumigatus and Candida albicans Calcineurin A, Calcineurin B, and Calmodulin Complexes

C. albicans calcineurin subunit A with an N-term MGSSHHHHHHSSGENLYFQGS tag (Uniprot Q5ABP4 residues 54-609, CaalA.00174.a.EX11, CID11764), C. albicans calcineurin subunit B with no tag (Uniprot C₄YS24 residues 1-173, CaalA.01011.a.EX111), and C. albicans calmodulin with no tag (Uniprot P23286 residues 1-149, CaalA.00464.a.EX11, CID11763) were separately codon-optimized by ATUM for insect expression, flanked with unique SapI sites (with bases TGATAA prior to the 3′ SapI site) and cloned by ATUM via SapT into a customized version of pBacgus-1 (Millipore Sigma, formerly Novagen) which includes two SapI sites within the MCS. One E. coli TOP10 transformant was fully sequence-verified over the Open Reading Frame (ORF) and flanking regions and DNA from this clone subjected to SF9 transfection via Bestbac protocols.

A. fumigatus calcineurin subunit A with an N-term MGSSHHHHHHSSGENLYFQGS tag (SEQ ID NO: 1) (Uniprot Q4WUR1 residues 2-434, CID11448, AsfuA.00174.a.FW11), A. fumigatus subunit B with no tag (Uniprot Q4WDF2 residues 21-193, CID11452, AsfuA.01011.a.FX11), and A. fumigatus/Coccidioides immitis calmodulin (100% identity) with no tag (Uniprot J3KLB7 residues 1-149, CID11453, CoimA.00464.a.FX11) were separately codon-optimized by ATUM for insect expression, flanked with unique BamHI/HindIII sites (with TGATAA prior to the HindIII) and intermediate plasmids delivered in a kanamycin-resistant shuttle vector. Genes were released via BamHI/HindIII digestion and ligated into BamHI/HindIII-digested and gel-purified insect transfer vector pFastbac-I (Life Technologies). A resulting TOP10 transformant for each construct was miniprepped and DNA sequenced across the ORF and flanking regions. Upon validation this DNA was subsequently transposed into DH10Bac cells (Life Technologies) and screened by PCR to identify clones with the desired gene insert. Bacmid DNA was prepped via standard protocols (Life Technologies) from one clone positive for insert by PCR for each construct and the bacmid DNA subjected to SF9 transfection via Bac-to-Bac protocols (Life Technologies).

Calcineurin A, calcineurin B and calmodulin complexes were expressed and purified using similar methods to those previously described (34). In brief, for each species of complex, calcineurin A, calcineurin B and calmodulin were co-transfected into Tni insect cells grown in ESF921 medium (Expression Systems) at roughly a 1:1:1 mixture of viruses such that the final multiplicity of infection (MOI) was 2-3. Cultures were harvested 48-72 h post-infection. Cells were centrifuged and stored at −80° C. Cell pastes were lysed via hypotonic lysis in 25 mM Tris pH 8.0, 0.02% CHAPS, and protease inhibitor tablets, EDTA-free (Roche), and clarified before applying them to two 5 mL Ni²⁺ charged HiTrap Chelating HP (GE Healthcare) columns. The complex of His-tagged calcineurin A, calcineurin B and calmodulin was eluted with a gradient of 25 mM Tris, pH 8.0, 200 mM NaCl, 1 mM TCEP, and 500 mM Imidazole. Fractions containing the complex were pooled and diluted in buffer to lower the NaCl concentration down to 50 mM, then injected onto HiTrap SP-FF and HiTrap Q-FF (GE Healthcare) ion exchange columns arranged in tandem. After loading and washing in 25 mM Tris pH 8.0, 50 mM NaCl, 1 mM TCEP and 5 mM CaCl₂), the columns were separated and eluted using a gradient up to 1 M NaCl. The cleanest protein complex eluted from the HiTrap Q-FF column and was pooled and concentrated to 10-15 mg/mL. Each complex was then injected onto a 120 mL Sephacryl 5-300 column (GE Healthcare) equilibrated in 25 mM HEPES, pH 8.0, 50 mM NaCl, 0.5 mM TCEP, and 5 mM CaCl₂). Fractions containing the complex in 1:1:1 stoichiometry were pooled and concentrated to 10 mg/mL, then flash frozen in liquid nitrogen and stored at −80° C. (CaalA.00174.a.EX11 [calcineurin A], CaalA.01011.a.EX11 [calcineurin B], CaalA.00464.a.EX11 [calmodulin], Batch ID—PD00426; AsfuA.00174.a.FW11 [calcineurin A], AsfuA.01011.a.FX11 [calcineurin B], CoimA.00464.a.FX11 [calmodulin], Batch ID—PD00411).

Biotinylated A. Fumigatus FKBP12 P90G and H. sapiens FKBP12

A. fumigatus FKBP12 with N-term MSGSHHHHHHHHGGENLYFQGSGLNDIFEAQKIEWHEGSSGSS tag (SEQ ID NO: 2) (Uniprot Q4WLV6 residues 2-112 with P90G, AsfuA.18272.a.TM11) and Homo sapiens FKBP12 with N-term MSGSHHHHHHHHGGENLYFQGSGLNDIFEAQKIEWHEGSSGSS tag (SEQ ID NO: 2) (Uniprot P62942 residues 2-108, HosaA.18272.a.TM11) were separately codon-optimized for E. coli expression by Genscript, flanked with unique NdeI/XhoI (including stop codons TGATAA prior to XhoI) and delivered in an ampicillin-resistant shuttle vector. The inserts were released with NdeI/XhoI and ligated into NdeI/XhoI-digested and gel-purified pET28a (modified with an NdeI site replacing NcoI). Following transformation of the ligation one E. coli TOP10, one clone per construct was fully sequence-verified over the ORF and flanking regions and subsequently transformed into BL21(DE3) for expression studies.

Bio-AJFKBP12 and Bio-HsFKBP12 were each co-transformed with BirA in E. coli BL21 (DE3) cells and expression was induced with 1 mM IPTG (GoldBio). Cultures were grown for 16 h at 25° C. then harvested by centrifugation (Beckman) at 10,000×g for 15 min and pellets stored at −80° C. Cells were lysed by sonication (Misonix) in 25 mM Tris pH 8.0, 200 mM NaCl, 50 mM L-Arginine monohydrochloride, 0.02% CHAPS (3-[(3-Cholamidopropyl)dimethylammonio]-1-propanesulfonate), 1 mM TCEP (tris(2-carboxyethyl)phosphine) (VWR), 100 mg Lysozyme, 250 U Benzonase and 1 protease inhibitor tablet, EDTA free (Ethylenediaminetetraacetic acid) (Roche). Lysate was clarified by centrifugation at 42,000 RPM (Beckman) for 45 min at 4° C. and filtered with a 0.2-0.8 μm gradient filter. The supernatant was applied to a 5 mL Ni²⁺ charged HiTrap chelating HP (GE Healthcare) columns and the protein was eluted by step gradient of 25 mM Tris pH 8.0, 200 mM NaCl, 1 mM TCEP and 300 mM imidazole. Fractions containing FKBP12 were pooled and digested with TEV protease and dialyzed (3.5 kDa MWCO dialysis cassettes) into 25 mM Tris pH 8.0, 200 mM NaCl, 1 mM TCEP overnight at 4° C. to remove the N-terminal 8× histidine (confirmed by SDS-PAGE). The dialyzed and TEV cleaved target was then re-applied to a 5 mL Ni²⁺ charged HiTrap chelating HP and FKBP12 was collected in the flow-through and early wash fractions. Again, fractions containing FKBP12 were pooled and dialyzed into the final buffer containing 25 mM Tris pH 8.0, 200 mM NaCl, 1 mM TCEP. The final samples were concentrated to 18.2 mg/mL (Bio-AJFKBP12) and 7.1 mg/mL (Bio-HsFKBP12) and flash frozen in liquid nitrogen and stored at −80° C. (AsfuA.18272.a.TM11, Batch ID—PD38408; HosaA.18272.a.TM11, Batch ID—PD38410).

H. sapiens and A. Fumigatus Truncated Calcineurin a (Calcineurin B Binding Helix)—Calcineurin B Fusion Constructs

A. fumigatus calcineurin subunit A—subunit B fusion with N-term MSGSHHHHHHHHGGENLYFQGS tag (SEQ ID NO: 3) (Uniprot Q4WUR1 residues 335-370 fused with Uniprot Q4WDF2 residues 1-193 on either side of a GGGSSGGSTSGGSSGGG residue linker, AsfuA.00174.a.TQ11+AsfuA.01011.a.TR11), and Homo sapiens calcineurin subunit A—subunit B fusion with MSGSHHHHHHHHGGENLYFQGS tag (SEQ ID NO: 3) (Uniprot Q08209 residues 337-372 fused with Uniprot P63098 2-170 on either side of a GGGSSGGSTSGGSSGGG linker (SEQ ID NO: 4), HosaA.00174.a.TU11+HosaA.01011.a.TV11), were cloned as discussed for FKBP12.

AfCnA-CnB or HsCnA-CnB were transformed individually into E. coli BL21 (DE3) cells and expression was induced with 1 mM IPTG (GoldBio) at 18° C. or 37° C., respectively. The culture was grown for 16 h and harvested by centrifugation (Beckman) at ~10,000 g for 15 min. Pellets were stored at −80° C. Cells were lysed and purified using methods described for AJFKBP12 through the secondary Ni-chromatography step, of which fractions containing each fusion were pooled and concentrated for SEC (HsCnA-CnB at 14.4 mg/mL, AfCnA-CnB at 3.3 mg/mL). Each complex was loaded onto a Superdex 75 16/60 PG column and eluted in 25 mM Tris pH 8.0, 200 mM NaCl, 1 mM TCEP, and 5 mM CaCl₂. Final sample was concentrated to 10.61 mg/mL (HsCnA-CnB) and 11.3 mg/mL (AfCnA-CnB), flash frozen in liquid nitrogen and stored at −80° C. (AsfuA.00174.a.TQI 1+AsfuA.01011.a.TR11 fusion, Batch ID—PD38406; HosaA.00174.a.TU11+HosaA.01011.a.TV11 fusion, Batch ID—PD38411)

Complexes of A. Fumigatus CnA-CnB+A. Fumigatus FKBP12-P90G with FK506 and H. sapiens CnA-CnB+Bio-H. sapiens FKBP12 with FK520

Complexes were formed by first mixing FKBP12 with 1.5×FK506/FK520 at 4° C. for 30 min followed by addition of CnA-CnB. The combined sample was incubated for an additional 30 min at 4° C., then injected onto a Superdex 200 column (GE Healthcare) equilibrated in 25 mM Tris (pH 8.0), 200 mM NaCl, 1 mM Tris(2-carboxyethyl)phosphine hydrochloride (TCEP), and 5 mM CaCl2. Fractions containing the 1:1 complex were pooled and concentrated to 20 to 24 mg/mL.

### Structure Determination by X-Ray Crystallography.

A. fumigatus FKBP12 was preincubated at room temperature for 30 min with 1.6 mM FK520 (100 mM dimethyl sulfoxide [DMSO] stock, compound to protein ratio of 1.26) prior to being placed in sparse matrix crystallization trials using sitting drop vapor diffusion at 18.2 mg/mL. Crystallization trays were stored at 14° C., and crystals grew over the course of 4 months. Crystals were grown in JCSG+ (Rigaku Reagents) condition A1 containing 0.2 M lithium sulfate (pH 4.5) and 50% vol/vol polyethylene glycol (PEG) 400, which also acted as a cryoprotectant during harvest. The data set was collected on 16 Sep. 2021 at the synchrotron Advanced Photon Source (APS) beamline 21-ID-F on an MAR 300 charge-coupled device (CCD) X-ray detector. Two copies of the compound-bound FKBP12 were placed per asymmetric unit. The structure was solved by molecular replacement using a previously solved structure of A. fumigatus FKBP12 bound to FK506. Molecular graphics and analyses performed with UCSF Chimera, developed by the Resource for Biocomputing, Visualization, and Informatics at the University of California, San Francisco, with support from NIH P41-GM103311 (see, Pettersen E F, et al., 2004, J Comput Chem 25:1605-1612).

Crystallization Conditions of A. Fumigatus CnA-CnB+A. Fumigatus FKBP12-P90G with FK506 and H. sapiens CnA-CnB+Bio-H. sapiens FKBP12 with FK520.

Proteins were placed in sparse matrix crystallization trials using sitting drop vapor diffusion. Crystallization trays were stored at 14° C. Crystals of A fumigatus CnA-CnB (AfCnA-CnB)+AJFKBP12-P90G with FK506 grew after 1 month in JCSG+condition C₆ (Rigaku Reagents) containing 0.1 M sodium phosphate dibasic/citric acid) (pH 4.2) and 40% (vol/vol) PEG 300 (also cryoprotectant). Crystals of H. sapiens CnA-CnB (HsCnA-CnB)+Bio-HsFKBP12 with FK520 grew after 1 day in MCSG-1 condition C₅ (Microlytics) containing 0.2 M magnesium acetate and 20% (wt/vol) PEG 3350 (cryopreserved in 20% ethylene glycol). Data sets were collected on 10 Dec. 2020 at the synchrotron APS beamline 21-ID-F on an MAR 300 CCD X-ray detector. A single copy of each complex was placed per asymmetric unit (HsCnA-CnB+Bio-HsFKBP12 with FK520; AfCnA-CnB+AfFKBP12-P90G with FK506). The structure of HsCnA-CnB+Bio-HsFKBP12 with FK520 was solved by molecular replacement using a previously solved structure of Bos taurus CnA, CnB, and FKBP12 bound to FK506, and the structure of AfCnA-CnB+AfFKBP12-P90G with FK506 was solved by molecular replacement using a previously solved structure of A. fumigatus CnA, CnB, and FKBP12 bound to FK506.

Diffraction data were reduced and scaled with XDS/XSCALE (see, Kabsch W. 2010. Acta Crystallogr D Biol Crystallogr 66:125-132). Structures were solved by molecular replacement using the program Phaser in the Phenix program suite (see, Adams P D, et al., 2010, Acta Crystallogr D Biol Crystallogr 66:213-221). Structures were refined using iterative cycles of Translation/Libration/Screw (TLS) and restrained refinement with Phenix Refine and model building using COOT (see, Emsley P, Lohkamp B, Scott W G, Cowtan K. 2010. Acta Crystallogr D Biol Crystallogr 66:486-501). The final structures were validated using MolProbity and deposited in the Protein Data Bank (see, Chen V B, et al., 2010, Acta Crystallogr D Biol Crystallogr 66:12-21; Berman H, Henrick K, Nakamura H. 2003, Nat Struct Biol 10:980; Berman H M, et al., 2000, Nucleic Acids Res 28:235-242). Diffraction data and refinement statistics are listed in Table 1. Diffraction images are available at the Integrated Resource for Reproducibility in Macromolecular Crystallography (http://proteindiffraction.org/) (see, Grabowski M, et al., 2019, Struct Dyn 6:e064301; Grabowski M, et al., 2016, Acta Crystallogr D Struct Biol 72:1181-1193).

### Synthesis of FK506 and FK520 Analogs.

FK506 and FK520 monohydrate starting materials were purchased from APIChem Technology (catalog no. AC-32477 and AC-5267). FK506 and FK520 analogs were prepared as follows. To a solution of FK506 or FK520 in anhydrous ethanol (EtOH; 0.1 M) was added acylhydrazine (6.0 equivalent) at 25° C. The resulting mixture was refluxed under N₂ atmosphere. After stirring for 36 to 48 h, the reaction mixture was cooled to 25° C. and concentrated in vacuo. The resulting mixture was diluted with ethyl acetate (EtOAc) and H₂O. The layers were separated, and the aqueous layer was extracted with EtOAc. The combined organic layers were washed with brine, dried over anhydrous Na₂SO₄, and concentrated in vacuo. The residue was purified by silica gel column chromatography. All compounds were confirmed by HRMS and ¹H NMR (Table 2 and FIG. 4).

### Antifungal Susceptibility Testing.

In vitro antifungal activities of FK506, FK520, and analogs were assessed in RPMI 1640 (Sigma-Aldrich) for all strains tested. MICs/minimum effective concentrations (MECs) for each drug were measured using modified Clinical and Laboratory Standards Institute (CLSI) M38-A2 and M27-A3 standard in vitro antifungal susceptibility protocols. Broth microdilution assays for MIC/MEC assessment were performed as described previously (see, Juvvadi P R, et al., 2019, Nat Commun 10:4275). All antifungal assays with C. neoformans were performed at 37° C. Antifungal susceptibility testing for C. albicans was performed in the presence of a uniform 1 μg/mL concentration of fluconazole to sensitize the organism to calcineurin inhibition.

### Biolayer Interferometry Binding Experiments.

Biolayer interferometry (BLI) experiments were completed using either A. fumigatus or C. albicans FKBP12 and CnA/CnB/calmodulin complexes, along with compounds FK506, APX879, FK520, JH-FK-02, and JH-FK-05. Initial buffer (25 mM Tris, 200 mM NaCl, and 1 mM TCEP [pH 8.0]) was prepared using 1 M Tris-HCl (pH 8.0, catalog no. T1080; Teknova), 5 M sodium chloride (catalog no. S0251; Teknova), TCEP (catalog no. M115; Soltec Ventures, Inc.), and was readjusted to pH 8 before sterile filtering. Bovine serum albumin (BSA)-containing buffer was prepared by addition of BSA stock (30% in Dulbecco's phosphate-buffered saline [DPBS], catalog no. A9576; Millipore Sigma) into the initial buffer to a final concentration of 1%. Compound-containing assay buffers were prepared by addition of compound stock (100 mM in DMSO-d₆) into BSA-containing buffer to a final concentration of 10 μM. FKBP12 solutions were prepared using a two-step process in which FKBP12 was first diluted to 100 g/mL with BSA-containing buffer, followed by dilution to 10 g/mL with compound-containing assay buffer. Initial 2,500 nM CnA/CnB/calmodulin complexes were prepared by direct dilution of protein stock with compound-containing assay buffer. Serial 1:1 dilutions were then performed with compound-containing assay buffer to prepare the remaining samples in the series (full series: 2,500 nM, 1,250 nM, 625 nM, 312.5 nM, 156.25 nM, 78.125 nM, 39.0625 nM, and 0 nM [sample blank]). FKBP12 and CnA/CnB/calmodulin complexes were incubated overnight prior to experiments.

Experiments were performed on an Octet Red96e instrument (Sartorius) utilizing Octet Data Acquisition software version 11.1.1.19, equilibrated to 25° C. Streptavidin biosensors (SA, catalog no. 18-5019; Sartorius) were hydrated for a minimum of 30 min in compound-containing assay buffer prior to the start of each experiment. Each experiment consisted of two identical assays, each performed using separate biosensors-one using sensors loaded with FKBP12 (sample sensors), and the other using unloaded sensors (parallel reference sensors). Each assay consisted of the following steps: initial baseline (120 s, compound-containing assay buffer); FKBP12 loading (170 s for Aspergillus, 180 s for Candida); sample baseline (120 s, compound-containing assay buffer); CnA/CnB/calmodulin complex association (300 s); dissociation (900 s, same compound-containing assay buffer wells as in the sample baseline step); sample baseline/association/dissociation cycle 2 (same times and wells as in previous steps); sample baseline/association/dissociation cycle 3 (same times and wells as in previous steps). Data were acquired for FKBP12-loaded sensors first and parallel reference sensors second.

Data were processed and analyzed using ForteBio Data Analysis software version 11.1.3.10. Data were processed using the “double reference subtraction” option, with the y axis aligned to baseline (from 0.1 to 119.8 s), interstep correction aligned to dissociation, and Savitzky-Golay filtering active. Kinetics data were analyzed using the “association and dissociation curve fitting” option utilizing a 1:1 model. Fitting was performed using the “global (full)” option with “Rₘₐₓ unlinked by sensor” (the one exception being Aspergillus sample JH-FK-05, which would not process cleanly unless the “Rₘₐₓ linked” option was selected instead). The window of interest spanned the full association and dissociation steps of each experiment. Steady-state analysis was completed using the response average from 290.0 to 295.0 s of the association step (because some binding responses did not reach equilibrium).

### In Vitro Immunosuppressive Activity Testing

Spleen and lymph nodes were collected from C₅₇BL/6 mice and homogenized through a 40-μm filter. Pan-CD4⁺ T cells were enriched using a MagniSort mouse CD4 T-cell kit (eBioscience) according to the manufacturer's protocol. Thereafter, CD4⁺CD25⁻ CD44^{lo}CD62L^{hi} naive T cells were sorted by fluorescence-activated cell sorting (FACS). The naive CD4⁺ T cells were grown in Iscove's modified Dulbecco's medium (IMDM) supplemented with glutamine, penicillin, streptomycin, gentamicin, 2-mercaptoethanol, and 10% fetal bovine serum (FBS). Cells were then cultured on anti-hamster IgG-coated plates in the presence of hamster anti-CD3 epsilon and anti-CD28 antibodies (BD Biosciences), neutralizing anti-IL-4 antibody (eBioscience), recombinant IL-12 (10 ng/mL), and recombinant IL-2 (50 U/mL) for 72 h. During this period, cells were grown in the presence of 2× serially diluted FK506, FK520, or analog suspended in DMSO for 72 h. During the last 4 h of culture, phorbol 12-myristate 13-acetate (PMA; Sigma), ionomycin (Sigma), and GolgiStop (BD Biosciences) were added to the culture to facilitate detection of intracellular cytokines via flow cytometry. Live cells were stained with fluorescein isothiocyanate (FITC)-conjugated anti-CD4 antibody (eBioscience) and fixable viability dye eFluor 506 (eBioscience) on ice, followed by fixation and permeabilization using the Foxp3 transcription factor staining kit (eBioscience). Intracellular staining was performed at room temperature using phycoerythrin (PE)-conjugated anti-IL-2 antibody (eBioscience), followed by analysis with a BD LSRFortessa X-20 flow cytometer.

### Preparation of Compounds for In Vivo Studies.

For in vivo studies on immunosuppressive activity, FK506 was purchased from Duke Pharmacy as Prograf (tacrolimus; Astellas) and was used as a solution of 1 mg/mL. Vehicle was prepared as a sterile solution of 90% phosphate-buffered saline (PBS), 5% Kolliphor EL (catalog no. C₅₁₃₅; Sigma), and 5% ethanol. JH-FK-05 stock solution of 120 mg/mL was prepared as a powder dissolved in sterile 50% Kolliphor EL and 50% ethanol. For administration to animals, JH-FK-05 stock was diluted to 12 mg/mL and 10% Kolliphor-ethanol in sterile PBS. Fluconazole was purchased from Duke University Hospital Pharmacy as a sterile solution of fluconazole 2 mg/mL in PBS.

### In Vivo Immunosuppressive Activity Assessment of JH-FK-05.

Groups of 6 female C₅₇BL/6 mice (Jackson Laboratory) were treated daily by intraperitoneal (i.p.) injection with about 100 μL vehicle, 5 mg/kg FK506, or 60 mg/kg JH-FK-05 beginning on day −1. On day 0 mice were immunized with NP-OVA in alum via subcutaneous injection. Mice were treated and monitored daily until they were sacrificed on day 7. Draining lymph nodes were harvested on day 7, and the populations of T follicular helper cells and GC B cells were analyzed by flow cytometry. Tfh and GC B cells were measured from T Cell Receptor β (TCRβ⁺) CD4⁺CD44^{hi} cells and TCRβ⁻CD19⁺ cells, respectively.

C. neoformans Cell Preparation for Murine Infection.

Wild-type H99 C. neoformans cells were grown overnight at 30° C. in a roller drum in yeast extract-peptone-dextrose (YPD) medium. Cells were pelleted at 3,000 rpm and washed 3 times with sterile PBS. H99 cells were counted with a hemacytometer and diluted to 2×10⁶ cells/mL in PBS for the pulmonary model and 2×10⁵ cells/mL in PBS for the disseminated model.

### Intranasal Model of Pulmonary Cryptococcosis.

Female A/J mice (3 to 4 weeks old; Jackson Laboratory) were anesthetized utilizing an isoflurane chamber. While anesthetized, 50 L of 2×10⁶ cells/mL suspension was dripped onto the nares of mice, allowing them to inhale the full inoculum. Treatment began 3 h following infection with i.p. dosing of vehicle, 60 mg/kg JH-FK-05, 12 mg/kg fluconazole, or combination. Animals were treated daily for 14 days. After 14 days, animals were either sacrificed for fungal burden analysis or monitored for health and survival until reaching a humane endpoint. Survival analysis was plotted and performed in GraphPad Prism 9. Statistical significance was determined using log-rank (Mantel-Cox) tests.

### Intravenous Model of Disseminated Cryptococcosis.

Male CD1 mice (3 to 4 weeks old; Charles River) were restrained in a 50-mL conical tube with holes at both ends for ventilation. Animal tails were warmed under a heat lamp to enhance vasodilation and sterilized with alcohol wipes. A 50-μL suspension of 2×10s cells/mL was injected into the lateral tail vein with an insulin syringe. Treatment proceeded as described in the intranasal infection model until day 7, when animals were sacrificed for fungal burden analysis.

### Cryptococcus Fungal Burden Analysis.

Mice were euthanized by CO₂ inhalation at the predetermined experimental endpoint. Organs from each animal were harvested and homogenized in sterile PBS using steel beads (Qiagen) and a bead beater. Organ homogenate was serially diluted in PBS. A 100-μL aliquot of each dilution was plated on antibiotic plates (YPD, 50 μg/mL ampicillin, and 30 g/mL chloramphenicol), incubated for 48 to 72 h at 30° C., and assessed for CFU. CFU per gram of organ was plotted using GraphPad Prism 9. Statistical significance was calculated using an ordinary one-way analysis of variance (ANOVA) with Tukey's multiple-comparison test.

### Molecular Dynamic Simulations.

MD simulations were performed to provide a better representation of the protein's conformational flexibility and to more accurately characterize the protein's solution structure bound to JH-FK-05. The following crystal structures were used as the starting conformations: hFKBP12 bound to FK506 and C. neoformans FKBP12 (cnFKBP12) (calcineurin A and B removed) (see, Juvvadi P R, et al., 2019, Nat Commun 10:4275; Van Duyne G D, et al., 1991, Science 252:839-842). JH-FK-05 small-molecule parameter and topology files were downloaded and created utilizing the Automated Topology Builder (ATB) and repository (see, Malde A K, et al., 2011, J Chem Theory Comput 7:4026-4037; Stroet M, et al., 2018, J Chem Theory Comput 14:5834-5845). All molecular dynamic (MD) simulations were performed with the GROMACS 5.0.1 software package utilizing 6 CPU cores and one Nvidia Tesla K80 GPU (see, Abraham M J, et al., 2015, SoftwareX 1-2:19-25). The single starting conformations used for all of the MD simulations were the X-ray-characterized crystal structures noted above. MD simulations were performed with the GROMOS54a7 force field and the flexible simple point-charge water model. The initial structures were immersed in a periodic water box with a dodecahedron shape that extended 1 nm beyond the protein in any dimension and were neutralized with counter ions. Energy minimization was accomplished through use of the steepest descent algorithm with a final maximum force below 100 kJ/mol/min (0.01-nm step size and cutoff of 1.2 nm for neighbor list, Coulomb interactions, and Van der Waals interactions). After energy minimization, the system was subjected to equilibration at 300 K and normal pressure for 1 ns. All bonds were constrained with the LINCS algorithm (cutoff of 1.2 nm for neighbor list, Coulomb interactions, and Van der Waals interactions). After temperature stabilization, pressure stabilization was obtained by utilizing a V-rescale thermostat to hold the temperature at 300 K, and a Berendsen barostat was used to bring the system to a pressure of 10⁵ Pa. Production MD calculations (500 ns) were performed under the same conditions, except that the position restraints were removed, and the simulation was run for 500 ns (cutoff of 1.1, 0.9, and 0.9 nm for neighbor list, Coulomb interactions, and Van der Waals interactions). These MD simulations were repeated 6 times. Alpha carbon (C-α) root mean square deviation (RMSD), radius of gyration (Rg), and center of mass (COM) all confirmed the stability and accuracy of the MD simulations by stabilizing after a 100-ns equilibration period (in most cases), allowing for analysis of the last 400 ns of the simulations. Only one of the 12 calculations (hFKBP12:JHFK05 complex MD sim no. 3) was rejected from the analysis due to unstable C-α RMSD, Rg, and/or COM (FIG. 10). GROMACS built-in and homemade scripts were used to analyze the MD simulation results and averaged over the 6 simulations. All images were produced using PyMOL (see, Schrödinger, LLC. 2016. PyMOL molecular graphics system, version 1.8. Schrödinger, LLC, New York, NY). Atom indices for JHFK05 are provided in Table 5.

### Example 3

### Chemical Synthesis of Novel FK520 Analogs Modified at the C-22 Position.

Previous studies determined the C-22 position on FK506/FK520 as a key point of differentiation between fungal versus human calcineurin-FK506-FKBP12 complexes (see, Juvvadi P R, et al., 2019. Nat Comnun 10:4275; Aria T, Kouama Y, Suenaga T, Honda H. 1962 J Antibiot 15:231-232). To selectively modify the C-22 position on FK520, the condensation reaction with hydrazine derivatives was examined. The condensation reaction with hydrazine derivatives exploits the unique reactivity of carbonyl groups, such as ketones and aldehydes, toward hydrazine derivatives in the presence of other functional groups. Based on this rationale, 12 new FK520 analogs were synthesized through one-step syntheses that can be readily scaled up for studies in animal infection models for pre-clinical analysis (FIG. 11) (see, Juvvadi P R, et al., 2019. Nat Commun 10:4275; U.S. Pat. No. 8,278,264). The synthesis of FK520 analogs with C-22 modifications features a condensation of FK520 with a hydrazide or a substituted hydrazine either under reflux or acidic conditions. After completion of the reaction, the reaction mixture was concentrated in vacuo and purified by silica gel column chromatography to give the desired C-22-modified FK520 analogs (33%-52% yield). Each analog was validated through ¹H nuclear magnetic resonance (NMR) and high-resolution mass spectrometry (HRMS) (Table 6).

TABLE 6
High-resolution mass spectrometry
(HRMS) data for FK520 analogs

| | Elemental | Calculated | Observed | Error | |
|---|---|---|---|---|---|
| Analog | Composition | Mass | Mass | (ppm) | Ion |
| JH-FK-08 | C₄₅H₇₃N₃O₁₃ | 864.5222 | 864.5217 | −0.6 | [M + H]⁺ |
| JH-FK-09 | C₄₉H₇₄N₄O₁₂ | 911.5376 | 911.5378 | 0.2 | [M + H]⁺ |
| JH-FK-12 | C₄₄H₇₂N₄O₁₂ | 849.5220 | 849.5223 | 0.4 | [M + H]⁺ |
| JF-FK-13 | C₅₃H₇₇N₃O₁₃S | 960.5250 | 960.5261 | 0.1 | [M + H]⁺ |
| JH-FK-14 | C₅₁H₇₇N₅O₁₃ | 968.5591 | 968.5575 | −1.7 | [M + H]⁺ |
| JH-FK-15 | C₅₄H₇₇N₃O₁₂ | 960.5580 | 960.5579 | −0.1 | [M + H]⁺ |
| JH-FK-16 | C₄₅H₇₅N₃O₁₁ | 834.5474 | 834.5482 | 1.0 | [M + H]⁺ |
| JH-FK-17 | C₅₀H₇₄FN₃O₁₂ | 928.5329 | 928.5341 | 1.3 | [M + H]⁺ |
| JH-FK-20 | C₅₀H₇₆N₄O₁₂ | 925.5533 | 925.5537 | 0.4 | [M + H]⁺ |
| JH-FK-21 | C₄₆H₇₃N₅O₁₂ | 864.5329 | 864.5319 | −1.2 | [M + H]⁺ |
| JH-FK-22 | C₄₅H₇₃N₃O₁₃ | 864.5216 | 864.5214 | −0.2 | [M + H]⁺ |
| JH-FK-23 | C₄₄H₇₃N₃O₁₃S | 884.4937 | 884.4930 | −0.8 | [M + H]⁺ |

### FK520 Analogs Display Conserved Antifungal Activity

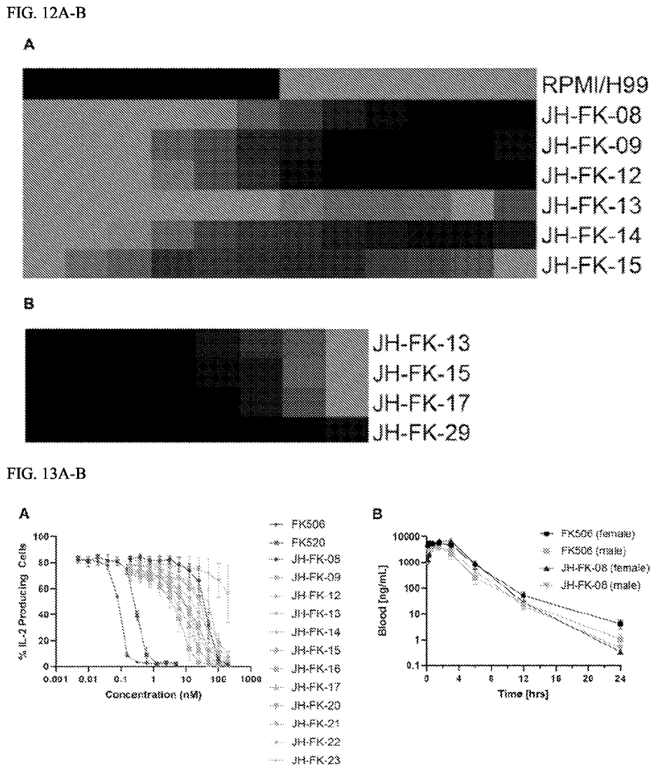

Eleven of these FK520 analogs were found to retain some level of antifungal activity. To assess antifungal activity, the panel of compounds was tested for fungal growth inhibition using minimum inhibitory concentration (MIC) assays against C. neoformans and C. albicans and minimum effective concentration (MEC) assays against A. fumigatus. The results from these broth microdilution assays showed a decrease in antifungal activity of the modified compounds compared to FK506 and FK520 (Table 7). Ten of these analogs exhibited an MIC value between 0.4 and 1.6 μg/mL, which is an 8- to 32-fold decrease in activity compared to FK506 or FK520 in C. neoformans and C. albicans. MEC values against A. fumigatus ranged from 0.6 to 2.5 μg/mL for 10 of the analogs tested. Notably, analogs JH-FK-13, JH-FK-15, and JH-FK-17 exhibited solubility issues during microdilution broth assays (FIG. 12) (see, Ascierto P A, Marincola F M. 2011. J Transl Med 9:115; Shaw K J, et al., 2018. Antimicrob Agents Chemother 62:e00523-18; Shaheen S K, et al., 2021. Antimicrob Agents Chemother 65:e0068221). Overall, a majority of these analogs maintain relatively potent antifungal activity across species and represent an active library for further investigation.

TABLE 7
MICs for FK506, FK520, and analogs

| | MIC/MEC (μg/mL) against | | |
|---|---|---|---|
| | Cryptococcus | Candida | Aspergillus |
| | neoformans | albicans | fumigatus |
| Compound | (H99) | (SC5314) | (akuB^{Ku80}) |
| FK506 | 0.05 | 0.02 | 0.04 |
| FK520 | 0.05 | 0.02 | 0.16 |
| JH-FK-08 | 0.8 | 0.4 | 1.3 |
| JH-FK-09 | 0.8 | 0.8 | 1.3 |
| JH-FK-12 | 0.8 | 0.8 | 2.5 |
| SH-FK-13 | >50 | >50 | >10 |
| JH-FK-14 | 1.6 | 0.8 | 0.6 |
| JH-FK-15 | 3.1 | 0.4 | 0.3 |
| SH-FK-16 | 0.4 | 0.4 | 1.3 |
| JH-FK-17 | 0.4 | 0.4 | 1.3 |
| JH-FK-20 | 0.8 | 0.8 | 0.6 |
| SH-FK-21 | 1.6 | 0.8 | 2.5 |
| BH-FK-22 | 1.6 | 1.6 | 2.5 |
| JH-FK-23 | 1.6 | 1.6 | 5 |

MiC₉₀ was tested at 37° C. for C. neoformans. MIC₈₀ was tested at 30° C. with 2 μg/mL supplementary fluconazole for C. albicans. MEC was collected for A. fumigatus strains.

Modifications at the C-22 position reduce inhibition of mammalian calcineurin signaling Mammalian calcineurin inhibition was then assessed and found to be reduced for these FK520 analogs. The inhibition of mammalian calcineurin was assessed by measuring the reduction of IL-2 expression from primary mouse CD4⁺ T cells cultured in the presence of FK506, FK520, or an analog. Calcineurin activity is required for T cell activation and cytokine production, and IL-2 production serves as an effective measure of mammalian calcineurin activity. After culturing mouse CD4′ T cells for 72 h, IL-2 expression was analyzed by flow cytometry to evaluate the immunosuppressive activity of the analogs. The results showed an overall reduction in in vitro immunosuppressive activity of the analogs compared to FK506 and FK520, as denoted by the increased concentration required to inhibit cytokine production (FIG. 13A). Previous studies measuring FK506 immunosuppressive activity have demonstrated FK506 to be cytotoxic and reduce the proliferation of T cells (41). In this study, decreased levels of cell proliferation were observed at high doses of compounds where IL-2 expression was decreased. However, compound concentrations that permit high IL-2 expression are less likely to result in cell death. Therefore, IC₅₀ values were determined from these data to quantify the reduction in immunosuppression due to the structural modification of FK520 (Table 8). The IC₅₀ values of the analogs demonstrate a minimum 65-fold reduction in mammalian calcineurin inhibition. Notably, IC₅₀ values increased from 0.09 nM for FK506 up to 70.5 nM for analog JH-FK-23 and to 42.6 nM for JH-FK-08. Analog JH-FK-13 showed an IC₅₀ greater than 200 nM and an MIC value greater than 50 μg/mL, indicating that targeting of both mammalian and fungal calcineurin has been compromised. The results demonstrate that structurally modified analogs have a reduced capacity to inhibit mammalian calcineurin, and therefore their immunosuppressive activity has been reduced.

TABLE 8
Therapeutic indices for FK506, FK520, and analogs

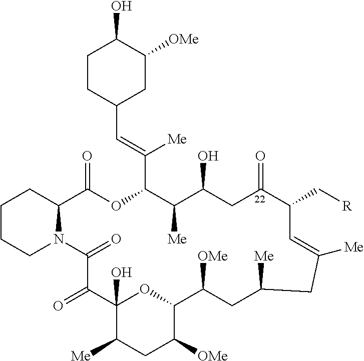

FK506: R = CHCH₂
FK520: R = CH₃

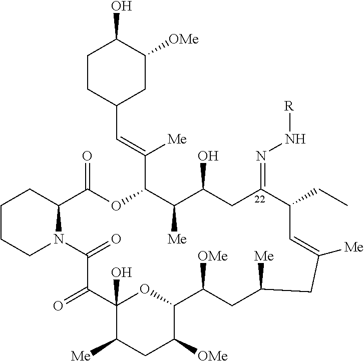

C22 modified FK520 analogs

| | | H99 | IL-2 | Therapeutic |
|---|---|---|---|---|
| Compound | R | MIC₉₀ (μg/mL) | IC₅₀ (nM) | index |
| FK506 | — | 0.05 | 0.09 | 1.0 |
| FK520 | — | 0.05 | 0.3 | 3.3 |
| JH-FK-08 | CO₂Me | 0.8 | 42.6 | 29.6 |
| JH-FK-09 | C(═O)-4-pyridine | 0.8 | 11.2 | 7.8 |
| JH-FK-12 | CONH₂ | 0.8 | 35.6 | 24.7 |
| JH-FK-13 | SO₂—4-Me—Ph | >50 | >200 | Undefined |
| JH-FK-14 | C(═O)—2-CONHNH₂—Ph | 1.6 | 14.0 | 4.9 |
| JH-FK-15 | C(═O)—2-naphthalene | 3.1 | 17.4 | 3.1 |
| JH-FK-16 | Et | 0.4 | 5.9 | 8.2 |
| JH-FK-17 | C(═O)—4-F—Ph | 0.4 | 9.4 | 13.1 |
| JH-FK-20 | C(═O)—4-NH₂—Ph | 0.8 | 17.8 | 12.3 |
| JH-FK-21 | CONHNH₂ | 1.6 | 54.5 | 18.9 |
| JH-FK-22 | COCH₂OH | 1.6 | 45.0 | 15.6 |
| JH-FK-23 | SO₂Me | 1.6 | 70.5 | 24.5 |

JH-FK-08 has been Identified as a Lead Compound

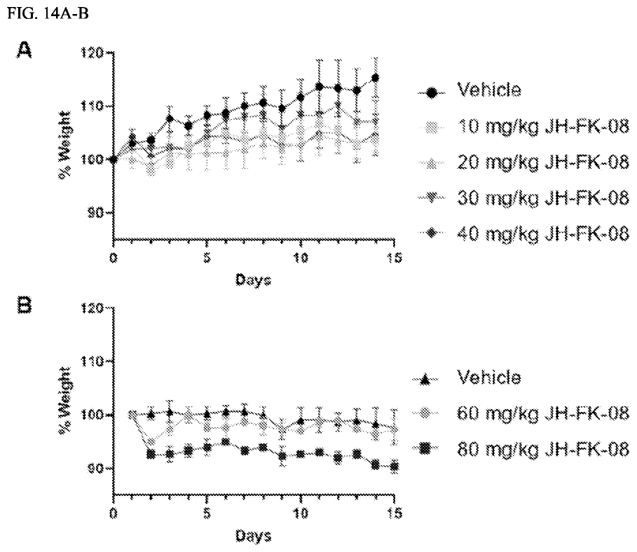

Based on the in vitro fungal and mammalian calcineurin inhibition data, the panel of FK520 analogs was assessed and ranked. Therapeutic index (TI) scores were used to quantify the effects of structural modification on the compounds (Table 8). Therapeutic index scores were calculated for each analog as the ratio of relative change in immunosuppressive and antifungal activities compared to FK506 as previously utilized (see, I-Toy M J, et at. 2022. mBio 13:e0104922). This ratio of therapeutic benefit and host toxicity, or fungal and mammalian calcineurin inhibition, allowed us to quantify and rank these analogs. Using the formula TI=(IC_{50[compound]}/IC_{50[FK506]})/(MIC_{[compound]}/MIC_{[FK506]}) and the in vitro immunosuppressive and antifungal activities against C. neoformans, the TI for JH-FK-08 is 29.6. This is the greatest TI found within the library and allowed us to identify JH-FK-08 as a lead candidate for an in vivo study. Tolerability of JH-FK-08 was assessed by monitoring animal responses to drug dosing across 14 days, including changes in weight, fur condition, and behavior. A maximum dose of 40 mg/kg was tested in once a day dosing, and due to solubility limitations, the tolerability of JH-FK-08 at 60 and 80 mg/kg total daily doses was assessed by dosing two equal doses per day (FIG. 14). Animals tolerated all dosages, and weight ranges remained within ~10% of vehicle-treated animals. It should be noted that analogs JH-FK-12 and JH-FK-23 are also promising candidates with respective Tis of 24.7 and 24.5.

For the lead compound JH-FK-08, the mechanism of action resulting in antifungal activity was tested for conservation of calcineurin inhibition. To probe the JH-FK-08 mechanism of action, MICs of JH-FK-08 were determined with fungal mutants that lack the drug-binding protein FKBP12 or have mutations in the binding site on calcineurin that prevent FKBP12-FK506 binding and inhibition. These results demonstrate the following: (i) FKBP12 is required for the antifungal activity of JH-FK-08, similar to the action of FK506 and FK520; (ii) mutations of calcineurin B that render calcineurin resistant to binding and inhibition by FKBP12-FK506 similarly confer resistance to JH-FK-08; and (iii) FK506, FK520, and JH-FK-08 exhibit potent antifungal activity against C. neoformans at 37° C. but not at 30° C., in accordance with the known requirement of calcineurin for growth at elevated temperatures (Table 9). Taken together, these findings support the hypothesis that JH-FK-08 requires FKBP12 for antifungal activity, and the target of the FKBP12-JH-FK-08 complex is calcineurin. The length of time that drugs are active in vivo is critical information relevant to drug development, and the time frame of drug activity can have a drastic impact on treatment regimen and efficacy. Therefore, the pharmacokinetic profiles of FK506 and JH-FK-08 were investigated. Three male and three female CD1 mice were administered a single dose of JH-FK-08 or FK506 at 40 mg/kg. Whole blood samples were taken over a 24-hour period, and the drug concentration remaining in the blood was determined. Pharmacokinetic parameters were calculated using a non-compartmental approach (see, Gabrielsson J, Weiner D. 2012. Methods Mot Biol Clifton NJ 929:377-389). No significant differences across groups were observed in whole blood samples of drug concentration over time, indicating a strong conservation of the pharmacokinetic profile of JH-FK-08 compared to that of FK506 (FIG. 13B). When assessing specific pharmacokinetic measures, sex differences began to emerge. When looking at the area under the curve (AUC), females showed roughly twice the value of males, and male clearance of both FK506 and JH-FK-08 was higher than female-treated animals (Table S3). Despite this, the half-life across all groups ranged from 1.14 to 1.48 h. Overall, JH-FK-08 exhibits a pharmacokinetic profile very similar to that of FK506, including sex-dependent trends seen in clearance and AUC.

TABLE 9
Antifungal activity of JH-FK-08 against natural and mutant strains

| Fungal strain | Background | MIC/MEC (μg/mL) |
|---|---|---|
| KN99α | Cryptococcus neoformans | <0.4 |
| frr1Δ | KN99α frr1Δ::NAT | >25 |
| JEC21 | Cryptococcus deneoformans | <0.4 |
| C21F2 | JEC21 CNB1-1 | >25 |
| C21F3 | JEC21 frr1 | >25 |
| SC5314 | Candida albicans | <0.4 |
| YAG171 | SC5314 rbp1Δ::URA3/rbp1Δ::HISG | >25 |
| YAG237 | SC5314 CNB1-1/CNB1 | >25 |
| akuB^{Ku80} | Aspergillus fumigatus | 1.25 |
| Δfkbp12 | akuB^{Ku80} fkbp12Δ | >20 |
| H99 | Cryptococcus neoformans | >4 |

MIC₉₀ was collected at 37° C. for KN99 and related strains. MIC₉₀ was collected at 30° C. for H99 JEC21 strains grown for 72 hours with 5 times starting amount of cells. MIC₈₀ was collected at 30° C. with 2 μg/mL supplementary fluconazole grown for 72 hours for C. albicans. MEC was collected for A. fumigatus strains.

### In Vivo Immunosuppressive Activity of JH-FK-08 is Reduced

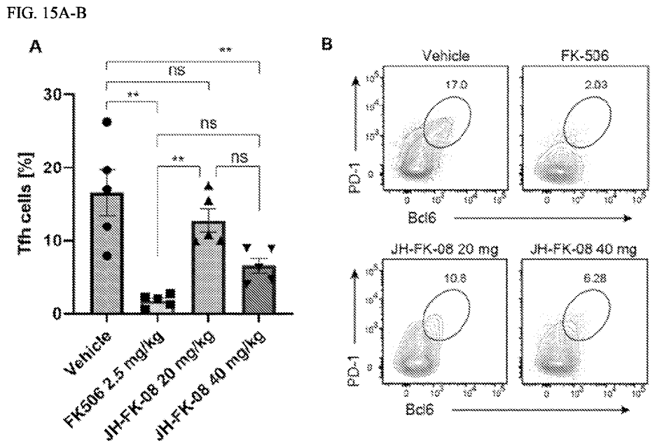

Whole animal studies were conducted to assess the in vivo implications of the reduced mammalian calcineurin inhibition demonstrated during in vitro immune cell testing. The in vivo immunosuppressive effect of JH-FK-08 was assessed by studying T follicular helper (Tfh) cell production in mice. Tfh cells play a role in protection against and response to fungal infections (see, Wong G K, et al., 1998. J Antibiot 51:487-491; Cruz M C, et al., 1999. Mol Cell Biol 19:4101-4112). For this study, immunocompetent C₅₇BL/6 mice received 8 days of twice-daily dosing of vehicle, FK506 (2.5 mg/kg), or JH-FK-08 (20 or 40 mg/kg). Twenty-four hours after the first dose, mice were immunized with NP-OVA to induce an immune response. Cells were analyzed 7 days post immunization, and cell types were analyzed by flow cytometry (FIG. 15B).

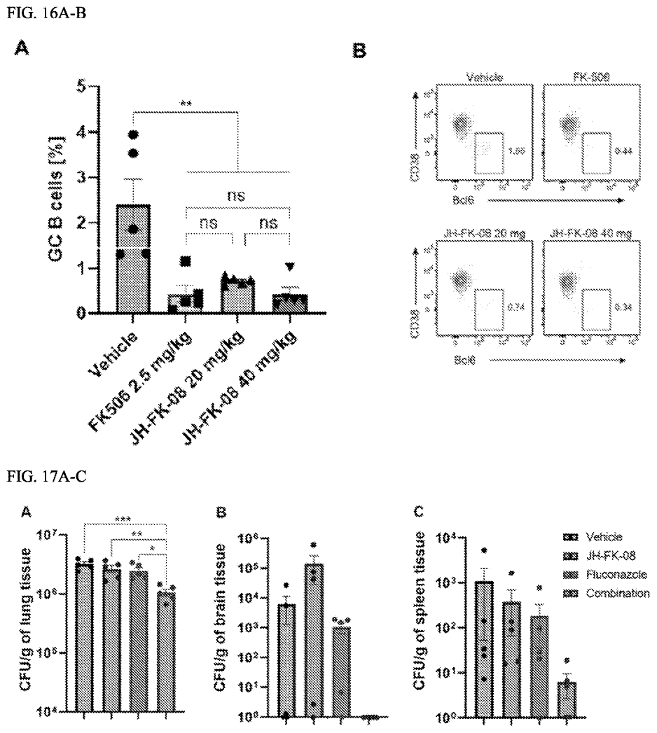

Treatment with JH-FK-08 at 20 mg/kg had no significant effect on the percent of Tfh cells produced compared to vehicle (FIG. 15A). However, the production of Tfh cells was reduced at a dosage of 40 mg/kg of JH-FK-08. The immune response was also significantly reduced in mice treated with 2.5 mg/kg FK506. In germinal center B cells, JH-FK-08 showed similar levels of immunosuppressive activity compared to FK506 (FIG. 16). Overall, these findings indicate that Tfh cells are impacted by JH-FK-08 in a dose-dependent manner, and FK506 significantly inhibits Tfh cell production at 1/16 of the dose of JH-FK-08. These results in whole animals are well correlated with the finding that the analog JH-FK-08 has significantly reduced mammalian calcineurin inhibition in vitro compared to FK506.

### JH-FK-08 Reduces Fungal Burden During Murine Cryptococcal Infection

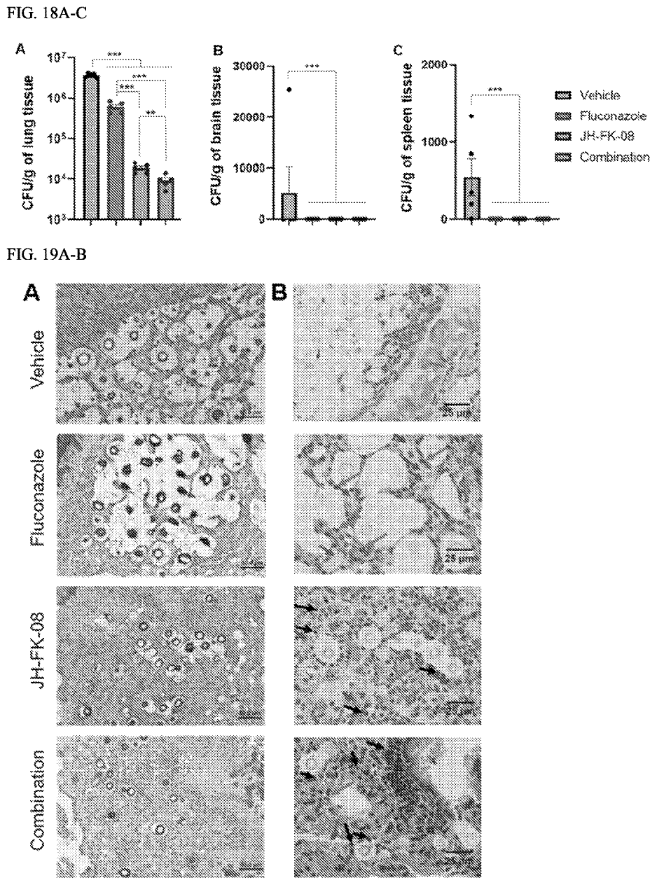

In vivo antifungal activity and therapeutic benefit were assayed by investigating the impact of the FK520 analog on organ fungal burden during cryptococcosis. Specifically, the in vivo efficacy of JH-FK-08 on fungal burden was assessed using A/J female mice. Immunodeficient A/J mice were selected in part due to their defect in complement component five and their susceptibility to cryptococcal infection. Treatment groups included vehicle of 10% Kolliphor: ethanol (1:1) in phosphate-buffered saline (PBS), 6 mg/kg fluconazole, 40 mg/kg JH-FK-08, or the fluconazole and JH-FK-08 combination, all given via intraperitoneal (i.p.) injection twice per day beginning at 4 hours post infection. Single-day dosing with vehicle, 12 mg/kg fluconazole, 40 mg/kg JH-FK-08, and JH-FK-08 plus fluconazole combination resulted in limited efficacy in reducing lung fungal burden, and dosing twice per day was implemented to augment treatment efficacy (FIG. 17). Mice were intranasally infected with C. neoformans strain H99, and fungal burden in the lung, spleen, and brain was assessed after 14 days of treatment. Treatment with fluconazole, JH-FK-08, or a combination resulted in no detectable fungal burden in the brain or spleen. The average lung fungal burden in untreated animals was approximately 3.7 million fungal cells per gram of lung tissue. This lung fungal burden was significantly reduced by ~6-, ~200-, and >400-fold in treatments with fluconazole, JH-FK-08, or the combination, respectively (FIG. 18). Additionally, recruitment of immune cells to the site of infection in lung tissue occurred based on histopathology analysis. The expected granulomatous inflammatory response admixed with mature lymphocytes and neutrophils was seen during JH-FK-08 and combination treatment (FIG. 19). This indicates that the reduction of JH-FK-08 immunosuppression was sufficient such that immune responses were still mounted during infection and JH-FK-08 treatment had a direct impact on reducing organ fungal burden during cryptococcal infection.

Treatment with JH-FK-08 Extends Survival During Cryptococcal Infection

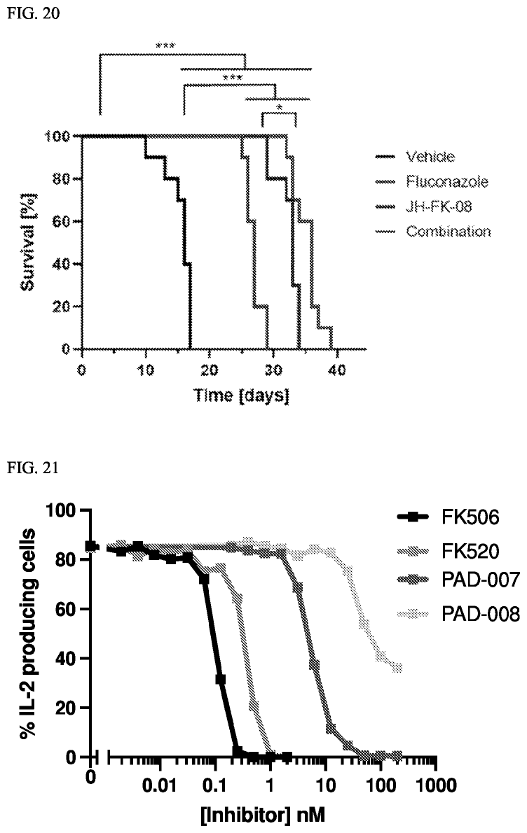

Finally, whether the antifungal efficacy translates into extended survival during infection was assessed. To test the impact of treatment on survival, murine survival was monitored in the cryptococcosis infection model. Following the same protocol of infection and dosing as in the fungal burden experiment, animal survival was monitored following infection and 2 weeks of intraperitoneal dosing. Fluconazole treatment extended median survival from 16 to 27 days. JH-FK-08 treatment alone more than doubled median survival from 16 days in vehicle-treated mice to 33 days in treated animals. This beneficial impact on survival was further enhanced by concomitant fluconazole treatment, which resulted in an increased median survival from 16 to 36 days (FIG. 20). These results further emphasize the therapeutic benefit JH-FK-08 offers by demonstrating extended survival in animals treated with JH-FK-08 alone or in combination with fluconazole.

### ¹H Nuclear Magnetic Resonance (NMR) Data for FK520 Analogs

JH-FK-08: JH-FK-08 was synthesized from FK520 (50 mg, 0.065 mmol) and methyl carbazate (6 eq, 34.1 mg, 0.38 mmol) in ethanol (0.4 mL, 0.15 M) according to General Procedure A. Purification by column chromatography (silica gel, CH₂Cl₂/MeOH, 25/1) afforded JH-FK-08 as a yellow solid (27.9 mg, 51.1%). JH-FK-08 was synthesized from FK520 (100 mg, 0.13 mmol) and methyl carbazate (6 eq, 68.2 mg, 0.76 mmol) in methanol (1.3 mL, 0.1 M) according to General Procedure B. Purification by column chromatography (silica gel, CH₂Cl₂/MeOH, 25/1) afforded JH-FK-08 as a yellow solid (92.3 mg, 85%): ¹H NMR (500 MHz, acetone-d6): δ 9.99 (s, 1H), 8.65 (s, 1H), 5.47 (s, 1H), 5.25 (s, 1H), 5.14 (t, J=7.5 Hz, 2H), 4.35 (s, 1H), 4.33 (d, J=17 Hz, 1H), 4.27 (d, J=4.5 Hz, 1H), 3.65 (s, 3H), 3.63 (s, 3H), 3.45 (dd, J=11, 15 Hz, 2H), 3.36 (s, 6H), 3.33 (s, 3H), 3.22 (q, J=7.0 Hz, 1H), 3.01-2.94 (m, 2H), 2.35 (d, J=16 Hz, 2H), 2.29-2.22 (m, 2H), 2.17 (d, J=14 Hz, 1H), 2.13-2.10 (m, 4H), 2.03-1.98 (m, 1H), 1.92 (s, 1H), 1.90 (s, 2H), 1.81-1.74 (m, 2H), 1.66 (s, 3H), 1.65 (s, 3H), 1.61-1.55 (m, 3H), 1.53-1.47 (m, 2H), 1.40-1.29 (m, 5H), 1.11-1.08 (m, 1H), 1.05-1.03 (m, 1H), 1.02 (d, J=9.0 Hz, 3H), 0.97 (d, J=7.5 Hz, 3H), 0.94-0.90 (m, 1H), 0.86 (t, J=7.5 Hz, 3H), 0.85 (t, J=6.5 Hz, 3H).

JH-FK-09: JH-FK-09 was synthesized from FK520 (50 mg, 0.065 mmol) and isonicotinic acid hydrazide (6 eq, 52 mg, 0.38 mmol) in ethanol (0.4 mL, 0.15 M) according to General Procedure A. Purification by column chromatography (silica gel, CH₂Cl₂/MeOH, 25/1) afforded JH-FK-09 as a white solid (24.7 mg, 49%): ¹H NMR (500 MHz, acetone-d6): δ 11.62 (s, 1H), 8.68 (d, J=6.0 Hz, 2H), 7.76 (d, J=6.0 Hz, 2H), 5.89 (s, 1H), 5.71 (s, 1H), 5.18 (d, J=9.0 Hz, 1H), 5.14 (d, J=9.0 Hz, 1H), 4.36 (s, 1H), 4.27 (m, 2H), 4.16 (d, J=10 Hz, 1H), 3.47 (d, J=10 Hz, 1H), 3.41 (t, J=9.5 Hz, 1H), 3.37 (s, 3H), 3.36 (s, 3H), 3.33 (s, 3H), 3.31 (s, 5H), 3.00 (m, 4H), 2.91 (t, J=13 Hz, 2H), 2.51 (d, J=14 Hz, 1H), 2.38 (m, 2H), 2.23-2.09 (m, 6H), 2.02 (m, 1H), 1.96-1.85 (m, 3H), 1.81-1.72 (m, 4H), 1.69 (s, 6H), 1.64-1.54 (m, 4H), 1.37-1.28 (m, 4H), 1.10 (qd, J=3.5, 12 Hz, 2H), 1.02 (t, J=4.5 Hz, 6H), 0.97-0.94 (m, 2H), 0.90 (t, J=7.0 Hz, 3H), 0.78 (d, J=6.5 Hz, 2H).

JH-FK-12: JH-FK-12 was synthesized from FK520 (100 mg, 0.13 mmol) and semicarbazide (6 eq, 56.8 mg, 0.76 mmol) in methanol (1.3 mL, 0.1 M) according to General Procedure B. Purification by column chromatography (silica gel, CH₂Cl₂/MeOH, 25/1) afforded JH-FK-12 as a white solid (41 mg, 38%): ¹H NMR (500 MHz, acetone-d6): δ 9.33 (s, 1H), 6.07 (br s, 2H), 5.30 (d, J=6.5 Hz, 1H), 5.22 (d, J=9.5 Hz, 1H), 5.13 (d, J=9.5 Hz, 1H), 4.74 (s, 2H), 4.53 (s, 1H), 4.36 (d, J=14 Hz, 1H), 3.96 (s, 1H), 3.68-3.58 (m, 4H), 3.46 (m, 2H), 3.39 (s, 3H), 3.37 (s, 3H), 3.36 (s, 3H), 3.33 (m, 5H), 3.13 (m, 3H), 2.98 (m, 1H), 2.41-2.31 (m, 3H), 2.25 (s, 2H), 2.19 (m, 2H), 1.98 (s, 2H), 1.91 (m, 2H), 1.78 (s, 2H), 1.69 (s, 4H), 1.62 (s, 3H), 1.44-1.30 (m, 6H), 0.96 (m, 6H), 0.90 (m, 6H).

JH-FK-13: JH-FK-13 was synthesized from FK520 (100 mg, 0.13 mmol) and tosylhydrazine (6 eq, 141 mg, 0.76 mmol) in methanol (1.3 mL, 0.1 M) according to General Procedure B. Purification by column chromatography (silica gel, CH₂Cl₂/MeOH, 25/1) afforded JH-FK-13 as a white solid (40 mg, 33%): ¹H NMR (500 MHz, acetone-d6): δ 9.83 (s, 1H), 7.79 (d, J=8.5 Hz, 2H), 7.36 (d, J=11 Hz, 2H), 5.35 (d, J=3.5 Hz, 1H), 5.15 (d, J=9.0 Hz, 1H), 5.06 (d, J=9.0 Hz, 2H), 4.60 (s, 1H), 4.40 (d, J=6.0 Hz, 1H), 4.34 (d, J=12 Hz, 1H), 3.91 (d, J=11 Hz, 1H), 3.63 (d, J=2.5 Hz, 1H), 3.52 (d, J=6.5 Hz, 1H), 3.50 (d, J=6.5 Hz, 1H), 3.37 (s, 2H), 3.35 (s, 2H), 3.33 (s, 2H), 3.09 (m, 2H), 3.07-2.95 (m, 2H), 2.43 (s, 1H), 2.41 (s, 2H), 2.38-2.32 (m, 1H), 2.31 (dd, J=2.5, 14 Hz, 1H), 2.28-2.20 (m, 2H), 2.19-2.10 (m, 3H), 2.087 (s, 2H), 2.00-1.94 (m, 2H), 1.91-1.85 (m, 2H), 1.82-1.71 (m, 2H), 1.66 (d, J=16 Hz, 1H), 1.63 (s, 3H), 1.61 (s, 1H), 1.57 (s, 3H), 1.43-1.30 (m, 7H), 1.13-1.06 (m, 2H), 0.96 (d, J=6.5 Hz, 4H), 0.91 (d, J=6.5 Hz, 3H), 0.89-0.085 (m, 2H), 0.83 (d, J=7.0 Hz, 2H), 0.71 (t, J=7.0 Hz, 1H), 0.62 (t, J=7.5 Hz, 3H).

JH-FK-14: JH-FK-14 was synthesized from FK520 (100 mg, 0.13 mmol) and isophthalic hydrazide (6 eq, 147 mg, 0.76 mmol) in methanol (1.3 mL, 0.1 M) according to General Procedure B. Purification by column chromatography (silica gel, CH₂Cl₂/MeOH, 25/1) afforded JH-FK-14 as a white solid (45 mg, 37%): ¹H NMR (500 MHz, acetone-d6): δ 11.76 (s, 1H), 10.04 (s, 1H), 8.30 (s, 1H), 7.98 (d, J=7.5 Hz, 1H), 7.93 (d, J=8.0 Hz, 1H), 7.47 (t, J=8.0 Hz, 1H), 6.19 (br s, 1H), 5.61 (s, 1H), 5.19 (d, J=9.0 Hz, 1H), 5.13 (s, J=9.0 Hz, 1H), 4.48 (s, 1H), 4.25 (m, 3H), 3.45 (t, J=10 Hz, 2H), 3.36 (d, J=3.0 Hz, 6H), 3.33 (s, 2H), 3.31 (s, 1H), 3.00-2.95 (m, 2H), 2.92 (m, 4H), 2.51 (d, J=14 Hz, 1H), 2.35 (m, 1H), 2.27-2.07 (m, 9H), 2.00 (m, 1H), 1.93 (t, J=12 Hz, 1H), 1.86 (m, 1H), 1.79 (m, 2H), 1.71 (s, 3H), 1.67 (s, 3H), 1.55 (m, 2H), 1.39-1.26 (m, 5H), 1.12-1.05 (m, 2H), 1.02 (t, J=10 Hz, 3H), 1.00 (t, J=7.5 Hz, 4H), 0.925 (m, 4H), 0.806 (d, J=6.5 Hz, 3H).

JH-FK-15: JH-FK-14 was synthesized from FK520 (100 mg, 0.13 mmol) and 2-naphthoic acid hydrazide (6 eq, 141 mg, 0.76 mmol) in methanol (1.3 mL, 0.1 M) according to General Procedure B. Purification by column chromatography (silica gel, CH2Cl2/MeOH, 25/1) afforded JH-FK-15 as a white solid (57 mg, 47%): ¹H NMR (500 MHz, acetone-d6): δ 11.56 (s, 1H), 8.52 (s, 1H), 8.06 (d, J=9.5 Hz, 1H), 7.98 (dd, J=9.0, 1.5 Hz, 1H), 7.92 (m, 2H), 7.57 (td, J=2.0, 7.0 Hz, 1H), 7.54 (td, J=2.0 Hz, 7.0 Hz, 1H), 5.74 (s, 2H), 5.19 (d, J=9.0 Hz, 1H), 5.17 (d, J=11 Hz, 1H), 4.45 (s, 1H), 4.37 (s, 1H), 4.29 (d, J=14 Hz, 1H), 4.19 (d, J=11 Hz, 1H), 3.37 (s, 1H), 3.49 (d, J=11 Hz, 1H), 3.46 (d, J=9.5 Hz, 1H), 3.36 (d, J=4.0 Hz, 6H), 3.34 (s, 2H), 3.32 (s, 2H), 3.17 (br s, 1H), 2.98 (m, 2H), 2.89 (s, 1H), 2.53 (d, J=14 Hz, 1H), 2.37 (m, 1H), 2.29-2.16 (m, 5H), 2.10 (m, 1H), 2.01 (m, 1H), 1.93 (t, J=11 Hz, 1H), 1.90-1.79 (m, 3H), 1.76 (m, 2H), 1.71 (s, 3H), 1.69 (s, 3H), 1.65-1.47 (m, 5H), 1.39-1.27 (m, 4H), 1.04 (m, 4H), 0.96 (q, J=12 Hz, 2H), 0.89 (m, 3H), 0.79 (d, J=6.5 Hz, 3H).

JH-FK-16: JH-FK-16 was synthesized from FK520 (100 mg, 0.13 mmol) and ethylhydrazine hydrochloride (6 eq, 73 mg, 0.76 mmol) in methanol (1.3 mL, 0.1 M) according to General Procedure B. Purification by column chromatography (silica gel, CH₂Cl₂/MeOH, 25/1) afforded JH-FK-16 as a white solid (44 mg, 42%): ¹H NMR (500 MHz, acetone-d6): δ 7.62 (s, 1H), 6.14 (t, J=6.0 Hz, 1H), 5.33 (s, 1H), 4.99 (d, J=8.0 Hz, 1H), 4.72 (m, 2H), 4.41 (d, J=6.0 Hz, 1H), 4.30 (d, J=15 Hz, 1H), 3.90 (d, J=7.5 Hz, 1H), 3.61 (d, J=3.0 Hz, 1H), 3.60 (dd, J=3.5, 7.5 Hz, 1H), 3.45 (d, J=3.5 Hz, 1H), 3.44 (s, 1H), 3.36 (m, 7H), 3.34-3.28 (m, 4H), 3.09 (m, 2H), 3.00-2.94 (m, 4H), 2.36 (m, 2H), 2.27-2.14 (m, 5H), 2.11 (s, 1H), 1.99 (m, 1H), 1.90-1.77 (m, 6H), 1.75 (s, 3H), 1.66 (s, 3H), 1.61 (s, 1H) 1.56 (m, 2H), 1.40-1.29 (m, 9H), 1.14 (t, J=7.0 Hz, 3H), 1.03 (d, J=6.0 Hz, 2H), 0.92 (s, 1H), 0.91 (s, 1H), 0.86 (m, 1H), 0.84 (d, J=3.0 Hz, 2H), 0.83 (m, 2H).

JH-FK-17: JH-FK-17 was synthesized from FK520 (100 mg, 0.13 mmol) and 4-fluorobenzoic acid hydrazide (6 eq, 147 mg, 0.76 mmol) in methanol (1.3 mL, 0.1 M) according to General Procedure B. Purification by column chromatography (silica gel, CH₂Cl₂/MeOH, 25/1) afforded JH-FK-17 as a white solid (53 mg, 45%): ¹H NMR (500 MHz, acetone-d6): δ 11.29 (s, 1H), 7.83 (dd, J=5.5, 9.0 Hz, 2H), 7.05 (d, J=10 Hz, 1H), 7.03 (d, J=9 Hz, 1H), 5.65 (s, 1H), 5.49 (s, 1H), 5.05 (d, J=11 Hz, 1H), 5.02 (d, J=9.0 Hz, 1H), 4.36 (s, 1H), 4.18 (m, 2H), 4.02 (d, J=6.0 Hz, 1H), 3.63 (s, 1H), 3.32 (m, 2H), 3.23 (m, 5H), 3.21 (s, 2H), 3.19 (s, 13H), 3.11 (br s, 5H), 2.84 (m, 5H), 2.37 (d, J=14 Hz, 1H), 2.24 (m, 1H), 2.10-1.99 (m, 7H), 1.89 (m, 1H), 1.81-1.59 (m, 5H), 1.56 (s, 3H), 1.55 (s, 3H), 1.50 (m, 2H), 1.44 (m, 2H), 1.26-1.16 (m, 4H), 0.89 (d, J=5.5 Hz, 3H), 0.88 (d, J=7.5 Hz, 3H), 0.77 (t, J=7.5 Hz, 3H).

JH-FK-20: JH-FK-20 was synthesized from FK520 (100 mg, 0.13 mmol) and 4-aminobenzoic hydrazide (6 eq, 114 mg, 0.76 mmol) in methanol (1.3 mL, 0.1 M) according to General Procedure B. Purification by column chromatography (silica gel, CH₂Cl₂/MeOH, 25/1) afforded JH-FK-20 as a white solid (50 mg, 43%): ¹H NMR (500 MHz, acetone-d6): δ 11.01 (s, 1H), 7.68 (d, J=9.5 Hz, 2H), 6.62 (d, J=9.0 Hz, 2H), 5.54 (s, 1H), 5.40 (s, 1H), 5.18 (d, J=9.5 Hz, 1H), 5.15 (d, J=9.5 Hz, 1H), 5.06 (s, 2H), 4.56 (s, 1H), 4.29 (m, 2H), 4.11 (m, 1H), 3.63 (d, J=2.5 Hz, 1H), 3.47 (d, J=9.0 Hz, 1H), 3.45 (d, J=7.5 Hz, 1H), 3.36 (m, 6H), 3.33 (s, 3H), 3.31 (m, 2H) 3.00-2.94 (m, 2H), 2.45 (d, J=14 Hz, 1H), 2.38 (m, 1H), 2.25-2.08 (m, 8H), 2.03 (m, 1H), 1.95-1.73 (m, 6H), 1.69 (s, 3H), 1.68 (s, 3H), 1.66-1.50 (m, 5H), 1.39-1.29 (m, 4H), 1.14-1.05 (m, 2H), 1.01 (d, J=2.5 Hz, 2H), 1.00 (d, J=3.0 Hz, 2H), 0.95 (q, J=13 Hz, 1H), 0.89 (t, J=7.0 Hz, 3H).

JH-FK-21: JH-FK-21 was synthesized from FK520 (100 mg, 0.13 mmol) and carbohydrazide (6 eq, 68.1 mg, 0.76 mmol) in methanol (1.3 mL, 0.1 M) according to General Procedure B. Purification by column chromatography (silica gel, CH₂Cl₂/MeOH, 25/1) afforded JH-FK-21 as a white solid (37 mg, 34%): ¹H NMR (500 MHz, acetone-d6): δ 10.88 (m, 1H), 9.23 (m, 1H), 5.24 (m, 2H), 5.12 (d, J=9.0 Hz, 2H), 5.01-4.57 (m, 2H), 4.33 (d, J=18 Hz, 1H), 3.91 (m, 1H), 3.71 (m, 1H), 3.58 (s, 1H), 3.45 (m, 1H), 3.67 (s, 3H), 3.34 (s, 2H), 3.33 (s, 3H), 3.31 (s, 3H), 3.30 (s, 3H), 3.24 (m, 3H), 2.95 (s, 4H), 2.40 (m, 1H), 2.28 (m, 1H), 2.28-2.15 (m, 5H), 2.00 (s, 2H), 1.98-1.93 (m, 3H), 1.90 (m, 3H), 1.74 (m, 3H), 1.70 (s, 3H), 1.58 (m, 5H), 1.41-1.28 (m, 3H), 0.99 (d, J=7.0 Hz, 3H), 0.90 (m, 8H).

JH-FK-22: JH-FK-22 was synthesized from FK520 (100 mg, 0.13 mmol) and 2-hydroxyacetic acid hydrazide (6 eq, 45 mg, 0.76 mmol) in methanol (1.3 mL, 0.1 M) according to General Procedure B. Purification by column chromatography (silica gel, CH₂Cl₂/MeOH, 25/1) afforded JH-FK-22 as a white solid (48 mg, 41%): ¹H NMR (500 MHz, acetone-d6): δ 11.01 (s, 1H), 10.27 (s, 1H), 5.53-5.45 (s, 1H) 5.18 (m, 2H), 5.02 (s, 1H), 4.46 (s, 1H), 4.41 (d, J=5.0 Hz, 1H), 4.36 (s, 1H), 4.34 (s, 1H), 4.31 (m, 1H), 4.17 (m, 1H), 4.06 (m, 1H), 3.65 (s, 1H), 3.56 (d, J=10 Hz, 1H), 3.48 (d, J=8.5 Hz, 1H), 3.44 (m, 3H), 3.38 (m, 6H), 3.35 (s, 3H), 3.32 (m, 1H), 3.21 (q, J=7.5 Hz, 1H), 3.11 (m, 1H), 2.99 (m, 1H), 2.38 (m, 2H), 2.26-2.10 (m, 6H), 1.97 (d, J=2.0 Hz, 1H), 1.99-1.86 (m, 2H), 1.82-1.76 (m, 2H), 1.68 (m, 4H), 1.66 (s, 1H), 1.64-1.57 (m, 4H), 1.39-1.29 (m, 4H), 1.19-1.08 (m, 2H), 1.02 (m, 6H), 0.91-0.84 (m, 6H).

JH-FK-23: JH-FK-23 was synthesized from FK520 (100 mg, 0.13 mmol) and methanesulfonyl hydrazide (6 eq, 83.3 mg, 0.76 mmol) in methanol (1.3 mL, 0.1 M) according to General Procedure B. Purification by column chromatography (silica gel, CH₂Cl₂/MeOH, 25/1) afforded JH-FK-23 as a white solid (58 mg, 52%): ¹H NMR (500 MHz, acetone-d6): δ 9.51 (s, 1H), 5.40 (d, J=3.5 Hz, 1H), 5.30 (s, 1H), 5.14 (t, J=7.5 Hz, 2H), 4.68 (s, 1H), 4.38 (d, J=4.0 Hz, 1H), 4.33 (d, J=12 Hz, 1H), 4.06 (d, J=9.5 Hz, 1H), 3.65 (d, J=3.0 Hz, 1H), 3.49 (d, J=9.5 Hz, 1H), 3.46 (d, J=9.5 Hz, 1H), 3.37 (s, 3H), 3.36 (s, 3H), 3.34 (s, 3H), 3.30 (m, 1H), 3.22 (q, J=8.0 Hz, 1H), 3.03-2.99 (m, 1H), 2.98 (s, 3H), 2.95 (m, 1H), 3.28 (m, 1H), 2.26 (m, 1H), 2.20-2.10 (m, 5H), 2.00 (m, 1H), 1.93 (m, 1H), 1.90 (m, 1H), 1.87 (m, 1H), 1.18 (t, J=13 Hz, 2H), 1.71 (m, 1H), 1.67 (s, 3H), 1.64 (s, 3H), 1.61-1.51 (m, 4H), 1.38-1.28 (m, 4H), 1.10-1.03 (m, 3H), 0.99 (d, J=6.5 Hz, 3H), 0.98 (d, J=8.0 Hz, 3H), 0.89 (t, J=7.5 Hz, 4H), 0.86 (d, J=6.5 Hz, 2H).

### Discussion

In the experiments described in this Example, an expanded library of structurally validated FK520 analogs was generated by one-step chemical synthesis and screened for their antifungal and immunosuppressive activity to develop non-immunosuppressive fungal-specific calcineurin inhibitors. The research and development of novel antifungal therapeutics are relevant due to rapidly emerging resistance to the current antifungal arsenal (see, Sionov E, et al., 2010. PLoS Pathog 6:e1000848; Rocha M F G, et al., 2016. Mycoses 59:281-290). Additionally, cryptococcal infections are associated with high mortality, and the treatment options for the population most devastated by this human fungal pathogen are typically limited to orally available but fungistatic fluconazole (see, Bongomin F, et al., 2017. J Fungi (Bosel) 3:57; Rajasingham R, et al, 2022. Lancet Infect Dis 22:1748-1755). FK506 is an orally active drug, making the development of fungal-specific analogs a potential avenue to better serve these patients.

By first assessing the in vitro antifungal and immunosuppressive activity of the various C-22-modified FK520 analogs, it was possible to confirm fungal specificity. During antifungal in vitro screening, fungal calcineurin inhibition was measured through microdilution broth growth assays. These results demonstrated that many compounds were able to maintain potent antifungal activity, indicating that these structural modifications did not prevent fungal calcineurin targeting. Reduced mammalian calcineurin inhibition by the FK520 analogs was confirmed in cytokine production studies. During this in vitro immunosuppressive assay, production of the cytokine IL-2, which is known to be inhibited by FK520, was shown to be suppressed to a lesser degree by the FK520 analogs. This finding confirms reduced targeting of mammalian calcineurin by the FK520 analogs. Using this information, therapeutic index scores were calculated to quantify the degree to which fungal selectivity was achieved.

JH-FK-08 was identified as the lead compound following in vitro screening. The introduced structural modifications did not significantly alter the pharmacokinetic (PK) properties (e.g., Cₘₐₓ, half-life, whole-blood drug concentration over time) compared to FK506 in both male and female mice. A pattern of higher plasma exposure (higher AUC, decreased clearance) in females was also observed in JH-FK-08- and FK506-treated animals relative to male-treated animals (Table 10). To further study the antifungal effect of calcineurin inhibition during infection, JH-FK-08 was then investigated for in vivo antifungal efficacy. Animals were intranasally infected with C. neoformans to replicate the conventional infection route in humans. In this study, JH-FK-08 was found to significantly reduce lung fungal burden by approximately 200-fold during 2 weeks of treatment and more than double murine survival time during infection compared to untreated infected animals. These benefits were further enhanced when animals were treated with JH-FK-08 in combination with fluconazole.

TABLE 10
Pharmacokinetic analysis of FK506 and JH-FK-08

| | FK506 Females | | FK506 Males | | JH-FK-08 Females | | JH-FK-08 Males | |
|---|---|---|---|---|---|---|---|---|
| | Value | st dev | Value | st dev | Value | st dev | Value | st dev |
| Tₘₐₓ [h] | 1.11 | 0.67 | 0.75 | 0.00 | 2.50 | 0.87 | 1.25 | 0.43 |
| Cₘₐₓ [μg/mL] | 5.96 | 1.11 | 4.51 | 0.62 | 7.50 | 1.33 | 4.07 | 1.22 |
| AUC (area under the | 27.10 | 7.28 | 15.17 | 5.72 | 31.42 | 4.32 | 15.64 | 5.05 |
| curve) [μg mL − 1 h] | | | | | | | | |
| t_{1/2} (3-12 h) (half- | 1.35 | 0.16 | 1.48 | 0.14 | 1.14 | 0.02 | 1.29 | 0.05 |
| life) [h] | | | | | | | | |
| t_{1/2} (12-24 h) (half- | 3.59 | 0.95 | 2.46 | 0.22 | 1.88 | 0.03 | 2.26 | 0.29 |
| life) [h] | | | | | | | | |
| CI/F (clearance as | 1555 | 452 | 2961 | 1310 | 1291 | 192 | 2786 | 1065 |
| Dose/AUCINF) [mL | | | | | | | | |
| h⁻¹ kg] | | | | | | | | |

During these experiments, dosing animals with either JH-FK-08 alone or in combination with fluconazole significantly reduced fungal burden and extended survival during cryptococcal infection. However, during this treatment time frame, infection was not fully cleared, and therefore infection progression led to mortality even in treated animals. One potential future direction is to extend the treatment time to assess if clearance can be achieved.

Overall, the preclinical efforts presented here advance JH-FK-08 as a promising candidate for a potential clinical study. The activity of JH-FK-08 far exceeds the previous two libraries' top candidates, APX879 and JH-FK-05, based on therapeutic index score comparison and in vivo efficacy (see, Juvvadi P R, et al., 2019. Nat Commun 10:4275; Aria T, Kouama Y, Suenaga T. Honda H. 1962. J Antibiot 15:231-232). These results support further study of calcineurin inhibition as a potential effective antifungal approach. Exciting research avenues for future study include (i) investigating the oral activity of JH-FK-08; (ii) researching how JH-FK-08 can interact with and cross the blood-brain barrier; and (iii) investigating possible combination therapies. Current treatments for fungal infections make use of combinations of therapies, and using multiple drugs during one therapeutic application can increase treatment efficacy as well as reduce emerging resistance and mitigate potential side effects (see, Perfect J R, et al., 2010. Clin Infect Dis 50:291-322; Ascierto P A, Marincola F M. 2011. J Transl Med 9:115). One potential combination partner is APX001A and its analogs, as this compound is under clinical investigation as a novel antifungal class (see, Liston S D, et al., 2022. J Fungi 8:1102; Shaw K J, et al., 2018. Antimicrob Agents Chemother 62:e00523-18; Shaheen S K, et al., 2021. Antimicrob Agents Chemother 65:e0068221). Furthermore, this approach of designing fungal-specific analogs of existing therapies can be utilized with other natural FKBP12-binding products, such as rapamycin, which demonstrates potent antifungal activity against high-priority fungal pathogens (see, Gabrielsson J, Weiner D. 2012. Methods Mol Biol Clifton NJ 929:377-389; Wong G K, et al. 1998 J Antibiot 51:487-491).

### Example 4

Recent studies have made significant breakthroughs in applying structure-guided drug design to increase the selectivity of calcineurin inhibitors for their fungal target. The C₂₂-modified FK506 analog, APX879, was found to be approximately 4-fold increased for its fungal selectivity compared to FK506 and significantly reduced fungal burden in an animal model of cryptococcosis. Moreover, structural evidence from Gobeil et al. suggests that APX879 binds to the immunophilin FKBP12 similarly to its parent molecule (see, Gobeil S M, et al., 2021, mBio 12:e0300021). This structural data as well as molecular dynamic (MD) simulations laid the groundwork for additional modifications of FK506 and FK520.

The FK520 analog, JH-FK-05, was developed based on recently solved crystal structures of FK520 bound to Aspergillus fumigatus FKBP12. This marked a significant advancement in the iterative design process of generating second-generation analogs based on APX879. Both the studies with APX879 and JH-FK-05 demonstrated that C₂₂ and C₂₁ are key exploitable residues on the molecule. Additionally, it was shown that both FK506 and FK520 are compatible with single-step synthesis selectively targeting substitution at the C₂₂ residue. Previous efforts to design fungal-specific calcineurin inhibitors have focused on modifications made to C₁₈, a region of FK506/FK520 distinct from C₂₂/C₂₁. A C₁₈-hydroxylated FK520 analog called L-685,818 was developed by Merck as an FK506 antagonist and was reported to be non-immunosuppressive while maintaining robust antifungal activity. Protein crystal structures revealed that L-685,818 binds human FKBP12 and the C₁₈ and C₂₁ modifications are both exposed on the calcineurin interacting effector surface. Although it is unclear how exactly this C₁₈ modification selectively disrupts mammalian activity, it has been suggested that the introduction of the polar hydroxyl group may disrupt a hydrophobic interface that would otherwise dock with calcineurin. However, compound availability and the complex chemistry to selectively hydroxylate the C₁₈ residue limited further study. With recent advancements in chemical synthesis and increased FK520 availability, Cl18 represents an excellent target for the development of additional fungal specific FK506/FK520 analogs.

### Synthesis and In Vitro Testing of Novel C₁₈-Modified FK506/FK520 Analogs:

Previous efforts to modify FK506 and FK520 identified a promising C₁₈-modified FK520 analog called L-685,818. This compound developed by Merck was hydroxylated at the C₁₈ residue of FK520 and demonstrated a high degree of fungal selectivity. L-685,818 (JH-FK-11 (PAD-008);

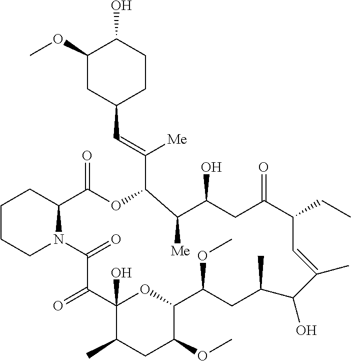 [SMILES: CC[C@@H]1/C=C(\C)C(O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

was resynthesized through an allylic oxidation (SeO2-THBP) and obtained the C₁₈ mono-hydroxylated FK520 as a single diastereomer. Additionally, FK506 was utilized as starting material for the same reaction and obtained the C₁₈ hydroxylated FK506 compound JH-FK-10 (PAD-007)

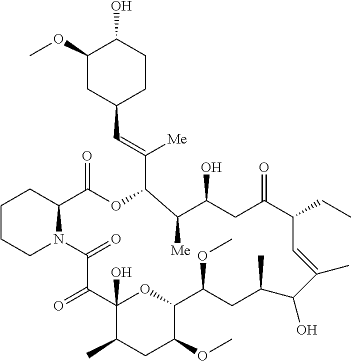 [SMILES: CC[C@@H]1/C=C(\C)C(O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

Both JH-FK-10 and JH-FK-11 maintained in vitro antifungal activity against C. neoformans with MICs of 0.8 and 1.6 μg/mL, respectively (Table 11). In the primary murine T cell model of immunosuppression, both JH-FK-10 and JH-FK-11 exhibited detectable immunosuppressive activities (FIG. 21). JH-FK-11 was significantly reduced for immunosuppressive activity compared to its FK506 counterpart JH-FK-10. However, both C₁₈-modified analogs were significantly reduced for immunosuppressive activity compared to their respective parental molecules. Therapeutic Index calculations demonstrate that JH-FK-11 has a significant fungal selectivity advantage over JH-FK-10 (JH-FK-10 TI_{score}=11.8, JH-FK-10 TI_{score}=3.2) (Table 12).

TABLE 11
Minimum Inhibitory Concentrations (MICs) against
C. neoformans for C18-modified analogs.

| Compound | C. neoformans MIC (μg/mL) |
|---|---|
| FK506 | 0.05 |
| FK520 | 0.05 |
| JH-FK-10 | 0.8 |
| JH-FK-11 | 3.2 |

C. neoformans MICs performed at 37° C.

TABLE 12
Therapeutic Index Scores for C18-modified analogs.

| | C. neoformans | IL-2 | |
|---|---|---|---|
| | MIC | IC50 | Therapeutic |
| Compound | (μg/mL) | (nM) | Index Score |
| FK506 | 0.05 | 0.11 | 1.0 |
| FK520 | 0.05 | 0.48 | 4.4 |
| JH-FK-10 | 0.8 | 5.7 | 3.2 |
| JH-FK-11 | 1.6 | 41.4 | 11.8 |

C. neoformans MICs performed at 37° C.

Combinatorial Synthesis and Activity of C₂₂- and C₁₈-Modified FK520 Analog JH-FK-18

To determine if the fungal selectivity observed with FK520 C₂₂ modifications (JH-FK-05) and FK520 C₁₈ modifications (JH-FK-11) could be enhanced when combined, both modifications were incorporated by synthesizing JH-FK-18

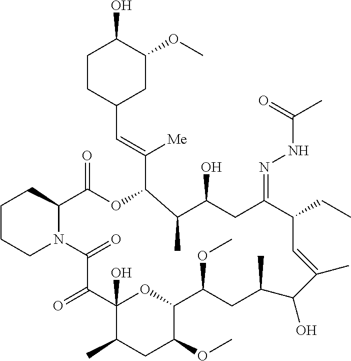 [SMILES: CC[C@@H]1/C=C(\C)C(O)[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/NC(C)=O)[C@H](C)C[C@@H]2OC]

This multi-step synthesis involved a primary C₂₂ modification of FK520 followed by the selective C₁₈ modification. Unfortunately, JH-FK-18 displayed no detectable antifungal or immunosuppressive activity (Table 5). JH-FK-18 did not inhibit C. neoformans growth up to a maximum concentration of 50 μg/mL, which is significantly higher than either C₁₈- or C₂₂-modified compounds alone. Primary murine T cells treated with 200 nM JH-FK-18 displayed no changes in IL-2 production.

TABLE 13
Therapeutic Index Scores for combinatorial
C22/C18-modified analog JH-FK-18.

| | C. neoformans | IL-2 | |
|---|---|---|---|
| | MIC | IC50 | Therapeutic |
| Compound | (μg/mL) | (nM) | Index Score |
| FK506 | 0.05 | 0.11 | 1.0 |
| FK520 | 0.05 | 0.48 | 4.4 |
| JH-FK-05 | 1.0 | 20.0 | 9.1 |
| JH-FK-11 | 1.6 | 41.4 | 11.8 |
| JH-FK-18 | >50 | >200 | — |

C. neoformans MICs performed at 37° C.

### Discussion

C₂₂ modifications of FK506 and FK520 have been successful at introducing fungal selectivity. Here, it is reported that multiple C₂₂ analogs of FK520 with diverse modifications and a high degree of biological activity variability. Differences in fungal selectivity and the balance of antifungal/immunosuppressive activities will allow for future studies to expand upon the SAR at this residue. Interestingly, none of the compounds containing bulky aromatic groups introduced any significant selectivity above a TI_{score} of 4. One compound, JH-FK-08, emerged as a highly fungal selective candidate with a TI_{score}=15.5. JH-FK-08 is structurally quite similar to JH-FK-05, which exhibited robust in vivo antifungal activity. 660 mg of JH-FK-18 was synthesized for testing in murine MTD and antifungal efficacy studies. Although JH-FK-18 is slightly increased for immunosuppressive activity in vitro compared to JH-FK-05, it maintains the highest antifungal activity (MIC 0.4 μg/mL) of all FK506/FK520 analogs that have been advanced to animal testing to date. Animal models of survival and fungal burden will allow us to assess if the increased in vitro antifungal activity will translate into therapeutic efficacy in animal models of fungal infection.

In addition to C₂₂ modifications of FK520, it was shown that C₁₈ modifications could impart robust fungal selectivity. L-685,818 was originally a C₁₈-modified calcineurin inhibitor developed by Merck as an FK506 antagonist. Multiple groups showed that L-685,818 exhibited broad-spectrum antifungal activity and was reduced for immunosuppression in vitro. However, likely due to complex synthetic procedures, L-685,818 was halted in development. Recent advances in availability of bulk starting material and chemical synthetic procedures allowed us to re-synthesize L-685,818 as JH-FK-11. Applying this same method, JH-FK-10 was generated which is the FK506 counterpart to JH-FK-11 and found that both compounds were increased for fungal selectivity, though the selectivity of JH-FK-11 was significantly more pronounced. Both JH-FK-10 and JH-FK-11 are identical molecules except for their C₂₁ residues, reaffirming the importance of C₂₁ in conferring fungal selectivity.

Because of clear differences observed in the activities of the two C₁₈-modified compounds, it was asked if introducing combinatorial modifications at another validated residue, C₂₂, would enhance fungal selectivity. Unfortunately, despite the fungal selectivity that these C₁₈ and C₂₂ modifications introduce individually, a compound with both modifications (JH-FK-18) has no detectable biological activity. This emphasizes the importance of SAR and modeling to better understand how modifications interact individually and in combination. Recent studies in molecular dynamic simulations have shown that a single chemical modification on FK506/FK520 can drastically influence predicted drug-protein contacts. Thus, it may be unlikely that simply combining modifications that have positive individual activity profiles will result in a compound with enhanced activity or specificity.

### Example 5

This example provides additional FK520 Analog structures (JH-FK-29, JH-FK-30, JH-FK-31, JH-FK-33, JH-FK-35, JH-FK-37, JH-FK-38, and JH-FK-40).

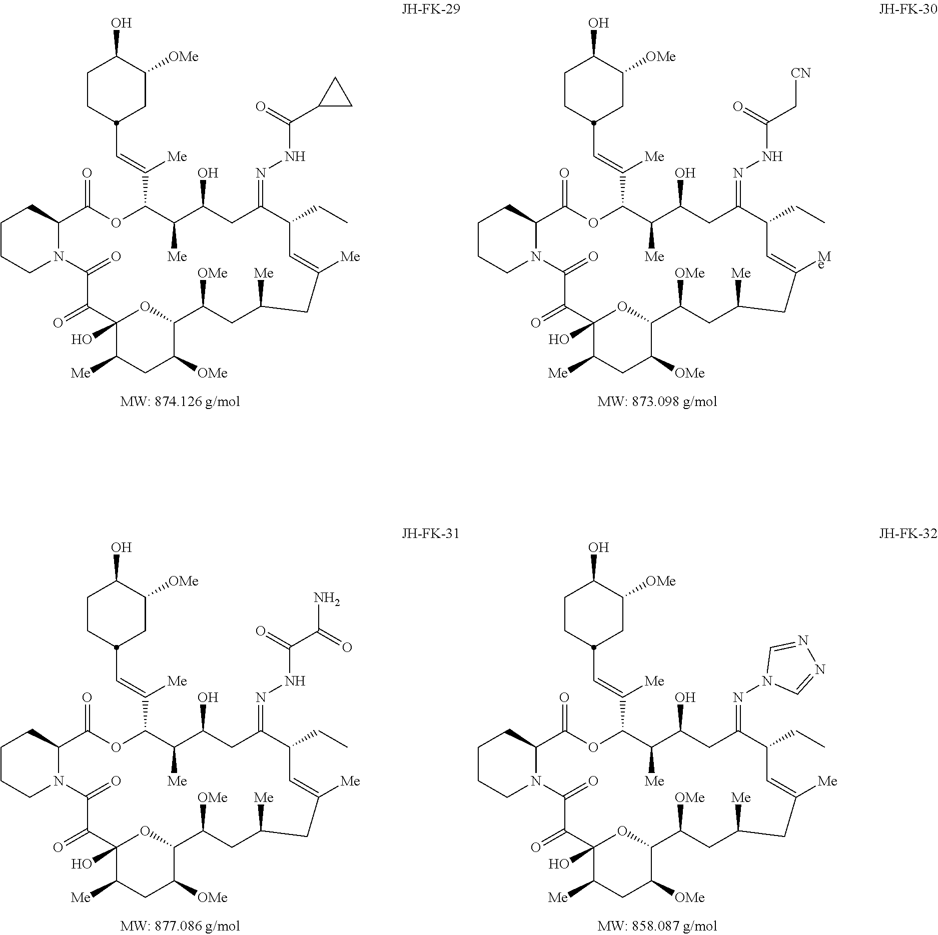

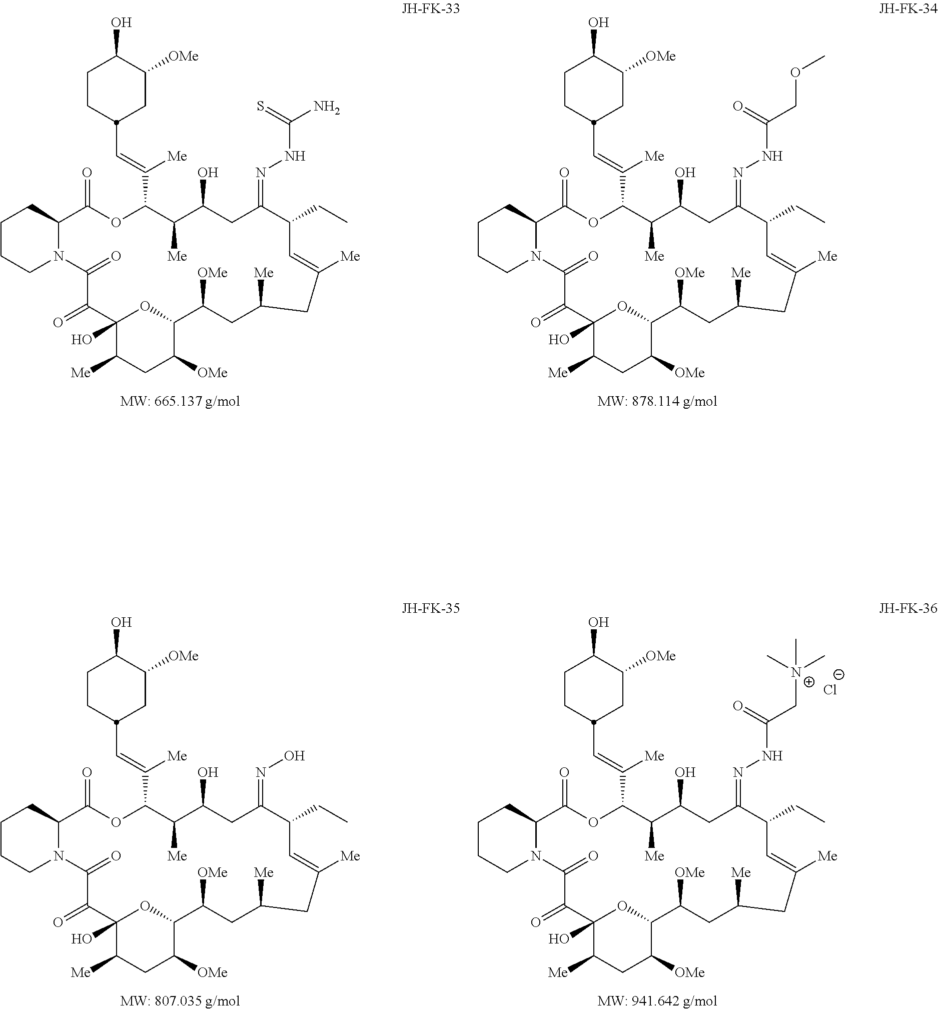

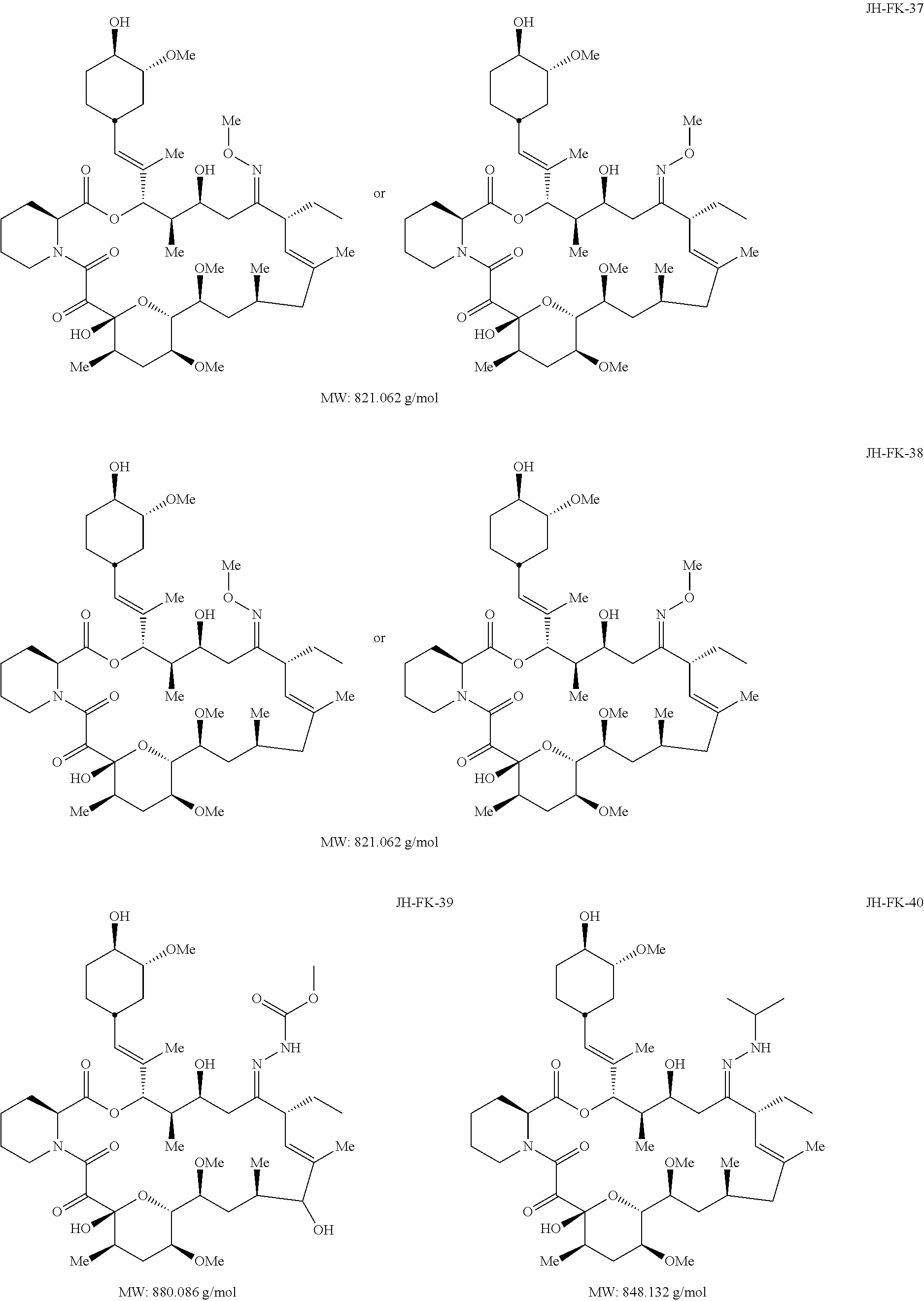

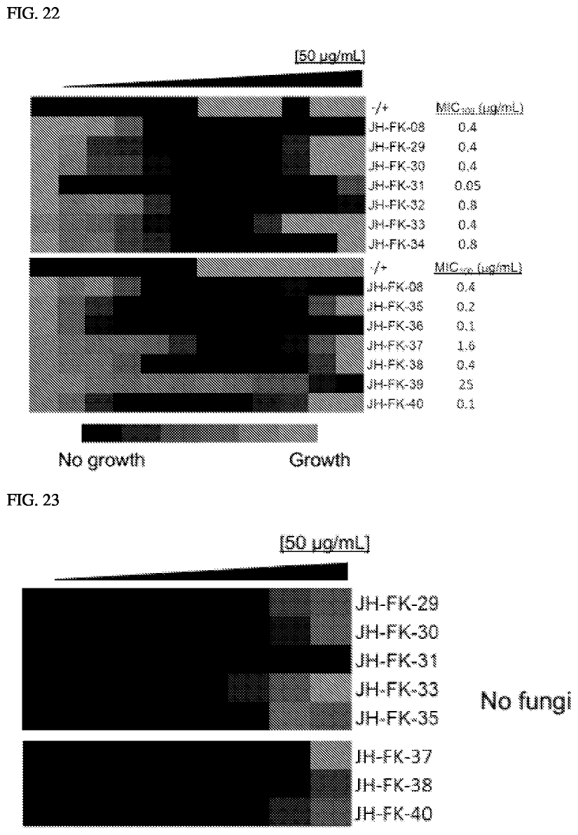

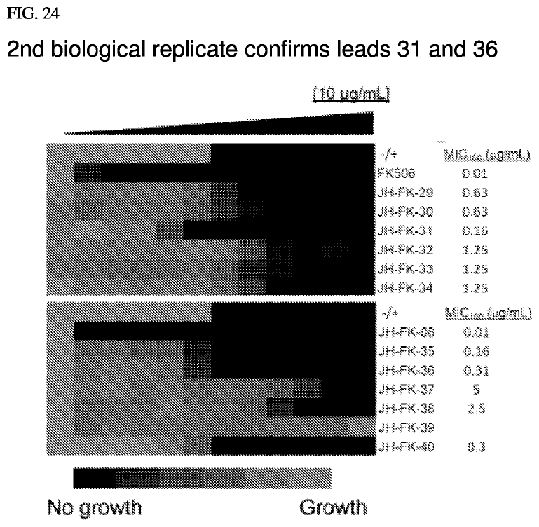

FIGS. 22, 23 and 24 present antifungal activity for JH-FK-29, JH-FK-30, JH-FK-31, JH-FK-33, JH-FK-35, JH-FK-37, JH-FK-38, and JH-FK-40.

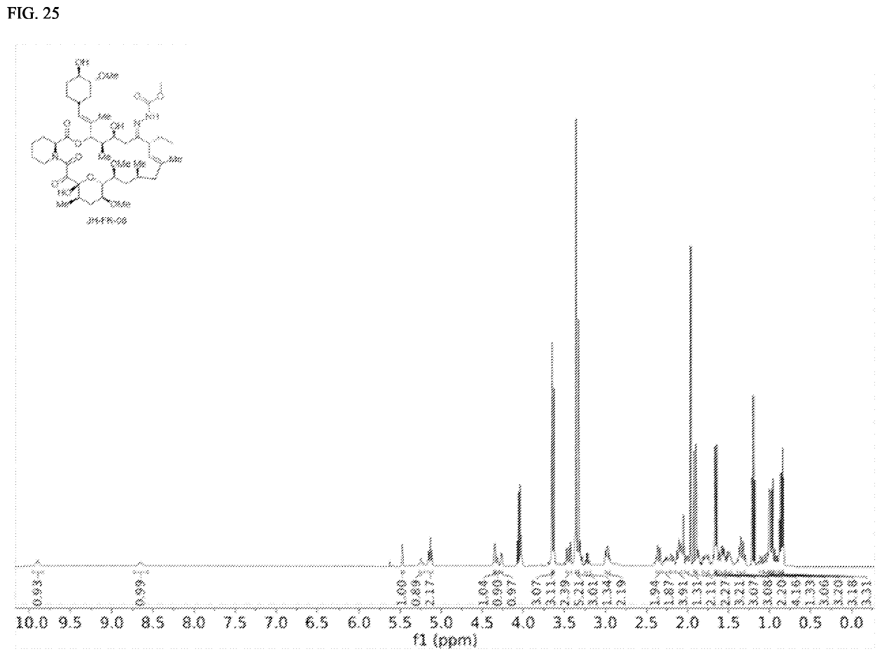

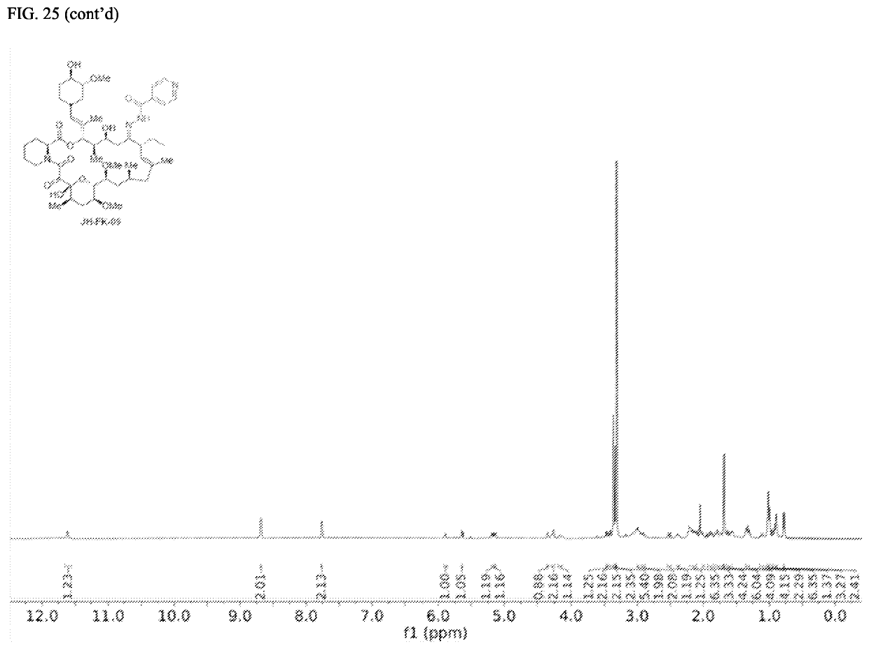

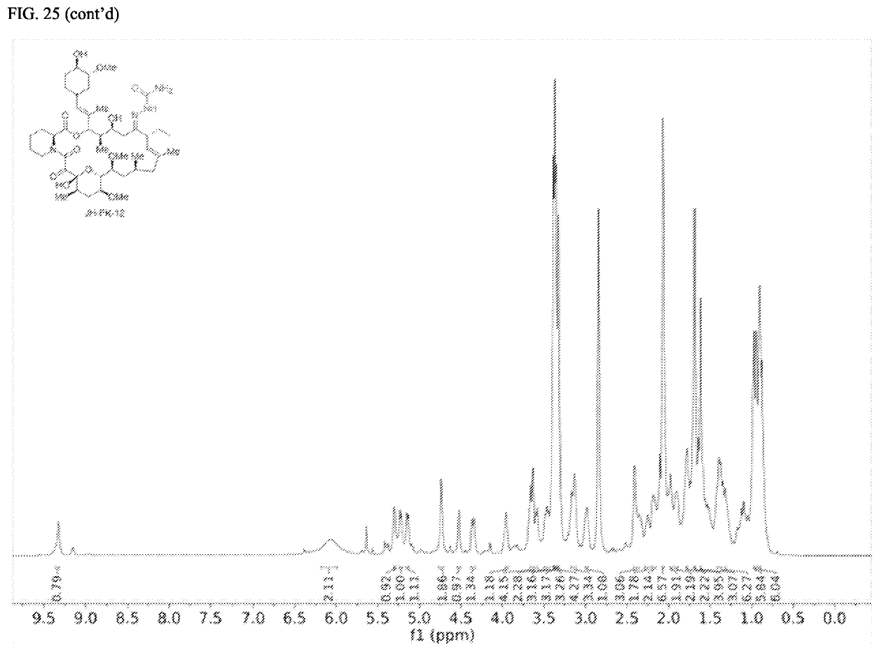

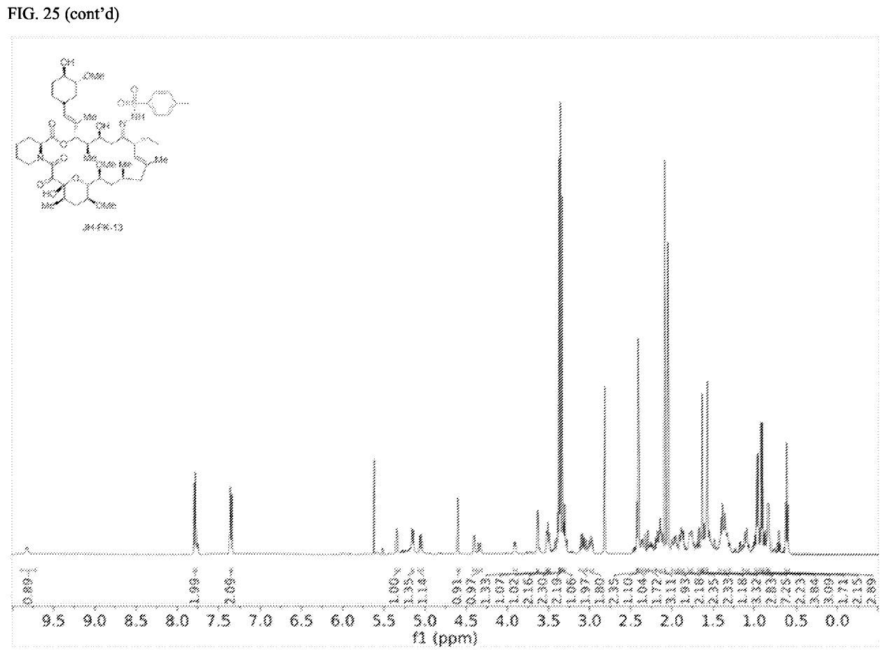

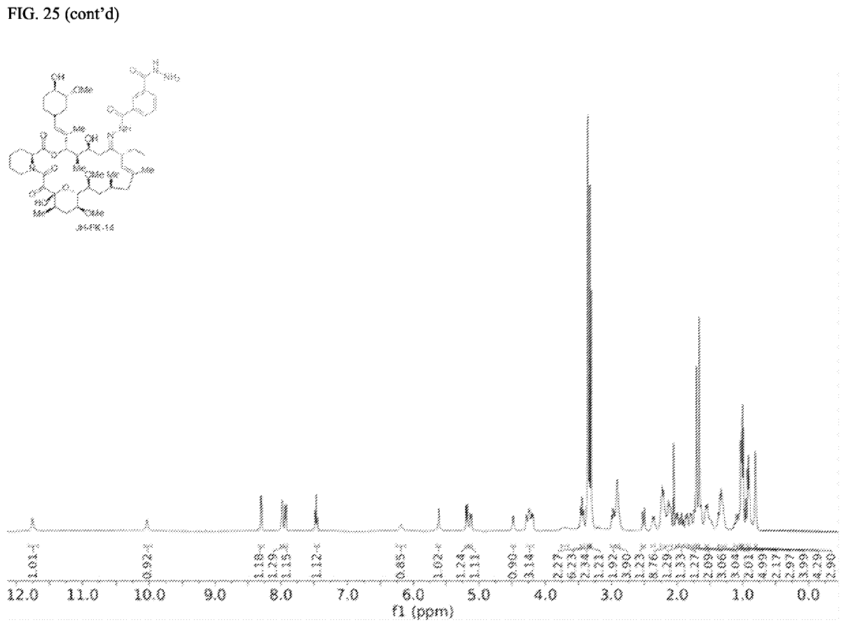

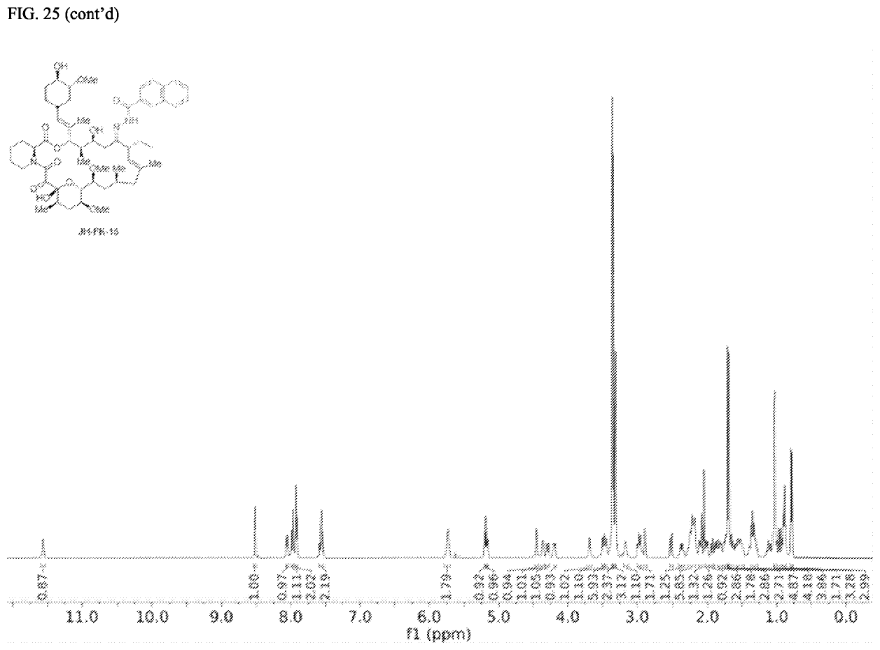

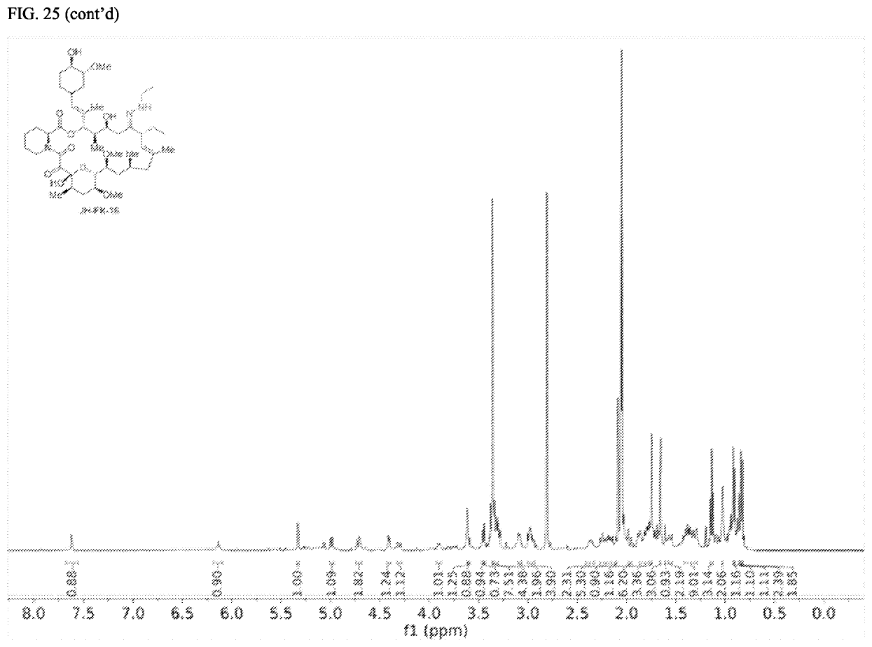

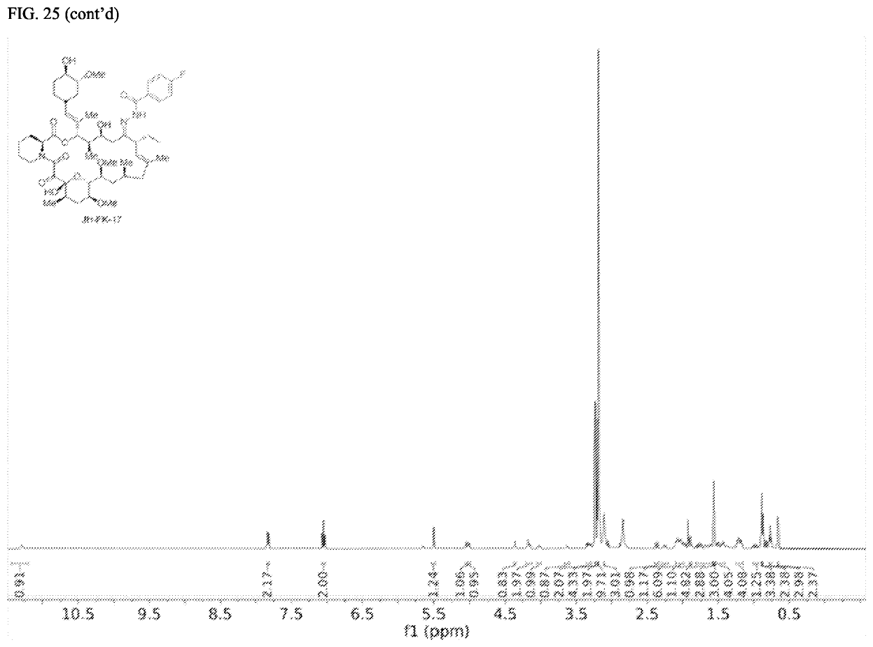

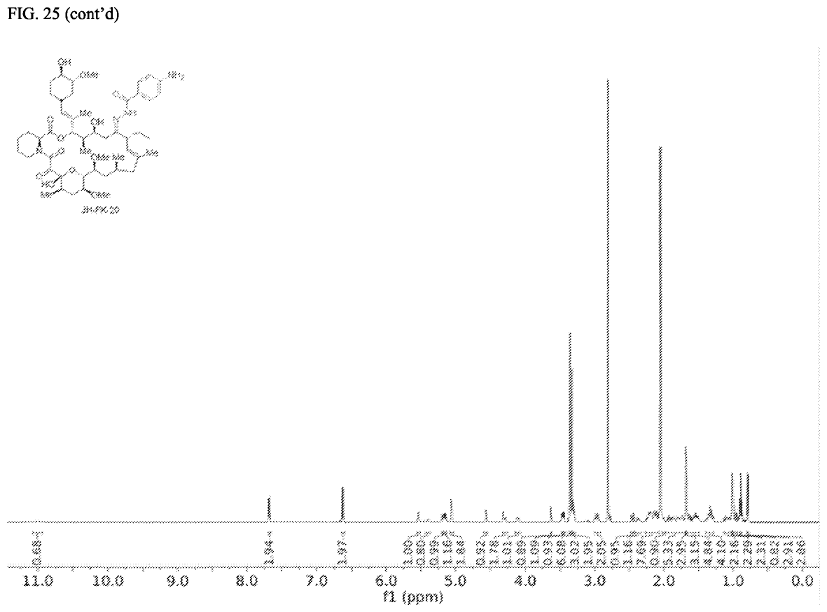

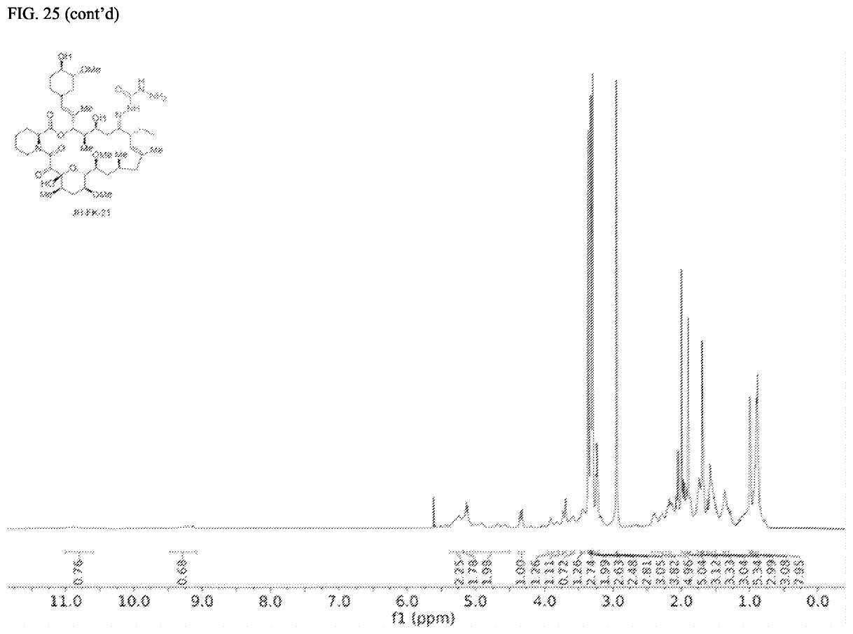

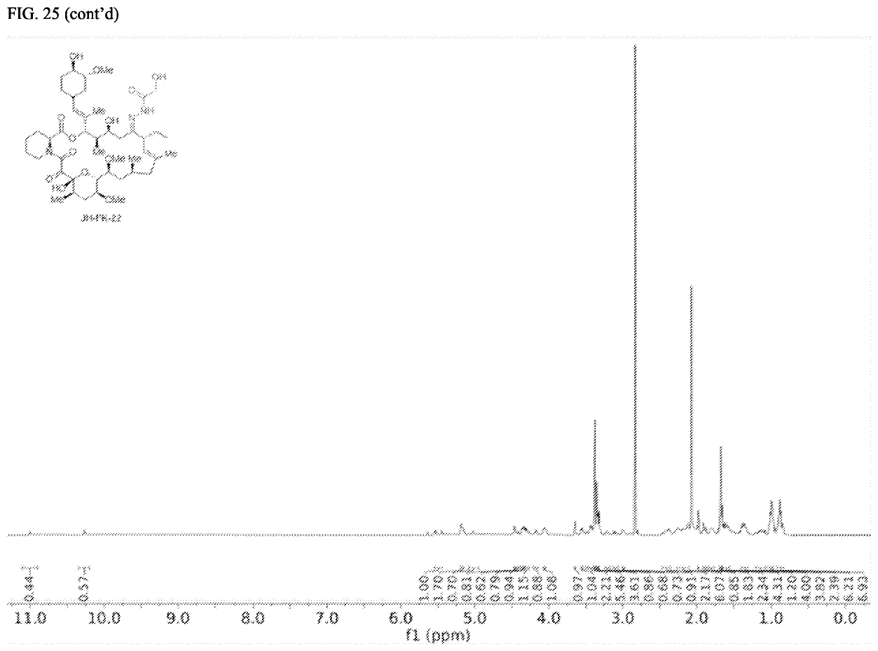

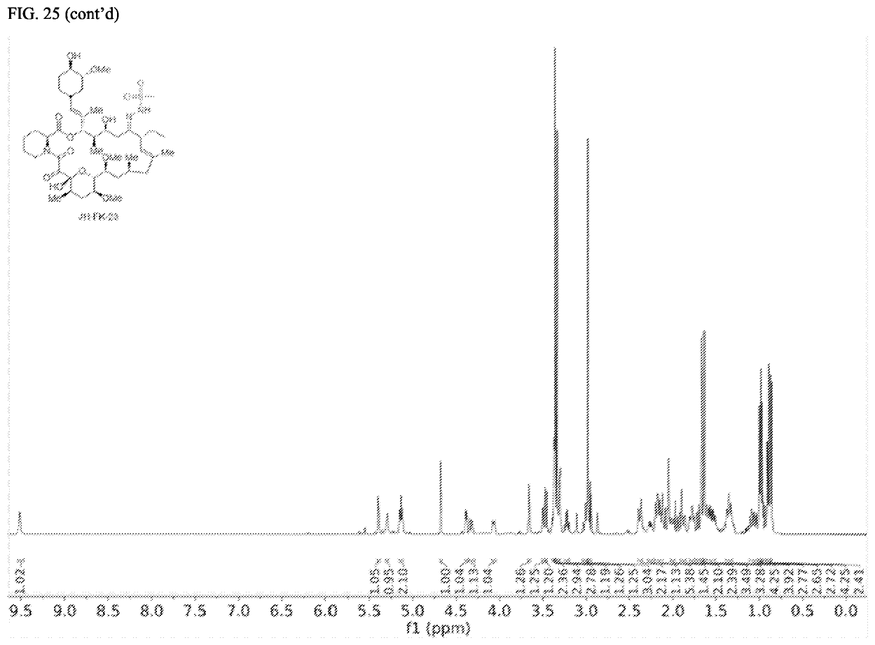

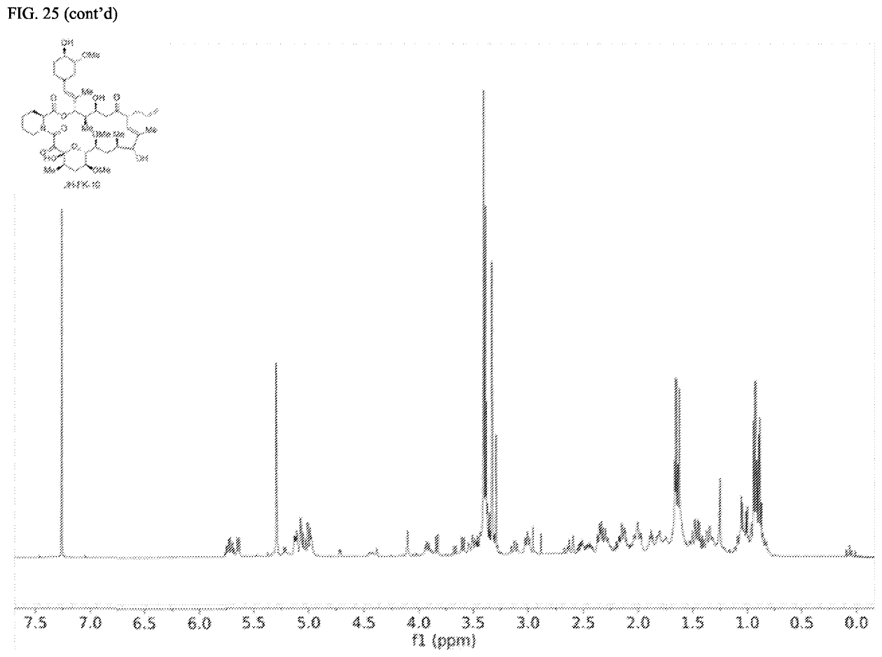

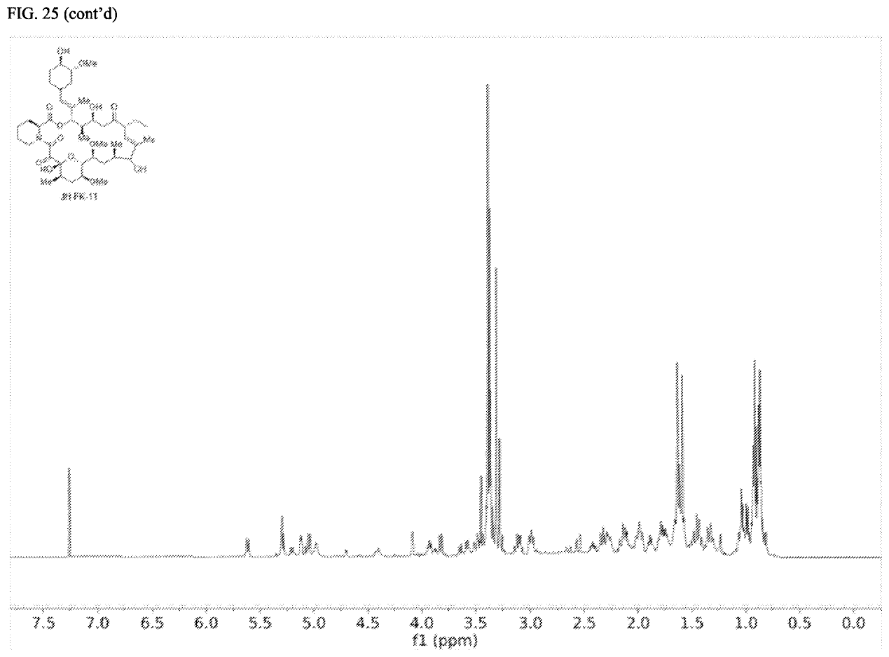

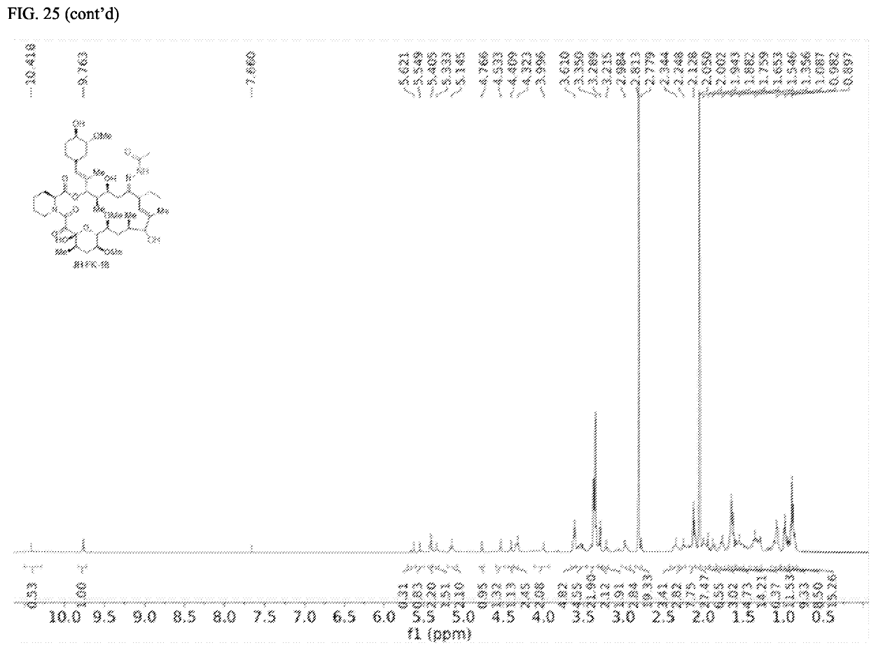

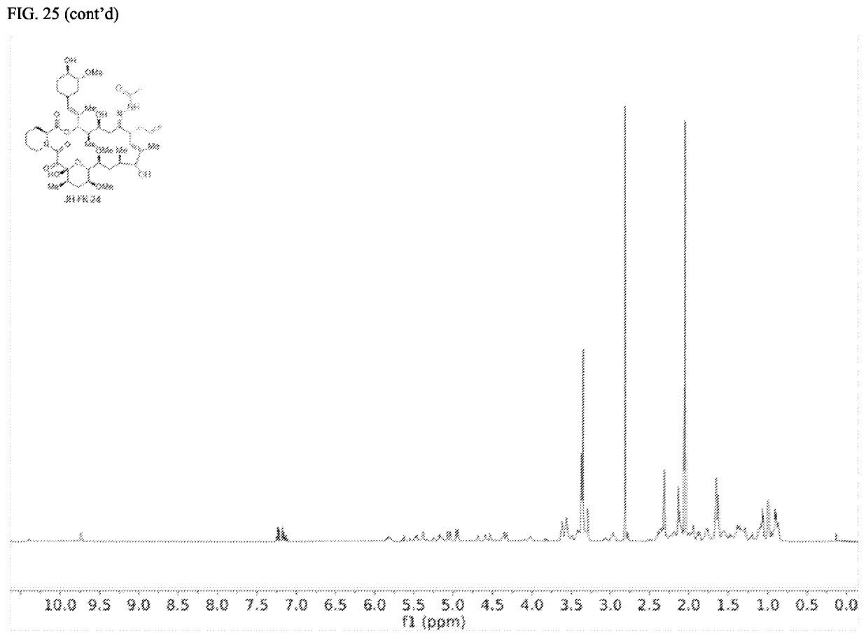

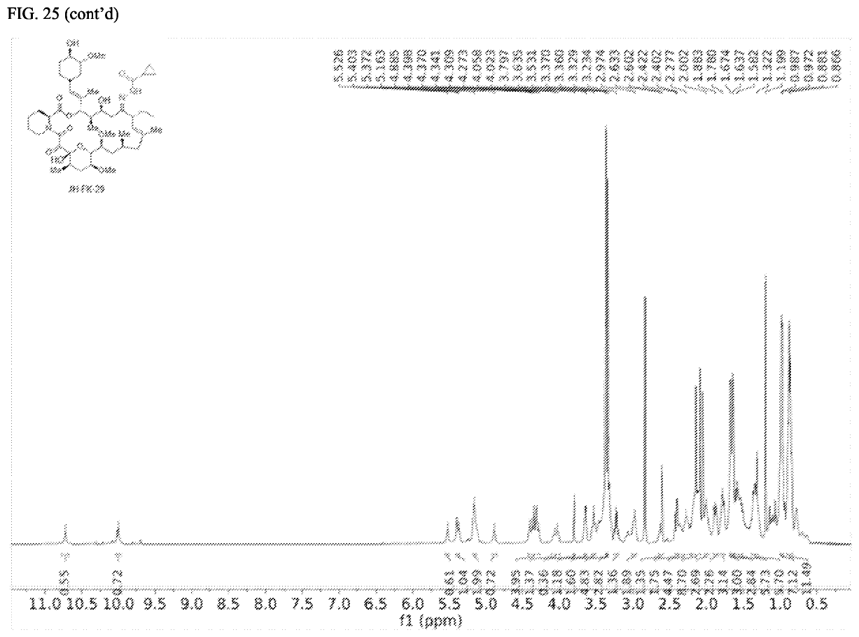

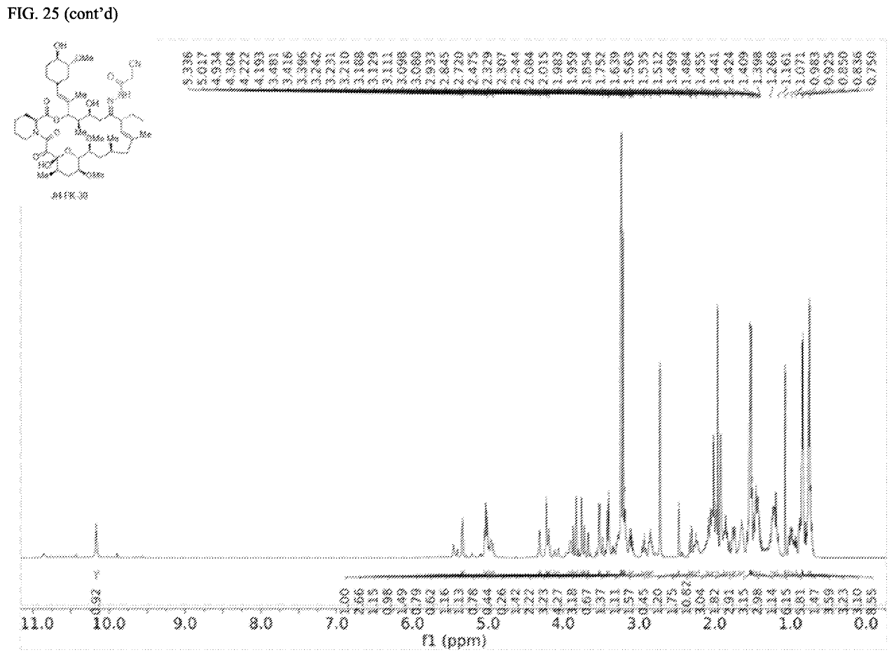

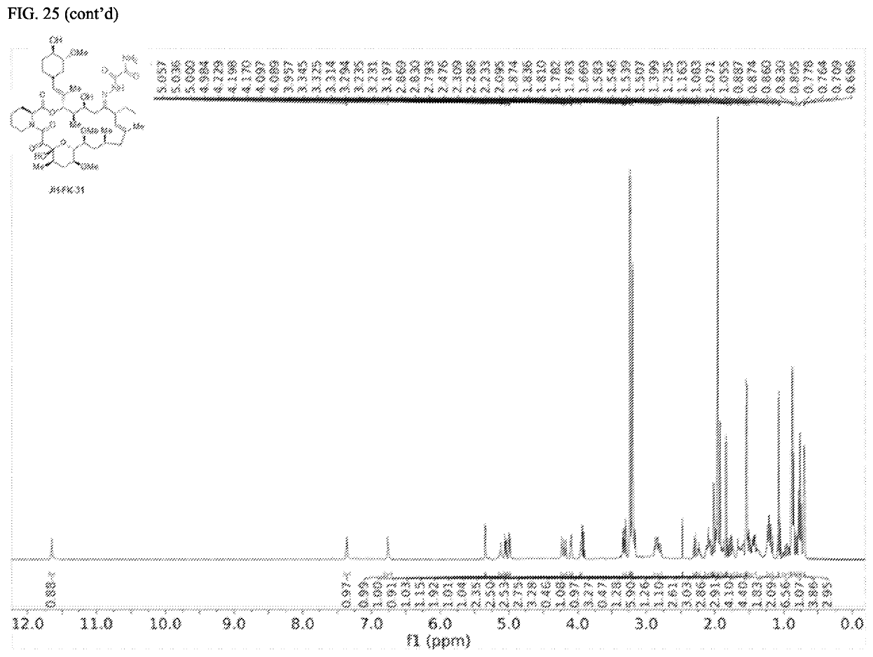

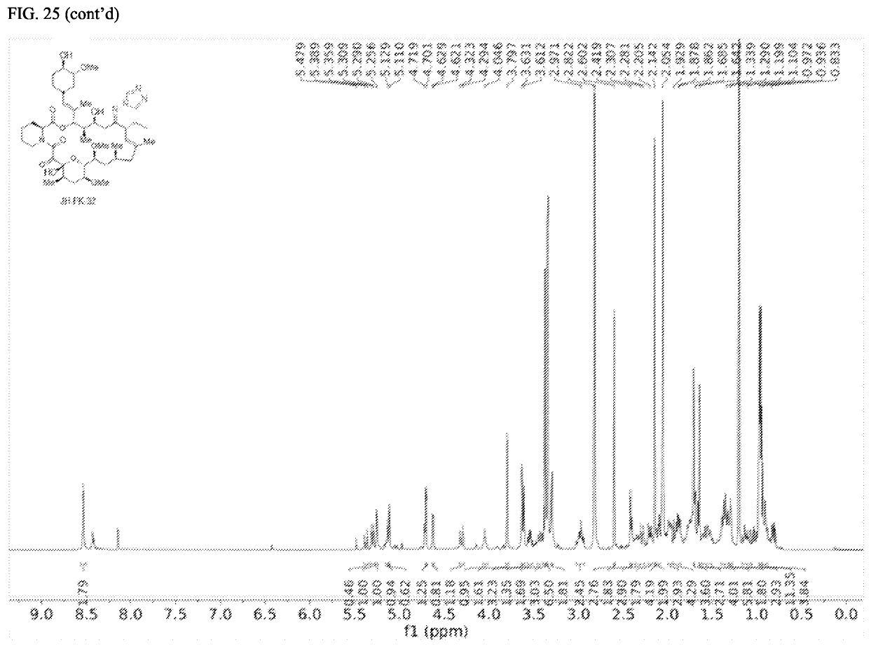

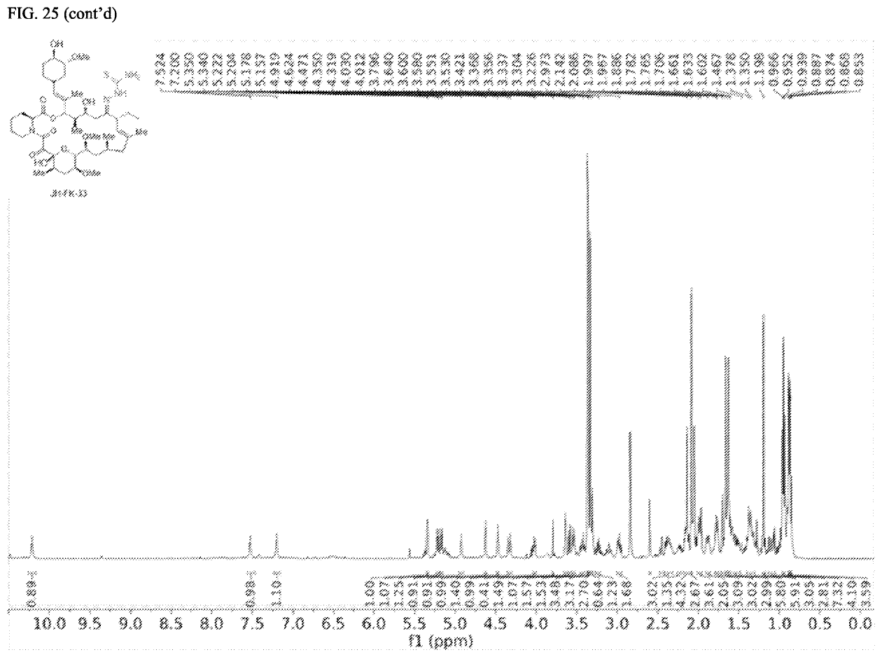

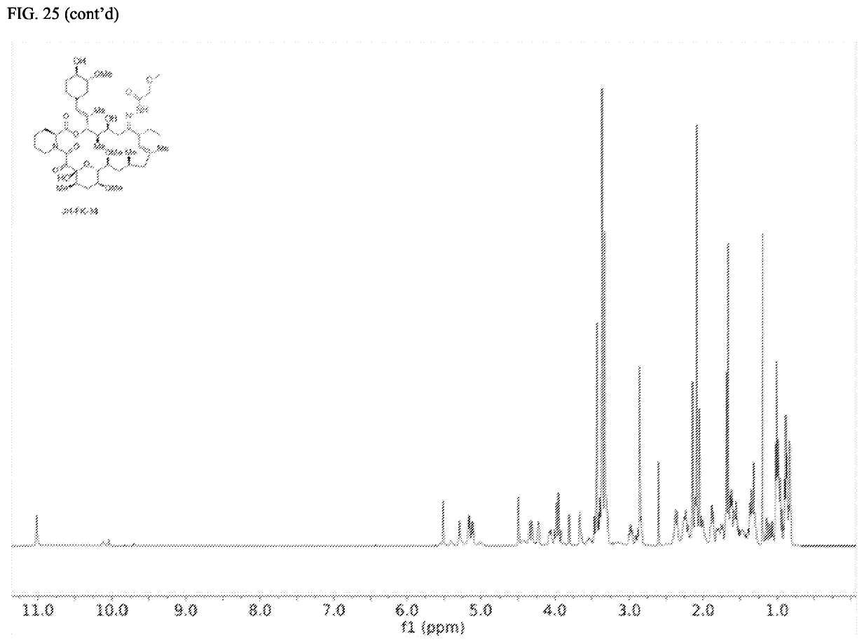

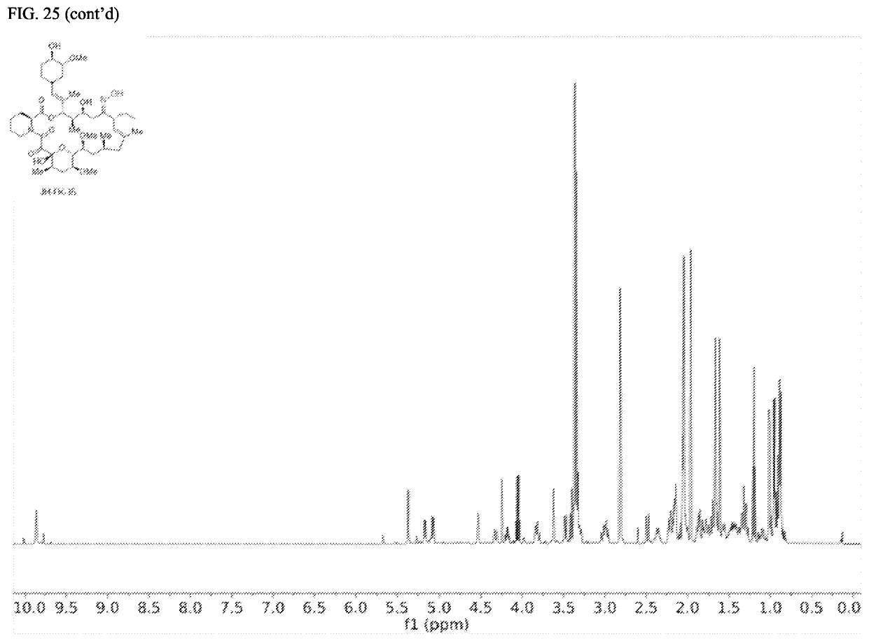

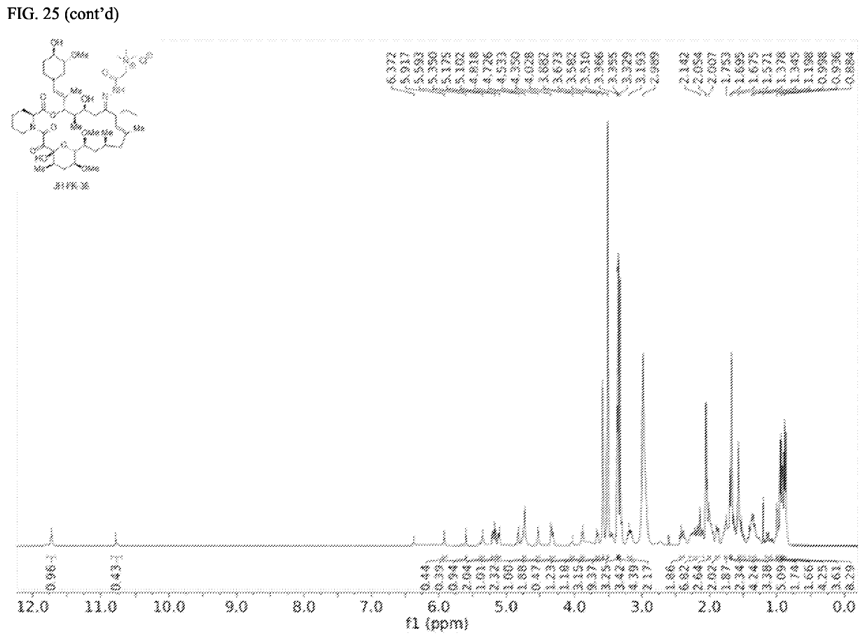

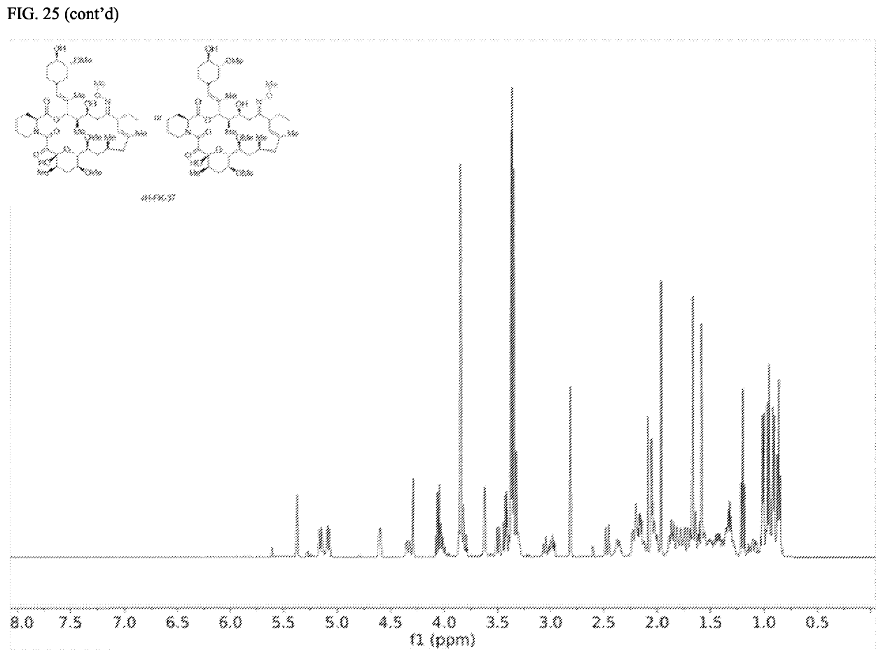

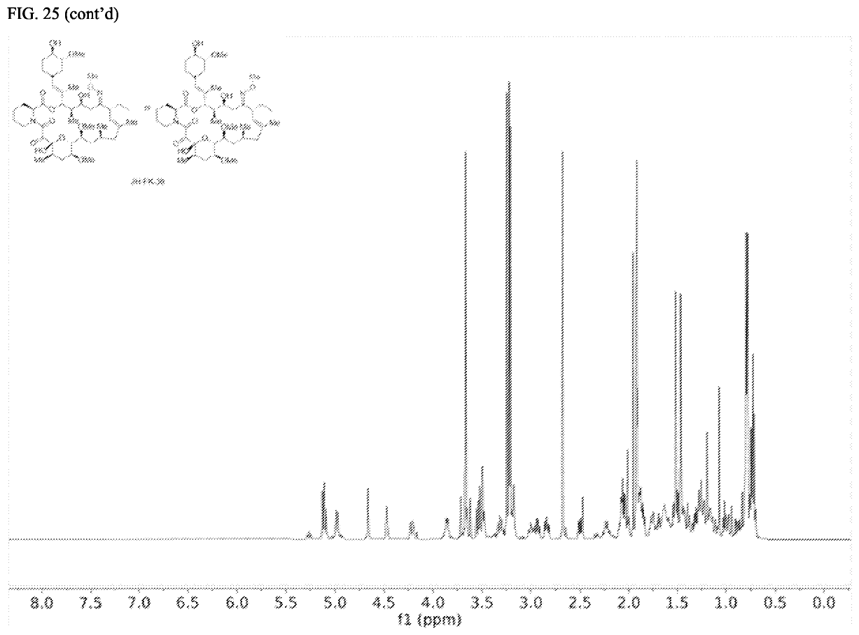

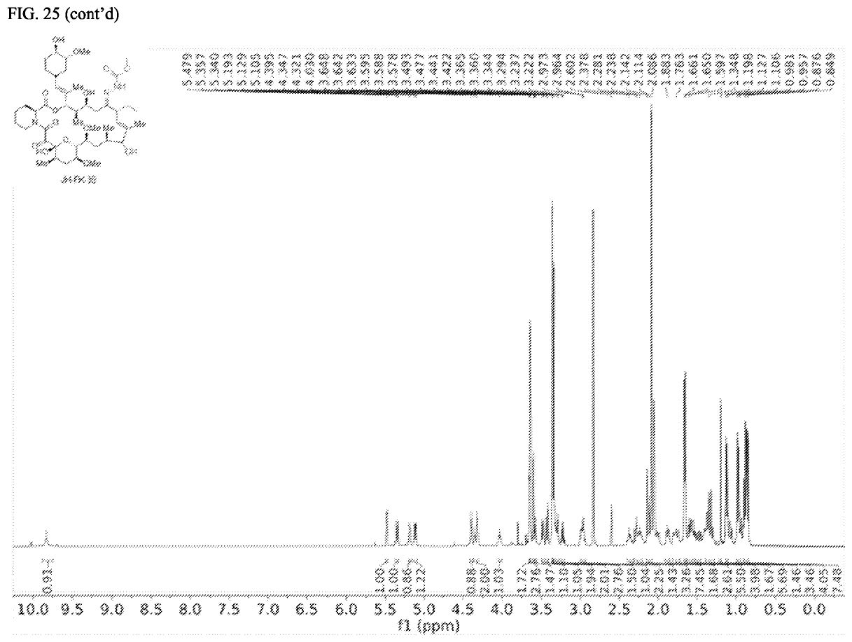

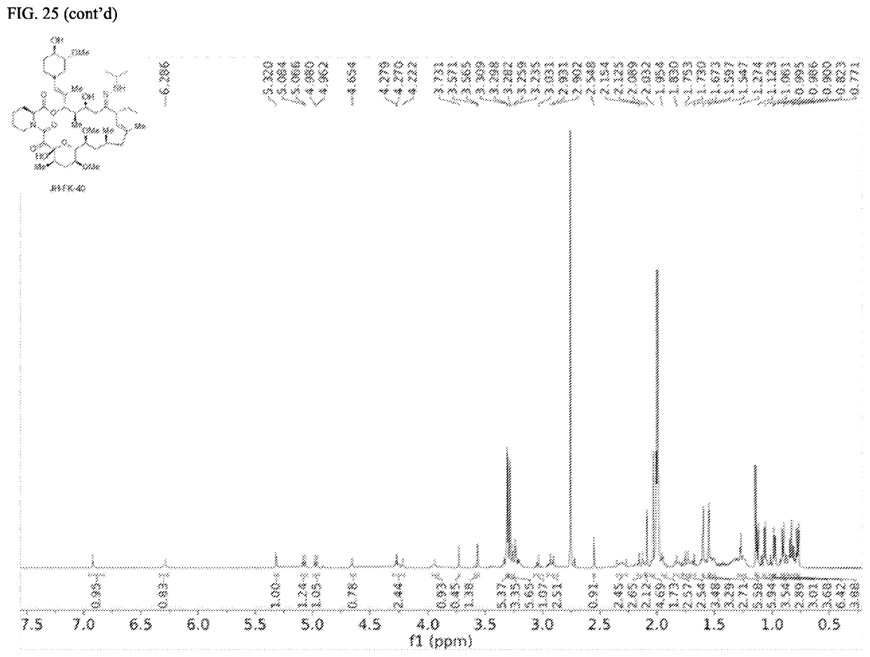

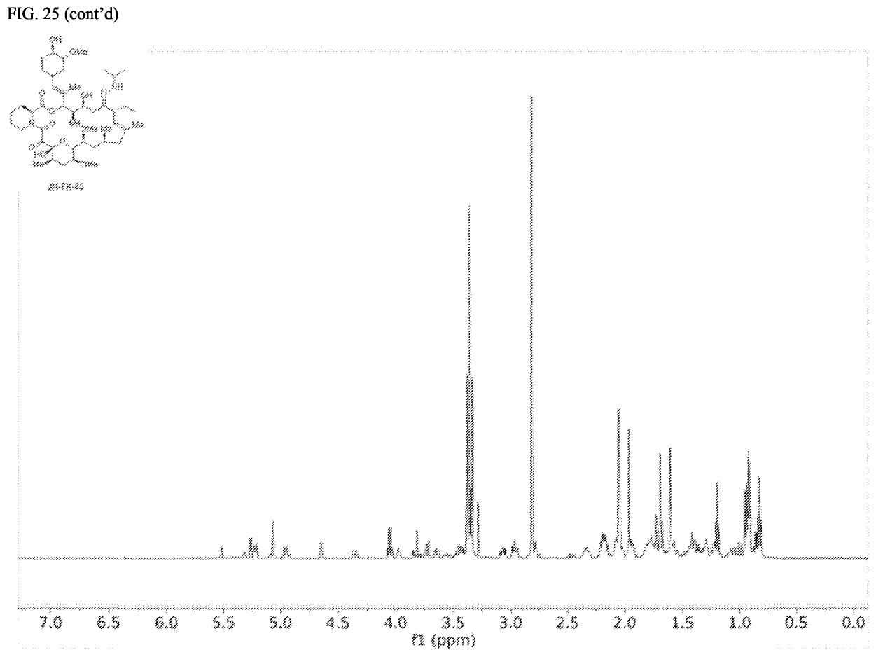

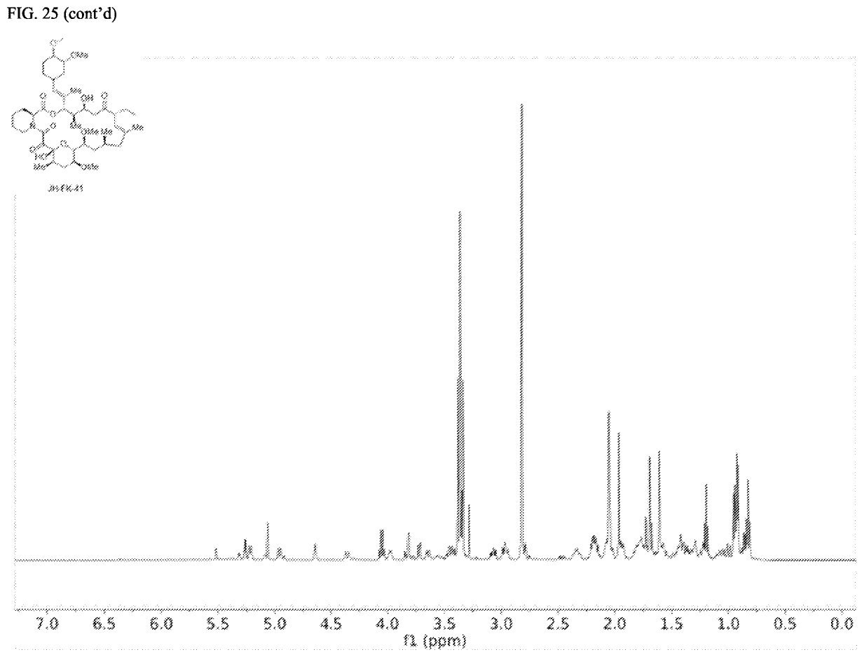

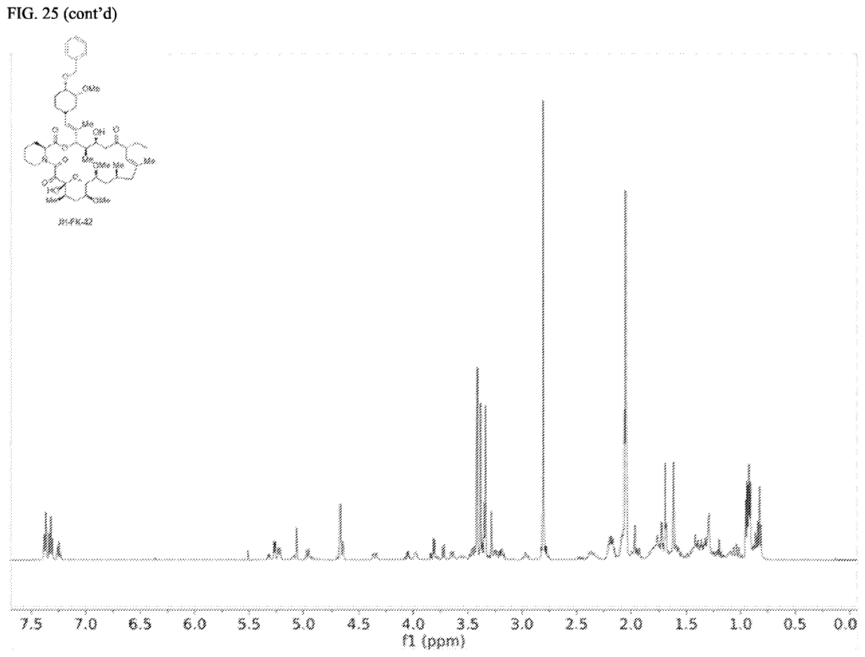

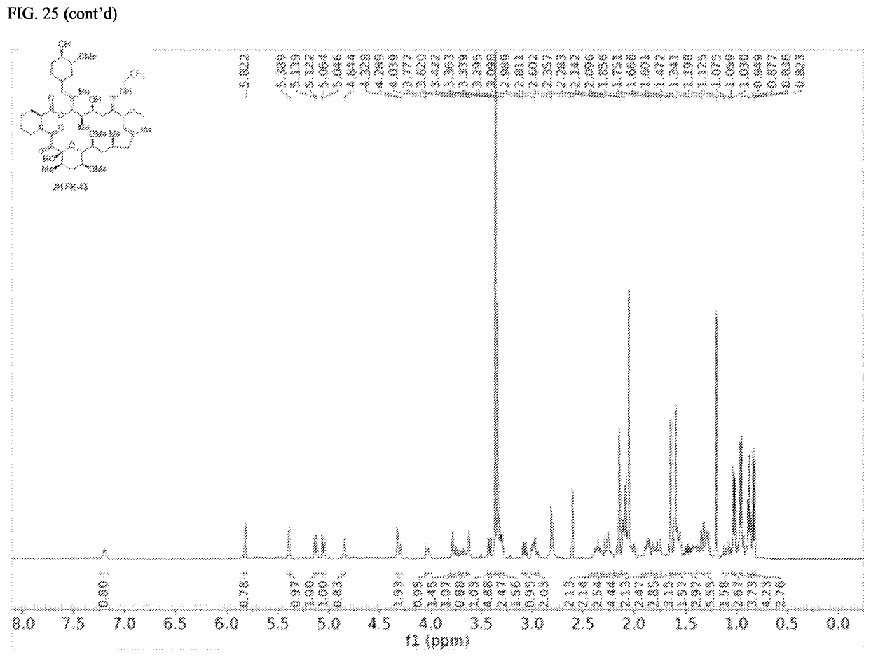

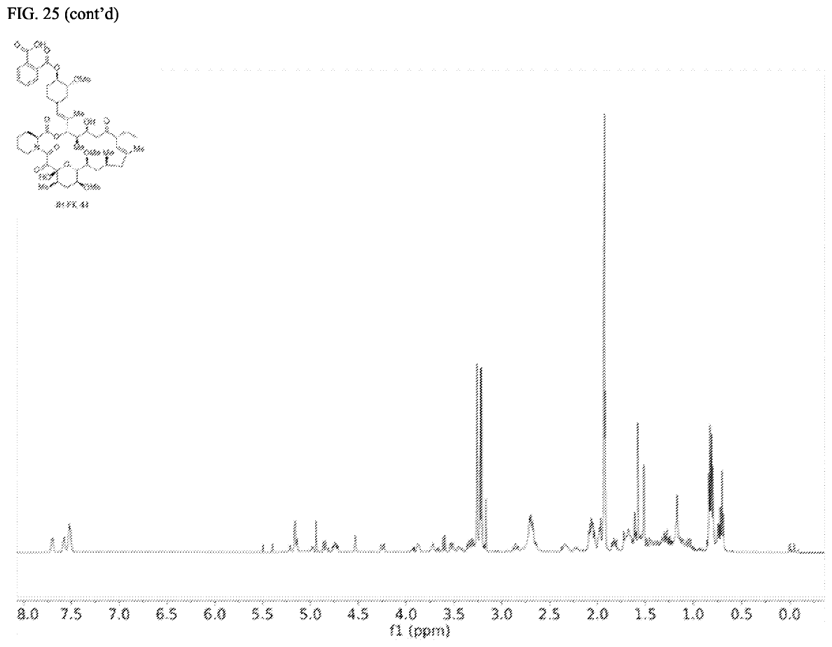

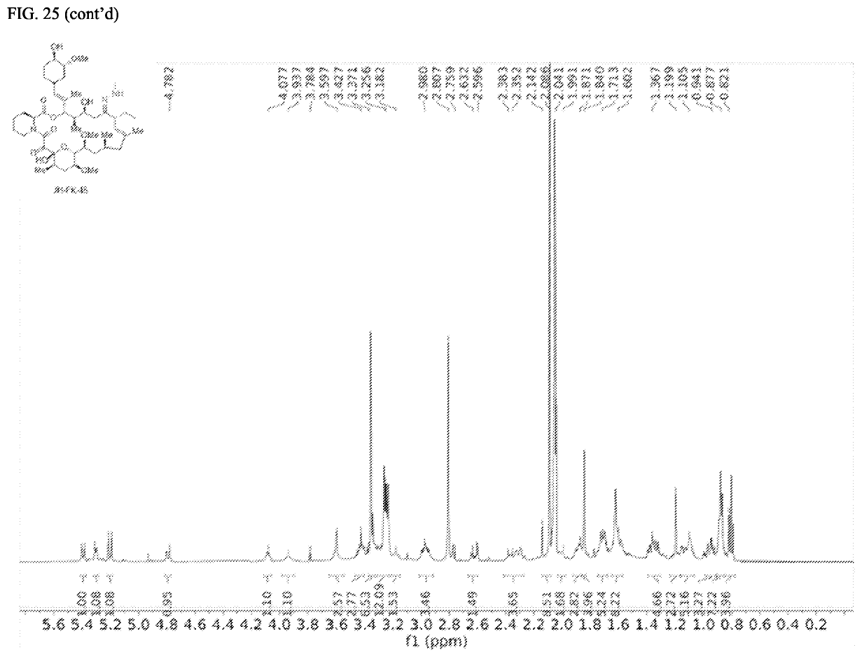

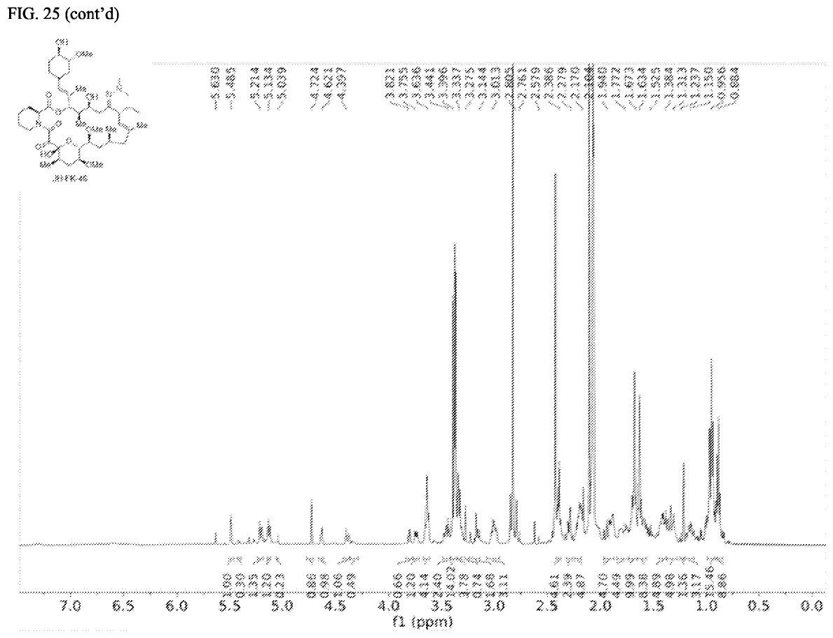

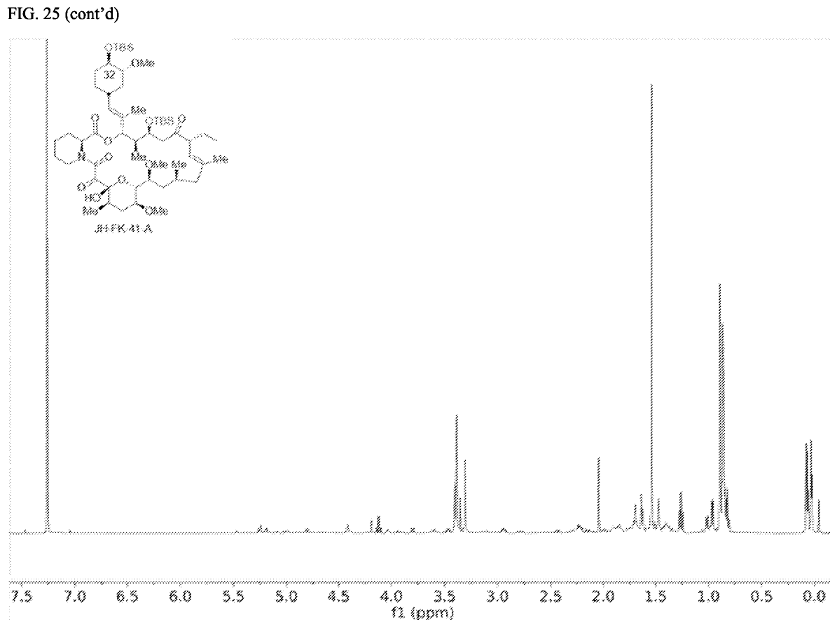

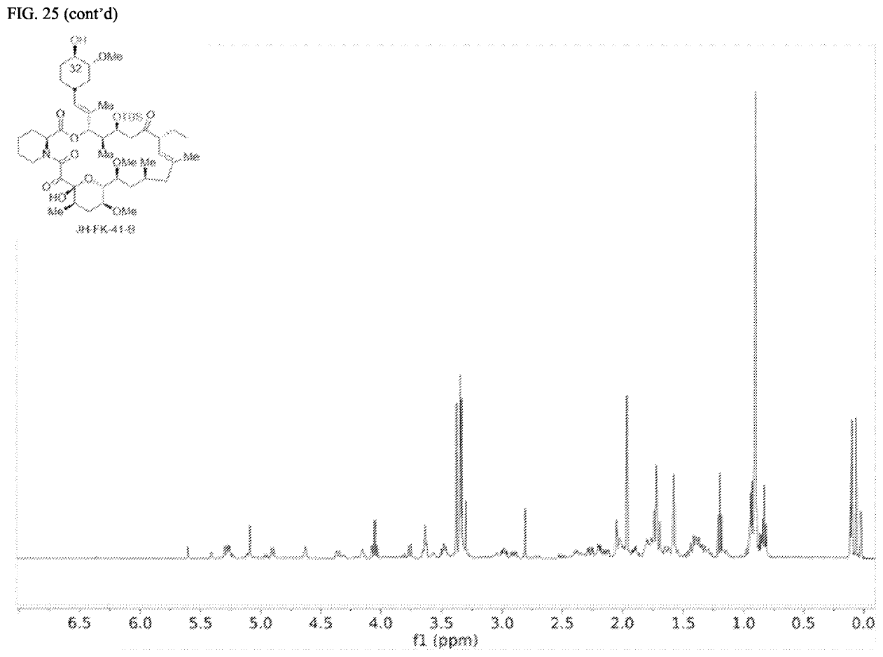

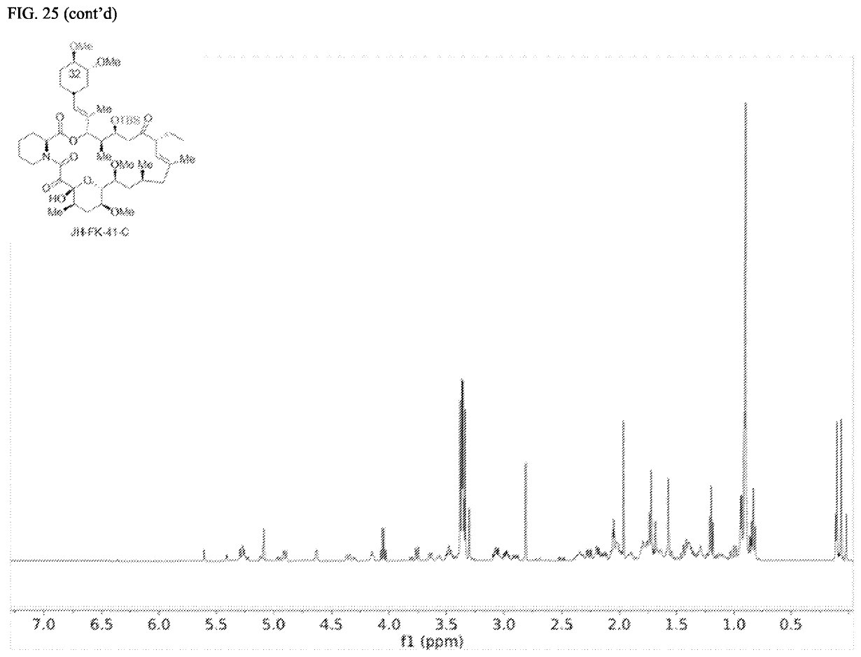

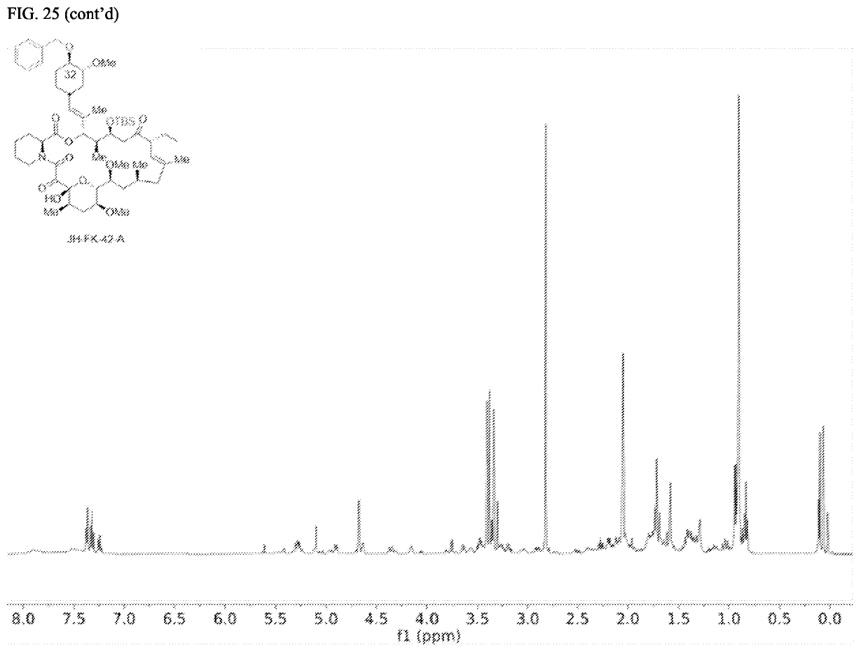

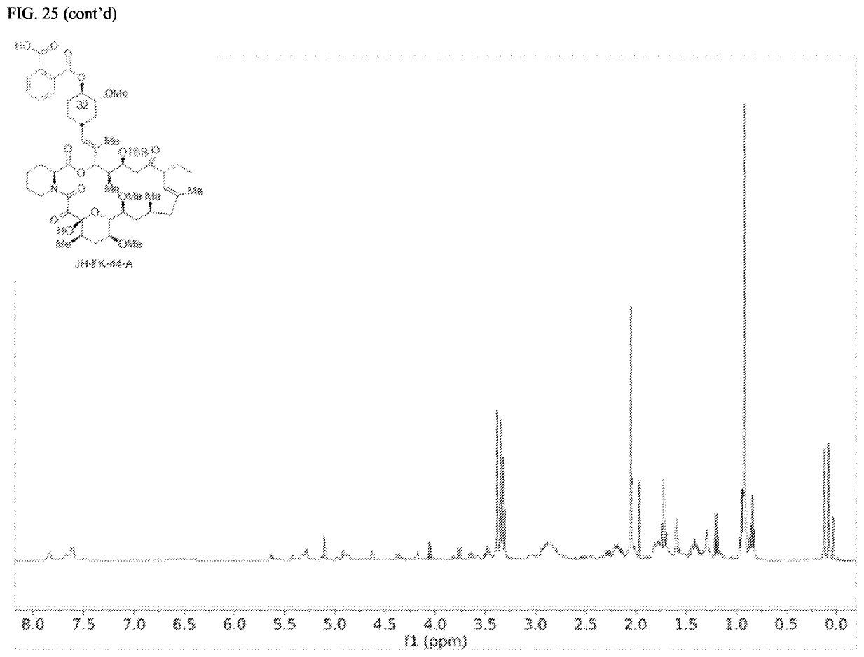

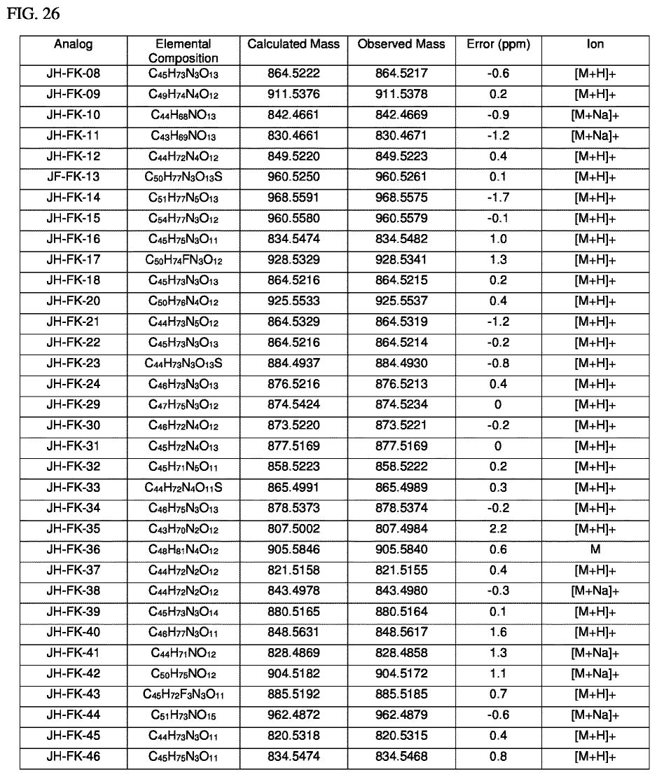

FIG. 25 provides NMR spectra data for JH-FK-8-13, 14-18, 20-24, 29-46, 41A, 41B, 41C, 42A, and 44A.

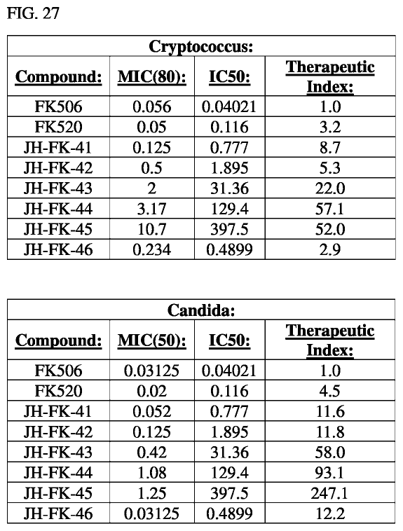

The therapeutic index for JH-FK-41, 42, 43, 44, 45, 46, FK506, and FK520 against Cryptococcus and Candida is shown in FIG. 27.

### Example 6

This example provides the materials and methods utilized in Examples 3, 4 and 5.

### Synthesis of FK506 and FK520 Analogs

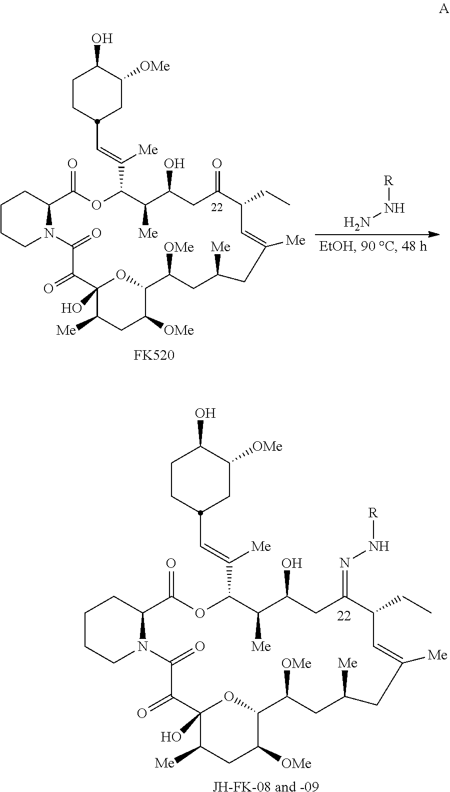

General procedure A: To a solution of FK520 in ethanol was added hydrazine in a sealed vial. The reaction mixture was stirred at 90° C. for 48 hours. After cooling to room temperature, the reaction vessel was unsealed and concentrated in vacuo. The crude mixture was reconstituted in ethyl acetate and washed with water and brine, dried over anhydrous Na₂SO₄, and condensed in vacuo. The residue was purified by column chromatography (silica gel, CH2CI2/MeOH) to afford the desired hydrazone.

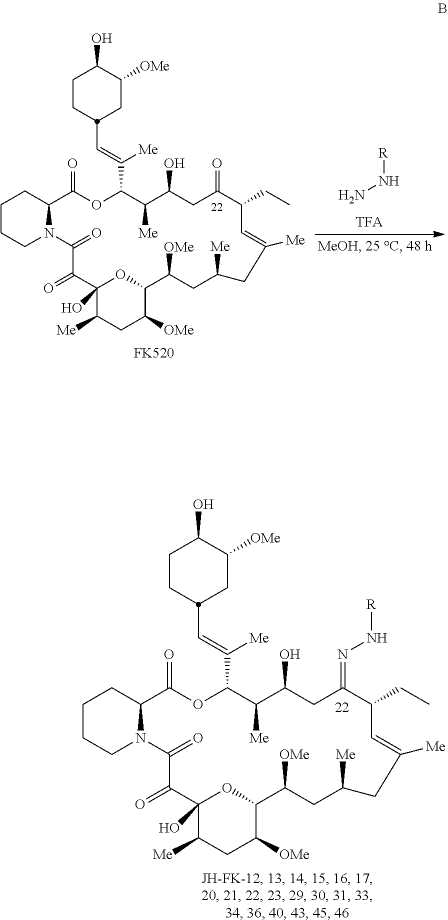

General procedure B: To a solution of FK520 in methanol was added hydrazine and catalytic TFA. The reaction was stirred at 25° C. for 48 hours. The reaction mixture was concentrated in vacuo and reconstituted in ethyl acetate. The reaction mixture was washed with water, brine, and 10% NaHCO₃ solution. The organic fraction was dried over anhydrous Na₂SO₄ and condensed in vacuo. The residue was purified by column chromatography (silica gel, CH2Cl2/MeOH) to afford desired hydrazone.

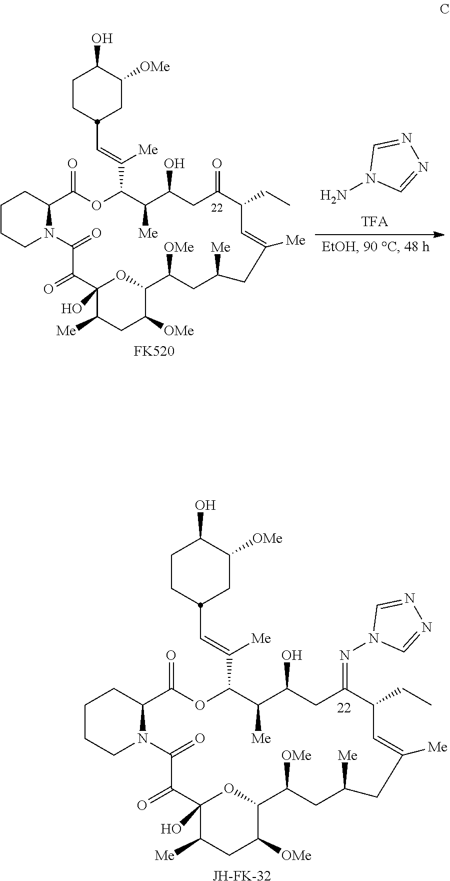

General Procedure C: To a solution of FK520 (40.0 mg, 0.051 mmol) in ethanol (0.5 mL) was added 4H-1,2,4-triazol-4-amine (25.7 mg, 0.306 mmol) and TFA (1 μL) in a sealed vial. The reaction mixture was stirred at 90° C. for 48 hours. After cooling to room temperature, the reaction vessel was unsealed and concentrated in vacuo. The crude mixture was reconstituted in ethyl acetate and washed with water and brine, dried over anhydrous Na₂SO₄, and condensed in vacuo. The residue was purified by column chromatography (silica gel, Hex/Acetone, 2/1) to afford JH-FK-32.

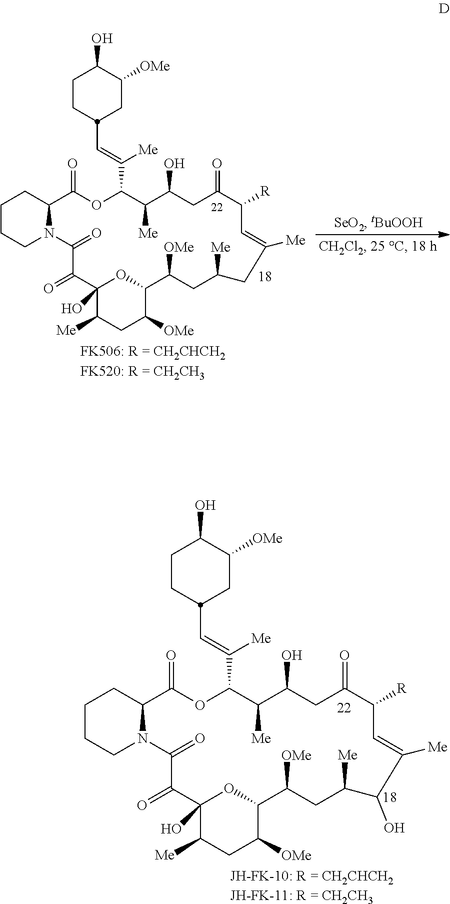

General Procedure D: To a solution of FK506 or FK520 and selenium dioxide in anhydrous dichloromethane was added ^{t}BuOOH in decanes (5-6 M) under a nitrogen atmosphere. The reaction was stirred at 25° C. for 18 hours. The reaction was quenched with addition of brine and extracted four times with dichloromethane, dried over anhydrous Na₂SO₄, and condensed in vacuo. The residue was purified by column chromatography (silica gel, CH2Cl2/MeOH) to afford desired C-18 hydroxylated FK506 or FK520.

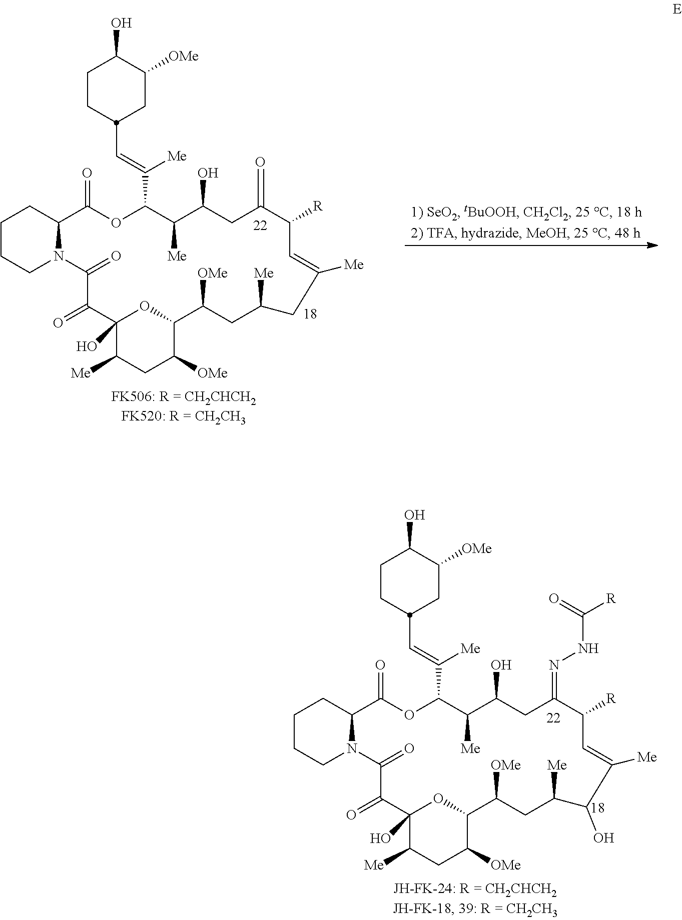

General Procedure E: C-18 hydroxylated FK506 or FK520 was prepared according to General Procedure D and then subjected to General Procedure B to afford the desired C₁₈, C₂₂-disubstituted hydrazone.

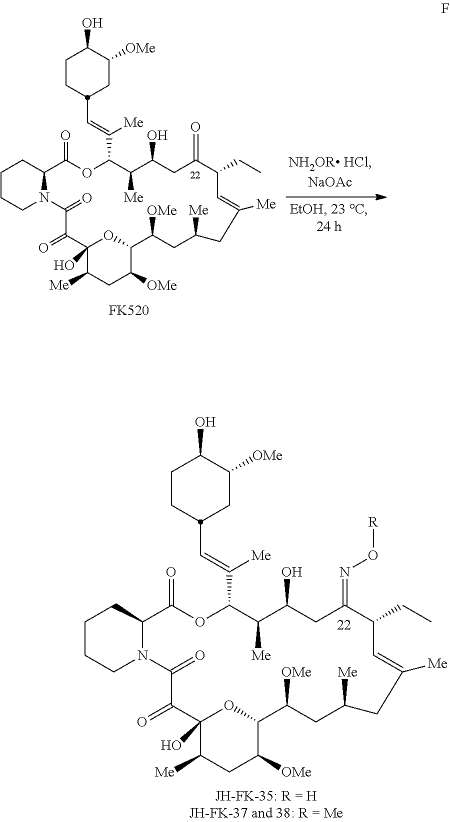

General Procedure F: FK520 and substituted hydroxylamine hydrochloride was dissolved in anhydrous ethanol under nitrogen atmosphere. Allowed to react for ten minutes, then added sodium acetate. The reaction was stirred at 23° C. for 24 hours. The reaction mixture was concentrated in vacuo, reconstitute in water and extracted three times with ethyl acetate. The combined organic fractions were dried over anhydrous Na₂SO₄ and condensed in vacuo. The residue was purified by column chromatography (silica gel, Hex/Acetone, 2/1) to afford the desired oxime.

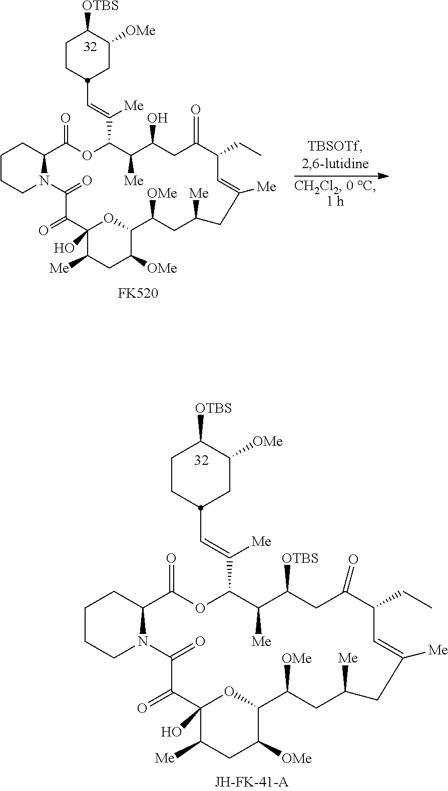

JH-FK-41-A: To a solution of FK520 (504 mg, 0.636 mmol) and 2,6-lutidine (341 mg, 3.18 mmol) in dichloromethane (13 mL) was added TBSOTf (588 mg, 2.23 mmol) at 0° C. The reaction was stirred at 0° C. for 45 minutes, followed by the addition of TBSOTf (168 mg, 0.636 mmol). The reaction was stirred at 0° C. for an additional 15 minutes. The reaction was quenched by addition of methanol and warmed to room temperature. Volume was increased with ethyl acetate and washed with 10% NaHCO₃ solution, water, and brine. The organic phase was dried over anhydrous and concentrated in vacuo. The residue was purified by column chromatography (silica gel, Hexanes/EtOAc: 3/1) to afford JH-FK-41-A as a colorless oil (685 mg, quantitative).

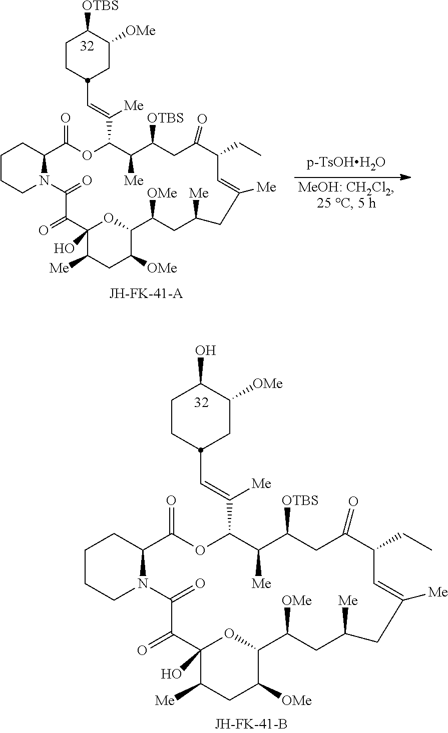

JH-FK-41-B: To a solution of JH-FK-41-A (685 mg, 0.671 mmol) in dichloromethane/methanol (1/1 v/v, 22 mL) was added p-toluenesulfonic acid monohydrate. The reaction was stirred at 25° C. for 5 hours. Volume was increased with ethyl acetate and washed with 10% NaHCO₃ solution, water, and brine. The organic phase was dried over anhydrous and concentrated in vacuo. The residue was purified by column chromatography (silica gel, Hexanes/EtOAc: 1/1 to 1/4) to afford JH-FK-41-B as a colorless foam (609 mg, 89%).

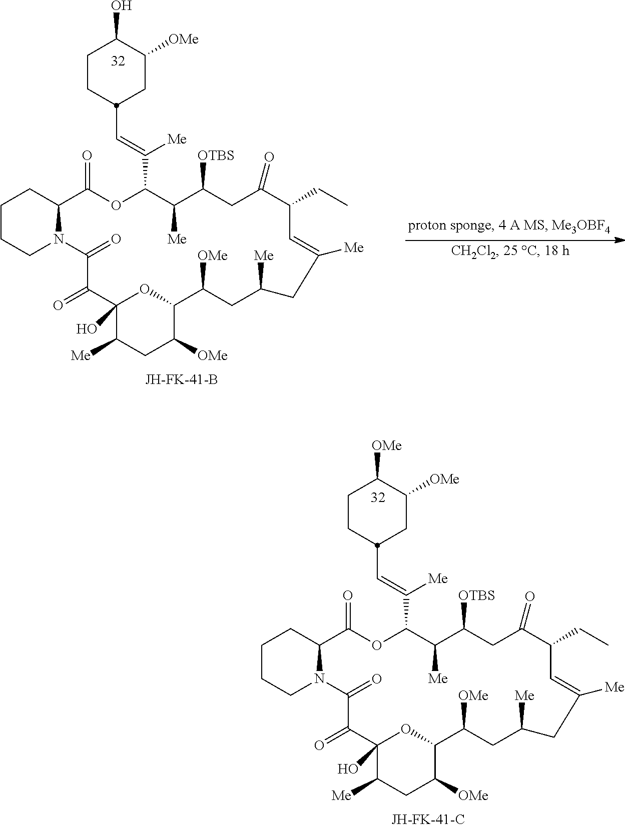

JH-FK-41-C: To a solution of JH-FK-41-B (62.5 mg, 0.069 mmol) in dichloromethane was added proton sponge (118 mg, 0.552 mmol) and freshly-dried 4 Å molecular sieves (80 mg). The solution was stirred for 1 hour at 25° C. Trimethyloxonium tetrafluoroborate (81.6 mg, 0.552 mmol) was added to the reaction. The solution was stirred for 18 hours at 25° C. The reaction mixture was diluted with dichloromethane and passed through a celite filter. The filtrate was washed twice with 1 N HCl and 10% NaHCO₃ solution, dried over anhydrous Na2SO4, and condensed in vacuo. The residue was purified by column chromatography (silica gel, Hexanes/EtOAc: 9/1 to 4/1) to afford JH-FK-41-C as a white solid (48.5 mg, 76%).

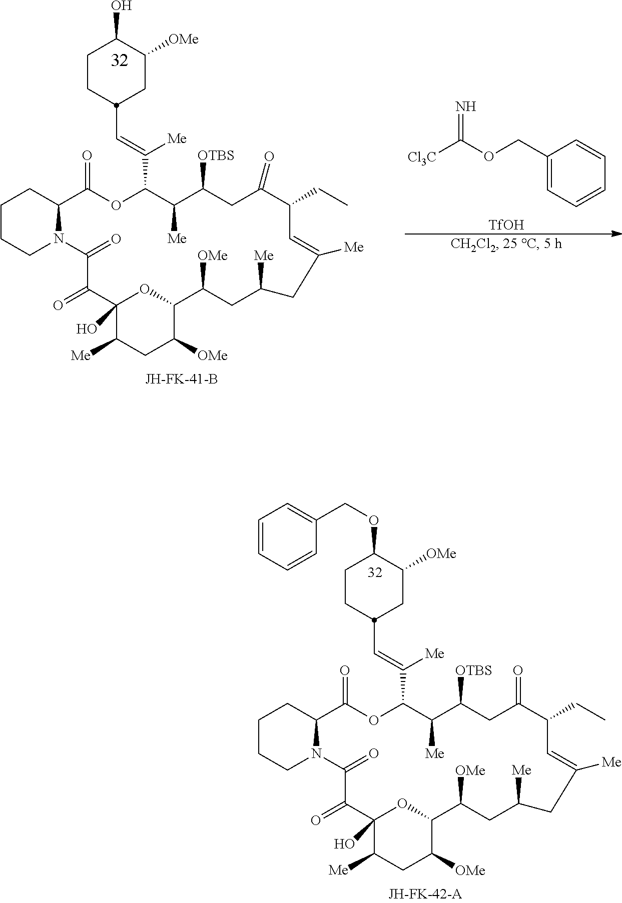

JH-FK-42-A: To a solution of JH-FK-41-B (50.9 mg, 0.056 mmol) was added benzyl 2,2,2-trichloroacetimidate (28.3 mg, 0.112 mmol) in dichloromethane (1.0 mL). Trifluoromethanesulfonic acid (1.7 mg, 0.0112 mmol) was added and the solution was stirred for 5 hours at 25° C. The reaction was quenched with saturated aqueous NaHCO₃, diluted with ethyl acetate, and extracted with ethyl acetate. The combined organic phases were dried over anhydrous Na₂SO₄, and condensed in vacuo. The residue was purified by column chromatography (silica gel, Hexanes/EtOAc) to afford JH-FK-42-A as a white solid (13.1 mg, 23%).

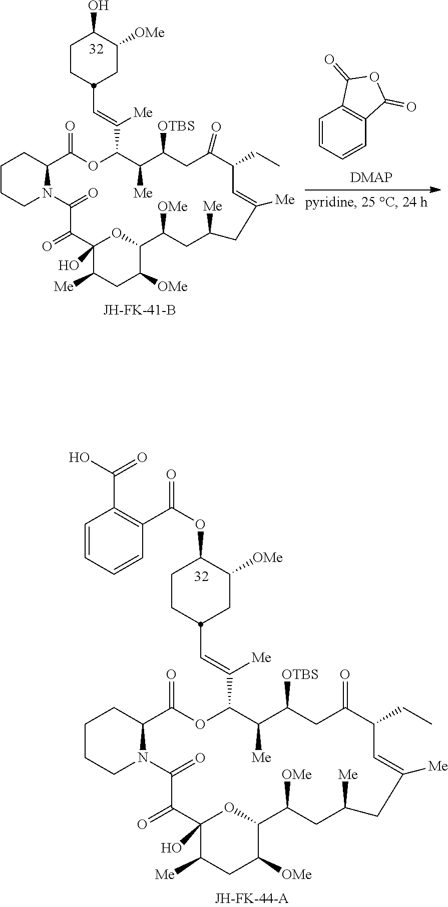

JH-FK-44-A: To a solution of JH-FK-41-B (52.5 mg, 0.0579 mmol) in pyridine (0.5 mL) was added DMAP (6.1 mg, 0.050 mmol) and phthalic anhydride (40.9 mg, 0.276 mmol). The solution was stirred for 24 hours at 25° C. The reaction was quenched by addition of water and extracted with dichloromethane. The aqueous phase was acidified with NaH₂PO₄ and extracted with dichloromethane. The combined organic phases were washed with saturated aqueous NaHCO₃, dried over anhydrous Na₂SO₄ and condensed in vacuo. The residue was purified by column chromatography (silica gel, Hexanes/EtOAc: 1/2) to afford JH-FK-44-A as a white solid (26.6 mg, 44%).

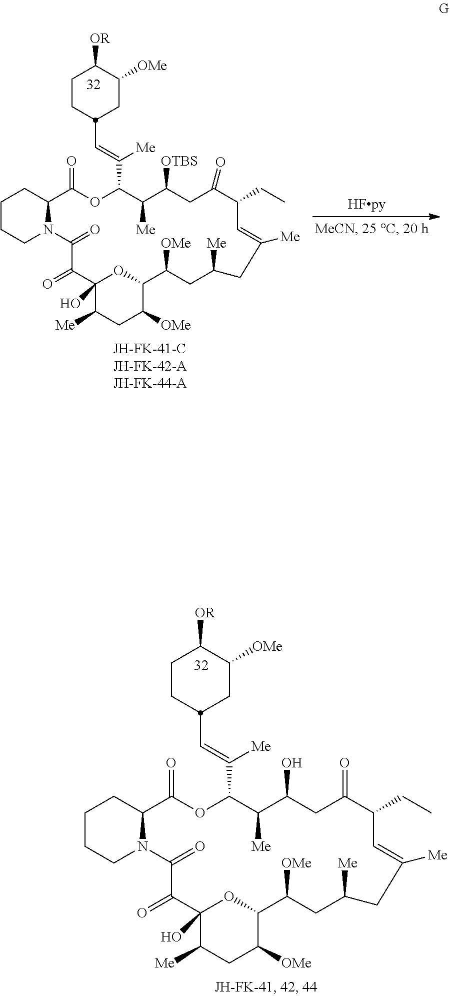

General Procedure G: To a solution of JH-FK-41-C, JH-FK-42-A, or JH-FK-44-A in acetonitrile was added HF-pyridine. The solution was stirred for 20 hours at 25° C. The reaction was quenched by addition of saturated aqueous NaHCO₃ and extracted with dichloromethane. The organic phase was dried over anhydrous Na2SO4 and condensed in vacuo. The residue was purified by column chromatography (silica gel, Hexanes/EtOAc) to afford JH-FK-41, JH-FK-42, or JH-FK-44 as a white solid.

### Antifungal Susceptibility Testing

In vitro antifungal activity was assessed for all fungal strains in RPMI 1640 (Sigma-Aldrich R₁₃₈₃). MICs were measured using Clinical and Laboratory Standards Institute (CLSI) M38-A2 and M27-A3 standard in vitro antifungal susceptibility protocols. C. neoformans and A. fumigatus testing was performed at 37° C. MECs for A. fumigatus were recorded after 48 h of growth. C. albicans testing was performed at 30° C. with a uniform concentration of 2 μg/mL of fluconazole to sensitize the cells to calcineurin inhibition.

### Fungal Strains

C. neoformans clinical isolate H99 and congenic-derived strain KN99α were used (see, Nielsen K, et al., 2003. Infect Immun 71:4831-4841). KN99α-derived frr1Δ deletion mutants with nourseothricin resistance markers as part of the Madhani deletion collection (NIH funding R₀₁AI100272) were used. C. deneoformans strains used include JEC21 and derived C₂₁F2 (CNB1-1) and C₂₁F3 (frr1-3) strains (see, Odom A, et al., 1997. EMBO J 16:2576-2589; Moore T D, Edman J C. 1993. Mol Cell Biol 13:1962-1970; Fox D S, et al., 2001. Mol Microbiol 39:835-849). C. albicans strain SC5314 is the wild-type strain from which mutants YAG171 (rbp1/rbp1) and YAG237 (CNB1-1/CNB1) were derived (see, Cruz M C, et al., 2001. Antimicrob Agents Chemother 45:3162-3170). A. fumigatus strain akuB^{Ku80} and a derived FKBP12 mutant fkbp12A were used during antifungal screening (see, Falloon K, et al., 2015. PLoS One 10:e0137869).

### In Vitro Immunosuppressive Activity Testing

Spleen and lymph nodes from C₅₇BL/6 mice were collected and homogenized through a 40-μm filter. Pan-CD4⁺ T cells were enriched using the MagniSort Mouse CD4 T Cell Enrichment Kit (Invitrogen). Thereafter, CD4⁺CD25⁻CD62L^{hi}CD44^{lo} naïve CD4⁺ T cells were FACS-sorted using a MoFlo Astrios cell sorter (Beckman Coulter). Naïve CD4⁺ T cells were cultured in Iscove's Modified Dulbecco's Medium supplemented with 10% fetal bovine serum (FBS), penicillin (10 U/mL), streptomycin (10 μg/mL), glutamine (2 mM), gentamicin (50 μg/mL), and β-mercaptoethanol (55 μM). Cells were cultured for 72 h on anti-hamster IgG-coated plates with hamster anti-CD3s antibodies, hamster anti-CD28 antibodies, neutralizing anti-IL-4 antibodies, recombinant IL-12 (10 ng/mL), and recombinant IL-2 (50 U/mL). During this culture, cells were incubated with serially diluted drugs suspended in DMSO.

### Flow Cytometry

Cell surface staining of single cell suspensions was performed in staining buffer (PBS supplemented with 2 mM EDTA and 0.5% BSA). Anti-FcγR (93; Biolegend) was treated to block the Fe receptor before surface staining of primary cells isolated from lymph nodes of NP-OVA-immunized mice. Cells were stained with fixable viability dye (eBioscience) before surface staining to exclude dead cells from analysis. For the detection of intracellular cytokines, cells were additionally incubated with phorbol 12-myristate 13-acetate and ionomycin in the presence of GolgiStop (BD Biosciences) during the final 4 h of incubation. For intracellular staining, cells were fixed and permeabilized with Fixation/Permeabilization Buffer (eBioscience). Flow cytometric analysis was performed utilizing BD FACSCantoII or BD LSRFortessaX20 (BD Biosciences) and FlowJo software (BD Biosciences).

### Compound Preparation for In Vivo Studies

For in vivo studies, fluconazole was purchased from the Duke Pharmacy Storeroom. Vehicle was a sterile solution of 10% Kolliphor EL (Sigma-Aldrich C₅₃₁₅), 10% ethanol, and 80% PBS (Sigma-Aldrich D8537). For administration to animals, all other drugs were obtained in powder form and diluted in a sterile mixture of 50% Kolliphor EL and 50% ethanol before dilution to 80% PBS.

### In Vivo Immunosuppressive Assessment

Groups of five female C₅₇BL/6 mice were treated twice daily for 8 days with vehicle, FK506 (2.5 mg/kg), JH-FK-08 (20 mg/kg), or JH-FK-08 (40 mg/kg) via i.p. injection. Twenty-four hours after the first dose, animals were immunized with NP-OVA (100 μg) in alum via subcutaneous injection. Seven days post immunization, mice were sacrificed, and draining lymph nodes were harvested for the analysis of Tfh cells and GC B cells by flow cytometry.

C. neoformans Murine Infection

C. neoformans inoculum was prepared by growing a 5-mL overnight YPD culture spinning in a tissue culture roller drum at 30° C. Cells were pelleted by centrifugation and washed in sterile PBS three times. Cell density was determined by hemocytometer, and cells were diluted to 2×10⁶ cells/mL. Mice (10 to 20 g) were infected with 10⁵ cells of C. neoformans H99. For inhalation infection, animals were anesthetized with isoflurane before 50 μL of inoculum was pipetted onto the upright nasal passages of each animal. Following inhalation of the inoculum, animals were monitored for post anesthesia recovery. Treatment of animals began 4 hours post infection and was continued for 14 days. Fungal burden was assessed 24 h after the final dose.

### Histology Preparation

Lungs were inflated with and immersed in 10% neutral buffered formalin, processed routinely to paraffin embedment, and sectioned at 5 mm. Sections were stained either with Hematoxylin & Eosin or Periodic-acid Schiff. All tissue sections were evaluated and photographed with an Olympus BX51 microscope and a DP70 camera using Olympus DP Manager software v.3.3.112. Samples were processed by the Department of Pathology.

### Pharmacokinetic Experiment

Male (n=3, avg. 28 g) and female (n=3, avg. 25 g) CD-1 mice were injected intraperitoneally with 40 mg/kg of either JH-FK-08 or FK506. The drug formulation was 10% Kolliphor/10% ethanol/80% PBS given as 10 μL/g body weight (i.e., 200 μL per 20 g mouse). Blood (~30 μL) was collected serially (“tail snip”) at 10, 20, and 45 min and 1.5, 3, 6, 12, and 24 h into vials containing 1 mL of 75 mg/mL K₂EDTA in water and immediately frozen until the day of liquid chromatography-tandem-mass spectroscopy (LC/MS/MS) analysis. Blood concentration/time data were utilized to calculate relevant PK parameters using the non-compartmental approach and extravascular administration model within WinNonlin (2.1) software.

### LC/MS/MS Analysis

In a 0.5-mL polypropylene vial, 10 μL blood and 20 μL of acetonitrile fortified with 100 ng/mL FK520 (internal standard) were vigorously agitated in the FastPrep FP120 apparatus (Thermo-Savant) at speed four for 45 s. After centrifugation for 5 min at 16,000 rpm, 20 μL of supernatant was transferred to a polypropylene autosampler vial. LC/MS/MS (Agilent 1200 series HPLC and Sciex/Applied Biosystems API 5500 QTrap) was utilized to quantify JH-FK-08 and FK506 in mouse blood. Analytical column: Agilent Eclipse Plus (C18, 1.8 μm, 50×4.6 mm), at 40° C. Mobile phase: (A) 0.1% formic acid, 10 mM ammonium acetate, 2% acetonitrile in water, and (B) methanol. Elution gradient: 0-0.1 min 30%-98% B, 0.1-1.5 min 98% B, 1.5-1.6 min 98%-30% B, and 1.6-2.1 min 30% B. Run time: 5 min. Injection volume: 10 μL. Mass spectrometer parameters (voltages, gas flow, and temperature) were optimized by infusion of 100 ng/mL of analytes in the mobile phase at 10 μL/min using the Analyst 1.6.2 software tuning module. The MS/MS (m/z) transitions used for quantification were 864.4/846.5 (JH-FK-08), 821.4/768.5 (FK506), and 809.4/756.5 (FK520, internal standard). A set of calibrator samples in drug-free blood was prepared by adding appropriate amounts of pure analyte (JH-FK-08 or FK506) in the 2.43-1,000 μL/mL range. The calibration samples were analyzed alongside the experimental samples. The accuracy acceptance criterion was 85% for each but the lowest level (2.43 ng/mL, 80%, LLOQ). Analyst 1.6.2 software was used for data acquisition, integration of the chromatograms, calibration curve calculation, and quantification of the study samples.

### Cryptococcus Fungal Burden and Survival Analysis

For survival curve analysis, animals were monitored until reaching defined humane end points, including social isolation, hunched posture, weight loss, lack of grooming, and labored breathing, and were then euthanized via CO₂ inhalation. For fungal burden analysis, mice were euthanized following 2 weeks of treatment with a designated antifungal, vehicle, or combination. Brain, lung, and spleen were harvested from animals, weighed, and homogenized in sterile PBS using steel beads and a bead beater. Each organ homogenate was serially diluted 10-fold to 10-4 in PBS. One hundred microliters of each dilution was plated on YPD plates supplemented with YPD 50 μg/mL ampicillin and 30 μg/mL chloramphenicol, incubated at 30° C. for 2 days, and assessed for colony-forming units. CFU per organ weight was calculated and plotted using GraphPad Prism 9.

### Antifungal Susceptibility Testing for JH-FK-10, JH-FK-11, JH-FK-18, JH-FK-24, and JH-FK-39

In vitro antifungal activity of FK506, FK520, and analogs was assessed in RPMI 1640 (Sigma-Aldrich) for all strains tested. Minimum inhibitory concentrations (MIC) for each drug were measured using modified Clinical and Laboratory Standards Institute (CLSI) M38-A2 and M27-A3 standard in vitro antifungal susceptibility protocols. Broth microdilution assays for MIC assessment were performed as previously described. All antifungal assays with C. neoformans were performed at 37° C.

### In Vitro Immunosuppressive Activity Testing for JH-FK-10, JH-FK-11, JH-FK-18, JH-FK-24, and JH-FK-39

Spleen and lymph nodes were collected from C₅₇BL/6 mice and homogenized through a 40 μm filter. Pan-CD4⁺ T cells were enriched using MagniSort Mouse CD4 T cell Kit (eBioscience) according to the manufacturer's protocol. Thereafter, CD4+CD25−CD44lo CD62Lhi naïve T cells were FACS-sorted. The naïve CD4+ T cells were grown in Iscove's Modified Dulbecco's Medium (IMDM) supplemented with glutamine, penicillin, streptomycin, gentamicin, 2-mercaptoethanol, and 10% FBS. Cells were then cultured on anti-hamster IgG coated plates in the presence of hamster anti-CD3 epsilon and anti-CD28 antibodies (BD Biosciences), neutralizing anti-IL-4 antibody (eBioscience), recombinant IL-12 (10 ng/mL), and recombinant IL-2 (50 U/mL) for 72 h. During this period, cells were grown in the presence of 2× serially diluted FK506, FK520, or analog suspended in DMSO for 72 h. During the last 4 h of culture, phorbol 12-myristate 13-acetate (PMA; Sigma), ionomycin (Sigma), and GolgiStop (BD Biosciences) were added to the culture to facilitate detection of intracellular cytokines via flow cytometry.

Live cells were stained with FITC-conjugated anti-CD4 antibody (eBioscience) and Fixable Viability Dye eFluor 506 (eBioscience) on ice, followed by fixation and permeabilization using the Foxp3 Transcription Factor Staining Kit (eBioscience). Intracellular staining was performed at room temperature using PE-conjugated anti-IL-2 antibody (eBioscience), followed by analysis with a BD LSRFortessa X-20 flow cytometer.

### INCORPORATION BY REFERENCE

The entire disclosure of each of the patent documents and scientific articles referred to herein is incorporated by reference for all purposes. The following references are herein incorporated by reference in their entireties:

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within one of the following formulas: 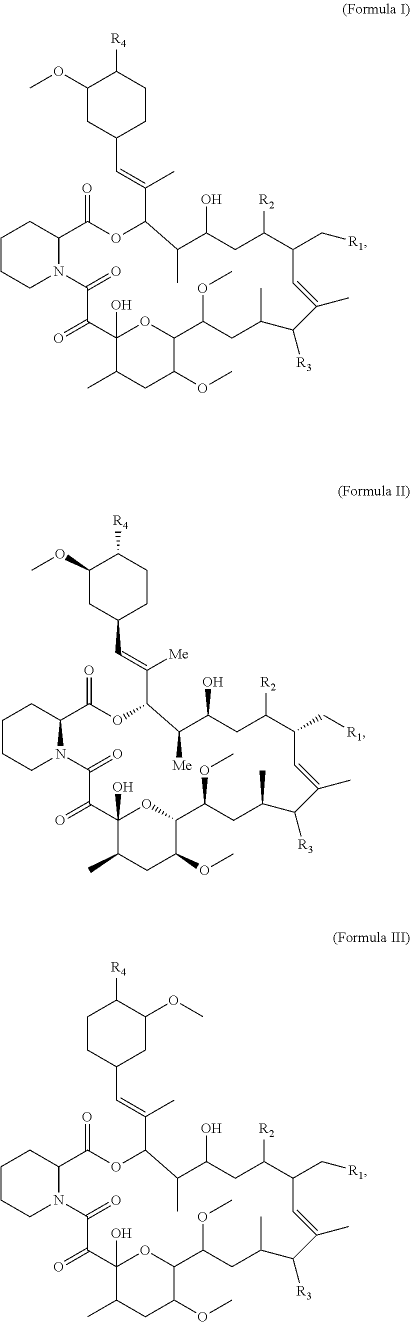 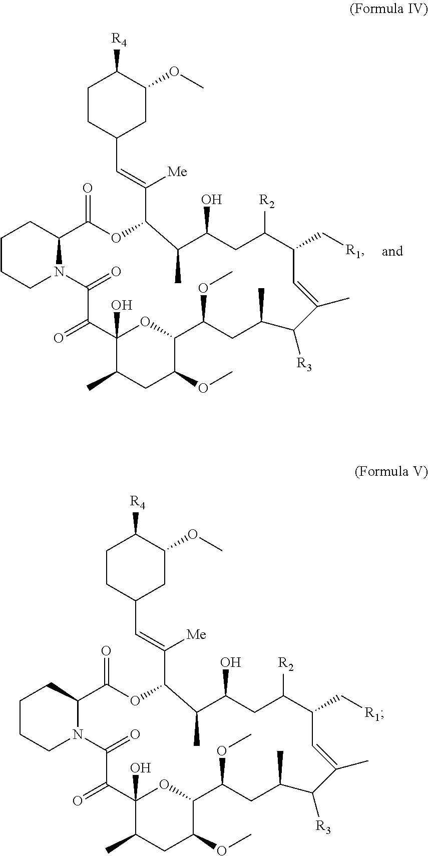
including pharmaceutically acceptable salts, hydrates, solvates, polymorphs, prodrugs, triphenylphosphonium (TPP) tagged derivatives, proteolysis targeting chimera (PROTAC) derivatives, mitochondria tagged derivatives, biotin tagged derivatives, boron-dipyrromethene (BODIPY) tagged derivatives, stereoisomers, and/or deuterated analogues thereof;
wherein each of R₁, R₂, R₃, and R₄ independently include any chemical moiety that renders the resulting compound capable of inhibiting fungal activity.

2. The compound of claim 1, each of R₁, R₂, R₃, and R₄ independently include any chemical moiety that renders the resulting compound capable of one or more of the following:
inhibiting calcineurin;
inhibiting fungal activity associated with calcineurin;
preventing fungal growth;
inhibiting calcineurin activity;
associating with (e.g., binding with) FKBP12;
associating with (e.g., binding with) FKBP12 to form a complex that is capable of inhibiting calcineurin activity;
associating with (e.g., binding with) FKBP12 to form a complex that is capable of inhibiting fungal activity;
associating with (e.g., binding with) FKBP12 to form a complex that is capable of inhibiting Cryptococcus neoformans activity.

3. The compound of claim 1, wherein R₁ is selected from hydrogen,

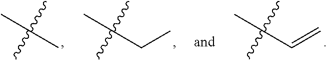

4. The compound of claim 1, wherein R₂ is selected from hydrogen,

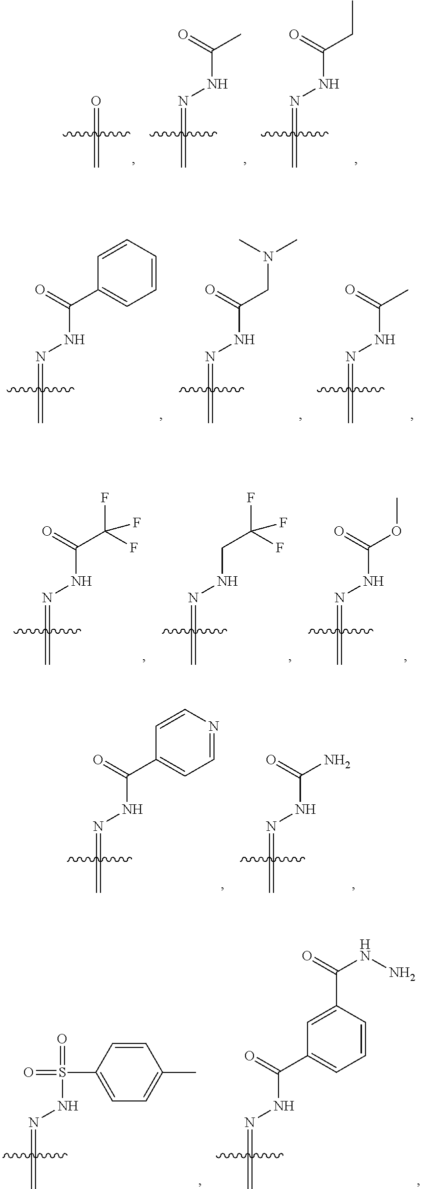

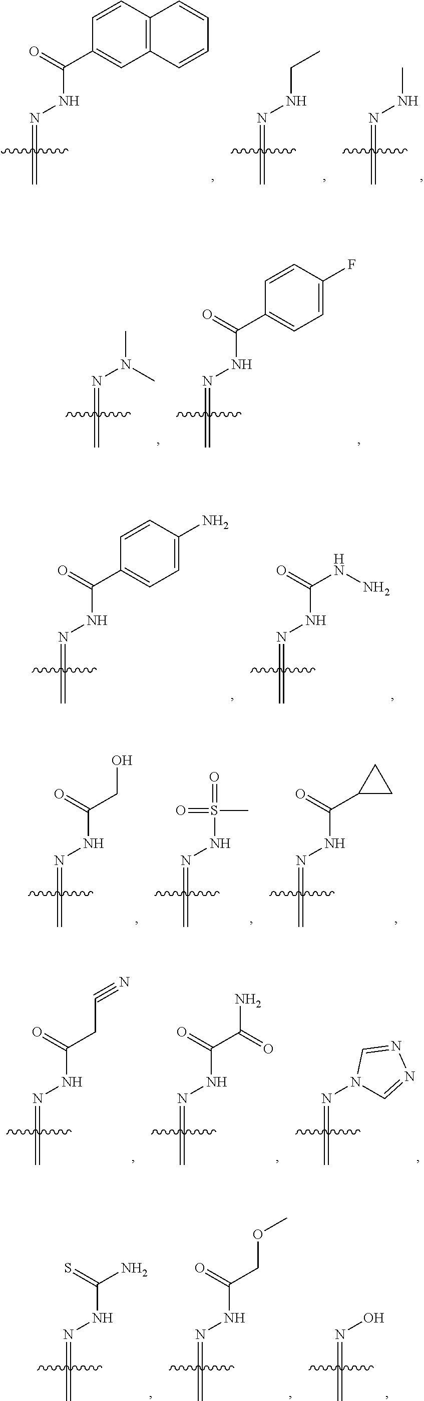

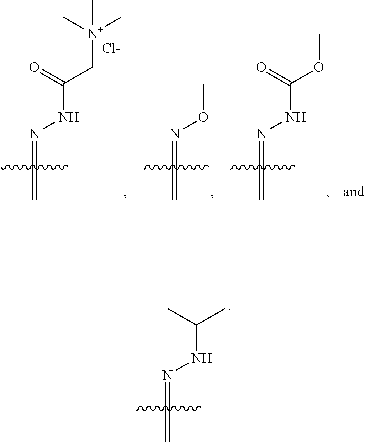

5. The compound of claim 1, wherein R3 is selected from hydrogen, and hydroxyl (e.g., OH).

6. The compound of claim 1, wherein R4 is selected from hydrogen, hydroxyl (e.g., OH),

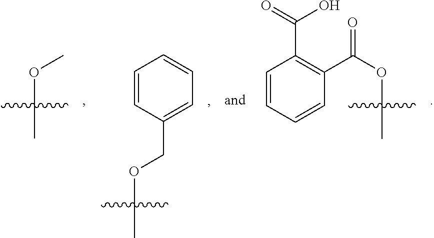

7. The compound of claim 1, wherein the compound is selected from:

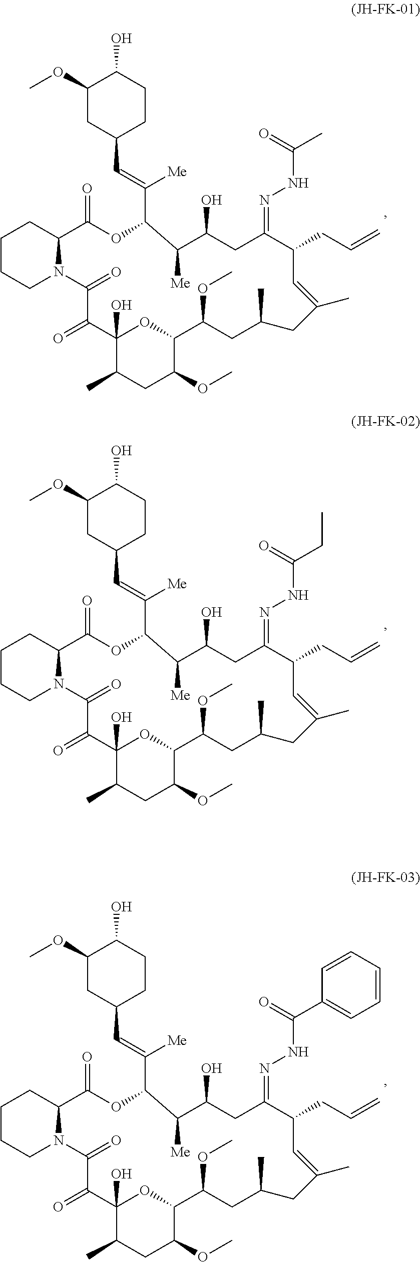

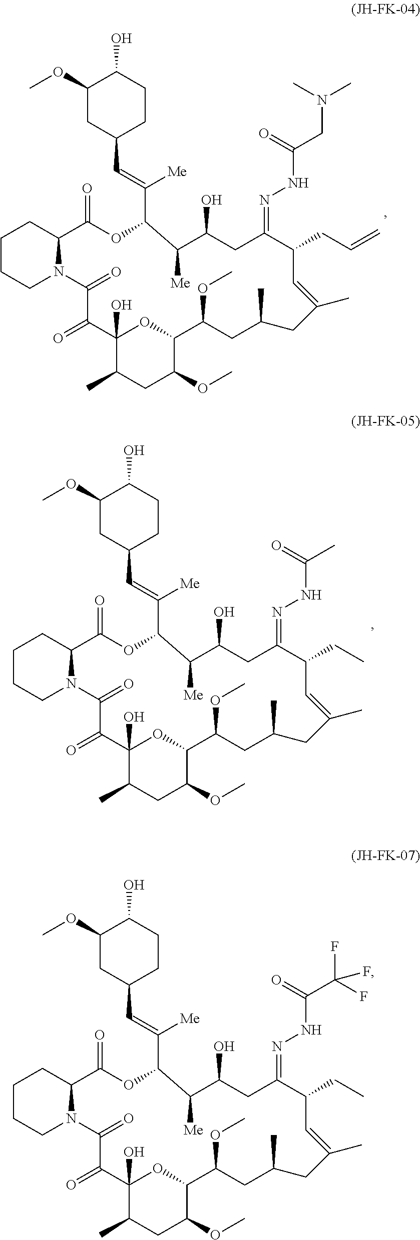

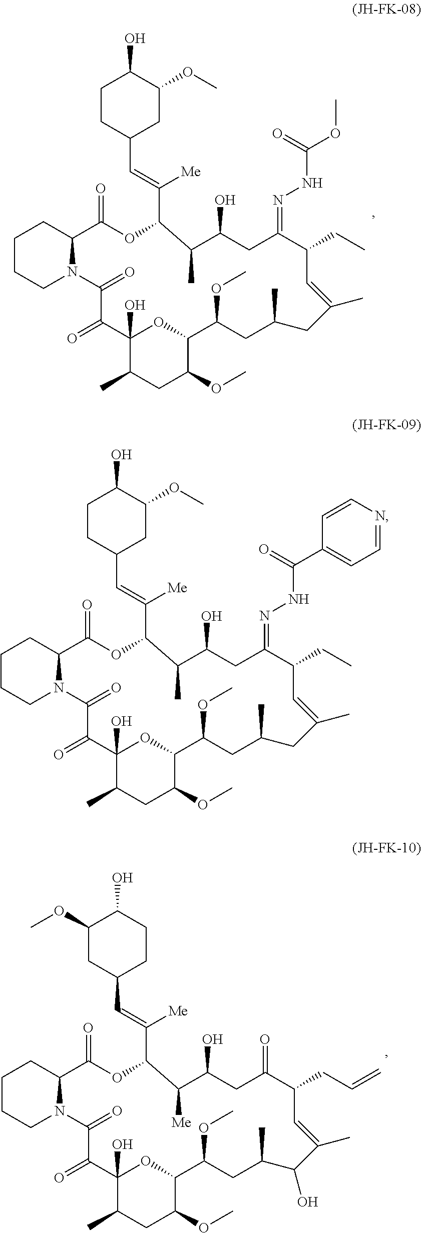

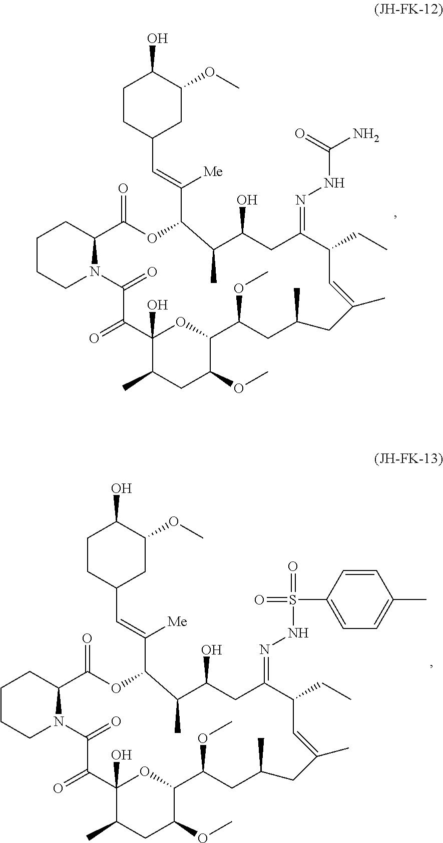 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/NC(N)=O)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/NS(=O)(=O)c1ccc(C)cc1)[C@H](C)C[C@@H]2OC]

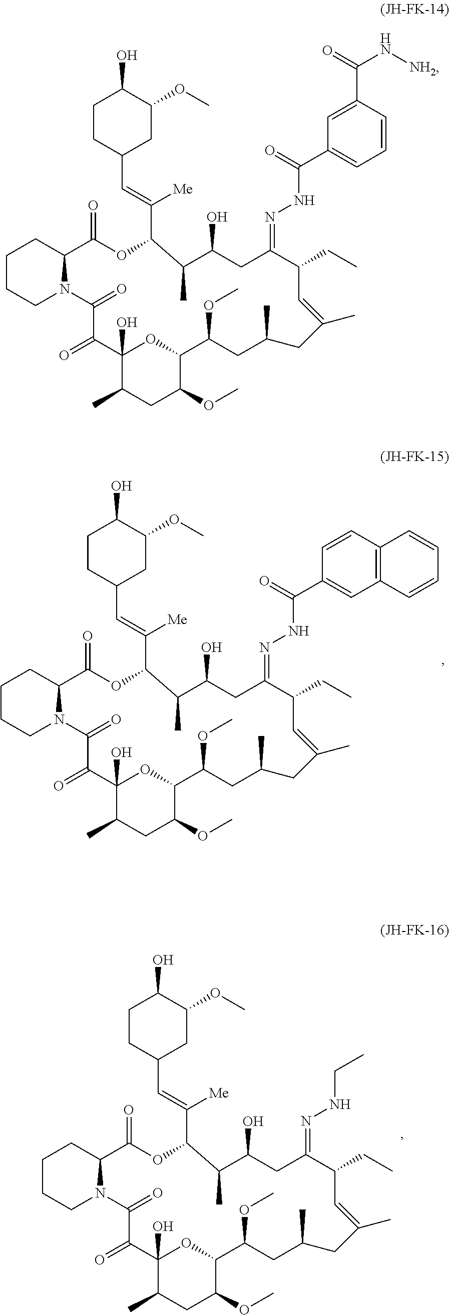

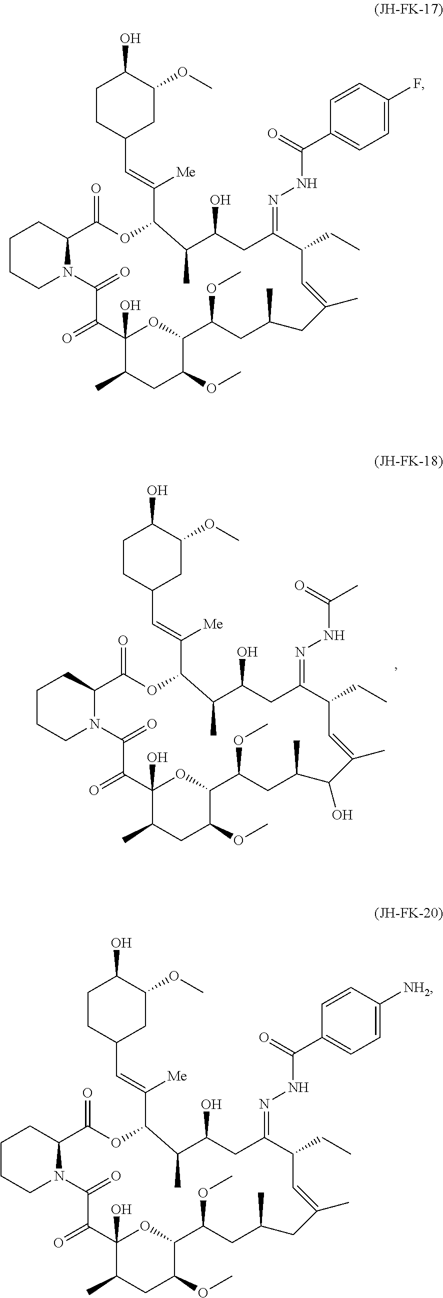

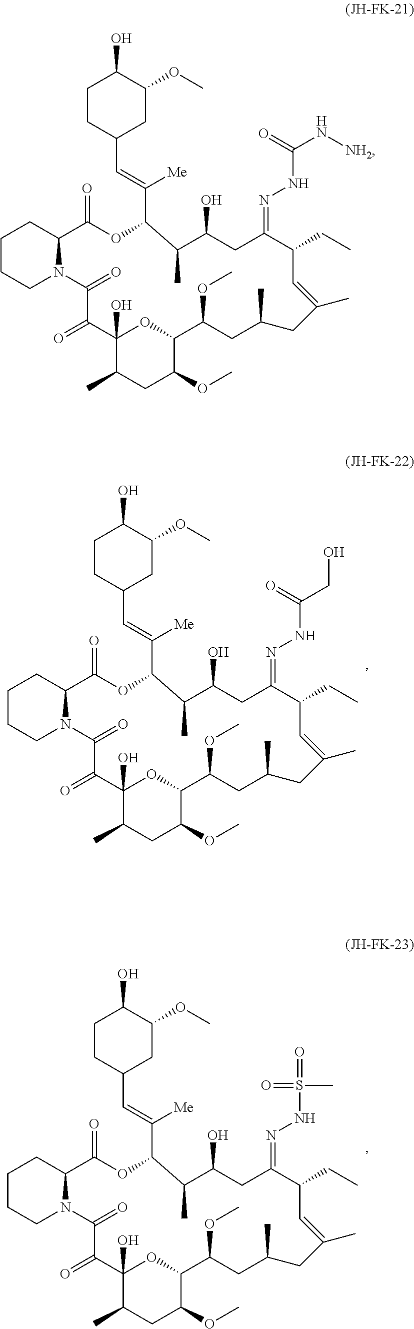

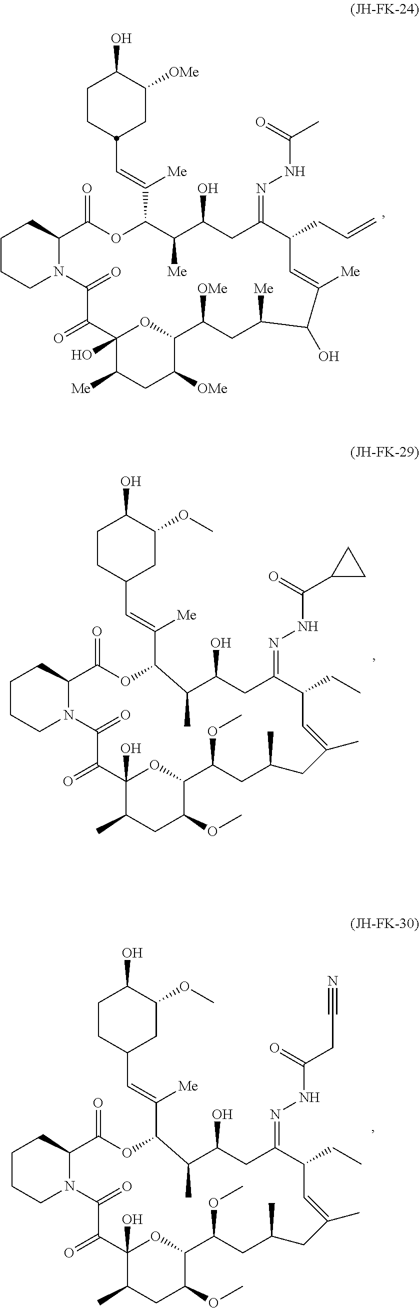

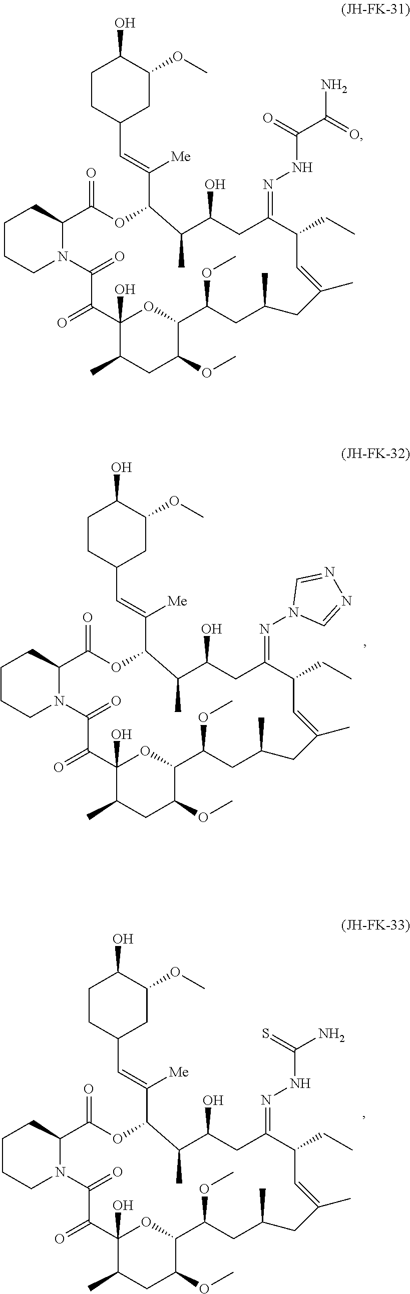

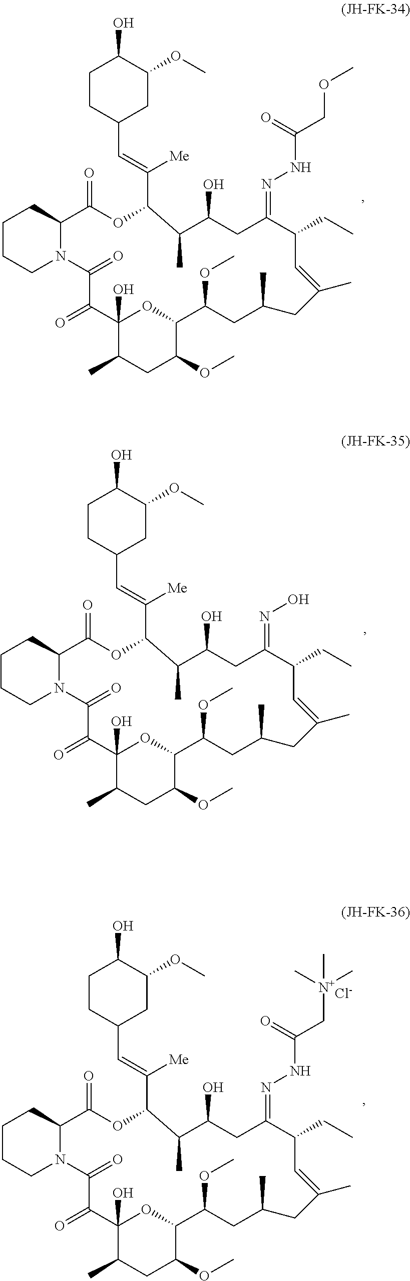

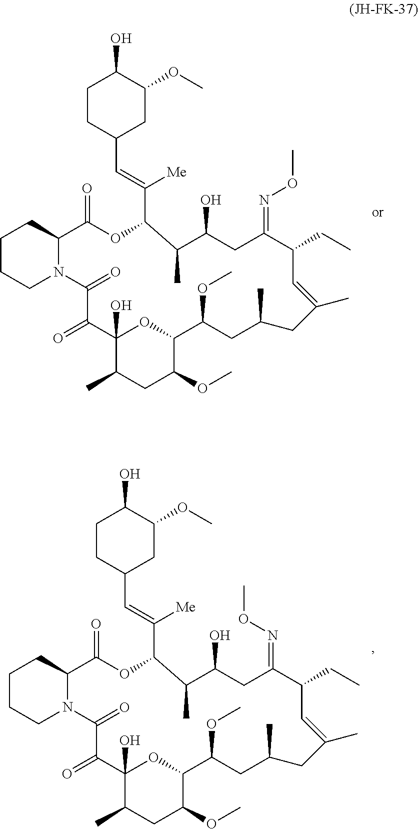 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/OC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N\OC)[C@H](C)C[C@@H]2OC]

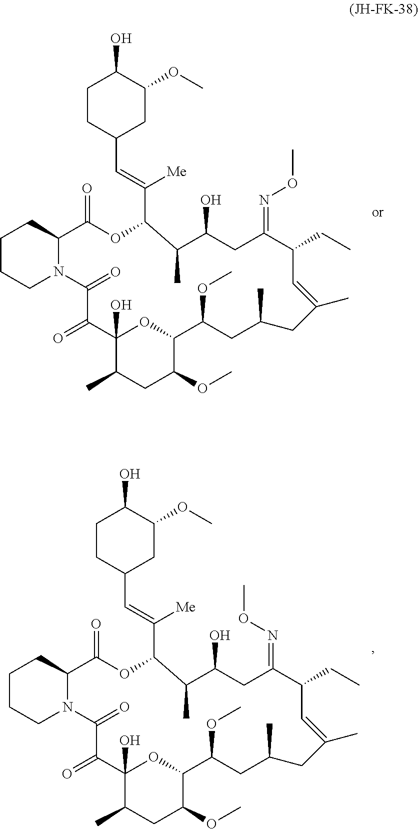 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N/OC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/C3CC[C@@H](O)[C@H](OC)C3)[C@H](C)[C@@H](O)C/C1=N\OC)[C@H](C)C[C@@H]2OC]

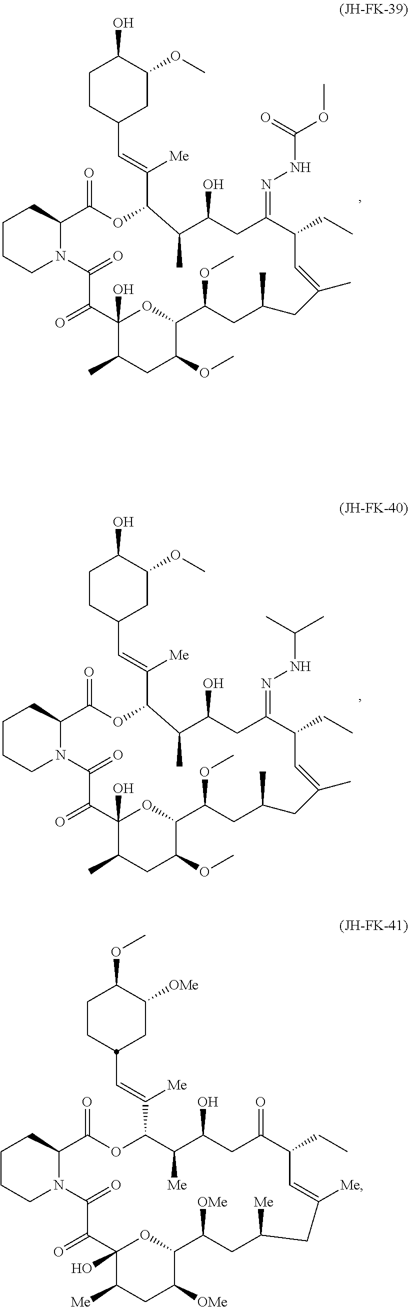

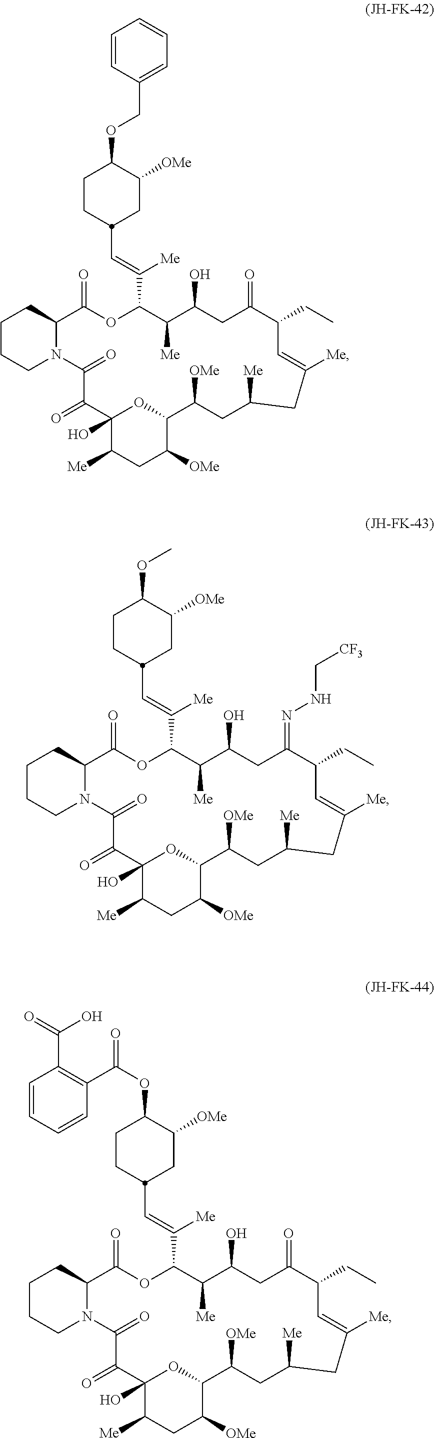

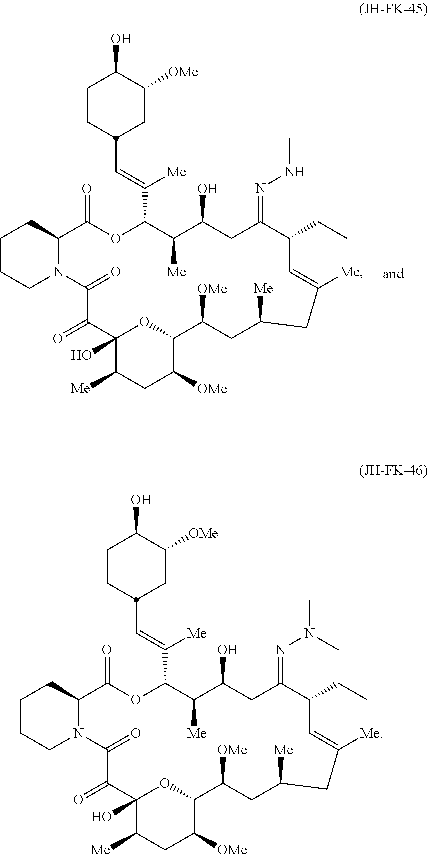 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/NC)[C@H](C)C[C@@H]2OC ; CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)C/C1=N/N(C)C)[C@H](C)C[C@@H]2OC]

8. A pharmaceutical composition comprising an effective amount of a compound of any one of claims 1-7, or a pharmaceutically acceptable salt thereof, and a pharmaceutically acceptable carrier.

9. The pharmaceutical composition of claim 8, wherein the composition further comprises at least one additional therapeutic agent.

10. The pharmaceutical composition of claim 9, wherein the at least one additional therapeutic agent comprises any type or kind of therapeutic agent capable of inhibiting fungal activity.

11. The pharmaceutical composition of claim 9, wherein the at least one additional therapeutic agent is selected from the following: a polyene, imidazole, triazole, thiazole, allylamine, echinocandin, among others. Examples include Amphotericin B, Candicidin, Filipin, Hamycin, Natamycin, Nystatin, Rimocidin, Bifonazole, Butoconazole, Clotrimazole, econazole, fenticonazole, isoconazole, kentoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, polygodial, tolnaftate, undecylenic acid, and crystal violet.

12. A method of treating or preventing a fungal infection in a mammal comprising administering to the mammal in need thereof a therapeutically effective amount of one or more of the compounds or pharmaceutical compositions recited in claims 1-11.

13. The method of claim 12, wherein the mammal is a human being.

14. The method of claim 12, wherein the mammal is a human being suffering from or at risk of suffering from a fungal infection.

15. The method of claim 12, wherein the fungal infection is an Aspergillus spp., Fusarium spp., Malassezia spp., Candida spp., or Cryptococcus spp. infection.

16. The method of claim 12, wherein the fungal infection is an Aspergillus spp. infection.

17. The method of claim 16, wherein the Aspergillus spp. infection is selected from an Aspergillus fumigatus infection, an Aspergillus favus infection, an Aspergillus niger infection, an Aspergillus terreus infection.

18. The method of claim 12, wherein the fungal infection is a Fusarium spp. infection.

19. The method of claim 18, wherein the Fusarium spp. infection is selected from a Fusarium solani infection, a Fusarium moniliforme infection, and a Fusarium proliferatum infection.

20. The method of claim 12, wherein the fungal infection is an Malassezia spp. infection.

21. The method of claim 20, wherein the Malassezia spp. infection is a Malassezia pachydermatis infection.

22. The method of claim 12, wherein the fungal infection is a Candida spp. infection.

23. The method of claim 22, wherein the Candida spp. infection is selected from a Candida albicans infection, a Candida glabrata infection, a Candida tropicalis infection, a Candida krusei infection, and a Candida auris infection.

24. The method of claim 12, wherein the fungal infection is a Cryptococcus spp. infection.

25. The method of claim 24, wherein the Cryptococcus spp. infection is a Cryptococcus neoformans infection.

26. The method of claim 12, wherein the fungal infection is a Chrysosporium parvum, Metarhizium anisopliae, Phaeoisaria clematidis, or Sarcopodium oculorum infection.

27. The method of claim 12, wherein the fungal infection is a Mucorales infection.

28. The method of claim 27, wherein the Mucorales infection is a Mucor spp., Rhizopus spp., Lichtheimia spp., or Rhizomucor spp. infection.

29. The method of claim 28, wherein the Mucor spp. infection is a M. circinelloides infection; wherein the Rhizopus spp. infection is a Rhizopus delemar infection or a Rhizopus oryzae infection; wherein the Lichtheimia spp. infection is a Lichtheimia corymbifera infection.

30. The method of claim 12, further comprising administering to the mammal in need thereof at least one additional therapeutic agent capable of inhibiting fungal activity.

31. The method of claim 30, wherein the at least one additional therapeutic agent is selected from the following: a polyene, imidazole, triazole, thiazole, allylamine, echinocandin, among others. Examples include Amphotericin B, Candicidin, Filipin, Hamycin, Natamycin, Nystatin, Rimocidin, Bifonazole, Butoconazole, Clotrimazole, econazole, fenticonazole, isoconazole, kentoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, polygodial, tolnaftate, undecylenic acid, and crystal violet.

32. A method of killing or inhibiting the growth of a fungus comprising contacting the fungus with a therapeutically effective amount of one or more of the compounds or pharmaceutical compositions recited in claims 1-11.

33. The method of claim 32, wherein the fungus is selected from Aspergillus spp., Fusarium spp., Malassezia spp., Candida spp., or Cryptococcus spp.

34. The method of claim 32, wherein the fungus is selected from Aspergillus fumigatus, Aspergillus flavus, Aspergillus niger, Aspergillus terreus, Fusarium solani, Fusarium moniliforme, Fusarium proliferatum, Malassezia pachydermatis, Candida albicans, Candida glabrata infection, Candida tropicalis, Candida krusei, Candida auris, Cryptococcus neoformans, Chrysosporium parvum, Metarhizium anisopliae, Phaeoisaria clematidis, Sarcopodium oculorum, M. circinelloides, Rhizopus delemar, Rhizopus orvzae, and Lichtheimia corymbifera.

35. Use of a compound of any one of claims 1-7, or a pharmaceutically acceptable salt thereof in the manufacture of a medicament for the treatment or prevention of a fungal infection.

36. The use of claim 35, wherein the fungal infection is related to one or more of: Aspergillus spp., Fusarium spp., Malassezia spp., Candida spp., and Cryptococcus spp.

37. The use of claim 35, wherein the fungal infection is related to one or more of: Aspergillus fumigatus, Aspergillus flavus, Aspergillus niger, Aspergillus terreus, Fusarium solani, Fusarium moniliforme, Fusarium proliferatum, Malassezia pachydermatis, Candida albicans, Candida glabrata infection, Candida tropicalis, Candida krusei, Candida auris, Cryptococcus neoformans, Chrysosporium parvum, Metarhizium anisopliae, Phaeoisaria clematidis, Sarcopodium oculorum, M. circinelloides, Rhizopus delemar, Rhizopus oryzae, and Lichtheimia corymbifera.
